(19) United States
(12) Patent Application Publication
(10) Pub. No.: US 2025/0251403 A1
(43) Pub. Date: Aug. 7, 2025

(54) COMPOSITIONS AND METHODS FOR GENERATING AND CHARACTERIZING RECOMBINANT ANTIGEN BINDING MOLECULES

(71) Applicant: 10X GENOMICS, INC., Pleasanton, CA (US)

(72) Inventors: Wyatt James McDonnell, Pleasanton, CA (US); Michael John Terry Stubbington, Bristol (GB); Christina Galonska, Stockholm (SE); Malte Kühnemund, Stockholm (SE); Ariel Royall, Pleasanton, CA (US); Camilla Engblom, Pleasanton, CA (US); Kim A. Thrane, Stockholm (SE); Jeffrey Eron Mold, Stockholm (SE); Jonas Frisen, Stockholm (SE); Joakim Lundeberg, Stockholm (SE); Qirong Lin, Stockholm (SE); Zachary W. Bent, Pleasanton, CA (US); Marlon Stoeckius, Stockholm (SE); Caroline Julie Gallant, Stockholm (SE); Katherine Pfeiffer, Pleasanton, CA (US)

(21) Appl. No.: 18/856,500

(22) PCT Filed: Apr. 11, 2023
(86) PCT No.: PCT/US2023/065638

§ 371 Date: Oct. 11, 2024

Related U.S. Application Data
(60) Provisional application No. 63/330,302, filed on Apr. 12, 2022.

(51) Int. Cl. G01N 33/68; C12N 15/10

(52) U.S. Cl. CPC G01N 33/6845; C12N 15/1065

(57) ABSTRACT

The present disclosure relates generally to the field of immunology, and particularly relates to compositions, methods, and systems for the analysis and generation of antigen-binding molecules produced by immune cells obtained from tissue samples (e.g., antibodies produced by B cells in tumor tissue samples or TCRs produced by T cells in tumor tissue samples) using spatial methodologies, and for the production and characterization of recombinant antigen-binding molecules (e.g., antibodies, TCRs) with desired properties.

### CROSS-REFERENCE TO RELATED APPLICATION

The present application claims priority to U.S. Provisional Patent Application Ser. No. 63/330,302, filed on Apr. 12, 2022. The content of the above-referenced application is herein expressly incorporated by reference in its entirety, including any drawings.

### FIELD

The present disclosure relates generally to the field of immunology, and particularly relates to compositions, methods, and systems for the analysis and generation of antigen-binding molecules produced by immune cells in a tissue sample (e.g., antibodies produced by B cells in tumor tissue samples or TCRs produced by T cells in tumor tissue samples) using spatial profiling methodologies, and for the production and characterization of recombinant antigen-binding molecules (e.g., antibodies, TCRs) with desired properties.

### BACKGROUND

The identification and evaluation of therapeutic proteins, especially antigen-binding molecules, e.g., therapeutic antibodies and TCRs is a core strategy for a number of pharmaceutical and biotechnology companies. For example, antibody-based therapy has become established over the past several years and is currently one of the most successful and important strategies for treating patients with hematological malignancies and solid tumors. In particular, the use of monoclonal antibodies (mAbs) for cancer therapy has achieved considerable success in recent years in the field of pharmaceutical biotechnology. Several monoclonal antibodies (mAbs) have been identified for use as therapeutic compounds in the treatment of various types of health condition and diseases. There are, for example, more than forty-five mAbs marketed in various fields such as oncology, immunology, ophthalmology and cardiology. In particular, monoclonal antibodies have provided important medical results in the treatment of several major diseases including autoimmune, cardiovascular and infectious diseases, cancer and inflammation, and even in clinical trials.

A number of processes and systems are currently available for the isolation and characterization of antigen-binding molecules (e.g., monoclonal antibodies), including hybridoma capture, phage display of human antibody libraries, yeast display of antibody libraries, and direct capture. Similarly, several approaches and techniques have been developed for the isolation of circulating tumor cells (CTCs) using antibody capture, microfluidics, and combinations thereof. However, it has been reported that rapid isolation of cancer-specific antibodies, patient-specific antibodies, potentially inter-patient cancer-specific antibodies, and direct screening of these isolated antibodies for tumor-specificity poses multiple challenges. Limitations of current approaches include, e.g., (i) a lack of heavy-light chain pairing (bulk approaches), (ii) inability to efficiently amplify B cell receptor sequences due to poor RNA quality or sample preparation conditions, (iii) low-throughput due to inability to combine and analyze samples from multiple individuals, and (iv) generation of antibodies that are not fully humanized (e.g., (e.g. “humanized” VDJ mice which still require additional humanization), unlike those antibodies found natively in tumors.

Therefore, there is a need for alternative approaches that couple high-throughput phenotypic screening with high-throughput sequencing of antigen-binding molecules, such as B cells and T cells, from tissue samples, in a flexible format that enables direct screening for functional activities.

### SUMMARY

In some embodiments, the present disclosure provides a method for identifying a tumor-specific antigen-binding molecule (ABM), the method comprising:
a) providing a tissue sample comprising one or more cells expressing an ABM; b) attaching an analyte of an ABM-expressing cell of the tissue sample to a capture domain of a first capture probe of a substrate comprising an array of capture probes attached thereto, the first capture probe comprising (i) a spatial barcode sequence and (ii) the capture domain, the capture domain comprising a capture sequence, wherein the analyte of the ABM-expressing cell comprises a sequence or portion of a sequence encoding the ABM expressed by the ABM-expressing cell or a reverse complement thereof, c) using the analyte of the ABM-expressing cell and the first capture probe attached thereto to generate a spatially barcoded polynucleotide comprising (i) a sequence of the analyte of the ABM-expressing cell or reverse complement thereof and (ii) the spatial barcode sequence or reverse complement thereof, and determining all or a part of the nucleic acid sequences of (i) and (ii); d) using the determined nucleic acid sequences of the analyte from (c) to produce a recombinant ABM; e) coupling the recombinant ABM to a reporter oligonucleotide comprising a reporter barcode sequence to generate a barcoded recombinant ABM; and f) contacting the barcoded recombinant ABM with a tumor tissue sample, and identifying the recombinant ABM as an ABM having specificity for the tumor tissue sample if the barcoded recombinant antibody is capable of binding to an antigen associated with the tumor tissue sample.

In one embodiment, the ABM expressed by the ABM-expressing cell is an immune cell receptor. In one embodiment, the immune cell receptor is a BCR or a TCR.

In one embodiment, the ABM expressed by the ABM-expressing cell is a secreted antibody.

In one embodiment, the capture sequence of the capture domain is a homopolymeric sequence.

In one embodiment, the capture sequence of the capture domain is a defined non-homopolymeric sequence. In one embodiment, the defined non-homopolymeric sequence is a sequence that binds to the analyte. In one embodiment, the defined non-homopolymeric sequence specifically binds to a nucleic acid sequence encoding a region of the ABM. In one embodiment, the ABM is selected from: a TCR alpha chain, a TCR beta chain, a TCR gamma chain, a TCR delta chain, an immunoglobulin kappa light chain, an immunoglobulin lambda light chain, an immunoglobulin heavy chain, or a combination thereof. In one embodiment, the region of the ABM is a constant region of the ABM or a variable region of the ABM.

In one embodiment, the homopolymeric sequence is a poly(T) sequence.

In some embodiments, the method further comprises sequencing the spatially barcoded polynucleotide if the recombinant ABM is identified as having specificity for the second tumor tissue sample.

In one embodiment, the tissue sample is in contact with the substrate comprising the array of capture probes during (b). In one embodiment, a portion of the tissue sample comprising the ABM-expressing cell is in contact with the first capture probe of the array of capture probes.

In one embodiment, the method comprises, following (a): releasing the first analyte from the ABM-expressing cell of the tissue sample; and migrating the first analyte to the substrate comprising the array of capture probes attached thereto.

In one embodiment, the substrate comprising the array of capture probes attached thereto is a second substrate, wherein the tissue sample is mounted on a first substrate during (a), and wherein the method comprises, following (a): mounting the first substrate on a first member of a support device, the first member configured to retain the first substrate; mounting the second substrate on a second member of the support device, the second member configured to retain the second substrate, applying a reagent medium to the first substrate and/or the second substrate, the reagent medium comprising a permeabilization agent, operating an alignment mechanism of the support device to move the first member and/or the second member such that a portion of the tissue sample comprising the ABM-expressing cell is aligned with a portion of the array of capture probes and within a threshold distance of the array of capture probes, and such that the portion of the tissue sample and the capture probe contact the reagent medium, wherein the permeabilization agent releases the first analyte from the ABM-expressing cell.

In one embodiment, method comprises, following (a): mounting the first substrate on a first member of a support device, the first member configured to retain the first substrate; mounting the second substrate on a second member of the support device, the second member configured to retain the second substrate; applying a reagent medium to the first substrate and/or the second substrate, the reagent medium comprising a permeabilization agent; operating an alignment mechanism of the support device to move the first member and/or the second member such that a portion of the tissue sample comprising the ABM-expressing cell is aligned with a portion of the array of capture probes and within a threshold distance of the array of capture probes, and such that the portion of the tissue sample and the capture probe contact the reagent medium, wherein the permeabilization agent releases the analyte from the ABM-expressing cell.

In one embodiment, the method further comprises aligning the first substrate with the second substrate, such that at least a portion of the tissue sample is aligned with at least a portion of the second substrate.

In one embodiment, the migrating comprises passive migration.

In one embodiment, the migrating comprises active migration, and optionally, wherein the active migration comprises electrophoresis.

In one embodiment, the analyte is RNA or DNA, optionally wherein the RNA is mRNA, or the DNA is cDNA or genomic DNA.

In one embodiment, the analyte is a nucleic acid analyte.

In one embodiment, the analyte comprises a sequence encoding a variable region and/or a constant region of the ABM. In one embodiment, the variable region comprises a VJ or a VDJ sequence. In some embodiments, the method comprises amplifying the spatiallybarcoded polynucleotide to generate a spatially barcoded nucleic acid library member comprising the sequence encoding the variable region and the constant region of the ABM. In some embodiments, the method further comprises removing all or a portion of the sequence encoding the constant region of the ABM from the spatially barcoded nucleic acid library member or amplicon thereof.

The present disclosure also provides a method for identifying a tumor-specific antibody, the method comprising: a) providing a first tumor tissue sample comprising one or more cells expressing one or more antibodies; b) determining all or a part of the nucleic acid sequences encoding the one or more antibodies; c) using the determined nucleic acid sequences to produce a recombinant antibody; d) coupling the recombinant antibody to a reporter oligonucleotide comprising a reporter barcode sequence to generate a barcoded recombinant antibody; and e) contacting the barcoded recombinant antibody with a second tumor tissue sample, and identifying the recombinant antibody as an antibody having specificity for the second tumor tissue sample if the barcoded recombinant antibody is capable of binding to an antigen associated with the second tumor tissue sample.

In one embodiment, the first and/or the second tumor tissue sample is derived from a solid tumor, a soft tissue tumor, a metastatic lesion, a non-solid tumor, a circulating tumor cell (CTC) population, a tumor cell line, or a patient derived xenograft (PDX).

In one embodiment, the first and the second tumor tissue samples are derived from the same subject.

In one embodiment, the first and the second tumor tissue samples are derived from the same tumor.

In some embodiments, step (e) further comprises contacting the barcoded recombinant antibody with a control tissue sample. In one embodiment, the control sample is (i) a non-tumor tissue sample or (ii) a tissue sample that the barcoded recombinant antibody is not expected to bind.

In some embodiments, the method further comprises contacting the second tumor tissue sample with a composition comprising one or more of the following: i) one or more barcoded immune-cell marker antibodies and/or barcoded tumor-cell marker antibodies; ii) one or more barcoded therapeutic antibodies; and iii) the barcoded recombinant antibody identified as having specificity for the second tumor sample. In one embodiment, the one or more therapeutic antibodies is selected from the group consisting of abciximab, abciximab, adalimumab, aducanumab, alacizumab, alemtuzumab, alirocumab, alirocumab, ascrinvacumab, atezolizumab, atinumab, bapineuzumab, basiliximab, basiliximab, belimumab, bevacizumab, blinatumomab, blosozumab, bococizumab, brentuximab, canakinumab, caplacizumab, capromab, certolizumab, cetuximab, crenezumab, daclizumab, daratumumab, demcizumab, denosumab, denosumab, dinutuximab, ecukinumab, eculizumab, eculizumab, efalizumab, elotuzumab, enoticumab, etaracizumab, evinacumab, evolocumab, evolocumab, fasinumab, fulranumab, gantenerumab, golimumab, ibritumomab, icrucumab, idarucizumab, idarucizumab, inciacumab, infliximab, ipilimumab, mepolizumab, natalizumab, necitumumab, nesvacumab, nivolumab, obinutuzumab, ofatumumab, omalizumab, opicinumab, orticumab, ozanezumab, palivizumab, palivizumab, panitumumab, pembrolizumab, pertuzumab, ponezumab, ralpancizumab, ramucirumab, ramucirumab, ranibizumab, raxibacumab, refanezumab, rinucumab, rituximab, romosozumab, siltuximab, solanezumab, stamulumab, tadocizumab, tanezumab, tocilizumab, trastuzumab, ustekinumab, vedolizumab, and vesencumab. In one embodiment, the one or more immune-cell marker antibodies is selected from the group consisting of antibodies having specificity for one or more of B cells, T cells, monocytes, macrophages, granulocytes (basophil, eosinophil, neutrophil), dendritic cells, NK cells, and NKT cells. In one embodiment, the one or more tumor-cell marker antibodies is selected from the group consisting of antibodies having specificity for ALK, alpha-fetoprotein (AFP), beta-2-microglobulin (B2M), beta-human chorionic gonadotropin (Beta-hCG), bladder tumor antigen (BTA), BRCA1, BRCA2, BCR-ABL fusion gene (Philadelphia chromosome), BRAF V600 mutations, C-kit/CD117, CA15-3/CA27.29, CA-125, CA 27.29, carcinoembryonic antigen (CEA), CD20, CD22, CD25, CD30, CD31, CD33, CD44, CD133, CD176, CD276, estrogen receptor (ER), E-cadherin, ESPR, EGFR, EPCAM, GD2, progesterone receptor (PR), fibrin/fibrinogen, HE4 gene variants, HER2 gene variants, JAK2 gene variants, KRAS gene variants, nuclear matrix protein 22, PCA3, PML/RARα fusion gene, programmed death-ligand 1 (PD-L1 or CD274), prostate-specific antigen (PSA), TEM7, TEM8, and VEGF receptor family members.

In some embodiments, identifying the produced antibody as an antibody having specificity for the second tumor tissue sample further comprises quantifying levels of gene expression and protein marker expression in the second tumor tissue sample. In one embodiment, the method further comprises using the quantified levels for identification of biomarkers specific for the second tumor tissue sample and/or a subject from whom the second tumor sample is obtained.

In some embodiments, the method further comprises quantifying binding affinity of one or more therapeutic antibodies to the second tumor tissue sample. In one embodiment, the method further comprises using the quantified binding affinity as an indicator of efficacy of treating a tumor with the one or more therapeutic antibodies. In some embodiments, the method further comprises using the quantified binding affinity to monitor antigen escape of a tumor from the one or more therapeutic antibodies over time.

In one embodiment, identifying the produced antibody as an antibody having specificity for the second tumor tissue sample further comprises comparing the determined nucleic acid sequences encoding the antibody to a genomic DNA sequence from the second tumor tissue sample to confirm antigen specificity of the antibody. In one embodiment, the genomic DNA sequence is obtained from a single cell in the second tumor sample. In one embodiment, the genomic DNA sequence is obtained from a plurality of cells in the second tumor sample. In some embodiments, the genomic DNA sequence is obtained by whole-genome sequencing.

In some embodiments, identifying the produced antibody as an antibody having specificity for the second tumor tissue sample further comprises comparing the determined nucleic acid sequences encoding the barcoded antibody to a sequence of a ribonucleic acid (RNA) molecule from the second tumor sample to confirm antigen specificity of the antibody. In one embodiment, the RNA molecule is obtained from a single cell in the second tumor sample. In one embodiment, the RNA molecule is obtained from a plurality of cells in the second tumor sample. In some embodiments, the method further comprises obtaining the sequence of the RNA molecule.

In some embodiments, the method further comprises determining a nucleic acid sequence of a messenger RNA (mRNA) from a single B cell and/or from a single tumor cell in the tissue sample. In one embodiment, the determining comprises binding one or more nucleic acid barcode molecules to the mRNA and optionally generating a complementary DNA (cDNA) via reverse transcription. In one embodiment, the one or more nucleic acid barcode molecules independently comprise one or more barcode sequences. In one embodiment, the one or more barcode sequences is selected from the group consisting of a sample barcode, a tissue barcode, a cell barcode, a spatial barcode, and a unique molecular identifier (UMI). In some embodiments, the one or more nucleic acid barcode molecules are coupled to a microcapsule. In one embodiment, the microcapsule comprises a bead. In some embodiments, the determining includes whole transcriptome sequencing. In some embodiments, the determining comprises next-generation sequencing (NGS).

In one embodiment, the method further comprises generating a chimeric antigen receptor (CAR) using the nucleic acid sequence of the recombinant antibody.

In some embodiments, the method further comprises administering a composition comprising the recombinant antibody or a fragment thereof to a subject in need thereof.

In some embodiments, the method further comprises administering a composition comprising an immune cell expressing the recombinant antibody or a fragment thereof to a subject in need thereof.

In some embodiments, the method further comprises comparing the determined nucleic acid sequence of the recombinant antibody to sequences of known antibodies in order to identify the antibody as a tumor-specific antibody.

In some embodiments, the method further comprises using a filter that takes into account clonal expansions to identify the recombinant antibody as a tumor-specific antibody.

In some embodiments, the method further comprises using a filter that takes into account gene expression profiles of B cells to identify the recombinant antibody as a tumor-specific antibody.

In some embodiments, the method further comprises using a filter that takes into account somatic hypermutation and isotype usage to identify the recombinant antibody as a tumor-specific antibody.

The present disclosure also provides a method for generating a recombinant antibody, the method comprising: a) providing a first tumor tissue sample; b) determining all or a part of the nucleic acid sequences encoding one or more antibodies produced by one or more cells of the first tumor tissue sample; and c) using the determined nucleic acid sequences to produce a recombinant antibody.

In one embodiment, the method further comprises coupling a reporter oligonucleotide comprising a reporter barcode sequence to the recombinant antibody to generate a barcoded recombinant antibody. In one embodiment, the reporter barcode sequence of the reporter oligonucleotide comprises one or more unique identifiers for the recombinant antibody. In some embodiments, the method further comprises determining all or a part of the nucleic acid sequence of the one or more oligonucleotide identifiers to identify the barcoded recombinant antibody. In some embodiments, the reporter oligonucleotide comprise an adapter region that allows for downstream analysis of the recombinant antibody. In one embodiment, the adapter region comprises a primer binding site and/or a cleavage site.

In some embodiments, the method further comprises contacting the barcoded recombinant antibody to a tumor cell obtained from a second tumor tissue sample. In one embodiment, the method further comprises identifying the recombinant antibody as an antibody having specificity for the second tumor tissue sample if the barcoded recombinant antibody is capable of binding to an antigen associated with the second tumor tissue sample. In some embodiments, the first and/or the second tumor tissue sample is derived from a solid tumor, a soft tissue tumor, a metastatic lesion, a non-solid tumor, a circulating tumor cell (CTC) population, a tumor cell line, or a patient derived xenograft (PDX). In some embodiments, the first and the second tumor tissue samples are derived from the same subject. In some embodiments, the first and the second tumor tissue samples are derived from the same tumor. In some embodiments, the method comprises contacting the second tumor tissue sample with a composition comprising one or more of the following: i) one or more barcoded immune-cell marker antibodies and/or barcoded tumor-cell marker antibodies; ii) one or more barcoded therapeutic antibodies; and iii) the barcoded recombinant antibody identified as having specificity for the second tumor tissue sample. In one embodiment, the one or more therapeutic antibodies is selected from the group consisting of abciximab, abciximab, adalimumab, aducanumab, alacizumab, alemtuzumab, alirocumab, alirocumab, ascrinvacumab, atezolizumab, atinumab, bapineuzumab, basiliximab, basiliximab, belimumab, bevacizumab, blinatumomab, blosozumab, bococizumab, brentuximab, canakinumab, caplacizumab, capromab, certolizumab, cetuximab, crenezumab, daclizumab, daratumumab, demcizumab, denosumab, denosumab, dinutuximab, ecukinumab, eculizumab, eculizumab, efalizumab, elotuzumab, enoticumab, etaracizumab, evinacumab, evolocumab, evolocumab, fasinumab, fulranumab, gantenerumab, golimumab, ibritumomab, icrucumab, idarucizumab, idarucizumab, inciacumab, infliximab, ipilimumab, mepolizumab, natalizumab, necitumumab, nesvacumab, nivolumab, obinutuzumab, ofatumumab, omalizumab, opicinumab, orticumab, ozanezumab, palivizumab, palivizumab, panitumumab, pembrolizumab, pertuzumab, ponezumab, ralpancizumab, ramucirumab, ramucirumab, ranibizumab, raxibacumab, refanezumab, rinucumab, rituximab, romosozumab, siltuximab, solanezumab, stamulumab, tadocizumab, tanezumab, tocilizumab, trastuzumab, ustekinumab, vedolizumab, and vesencumab. In some embodiments, the one or more immune-cell marker antibodies is selected from the group consisting of antibodies having specificity for one or more of B cells, T cells, monocytes, macrophages, granulocytes (basophil, eosinophil, neutrophil), dendritic cells, NK cells, and NKT cells. In some embodiments, the one or more tumor-cell marker antibodies is selected from the group consisting of antibodies having specificity for ALK, alpha-fetoprotein (AFP), beta-2-microglobulin (B2M), beta-human chorionic gonadotropin (Beta-hCG), bladder tumor antigen (BTA), BRCA1, BRCA2, BCR-ABL fusion gene (Philadelphia chromosome), BRAF V600 mutations, C-kit/CD117, CA15-3/CA27.29, CA-125, CA 27.29, carcinoembryonic antigen (CEA), CD20, CD22, CD25, CD30, CD31, CD33, CD44, CD133, CD176, CD276, estrogen receptor (ER), E-cadherin, ESPR, EGFR, EPCAM, GD2, progesterone receptor (PR), fibrin/fibrinogen, HE4 gene variants, HER2 gene variants, JAK2 gene variants, KRAS gene variants, nuclear matrix protein 22, PCA3, PML/RARα fusion gene, programmed death-ligand 1 (PD-L1 or CD274), prostate-specific antigen (PSA), TEM7, TEM8, and VEGF receptor family members.

The present disclosure further provides a recombinant antibody or a functional fragment thereof generated or identified by a method according to the present disclosure.

The present disclosure further provides a recombinant nucleic acid comprising a nucleic acid sequence that encodes the recombinant antibody of the present disclosure or a functional fragment thereof. In one embodiment, the recombinant nucleic acid is further configured as an expression cassette in a vector. In one embodiment, the vector is a plasmid vector or a viral vector.

The present disclosure further provides a recombinant cell comprising a recombinant nucleic acid according to the present disclosure. In one embodiment, the recombinant cell is a prokaryotic cell or a eukaryotic cell.

The present disclosure further provides a composition comprising a pharmaceutically acceptable excipient and one or more of the following: a) a recombinant antibody of the present disclosure; b) a recombinant nucleic acid according to the present disclosure; or c) a recombinant cell according to the present disclosure.

The present disclosure further provides a composition comprising one or more of the following: a) one or more barcoded immune-cell marker antibodies and/or barcoded tumor-cell marker antibodies; b) one or more barcoded therapeutic antibodies; or c) a barcoded recombinant antibody of the present disclosure identified as having specificity for the second tumor sample.

The present disclosure further provides a kit comprising one or more of the following: a) a recombinant antibody or a functional fragment thereof of the present disclosure; b) a recombinant nucleic acid according to the present disclosure; or c) a recombinant cell according to the present disclosure; or instructions for use thereof.

The present disclosure further provides a method for characterizing antibody specificity or target specificity, the method comprising: a) providing a first tumor tissue sample; b) determining all or a part of the nucleic acid sequences encoding one or more antibodies produced by B cells in the tumor tissue sample; c) using the determined nucleic acid sequences to produce a recombinant antibody; d) coupling the recombinant antibody to a reporter oligonucleotide comprising a reporter barcode sequence to generate a barcoded recombinant antibody; e) contacting the barcoded recombinant antibody with a second tumor tissue sample, and identifying the recombinant antibody as an antibody having specificity for the tumor sample if the barcoded recombinant antibody is capable of binding to an antigen associated with the second tumor tissue sample; and f) analyzing RNA expression and protein marker expression for the first and/or second tumor tissue samples to determine the recombinant antibody specificity and target specificity.

The present disclosure further provides a method for enhanced identification of patient-specific or population-specific biomarkers, the method comprising: a) providing a tumor tissue sample comprising a plurality of B cells; b) determining all or a part of the nucleic acid sequences encoding one or more antibodies produced by B cells in the tumor tissue sample; c) using the determined nucleic acid sequences to produce a recombinant antibody; d) coupling the recombinant antibody to a reporter oligonucleotide comprising a reporter barcode sequence to generate a barcoded recombinant antibody; e) contacting the barcoded recombinant antibody with a second tumor tissue sample, and identifying the recombinant antibody as an antibody having specificity for the tumor sample if the barcoded recombinant antibody is capable of binding to an antigen associated with the second tumor tissue sample; and f) analyzing RNA expression and protein marker expression for the second tumor tissue sample to identify one or more biomarkers specific for the second tumor tissue sample or for a population of tumor tissue samples.

The present disclosure further provides a method for monitoring antigen escape in an individual who has been treated with an antibody-based therapy, the method comprising: a) providing a tumor tissue sample comprising a plurality of B cells; b) determining all or a part of the nucleic acid sequences encoding one or more antibodies produced by the B cells in the tumor tissue sample; c) using the determined nucleic acid sequences to produce a recombinant antibody; d) coupling the recombinant antibody to a reporter oligonucleotide comprising a reporter barcode sequence to generate a barcoded recombinant antibody; e) contacting the barcoded recombinant antibody with a second tumor tissue sample, and identifying the recombinant antibody as an antibody having specificity for the tumor tissue sample if the barcoded recombinant antibody is capable of binding to an antigen associated with the second tumor tissue sample; f) quantifying binding affinity of a barcoded therapeutic antibody to the second tumor tissue sample, wherein the quantified binding affinity is indicative of the therapeutic antibody's efficacy in treating the tumor; and g) optionally using the quantified binding affinity to monitor antigen escape from the therapeutic antibody over time.

The present disclosure further provides a method for characterizing a potential antigen for an antibody or fragment thereof, the method comprising: a) providing a tumor tissue sample comprising a plurality of B cells; b) determining all or a part of the nucleic acid sequences encoding one or more antibodies produced by the B cells in the tumor tissue sample; c) using the determined nucleic acid sequences to produce a recombinant antibody; d) coupling the recombinant antibody to a reporter oligonucleotide comprising a reporter barcode sequence to generate a barcoded recombinant antibody; e) contacting the barcoded recombinant antibody with a second tumor tissue sample, and identifying the recombinant antibody as an antibody having specificity for the tumor tissue sample if the barcoded recombinant antibody is capable of binding to an antigen associated with the second tumor tissue sample; and f) quantifying binding affinity of the one or more antibodies to the second tumor tissue sample, and using the quantified binding affinity to determine if the one or more antibodies compete with one another for binding to the second tumor tissue sample; and g) optionally co-associating the quantified binding affinity with RNA expression analysis to identify potential antigen.

The present disclosure further provides a system for antibody discovery/management, comprising: a logic processor; and a stored program code that is executable by the logic processor, wherein the program code configures the logic processor to receive information input pertaining to an antibody profile comprising a preselected set of data input in order to assign a relative performance score to the antibody's tumor specificity based at least in part on the antibody profile, whereby determining the likelihood of the antibody to exhibit one or more tumor specificity attributes as indicated by the assigned relative performance score.

In one embodiment, the system further comprises a data compiler communicatively coupled to the logic processor; and a report engine communicatively coupled to the logic processor, wherein reports produced by the report engine depend upon results from execution of the program code. In some embodiments, the data input includes one or more of the following: (a) antibody sequence data; (b) expression data of biomarkers in the tissue sample from which the antibody is derived; (c) transcriptomic data for the tissue sample from which the antibody is derived; (d) whole-exome data; (e) proteomic data; and (f) genomic DNA sequence data from whole-genome sequencing. In some embodiments, the system further comprises generating an antibody profile report that contains information relevant to the antibody identified as a tumor-specific antibody. In one embodiment, the antibody profile report is characterized as having an encoding selected from the group consisting of “.doc”; “.pdf”; “.xml”; “.html”; “.jpg”; “.aspx”; “.php”, and a combination of any thereof.

The present disclosure further provides a non-transitory computer readable medium containing machine executable instructions that when executed cause a processor to perform operations comprising: receiving an antibody profile comprising a preselected set of data input; assigning, based at least in part on the antibody profile, a relative performance score to the antibody's tumor specificity; and outputting an antibody profile report for the antibody based upon the assigned performance score.

The present disclosure further provides an antibody profile report generated by the system of the present disclosure. The present disclosure further provides a method for generating a recombinant antigen-binding molecule, the method comprising: a) providing a tumor tissue sample comprising a plurality of immune cells; b) determining all or a part of the nucleic acid sequences encoding one or more antigen-binding molecules produced by the immune cells in the tumor tissue sample; and c) using the determined nucleic acid sequences to produce a recombinant antigen-binding molecule. In one embodiment, the plurality of immune cells comprises a T cell and wherein the one or more antigen-binding molecules produced by the immune cells comprises a TCR. The present disclosure further provides a method for identifying a tumor-specific antibody, comprising: a) contacting a barcoded recombinant antibody with a tumor tissue sample; and b) identifying the barcoded recombinant antibody as a tumor-specific antibody if the barcoded recombinant antibody is capable of binding to an antigen associated with the tumor tissue sample, wherein the barcoded recombinant antibody comprises a recombinant antibody coupled to a reporter oligonucleotide comprising a reporter barcode sequence, wherein the recombinant antibody is identified and/or produced by (i) providing a tumor tissue sample comprising a plurality of B cells (ii) determining all or a part of the nucleic acid sequences encoding one or more antibodies produced by the B cell cells in the tumor tissue sample, and optionally (iii) using the determined nucleic acid sequences to recombinantly produce the recombinant antibody. In one embodiment, the barcoded recombinant antibody is produced by coupling the recombinant antibody to a reporter oligonucleotide comprising a reporter barcode sequence to generate a barcoded recombinant antibody.

In some embodiments, determining all or a part of the nucleic acid sequence encoding the one or more antibodies comprises: a) contacting the tumor tissue sample with a first primer comprising a nucleic acid sequence that hybridizes to a complementary sequence in the nucleic acid sequence encoding the one or more antibodies and a functional domain; (b) hybridizing the first primer to the nucleic acid sequence encoding the one or more antibodies and extending the first primer using the nucleic acid sequence encoding the one or more antibodies as a template to generate an extension product; (c) adding a polynucleotide sequence comprising at least three nucleotides to the 3′ end of the extension product; (d) hybridizing a second primer to the polynucleotide sequence comprising at least three nucleotides of the extension product of (c), wherein the second primer comprises a capture sequence; (e) extending the extension product using the second primer as a template, thereby incorporating a complement of the capture sequence into the extension product; (f) hybridizing the complement of the capture sequence of the extension product to a capture domain on an array, wherein the array comprises a plurality of capture probes, and wherein a capture probe of the plurality of capture probes comprises a spatial barcode and the capture domain; and (g) determining (i) the sequence of the spatial barcode, or a complement thereof, and (ii) all or a portion of the sequence of the nucleic acid sequence, or a complement thereof, and using the determined sequences of (i) and (ii) to determine all or a part of the nucleic acid sequence encoding the one or more antibodies.

In one embodiment, the first primer comprises a random sequence, optionally wherein the random sequence comprises a random hexamer or random decamer.

In one embodiment, the first primer comprises a homopolymer sequence, optionally wherein the homopolymer sequence comprises a poly(T) sequence.

In some embodiments, the first primer comprises a sequence substantially complementary to a sequence in the analyte or all or a part of the nucleic acid sequence encoding the one or more antibodies encoding a constant region of an immune cell receptor, optionally wherein the immune cell receptor comprises a B cell receptor or a T cell receptor.

In some embodiments, incorporating the polynucleotide sequence to the 3′ end of the extension product in step (c) comprises the use of a terminal deoxynucleotidyl transferase or of a reverse transcriptase.

In some embodiments, the second primer comprises RNA.

In some embodiments, the method further comprises removing the analyte or the nucleic acid sequence encoding the one or more antibodies, or any other nucleic acid hybridized to the extension product, before the complement of the capture sequence of the extension product hybridizes to the capture domain of the capture probe on the array, optionally wherein the removing comprises the use of an RNase, optionally wherein the RNase is RNaseH.

In some embodiments, the method further comprises a step of extending the 3′ end of the extension product of step (e) using the capture probe as a template, thereby generating an extended capture product, and/or extending the capture probe using the extension product of step (e) as a template.

In one embodiment, step (b) comprises generating one or more extension products using a plurality of primers, wherein a primer of the plurality of primers comprises a nucleic acid sequence that is substantially complementary to a sequence in the target nucleic acid and a functional domain, wherein the first primer is comprised in the plurality of primers; (a) hybridizing the plurality of primers to the the analyte or the nucleic acid sequence encoding the one or more antibodies and extending one or more primers from the plurality of primers using the target nucleic acid as a template to generate the one or more extension products; (b) attaching a polynucleotide sequence to the 3′ end of the one or more extension products; (c) hybridizing the second primer to the polynucleotide sequence of the one or more extension products of (b), wherein the second primer comprises a capture sequence; (d) extending the one or more extension products using the second primer as a template, thereby incorporating a complement of the capture sequence into the one or more extension products; (e) hybridizing the complement of the capture sequence of the one or more extension products to a capture domain on an array, wherein the array comprises a plurality of capture probes, and wherein the capture probe of the plurality of capture probes comprises a spatial barcode and the capture domain; and (f) determining (i) the sequence of the spatial barcode, or a complement thereof, and (ii) all or a portion of the sequence of the target nucleic acid, or a complement thereof, and using the determined sequences of (i) and (ii) to determine all or a part of the nucleic acid sequence of the analyte or the nucleic acid sequence encoding the one or more antibodies.

In some embodiments, determining all or a part of the nucleic acid sequence of the analyte or all or a part of the nucleic acid sequence encoding the one or more antibodies comprises: (a) contacting the tissue sample or tumor tissue sample with an array comprising a plurality of capture probes, wherein a capture probe of the plurality of capture probes comprises (i) a spatial barcode and (ii) a capture domain that hybridizes to a poly(A) sequence of the analyte or the nucleic acid sequence encoding the one or more antibodies; (b) hybridizing the capture domain to the analyte or nucleic acid sequence encoding the one or more antibodies; (c) extending the capture probe using the analyte or nucleic acid sequence encoding the one or more antibodies as a template to generate an extended capture probe comprising a sequence encoding a CDR3, or a complement thereof, of the ABM or one or more antibodies; (d) hybridizing one or more probes to the extended capture probe, or a complement thereof, in a portion encoding a constant region of the ABM or one or more antibodies, wherein the one or more probes comprises a binding moiety capable of binding a capture moiety; (e) enriching the extended capture probe or the complement thereof via an interaction between the binding moiety in the one or more probes and the capture moiety; and (f) determining (i) the sequence of the spatial barcode or a complement thereof, and (ii) all or a portion of the sequence of the analyte or nucleic acid sequence encoding the one or more antibodies, or a complement thereof, and using the determined sequences of (i) and (ii) to determine all or part of the nucleic acid sequence of the analyte or the nucleic acid sequence encoding the one or more antibodies.

In one embodiment, the one or more probes hybridizes to a nucleic acid sequence encoding a constant region of the ABM or one or more antibodies, or a complement thereof.

In some embodiments, the capture domain comprises a poly(T) sequence.

In some embodiments, the method further comprises generating the complement of the extended capture using the extended capture probe as a template, wherein the complement of the extended capture probe comprises (i) a sequence that is complementary to the spatial barcode, and (ii) a sequence that corresponds to all or a portion of the analyte or nucleic acid sequence encoding the one or more antibodies.

In some embodiments, the binding moiety comprises biotin and the capture moiety comprises streptavidin.

In some embodiments, the determining in step (f) comprises sequencing the extended capture probe or the complement thereof to determine (i) the sequence of the spatial barcode, or the complement thereof, and (ii) all or a portion of the sequence of the analyte or nucleic acid sequence encoding the one or more antibodies, optionally wherein the sequencing comprises long read sequencing.

In some embodiments, the capture probe further comprises an adaptor domain and the method further comprises after step (e), performing a polymerase chain reaction using i) a first primer complementary to the adaptor domain of the capture probe, and ii) a second primer complementary to a portion of the analyte or nucleic acid sequence encoding the one or more antibodies in a portion encoding a variable region of the immune cell receptor of the immune cell clonotype.

In one embodiments, the second primer is complementary to a nucleic acid sequence 5′ to the sequence encoding CDR3 of the ABM or the one or more antibodies.

In some embodiments, determining all or a part of the nucleic acid sequences of the analyte or all or a part of the nucleic acid sequence encoding the one or more antibodies comprises: (a) contacting the tissue sample or tumor tissue sample with an array comprising a feature, wherein the feature comprises an attached first and second probe, wherein: a 5′ end of the first probe is attached to the feature; the first probe comprises in a 5′ to a 3′ direction: a spatial barcode and a poly(T) capture domain, wherein the poly(T) capture domain binds specifically to the analyte or the nucleic acid sequence encoding the one or more antibodies; a 5′ end of the second probe is attached to the feature; a 3′ end of the second probe is reversibly blocked; and the second probe comprises a poly(GI) capture domain; (b) extending a 3′ end of the first probe to add a sequence that is complementary to a portion of the analyte or the nucleic acid sequence encoding the one or more antibodies; (c) ligating an adapter to the 5′ end of analyte or the nucleic acid sequence encoding the one or more antibodies specifically bound to the first probe; (d) adding a sequence complementary to the adapter to the 3′ end of the first probe; (e) adding non-templated cytosines to the 3′ end of the first probe to generate a poly(C) sequence, wherein the poly(C) sequence specifically binds to the poly(GI) capture domain of the second probe; (f) unblocking the 3′ end of the second probe and extending the 3′ end of the second probe to add a sequence comprising a sequence in the analyte or the nucleic acid sequence encoding the one or more antibodies and a sequence that is complementary to the spatial barcode; (g) cleaving a region of the second probe at a cleavage site that is 5′ to the poly(GI) capture domain, thereby releasing the second probe from the feature; and (h) determining (i) all or a part of the sequence of the spatial barcode, or a complement thereof, and (ii) all or a part of the sequence of the analyte or the nucleic acid sequence encoding the one or more antibodies, or a complement thereof, and using the sequences of (i) and (ii) to determine all or a part of the nucleic acid sequence of the analyte or the nucleic acid sequence encoding the one or more antibodies.

In one embodiment, a single strand of the double-stranded nucleic acid library member comprises: a first adaptor, a barcode, a capture domain, a sequence of the analyte or a complement thereof, and a second adaptor.

In one embodiment, removing all or a portion of the sequence encoding the constant region of the ABM from the spatially barcoded double-stranded nucleic acid library member comprises: (a) ligating to each end of the double-stranded member of the nucleic acid library a first restriction endonuclease recognition sequence; (b) contacting the double-stranded member of the nucleic acid library of step (a) with a first restriction endonuclease that cleaves the first restriction endonuclease recognition sequence at each end; (c) ligating the ends of the double-stranded member of the nucleic acid library of step (b) to generate a first double-stranded circularized nucleic acid; and (d) amplifying the double-stranded circularized nucleic acid using a first primer and a second primer to generate a double-stranded member of the nucleic acid library lacking all, or a portion of, the analyte sequence, wherein: the first primer comprises: (i) a sequence substantially complementary to a 3′ region of the analyte sequence, and (ii) a first functional domain comprising a sequence for attachment to a flow cell; and the second primer comprises: (i) a sequence substantially complementary to a 5′ region of the analyte sequence, and (ii) a second functional domain comprising a primer sequence to amplify the double-stranded member of the nucleic acid library lacking all, or a portion of, encoding the constant region of the ABM.

In one embodiment, the first primer comprises (i) the sequence substantially complementary to the 3′ region of the analyte sequence, and (ii) the sequence comprising the first functional domain, in 3′ to 5′ direction; and wherein the second primer comprises (i) the sequence substantially complementary to the 5′ region of the analyte sequence, and (ii) the sequence comprising the second functional domain, in a 3′ to 5′ direction.

In some embodiments, ligating in step (c) is performed using a DNA ligase or using template mediated ligation.

In some embodiments, the nucleic acid library is a DNA library or a cDNA library.

In some embodiments, the method further comprises amplifying the double-stranded member of the nucleic acid library lacking all, or a portion of, the sequence encoding the constant region of the ABM using a third primer and a fourth primer, wherein: the third primer is substantially complementary to the first functional domain, and the fourth primer is substantially complementary to the second functional domain.

In some embodiments, determining all or a part of the nucleic acid sequence encoding the one or more antibodies comprises: (a) contacting the tumor tissue sample with an array comprising a feature, wherein the feature comprises an attached first and second probe, wherein: a 5′ end of the first probe is attached to the feature; the first probe comprises in a 5′ to a 3′ direction: a spatial barcode and a poly(T) capture domain, wherein the poly(T) capture domain binds specifically to the nucleic acid sequence encoding the one or more antibodies; a 5′ end of the second probe is attached to the feature; a 3′ end of the second probe is reversibly blocked; and the second probe comprises a poly(GI) capture domain; (b) extending a 3′ end of the first probe to add a sequence that is complementary to a portion of the nucleic acid sequence encoding the one or more antibodies; (c) ligating an adapter to the 5′ end of the target nucleic acid specifically bound to the first probe; (d) adding a sequence complementary to the adapter to the 3′ end of the first probe; (e) adding non-templated cytosines to the 3′ end of the first probe to generate a poly(C) sequence, wherein the poly(C) sequence specifically binds to the poly(GI) capture domain of the second probe; (f) unblocking the 3′ end of the second probe and extending the 3′ end of the second probe to add a sequence comprising a sequence in the nucleic acid sequence encoding the one or more antibodies and a sequence that is complementary to the spatial barcode; (g) cleaving a region of the second probe at a cleavage site that is 5′ to the poly(GI) capture domain, thereby releasing the second probe from the feature; and (h) determining (i) all or a part of the sequence of the spatial barcode, or a complement thereof, and (ii) all or a part of the sequence of the nucleic acid sequence encoding the one or more antibodies, or a complement thereof, and using the sequences of (i) and (ii) to determine all or a part of the nucleic acid sequence encoding the one or more antibodies.

In some embodiments, the attaching in step (b) comprises hybridization.

In some embodiments, the analyte encodes V and J sequences of an immune cell receptor, preferably a BCR or TCR.

In some embodiments, the analyte encodes V, D, and J sequences of an immune cell receptor, preferably a BCR or TCR.

In some embodiments, the tissue sample is a tissue section, and optionally, wherein the tissue section is a fixed tissue section or a fresh, frozen tissue section.

In one embodiments, the fixed tissue section is a formalin-fixed paraffin-embedded tissue section, a paraformaldehyde-fixed tissue section, a methanol-fixed tissue section, or an acetone-fixed tissue section.

In some embodiments, the tissue sample is a human sample.

Each of the aspects and embodiments described herein are capable of being used together, unless excluded either explicitly or clearly from the context of the embodiment or aspect.

The foregoing summary is illustrative only and is not intended to be in any way limiting. In addition to the illustrative embodiments and features described herein, further aspects, embodiments, objects and features of the disclosure will become fully apparent from the drawings and the detailed description and the claims.

### BRIEF DESCRIPTION OF THE DRAWINGS

FIG. 1 shows a schematic diagram of an exemplary capture probe.

FIG. 2 is a schematic illustrating an exemplary cleavable capture probe.

FIG. 3 shows is a schematic diagram of an exemplary multiplexed spatially-barcoded feature.

FIG. 4 is a schematic diagram of an exemplary analyte capture agent.

FIG. 5 is a schematic diagram depicting an exemplary interaction between a feature-immobilized capture probe and an analyte capture agent.

FIG. 6 is a schematic diagram depicting an exemplary sandwiching process.

FIG. 7A is a perspective view of an example sample handling apparatus in a closed position in accordance with some example implementations.

FIG. 7B is a perspective view of the example sample handling apparatus in an open position in accordance with some example implementations.

FIG. 8A shows an exemplary sandwiching process where a first substrate including a biological sample and a second substrate (e.g., including spatially barcoded capture probes) are brought into proximity with one another.

FIG. 8B shows a fully formed sandwich configuration creating a chamber formed from one or more spacers, the first substrate, and the second substrate, in accordance with some example implementations.

FIGS. 9A-9C depict a side view and a top view of an angled closure workflow for sandwiching a first substrate and a second substrate in accordance with some example implementations.

FIG. 10A depicts an exemplary capture probe with a capture sequence that specifically binds to a nucleic acid sequence encoding a constant region of an antigen binding molecule.

FIG. 10B depicts an exemplary poly(A) capture of an analyte encoding an antigen binding molecule with a poly(T) capture domain.

FIG. 11 shows an exemplary analyte enrichment strategy following analyte capture on the array.

FIG. 12 shows a sequencing strategy with a primer specific complementary to the sequencing flow cell attachment sequence (e.g., P5) and a custom sequencing primer complementary to a portion of a constant region of an analyte.

FIG. 13 shows an exemplary nucleic acid library preparation method to remove a portion of an analyte sequence via double circularization of a member of a nucleic acid library.

FIG. 14 depicts another exemplary workflow for processing a double-stranded circularized nucleic acid product.

FIG. 15 shows an exemplary nucleic acid library preparation method to remove all or a portion of a nucleic acid analyte encoding a constant region of an ABM from a member of a nucleic acid library via circularization.

FIG. 16 shows an exemplary nucleic acid library method to reverse the orientation of an analyte sequence in a member of a nucleic acid library.

FIG. 17 is a schematic diagram showing an exemplary feature comprising an attached first probe comprising a poly(T) capture domain and second probe comprising a poly(GI) capture domain.

FIG. 18A is a workflow schematic illustrating exemplary steps for generating a spatially-barcoded sample for analysis and for use in further steps of the methods described herein.

FIG. 18B is a workflow schematic illustrating exemplary steps for specific binding of the extended first probe with the second probe.

FIG. 18C is a workflow schematic illustrating exemplary steps for generating a spatially-barcoded sample for analysis that allows for the sequencing of the target nucleic acid from both the 3′ end and the 5′ end.

FIG. 18D is a schematic diagram showing an exemplary spatially-barcoded sample for analysis generated using the methods described herein.

FIGS. 19A-19J depict an exemplary workflow for detecting and/or determining spatial location of a target polynucleotide of interest.

FIG. 20 is a schematic diagram showing reverse transcription of a target nucleic acid with a first primer and the addition of the complement of a capture sequence into an extension product which is capable of hybridizing to a capture domain of a capture probe.

FIG. 21 is a schematic diagram showing capture and extension on an array of the extension product (e.g., cDNA product) shown in FIG. 20 and extension of the capture probe and the captured extension product (e.g., cDNA product) followed by release of the extended capture product.

FIG. 22 is a schematic diagram showing reverse transcription of a target nucleic acid with a plurality of primers where the reverse transcription occurs with a reverse transcriptase with strand displacement activity or reverse transcription with a reverse transcriptase with a helicase or a superhelicase (top). In the embodiment shown, the primers hybridize to a region of the nucleic acid that encodes for a constant region of an immune cell receptor and generate one or more extension products of varying lengths that include V(D)J sequences depending on where the primers hybridize to the target nucleic acid that encodes for a constant region of an immune cell receptor (bottom).

FIGS. 23A-23B are mouse brain images showing fluorescently labeled cDNA post reverse transcription (FIG. 23A) and post permeabilization and cDNA extension (FIG. 23B).

FIGS. 24A-24B show mouse brain images. FIG. 24A shows a brightfield image and FIG. 24B shows fluorescently labeled extended cDNA generated by extension in the presence of Cy3-dCTPs.

FIGS. 25A-25C shows spatial gene expression clusters (FIG. 25A), the corresponding t-SNE plot (FIG. 25B), and spatial gene expression heat map (FIG. 25C).

FIGS. 26A-26D show spatial gene expression clustering with a first primer including a poly(T) sequence (FIG. 26A) and the corresponding t-SNE plot (FIG. 26B) and spatial gene expression clustering with a first primer including a random decamer (FIG. 26C) and the corresponding t-SNE plot (FIG. 26D).

FIG. 27 shows fluorescently labeled extended cDNA post permeabilization and cDNA extension in mouse brain tissue using a template switch ribonucleotide with an alternative handle.

FIGS. 28A-28B are graphs showing correlation between fresh frozen capture using standard Visium spatial gene expression (10× Genomics) and spatial 5′ end capture (FIG. 28A) and a graph showing normalized position of each mapped read within the full-length transcript and confirming successful 5′ enrichment with a primer including a random decamer (FIG. 28B).

FIG. 29 shows a computer system that is programmed or otherwise configured to implement methods provided herein.

### DETAILED DESCRIPTION OF THE DISCLOSURE

The present disclosure relates generally to the development of new or improved immuno-therapeutics, such as recombinant antigen-binding molecules (ABM) (e.g., antibodies) and pharmaceutical compositions comprising the same for use in treating diseases such as cancer. Some embodiments of the disclosure provide compositions and methods for the analysis and generation of antigen-binding molecules (e.g., antibodies produced by B cells) in tumor tissue samples, using spatial analysis, so as to produce recombinant antigen-binding molecules (e.g., antibodies) with desired properties. As described in greater detail below, one aspect of the disclosure relates to methods for characterization and/or generation of recombinant antibodies from tissue samples, such as tumor tissue samples. In some embodiments, the methods, compositions and systems disclosed herein are used to analyze the sequence of an ABM, e.g., a B cell receptor heavy chain (V_{H}), B cell receptor light chain (V_{L}), or any fragment thereof, e.g., variable regions including VDJ or VJ regions, constant regions, transmembrane regions, fragments thereof, combinations thereof, and combinations of fragments thereof.

In some embodiments, the recombinant antibodies generated by the disclosed methods are coupled to a reporter oligonucleotide comprising a reporter barcode sequence to generate barcoded recombinant antibodies, which can then be used for a multitude of downstream applications, including identification of antibodies having specificity for a tumor, specificity for an individual, or specificity for population of individuals. In some embodiments, the barcoded recombinant antibodies of the disclosure are used in a method of monitoring antigen escape in an individual who has been treated with an antibody-based therapy, such as a therapeutic antibody or an antibody-drug conjugate (ADC). In some embodiments, the recombinant antibodies of the disclosure are used in a method of treatment. Also provided in some embodiments of the disclosure are recombinant antibodies, compositions and methods useful for the production of such antibodies, as well as kits and systems for antibody discovery and/or management.

### Definitions

Unless otherwise defined, all terms of art, notations and other scientific terms or terminology used herein are intended to have the meanings commonly understood by those of skill in the art to which this disclosure pertains. In some cases, terms with commonly understood meanings are defined herein for clarity and/or for ready reference, and the inclusion of such definitions herein should not necessarily be construed to represent a substantial difference over what is generally understood in the art. Many of the techniques and procedures described or referenced herein are well understood and commonly employed using conventional methodology by those skilled in the art.

The singular form “a”, “an”, and “the” include plural references unless the context clearly dictates otherwise. For example, the term “a cell” includes one or more cells, comprising mixtures thereof. “A and/or B” is used herein to include all of the following alternatives: “A”, “B”, “A or B”, and “A and B”.

The term “biological particle,” as used herein, generally refers to a discrete biological system derived from a biological sample. The biological particle may be a macromolecule. The biological particle may be a small molecule. The biological particle may be a virus, e.g., a phage. The biological particle may be a cell or derivative of a cell. The biological particle may be an organelle. The biological particle may be a rare cell from a population of cells. The biological particle may be any type of cell, including without limitation prokaryotic cells, eukaryotic cells, bacterial, fungal, plant, mammalian, or other animal cell type, mycoplasmas, normal tissue cells, tumor cells, or any other cell type, whether derived from single cell or multicellular organisms. The biological particle may be a constituent of a cell. The biological particle may be or may include DNA, RNA, organelles, proteins, or any combination thereof. The biological particle may be or may include a matrix (e.g., a gel or polymer matrix) comprising a cell or one or more constituents from a cell (e.g., cell bead), such as DNA, RNA, organelles, proteins, or any combination thereof, from the cell. The biological particle may be obtained from a tissue of a subject. The biological particle may be a hardened cell. Such hardened cell may or may not include a cell wall or cell membrane. The biological particle may include one or more constituents of a cell, but may not include other constituents of the cell. An example of such constituents is a nucleus or an organelle. A cell may be a live cell. The live cell may be capable of being cultured, for example, being cultured when enclosed in a gel or polymer matrix, or cultured when comprising a gel or polymer matrix. In some embodiments, a biological particle is an analyte carrier, e.g., a cell or constituent of a cell, such as a cell nucleus or organelle.

An “adapter,” an “adaptor,” and a “tag” are terms that are used interchangeably in this disclosure, and refer to moieties that can be coupled to a polynucleotide sequence (in a process referred to as “tagging”) using any one of many different techniques including (but not limited to) ligation, hybridization, and tagmentation. Adapters can also be nucleic acid sequences that add a function, e.g., spacer sequences, primer sequences, primer binding sites, barcode sequences, and unique molecular identifier sequences.

The term “barcode” is used herein to refer to a label, or identifier, that conveys or is capable of conveying information (e.g., information about an analyte in a sample, a bead, and/or a nucleic acid barcode molecule). A barcode can be part of an analyte or nucleic acid barcode molecule, or independent of an analyte or nucleic acid barcode molecule. A barcode can be attached to an analyte or nucleic acid barcode molecule in a reversible or irreversible manner. A particular barcode can be unique relative to other barcodes. Barcodes can have a variety of different formats. For example, barcodes can include polynucleotide barcodes, random nucleic acid and/or amino acid sequences, and synthetic nucleic acid and/or amino acid sequences. A barcode can be attached to an analyte or to another moiety or structure in a reversible or irreversible manner. A barcode can be added to, for example, a fragment of a deoxyribonucleic acid (DNA) or ribonucleic acid (RNA) sample before or during sequencing of the sample. Barcodes can allow for or facilitates identification and/or quantification of individual sequencing-reads. In some embodiments, a barcode can be configured for use as a fluorescent barcode. For example, in some embodiments, a barcode can be configured for hybridization to fluorescently labeled oligonucleotide probes. Barcodes can be configured to spatially resolve molecular components found in biological samples, for example, at single-cell resolution (e.g., a barcode can be or can include a “spatial barcode”). In some embodiments, a barcode includes two or more sub-barcodes that together function as a single barcode. For example, a polynucleotide barcode can include two or more polynucleotide sequences (e.g., sub-barcodes). In some embodiments, the two or more sub-barcodes are separated by one or more non-barcode sequences. In some embodiments, the two or more sub-barcodes are not separated by non-barcode sequences.

In some embodiments, a barcode can include one or more unique molecular identifiers (UMIs). Generally, a unique molecular identifier is a contiguous nucleic acid segment or two or more non-contiguous nucleic acid segments that function as a label or identifier for a particular analyte, or for a nucleic acid barcode molecule that binds a particular analyte (e.g., mRNA) via the capture sequence.

A UMI can include one or more specific polynucleotides sequences, one or more random nucleic acid and/or amino acid sequences, and/or one or more synthetic nucleic acid and/or amino acid sequences. In some embodiments, the UMI is a nucleic acid sequence that does not substantially hybridize to analyte nucleic acid molecules in a biological sample. In some embodiments, the UMI has less than 80% sequence identity (e.g., less than 70%, 60%, 50%, or less than 40% sequence identity) to the nucleic acid sequences across a substantial part (e.g., 80% or more) of the nucleic acid molecules in the biological sample. These nucleotides can be completely contiguous, i.e., in a single stretch of adjacent nucleotides, or they can be separated into two or more separate subsequences that are separated by 1 or more nucleotides.

“Cancer” refers to the presence of cells possessing several characteristics typical of cancer-causing cells, such as uncontrolled proliferation, immortality, metastatic potential, rapid growth and proliferation rate, and certain characteristic morphological features. Some types of cancer cells can aggregate into a mass, such as a tumor, but some cancer cells can exist alone within a subject. A tumor can be a solid tumor, a soft tissue tumor, or a metastatic lesion. As used herein, the term “cancer” also encompasses other types of non-tumor cancers. Non-limiting examples include blood cancers or hematological malignancies, such as leukemia, lymphoma, and myeloma. Cancer can include premalignant, as well as malignant cancers.

The terms “cell”, “cell culture”, “cell line” refer not only to the particular subject cell, cell culture, or cell line but also to the progeny or potential progeny of such a cell, cell culture, or cell line, without regard to the number of transfers or passages in culture. It should be understood that not all progeny are exactly identical to the parental cell. This is because certain modifications may occur in succeeding generations due to either mutation (e.g., deliberate or inadvertent mutations) or environmental influences (e.g., methylation or other epigenetic modifications), such that progeny may not, in fact, be identical to the parent cell, but are still included within the scope of the term as used herein, so long as the progeny retain the same functionality as that of the originally cell, cell culture, or cell line.

As used herein, “isolated” antigen-binding polypeptides, antibodies or antigen-binding fragments thereof, polypeptides, polynucleotides and vectors, are at least partially free of other biological molecules from the cells or cell culture from which they are produced. Such biological molecules include nucleic acids, proteins, other antibodies or antigen-binding fragments, lipids, carbohydrates, or other material such as cellular debris and growth medium. An isolated antibody or antigen-binding fragment may further be at least partially free of expression system components such as biological molecules from a host cell or of the growth medium thereof. Generally, the term “isolated” is not intended to refer to a complete absence of such biological molecules or to an absence of water, buffers, or salts or to components of a pharmaceutical formulation that includes the antibodies or antigen-binding fragments.

As used herein, the term “functional fragment thereof” or “functional variant thereof” relates to a molecule having qualitative biological activity in common with the wild-type molecule from which the fragment or variant was derived. For example, a functional fragment or a functional variant of an antibody is one which retains essentially the same ability to bind to the same epitope as the antibody from which the functional fragment or functional variant was derived.

The term “operably linked”, as used herein, denotes a physical or functional linkage between two or more elements, e.g., polypeptide sequences or polynucleotide sequences, which permits them to operate in their intended fashion. For example, the term “operably linked” when used in context of the orthogonal DNA target sequences described herein or the promoter sequence in a nucleic acid construct, or in an engineered response element means that the orthogonal DNA target sequences and the promoters are in-frame and in proper spatial and distance away from a polynucleotide of interest coding for a protein or an RNA to permit the effects of the respective binding by transcription factors or RNA polymerase on transcription.

The term “recombinant” when used with reference to a cell, a nucleic acid, a protein, or a vector, indicates that the cell, nucleic acid, protein or vector has been altered or produced through human intervention such as, for example, has been modified by or is the result of laboratory methods. Thus, for example, recombinant proteins and nucleic acids include proteins and nucleic acids produced by laboratory methods. Recombinant proteins can include amino acid residues not found within the native (non-recombinant or wild-type) form of the protein or can be include amino acid residues that have been modified, e.g., labeled. The term can include any modifications to the peptide, protein, or nucleic acid sequence. Such modifications may include the following: any chemical modifications of the peptide, protein or nucleic acid sequence, including of one or more amino acids, deoxyribonucleotides, or ribonucleotides; addition, deletion, and/or substitution of one or more of amino acids in the peptide or protein; creation of a fusion protein, e.g., a fusion protein comprising an antibody fragment; and addition, deletion, and/or substitution of one or more of nucleic acids in the nucleic acid sequence. The term “recombinant” when used in reference to a cell is not intended to include naturally-occurring cells but encompass cells that have been engineered/modified to include or express a polypeptide or nucleic acid that would not be present in the cell if it was not engineered/modified.

As used herein, a “subject” or an “individual” includes animals, such as human (e.g., human individuals) and non-human animals. In some embodiments, a “subject” or “individual” is a patient under the care of a physician. Thus, the subject can be a human patient or an individual who has, is at risk of having, or is suspected of having a disease of interest (e.g., cancer) and/or one or more symptoms of the disease. The subject can also be an individual who is diagnosed with a risk of the condition of interest at the time of diagnosis or later. The term “non-human animals” includes all vertebrates, e.g., mammals, e.g., rodents, e.g., mice, non-human primates, and other mammals, such as e.g., rat, mouse, cat, dog, cow, pig, sheep, horse, goat, rabbit; and non-mammals, such as amphibians, chicken, reptiles, etc. A subject can be a mammal, preferably a human or humanized animal, e.g., an animal with humanized VDJC loci. The subject may be non-human animals with humanized VDJC loci and knockouts of a target of interest. The subject may be in need of prevention and/or treatment of a disease or disorder such as viral infection or cancer.

A “variant” of a polypeptide, such as an antibody or an immunoglobulin chain (e.g., VH, VL, HC, or LC), refers to a polypeptide comprising an amino acid sequence that has at least about 70-99.9% (e.g., 70%, 72%, 74%, 75%, 76%, 79%, 80%, 81%, 82%, 83%, 84%, 85%, 86%, 87%, 88%, 89%, 90%, 91%, 92%, 93%, 94%, 95%, 96%, 97%, 98%, 99%, 99.5%, 99.9%) sequence identity or similarity to a referenced amino acid sequence that is set forth herein. In some embodiments, the term “percent identity,” as used herein in the context of two or more proteins, refers to two or more sequences or subsequences that are the same or have a specified percentage of amino acids that are the same, e.g., about 70%, 72%, 74%, 75%, 76%, 79%, 80%, 81%, 82%, 83%, 84%, 85%, 86%, 87%, 88%, 89%, 90%, 91%, 92%, 93%, 94%, 95%, 96%, 97%, 98%, 99%, 99.5%, 99.9%, or higher identity over a specified region, when compared and aligned for maximum correspondence over a comparison window or designated region as measured using a BLAST or BLAST 2.0 sequence comparison algorithms with default parameters described below, or by manual alignment and visual inspection. See, e.g., the NCBI web site at ncbi.nlm.nih.gov/BLAST. Similarly, a “variant” of a nucleic acid molecule refers to a nucleic acid molecule comprising a nucleic acid sequence that has at least about 70-99.9% (e.g., 70%, 72%, 74%, 75%, 76%, 79%, 80%, 81%, 82%, 83%, 84%, 85%, 86%, 87%, 88%, 89%, 90%, 91%, 92%, 93%, 94%, 95%, 96%, 97%, 98%, 99%, 99.5%, 99.9%) sequence identity or similarity to a referenced nucleic acid sequence that is set forth herein. In some embodiments, this definition also refers to, or may be applied to, the complement of a query sequence. In some embodiments, this definition includes sequence comparison performed by a BLAST algorithm wherein the parameters of the algorithm are selected to give the largest match between the respective sequences over the entire length of the respective reference sequences. In some embodiments, this definition also includes sequences that have modifications such as deletions and/or additions (e.g., insertions), as well as those that have substitutions. Such modifications can occur naturally or synthetically. In some embodiments, sequence identity can be calculated over a region that is at least about 20 amino acids or nucleotides in length, or over a region that is 10-100 amino acids or nucleotides in length, or over the entire length of a given sequence. Sequence identity can be calculated using published techniques and widely available computer programs, such as the GCS program package (Devereux et al, Nucleic Acids Res (1984) 12:387), BLASTP, BLASTN, FASTA (Atschul et al., J Mol Biol (1990) 215:403). In some embodiments, sequence identity can be measured using sequence analysis software such as the Sequence Analysis Software Package of the Genetics Computer Group at the University of Wisconsin Biotechnology Center (1710 University Avenue, Madison, Wis. 53705), with the default parameters thereof. Additional methodologies that can suitably be utilized to determine similarity or identity amino acid sequences include those relying on position-specific structure-scoring matrix (P3SM) that incorporates structure-prediction scores from Rosetta, as well as those based on a length-normalized edit distance as described previously in, e.g., Setcliff et al., Cell Host & Microbe 23(6), May 2018.

Certain ranges are presented herein with numerical values being preceded by the term “about.” The term “about” is used herein to provide literal support for the exact number that it precedes, as well as a number that is near to or approximately the number that the term precedes. In determining whether a number is near to or approximately a specifically recited number, the near or approximating unrecited number may be a number which, in the context in which it is presented, provides the substantial equivalent of the specifically recited number. If the degree of approximation is not otherwise clear from the context, “about” means either within plus or minus 10% of the provided value, or rounded to the nearest significant figure, in all cases inclusive of the provided value. In some embodiments, the term “about” indicates the designated value ± up to 10%, up to ±5%, or up to ±1%.

It is understood that aspects and embodiments of the disclosure described herein include “comprising”, “consisting”, and “consisting essentially of” aspects and embodiments. As used herein, “comprising” is synonymous with “including”, “containing”, or “characterized by”, and is inclusive or open-ended and does not exclude additional, unrecited elements or method steps. As used herein, “consisting of” excludes any elements, steps, or ingredients not specified in the claimed composition or method. As used herein, “consisting essentially of” does not exclude materials or steps that do not materially affect the basic and novel characteristics of the claimed composition or method. Any recitation herein of the term “comprising”, particularly in a description of components of a composition or in a description of steps of a method, is understood to encompass those compositions and methods consisting essentially of and consisting of the recited components or steps.

Headings, e.g., (a), (b), (i) etc., are presented merely for ease of reading the specification and claims. The use of headings in the specification or claims does not require the steps or elements be performed in alphabetical or numerical order or the order in which they are presented.

Use of ordinal terms such as “first”, “second”, “third”, etc., in the claims to modify a claim element does not by itself connote any priority, precedence, or order of one claim element over another or the temporal order in which acts of a method are performed, but are used merely as labels to distinguish one claim element having a certain name from another element having a same name (but for use of the ordinal term) to distinguish the claim elements. Similarly, the use of these terms in the specification does not by itself connote any required priority, precedence, or order.

It is appreciated that certain features of the disclosure, which are, for clarity, described in the context of separate embodiments, may also be provided in combination in a single embodiment. Conversely, various features of the disclosure, which are, for brevity, described in the context of a single embodiment, may also be provided separately or in any suitable sub-combination. All combinations of the embodiments pertaining to the disclosure are specifically embraced by the present disclosure and are disclosed herein just as if each and every combination was individually and explicitly disclosed. In addition, all sub-combinations of the various embodiments and elements thereof are also specifically embraced by the present disclosure and are disclosed herein just as if each and every such sub-combination was individually and explicitly disclosed herein.

### Methods of the Disclosure

Some embodiments of the disclosure provide compositions and methods for the analysis and generation of antigen-binding molecules produced by immune cells in tumor tissue samples (e.g., antibodies produced by B cells in tumor tissue samples or TCRs produced by T cells in tumor tissue samples), using spatial analysis, so as to produce recombinant antigen-binding molecules (e.g., antibodies, TCRs) with desired properties. As described in greater detail below, one aspect of the disclosure relates to methods for characterization and/or generation of recombinant monoclonal antibodies from derived from tissue samples, for example, individual B cells within tumor tissue samples. In some embodiments, the methods, compositions and systems disclosed herein are used to determine and analyze the sequence of a B cell receptor heavy chain (V_{H}), B cell receptor light chain (V_{L}), or any fragment thereof, e.g., variable regions including VDJ or VJ regions, constant regions, transmembrane regions, fragments thereof, and combinations of fragments thereof. In some embodiments, the methods further include using the determined nucleic acid sequences to produce a recombinant antibody, and optionally couple the produced recombinant antibody with a reporter molecule, e.g., reporter oligonucleotide to generate a barcoded recombinant antibody, which then can be incorporated into a wide range of downstream applications. For example, in some embodiments, the barcoded recombinant antibodies disclosed herein can be used in methods of identifying tumor-specific antibodies, methods of characterizing antibody specificity or target specificity, methods for enhanced identification of patient-specific or population-specific biomarkers on tumor cells, e.g., circulating tumor cells, methods for monitoring antigen escape in an individual who has been treated with an antibody-based therapy, or methods for characterizing a potential antigen for an antibody or fragment thereof.

### A. Methods for Generating Recombinant Antibodies

In described in more detail below, one aspect of the disclosure relates to new approaches and methods for the analysis, characterization, and/or generation of recombinant antibodies derived from B cells in tissue (e.g., tumor tissue), using spatial analysis, so as to produce recombinant antibodies with desired properties. In some embodiments, the recombinant antibodies are recombinant human antibodies.

In some embodiments, the methods of the disclosure include (a) providing a biological sample comprising one or more cells expressing an antigen-binding molecule (ABM); (b) attaching an analyte of an ABM-expressing cell of the biological sample to a capture domain of a first capture probe of a substrate comprising an array of capture probes attached thereto, the first capture probe comprising (i) a spatial barcode sequence and (ii) the capture domain, the capture domain comprising a capture sequence, wherein the analyte of the ABM-expressing cell comprises a sequence or portion of a sequence encoding the ABM expressed by the ABM-expressing cell or a reverse complement thereof, (c) using the analyte of the ABM-expressing cell and the first capture probe attached thereto to generate a spatially barcoded polynucleotide comprising (i) a sequence of the analyte of the ABM-expressing cell or a reverse complement thereof and (ii) the spatial barcode sequence or a reverse complement thereof, and determining all or a part of the nucleic acid sequences of (i) and (ii); and (d) using the determined nucleic acid sequences to produce a recombinant antibody encoded by the determined sequences. The one or more cells expressing an ABM, e.g., the ABM-expressing cell, can include, for example, a B cell, natural killer (NK) cell, a T-Reg cell, a CAR-T cell, a lymphocyte, T cell or a combination thereof.

In preferred embodiments, the analyte is a nucleic acid analyte (e.g., DNA or RNA such as mRNA). In some embodiments, the biological sample is a tissue sample, such as a tumor tissue sample.

In some embodiments, the ABM expressed by the ABM-expressing cell is an immune cell receptor. The immune cell receptor can be a BCR or a TCR. The immune cell receptor can be an Fc receptor.

In some embodiments, the ABM expressed by the ABM-expressing cell is a secreted antibody.

In some embodiments, the tissue sample can include one or more ABMs. In some embodiments, the biological sample (e.g., tissue sample) is obtained from a subject (e.g., a human). The subject may be a subject that was previously exposed to or may be exposed to an antigen. For example, the subject may be currently undergoing or is a candidate for a cancer therapy. In some embodiments, methods provided herein can identify whether an antigen binding molecule in the subject can bind an antigen. Other exemplary subjects are described herein. In some embodiments, the tissue sample obtained from the subject is a sample comprising an immune cell. Exemplary samples that comprise immune cells include, e.g., spleen, lymph node, tonsil, bone marrow sample, tumor samples, and the like.

In some embodiments, the tissue sample obtained from the subject is a diseased tissue sample. For example, the tissue sample can comprise tumor cells. In some embodiments, the tissue sample obtained from the subject is a healthy tissue sample. In some embodiments, the tissue sample obtained from the subject is a tissue sample comprising or suspected of comprising an antigenic target to which an antibody is expected to bind.

In some embodiments, the tissue sample is mounted on the substrate comprising the array of capture probes during (a) and/or (b). In some embodiments, the tissue sample is in contact with the array of capture probes during (b). In some embodiments, a portion of the tissue sample comprising the ABM-expressing cell is in contact with the first capture probe of the array of capture probes. In some embodiments, the method comprises following (a), releasing the analyte from the ABM-expressing cell of the tissue sample; and optionally migrating the analyte to the substrate comprising the array of capture probes attached thereto.

In some cases, it may be advantageous to have the tissue sample mounted on a first substrate (e.g., regular slide), and migrate analytes from the slide-mounted tissue to the array of capture probes for capture in a manner that preserves spatial context. Accordingly, in some embodiments, the tissue sample is processed according to a “sandwiching process” for the release and migration of the analytes to the array of capture probes in a manner that preserves their spatial context. Sandwiching processes are disclosed in further detail herein.

B. Methods for generating recombinant TCRs

As described in more detail below, one aspect of the disclosure relates to new approaches and methods for the analysis, characterization, and/or generation of recombinant TCRs derived from T cells in tumor tissue samples, using spacial array methodologies, so as to produce recombinant TCRs with desired properties. In some embodiments, the recombinant TCRs are recombinant human TCRs.

In some embodiments, the methods of the disclosure include (a) providing a first tumor tissue sample; (b) determining all or a part of the nucleic acid sequences encoding one or more TCRs produced by the tumor tissue samples; and (c) using the determined nucleic acid sequences to produce a recombinant TCR.

An exemplary workflow for the approaches disclosed herein generally involves providing a tumor tissue sample comprising one or more cells expressing a TCR, attaching an analyte of a TCR-expressing cell of the tumor tissue sample to a capture domain of a first capture probe of a substrate comprising an array of capture probes attached thereto, the first capture probe comprising (i) a spatial barcode sequence and (ii) the capture domain, the capture domain comprising a first capture sequence, wherein the analyte of the TCR-expressing cell comprises a sequence or portion of a sequence encoding the TCR expressed by the TCR-expressing cell or a reverse complement thereof, using the analyte of the TCR-expressing cell and the first capture probe attached thereto to generate a spatially barcoded polynucleotide comprising (i) a sequence of the analyte of the TCR-expressing cell or reverse complement thereof and (ii) the spatial barcode sequence or reverse complement thereof, and determining all or a part of the nucleic acid sequences of the spatially barcoded polynucleotide, and using the determined nucleic acid sequences to produce a recombinant TCR encoded by the determined sequences. Exemplary methods and systems for spatial analysis are described herein.

Nucleic acid sequencing of the barcoded polynucleotide can be used to determine nucleic acid sequences that encode one or more TCRs produced by the tissue sample.

A plethora of different approaches, systems, and techniques for nucleic acid sequencing, including next-generation sequencing (NGS) methods, can be used to determine the nucleic acid sequences encoding the ABMs produced by the B or T cells within the tissue sample. More generally, sequencing can be performed using nucleic acid amplification, polymerase chain reaction (PCR) (e.g., digital PCR and droplet digital PCR (ddPCR), quantitative PCR, real time PCR, multiplex PCR, PCR-based singleplex methods, emulsion PCR), and/or isothermal amplification.

Non-limiting examples of nucleic acid sequencing methods include Maxam-Gilbert sequencing and chain-termination methods, de novo sequencing methods including shotgun sequencing and bridge PCR, next-generation methods including Polony sequencing, 454 pyrosequencing, Illumina sequencing, SOLiD™ sequencing, Ion Torrent semiconductor sequencing, HeliScope single molecule sequencing, and SMRT® sequencing.

Other examples of methods for sequencing genetic material include, but are not limited to, DNA hybridization methods, restriction enzyme digestion methods, Sanger sequencing methods, ligation methods, and microarray methods. Additional examples of sequencing methods that can be used include targeted sequencing, single molecule real-time sequencing, exon sequencing, electron microscopy-based sequencing, panel sequencing, transistor-mediated sequencing, direct sequencing, random shotgun sequencing, Sanger dideoxy termination sequencing, whole-genome sequencing, sequencing by hybridization, pyrosequencing, capillary electrophoresis, gel electrophoresis, duplex sequencing, cycle sequencing, single-base extension sequencing, solid-phase sequencing, high-throughput sequencing, massively parallel signature sequencing, co-amplification at lower denaturation temperature-PCR (COLD-PCR), sequencing by reversible dye terminator, paired-end sequencing, near-term sequencing, exonuclease sequencing, sequencing by ligation, short-read sequencing, single-molecule sequencing, sequencing-by-synthesis, real-time sequencing, reverse-terminator sequencing, nanopore sequencing, Solexa Genome Analyzer sequencing, MS-PET sequencing, and any combinations thereof.

Sequence analysis of the nucleic acid molecules, including barcoded polynucleotides or nucleic acid molecules (e.g., barcoded cDNA), can be direct or indirect. Thus, the sequence analysis substrate (which can be viewed as the molecule which is subjected to the sequence analysis step or process) can be the barcoded nucleic acid molecule or it can be a molecule which is derived therefrom (e.g., a complement thereof). Thus, for example, in the sequence analysis step of a sequencing reaction, the sequencing template can be the barcoded nucleic acid molecule or it can be a molecule derived therefrom. For example, a first and/or second strand DNA molecule can be directly subjected to sequence analysis (e.g., sequencing), i.e., can directly take part in the sequence analysis reaction or process (e.g., the sequencing reaction or sequencing process, or be the molecule which is sequenced or otherwise identified). Alternatively, the barcoded nucleic acid molecule can be subjected to a step of second strand synthesis or amplification before sequence analysis (e.g., sequencing or identification by another technique). The sequence analysis substrate (e.g., template) can thus be an amplicon or a second strand of a barcoded nucleic acid molecule.

In some embodiments, both strands of a double stranded molecule (e.g., cDNA) can be subjected to sequence analysis. In some embodiments, single stranded molecules (e.g., barcoded nucleic acid molecules) can be sequenced.

In some embodiments, all or a part of the nucleic acid sequences encoding one or more antigen-binding molecules produced by the immune cell in the tissue sample (e.g., encoding one or more antibodies produced by the B cell) can be determined by using a whole-exome sequencing technique (WES), which generally involves sequencing all of the protein-coding regions of genes in a cellular genome (often referred to as the exome). A general workflow of whole-exome sequencing includes two steps: the first step involves selecting only the subset of DNA that encodes proteins. These regions are known as exons (for example, humans have about 180,000 exons, constituting about 1% of the human genome). The second step involves sequencing the exonic DNA using any suitable high-throughput DNA sequencing technology.

Production of Recombinant Antigen-Binding Molecules (e.g. Recombinant Antibodies or Recombinant TCRs)

In some embodiments, the method further includes generating a recombinant antigen-binding molecule using the determined nucleic acid sequences of the spatially barcoded polynucleotide. In some embodiments, the method includes further generating a recombinant antibody using the determined nucleic acid sequences of the spatially barcoded polynucleotide. In some embodiments, the method includes further generating a recombinant TCR using the determined nucleic acid sequences of the spatially barcoded polynucleotide. One skilled in the art will appreciate that the determined nucleic acid sequences of the V_{H} and V_{L} mRNAs can be used to construct a full-length gene encoding a desired recombinant antibody. For example, a DNA oligomer containing a full-length nucleotide sequence coding for a given V_{H} and V_{L} domain of the desired antibody can be synthesized. In addition or alternatively, several small oligonucleotides coding for portions of the desired recombinant antibody can be synthesized and then ligated. The individual oligonucleotides generally contain 5′ or 3′ overhangs for complementary assembly.

In addition to generating desired antibodies or TCRs via expression of nucleic acid molecules that have been altered by recombinant molecular biological techniques, a subject recombinant antibody or TCR in accordance with the present disclosure can be chemically synthesized. Chemically synthesized polypeptides are routinely generated by those of skill in the art.

Once assembled (by synthesis, recombinant methodologies, site-directed mutagenesis or other suitable techniques), the DNA sequences encoding a recombinant antigen-binding molecule (e.g., antibody or TCR) as disclosed herein can be inserted into an expression vector and operably linked to an expression control sequence appropriate for expression of the recombinant antibody in the desired transformed host. Proper assembly can be confirmed by nucleotide sequencing, restriction mapping, and expression of a biologically active polypeptide in a suitable host. As is known in the art, in order to obtain high expression levels of a transfected gene in a host, care should be taken to ensure that the gene encoding the recombinant antibody is operably linked to transcriptional and translational expression control sequences that are functional in the chosen expression host.

In some embodiments, a method of the disclosure further involves including coupling a reporter oligonucleotide including a reporter barcode sequence to the recombinant antibody to generate a barcoded recombinant antibody. In some embodiments, the reporter barcode sequence of the reporter oligonucleotide includes a unique identifier for the recombinant antibody. In some embodiments, the unique identifier for the recombinant antibody is a nucleic acid identifier. In some embodiments, the method of the disclosure further includes determining all or a part of the nucleic acid identifier to identify the barcoded recombinant antibody. In some embodiments, the reporter oligonucleotide comprise an adapter region that allows for downstream analysis of the recombinant antibody. In some embodiments, the adapter region comprises a primer binding site and/or a cleavage site. In some embodiments, the barcoded recombinant antibody includes a linker. In some embodiments, the linker is a cleavable linker. In some embodiments, the cleavable linker is a photo-cleavable linker, a UV-cleavable linker, or an enzyme cleavable linker.

In some embodiments, a method of the disclosure further includes contacting the barcoded recombinant antibody to a second tumor sample, which can be a tissue sample. In some embodiments, a method further includes identifying the recombinant antibody as an antibody having specificity for the second tumor sample if the barcoded recombinant antibody is capable of binding to an antigen associated with the second tumor sample.

In some embodiments, a method of the disclosure further involves coupling a reporter oligonucleotide including a reporter barcode sequence to the recombinant TCR to generate a barcoded recombinant TCR In some embodiments, the reporter barcode sequence of the reporter oligonucleotide includes a unique identifier for the recombinant TCR. In some embodiments, the unique identifier for the recombinant is a nucleic acid identifier. In some embodiments, the method of the disclosure further includes determining all or a part of the nucleic acid identifier to identify the barcoded recombinant TCR. In some embodiments, the reporter oligonucleotide comprises an adapter region that allows for downstream analysis of the recombinant TCR. In some embodiments, the adapter region comprises a primer binding site and/or a cleavage site. In some embodiments, the barcoded recombinant TCR includes a linker. In some embodiments, the linker is a cleavable linker. In some embodiments, the cleavable linker is a photo-cleavable linker, a UV-cleavable linker, or an enzyme cleavable linker.

In some embodiments, the method of the disclosure further includes contacting the barcoded recombinant TCR to a second tumor sample, which can be a tissue sample. In some embodiments, the method further includes identifying the recombinant TCR as an TCR having specificity for the second tumor sample if the barcoded recombinant TCR is capable of binding to an antigen associated with the second tumor sample.

For either recombinant antibodies or recombinant TCRs, the associated reporter oligonucleotide can include a capture handle sequence which can hybridize to at least a portion or an entirety of a capture domain of a capture probe. In some embodiments, the capture handle sequence is complementary to a portion or entirety of a capture domain of a capture probe. In some embodiments, the capture handle sequence includes a poly (A) tail. In some embodiments, the capture handle sequence includes a sequence capable of binding a poly (T) domain. Other embodiments of reporter oligonucleotide couple to antibodies useful in spatial analyte detection are described herein.

Attachment (coupling) of the reporter oligonucleotides to a recombinant antibody or recombinant TCR can be performed via any of the methods described herein for attachment (coupling) of reporter oligonucleotides to labelling agents (such as a protein, e.g., an antibody or antibody fragment). For example, attachment (coupling) or the reporter oligonucleotides to a recombinant antibody or recombinant TCR can be performed using chemical conjugation techniques (e.g., Lightning-Link® antibody labelling kits available from Innova Biosciences), as well as other non-covalent attachment mechanisms, e.g., using biotinylated antibodies and oligonucleotides (or beads that include one or more biotinylated linker, coupled to oligonucleotides) with an avidin or an streptavidin linker in monomeric or multimeric form (e.g., tetramic form of streptavidin). Those of skill in the art will recognize that a streptavidin monomer encompasses streptavidin molecules with 1 biotin binding site, while a streptavidin multimer encompasses strepatavidin molecules with more than 1 biotin binding site. For example, a streptavidin tetramer has 4 biotin binding sites. However, a skilled artisan will also recognize that in a streptavidin tetramer does not necessarily comprise 4 streptavidins complexed together. Antibody and oligonucleotide biotinylation techniques are available. See, e.g., Fang, et al., “Fluoride-Cleavable Biotinylation Phosphoramidite for 5′-end-Labelling and Affinity Purification of Synthetic Oligonucleotides,” Nucleic Acids Res. Jan. 15, 2003; 31(2):708-715, which is entirely incorporated herein by reference for all purposes. Likewise, protein and peptide biotinylation techniques have been developed and are readily available. See, e.g., U.S. Pat. No. 6,265,552, which is entirely incorporated herein by reference for all purposes. Furthermore, click reaction chemistry such as a Methyltetrazine-PEG5-NHS Ester reaction, a TCO-PEG4-NHS Ester reaction, or the like, can be used to couple reporter oligonucleotides to labelling agents. Commercially available kits, such as those from Thunderlink and Abcam, and techniques common in the art can be used to couple reporter oligonucleotides to labelling agents as appropriate. In another example, a labelling agent is indirectly (e.g., via hybridization) coupled to a reporter oligonucleotide including a barcode sequence that identifies the labelling agent. For instance, the labelling agent can be directly coupled (e.g., covalently bound) to a hybridization oligonucleotide that includes a sequence that hybridizes with a sequence of the reporter oligonucleotide. Hybridization of the hybridization oligonucleotide to the reporter oligonucleotide couples the labelling agent to the reporter oligonucleotide. In some embodiments, the reporter oligonucleotides are releasable from the labelling agent, such as upon application of a stimulus. For example, the reporter oligonucleotide can be attached to the labeling agent through a labile bond (e.g., chemically labile, photolabile, thermally labile, etc.) as generally described for releasing molecules from supports elsewhere herein. In some instances, the reporter oligonucleotides described herein can include one or more functional sequences that can be used in subsequent processing, such as an adapter sequence, a unique molecular identifier (UMI) sequence, a sequencer specific flow cell attachment sequence (such as an P5, P7, or partial P5 or P7 sequence), a primer or primer binding sequence, a sequencing primer or primer biding sequence (such as an R1, R2, or partial R1 or R2 sequence).

### Tumor Samples

The first and/or second tumor sample (e.g., first and/or second tumor tissue sample) can be any biological sample comprising tumor cells. For example, the first and/or second tumor sample can be a sample, such as a biopsy, core biopsy, needle aspirate, or fine needle aspirate. In some embodiments, the first and/or the second tumor sample is independently derived from a solid tumor or cancer, a soft tissue tumor, a non-solid tumor, a metastatic lesion, or a circulating tumor cell (CTC) population. The first and/or second tumor sample can comprise an intact tissue sample. In some embodiments, the solid tumor or cancer is derived from breast, liver, kidney, lung, bone, ovaries, pancreas, prostate, thyroid, colon, or brain.

In some embodiments, the first and the second tumor samples are derived from the same subject. In some embodiments, the first and the second tumor samples are derived from the different subjects. In some embodiments, the first and the second tumor samples are derived from the same tumor type. In some embodiments, the first and the second tumor samples are derived from the different tumor types. In another example, the first sample and the second sample are different samples derived from the same tumor. For example, in some embodiments, the first tumor sample is obtained from a solid tumor and the second tumor sample is a CTC population. In some embodiments, the first and the second tumor samples are derived from the same type of tumor, but are collected at different times and/or under different conditions.

### C. Methods for Characterizing Tumor Response to One or More Antibody Therapeutics

In an aspect, provided herein are methods for characterizing response of a tumor to one or more antibody therapeutics. In some embodiments, the methods of the disclosure include contacting a tumor tissue sample (e.g., a first or second tumor tissue sample described herein) with a composition including one or more barcoded antibodies and/or functional fragments thereof (e.g., a barcoded antibody cocktail). In some embodiments, the tumor sample is a tissue sample. Non-limiting examples of barcoded antibodies suitable for the compositions and methods described herein include barcoded antibodies and functional fragments thereof having specificity for immune cell markers, tumor-cell markers, and/or specificity for a tumor sample. Accordingly, in some embodiments, the methods of the disclosure include contacting a tumor tissue sample with a composition including one or more of the following: (i) one or more barcoded immune-cell marker antibodies and/or functional fragments thereof; (ii) one or more barcoded tumor-cell marker antibodies and functional fragments thereof, (iii) one or more barcoded therapeutic antibodies and functional fragments thereof, and (iv) one or more barcoded recombinant antibodies identified in the present disclosure as having specificity for a tumor sample. In some embodiments, the tumor tissue sample is contacted with a composition including two or more of (i)-(iv), three or more of (i)-(iv), or all of (i)-(iv).

In some embodiments, the method comprises generating the barcoded recombinant antibodies of (iv) using determined nucleic acid sequences of B cells obtained from a first tumor tissue sample; and contacting a second tumor sample with the composition including one or more of (i)-(iv).

In some embodiments, the barcoded recombinant antibodies are each coupled to a reporter oligonucleotide including a reporter barcode sequence. In some embodiments, to facilitate downstream analyses, the reporter barcode sequence coupled to a barcoded antibody is distinguishable from reporter barcode sequences coupled to other barcoded antibodies.

In some embodiments, one or more of the antibodies are monoclonal antibodies or functional fragments thereof. In some embodiments, one or more of the antibodies are recombinant human antibodies or functional fragments thereof. In some embodiments, one or more of the antibodies are polyclonal antibodies or functional fragments thereof. In some embodiments, one or more of the antibodies are multi-specific antibodies (e.g., bispecific antibodies). Functional fragments of the antibodies suitable for the methods described herein can include F(ab) fragments, Fab′ fragments, F(ab′)2 fragments, Fv domains, and Fc domains.

### Therapeutic Antibodies

Therapeutic antibodies that can be used are antibodies that have been approved for human administration for the treatment of a disease, such as cancer or antibodies that are being tested for preclinical and/or clinical trials. In some embodiments, a therapeutic antibody is an antibody of known sequence that is contemplated for use in treating a physiological condition or disease, such as cancer, in a human.

In some embodiments, the method of the disclosure includes contacting the second tumor sample with a composition including one or more therapeutic antibodies that are drug candidates or FDA approved drugs or therapeutics, such as monoclonal antibodies that are approved by the FDA for therapeutic use. The one or more therapeutic antibodies may be barcoded. Non-limiting examples of FDA approved monoclonal antibodies are provided in Table 1 below.

TABLE 1
Exemplary FDA-approved therapeutic monoclonal antibodies.

| Antibody | Brand name | Type | Target |
|---|---|---|---|
| abciximab | ReoPro | chimeric Fab | GPIIb/IIIa |
| adalimumab | Humira | fully human | TNF |
| adalimumab-atto | Amjevita | fully | TNF |
| | | human, biosimilar | |
| ado-trastuzumab | Kadcyla | humanized, | HER2 |
| emtansine | | antibody-drug | |
| | | conjugate | |
| alemtuzumab | Campath, | humanized | CD52 |
| | Lemtrada | | |
| alirocumab | Praluent | fully human | PCSK9 |
| atezolizumab | Tecentriq | humanized | PD-L1 |
| atezolizumab | Tecentriq | humanized | PD-L1 |
| avelumab | Bavencio | fully human | PD-L1 |
| basiliximab | Simulect | chimeric | IL2RA |
| belimumab | Benlysta | fully human | BLyS |
| bevacizumab | Avastin | humanized | VEGF |
| bezlotoxumab | Zinplava | fully human | Clostridium |
| | | | difficile |
| | | | toxin B |
| blinatumomab | Blincyto | mouse, bispecific | CD19 |
| brentuximab | Adcetris | chimeric, | CD30 |
| vedotin | | antibody-drug | |
| | | conjugate | |
| brodalumab | Siliq | chimeric | IL17RA |
| canakinumab | Ilaris | fully human | IL1B |
| capromab | ProstaScint | murine, | PSMA |
| pendetide | | radiolabeled | |
| certolizumab pegol | Cimzia | humanized | TNF |
| cetuximab | Erbitux | chimeric | EGFR |
| daclizumab | Zenapax | humanized | IL2RA |
| daclizumab | Zinbryta | humanized | IL2R |
| daratumumab | Darzalex | fully human | CD38 |
| denosumab | Prolia, | fully human | RANKL |
| | Xgeva | | |
| dinutuximab | Unituxin | chimeric | GD2 |
| dupilumab | Dupixent | fully human | IL4RA |
| durvalumab | Imfinzi | fully human | PD-L1 |
| eculizumab | Soliris | humanized | Complement |
| | | | component 5 |
| elotuzumab | Empliciti | humanized | SLAMF7 |
| evolocumab | Repatha | fully human | PCSK9 |
| golimumab | Simponi | fully human | TNF |
| golimumab | Simponi | fully human | TNF |
| | Aria | | |
| ibritumomab | Zevalin | murine, | CD20 |
| tiuxetan | | radioimmu- | |
| | | notherapy | |
| idarucizumab | Praxbind | humanized Fab | dabigatran |
| infliximab | Remicade | chimeric | TNF alpha |
| infliximab-abda | Renflexis | chimeric, | TNF |
| | | biosimilar | |
| infliximab-dyyb | Inflectra | chimeric, | TNF |
| | | biosimilar | |
| ipilimumab | Yervoy | fully human | CTLA-4 |
| ixekizumab | Taltz | humanized | IL17A |
| mepolizumab | Nucala | humanized | IL5 |
| natalizumab | Tysabri | humanized | alpha-4 integrin |
| necitumumab | Portrazza | fully human | EGFR |
| nivolumab | Opdivo | fully human | PD-1 |
| nivolumab | Opdivo | fully human | PD-1 |
| obiltoxaximab | Anthem | chimeric | Protective |
| | | | antigen of the |
| | | | Anthrax toxin |
| obinutuzumab | Gazyva | humanized | CD20 |
| ocrelizumab | Ocrevus | humanized | CD20 |
| ofatumumab | Arzerra | fully human | CD20 |
| olaratumab | Lartruvo | fully human | PDGFRA |
| omalizumab | Xolair | humanized | IgE |
| palivizumab | Synagis | humanized | F protein |
| | | | of RSV |
| panitumumab | Vectibix | fully human | EGFR |
| pembrolizumab | Keytruda | humanized | PD-1 |
| pertuzumab | Perjeta | humanized | HER2 |
| ramucirumab | Cyramza | fully human | VEGFR2 |
| ranibizumab | Lucentis | humanized | VEGFR1, |
| | | | VEGFR2 |
| raxibacumab | Raxibacumab | fully human | Protective |
| | | | antigen of |
| | | | Bacillus |
| | | | anthracis |
| reslizumab | Cinqair | humanized | IL5 |
| rituximab | Rituxan | chimeric | CD20 |
| secukinumab | Cosentyx | fully human | IL17A |
| siltuximab | Sylvant | chimeric | IL6 |
| tocilizumab | Actemra | humanized | IL6R |
| tocilizumab | Actemra | humanized | IL6R |
| trastuzumab | Herceptin | humanized | HER2 |
| ustekinumab | Stelara | fully human | IL12 |
| ustekinumab | Stelara | fully human | IL12, IL23 |
| vedolizumab | Entyvio | humanized | integrin |
| | | | receptor |
| sarilumab | Kevzara | fully human | IL6R |
| rituximab and | Rituxan | chimeric, co- | CD20 |
| hyaluronidase | Hycela | formulated | |
| guselkumab | Tremfya | fully human | IL23 |
| inotuzumab | Besponsa | humanized, | CD22 |
| ozogamicin | | antibody-drug | |
| | | conjugate | |
| adalimumab-adbm | Cyltezo | fully | TNF |
| | | human, biosimilar | |
| gemtuzumab | Mylotarg | humanized, | CD33 |
| ozogamicin | | antibody-drug | |
| | | conjugate | |
| bevacizumab-awwb | Mvasi | humanized, | VEGF |
| | | biosimilar | |
| benralizumab | Fasenra | humanized | interleukin-5 |
| | | | receptor |
| | | | alpha subunit |
| emicizumab-kxwh | Hemlibra | humanized, | Factor IXa, |
| | | bispecific | Factor X |
| trastuzumab-dkst | Ogivri | humanized, | HER2 |
| | | biosimilar | |
| infliximab-qbtx | Ixifi | chimeric, | TNF |
| | | biosimilar | |
| ibalizumab-uiyk | Trogarzo | humanized | CD4 |
| tildrakizumab-asmn | Ilumya | humanized | IL23 |
| burosumab-twza | Crysvita | fully human | FGF23 |
| erenumab-aooe | Aimovig | fully human | CGRP |
| | | | receptor |

In some embodiments, the method of the disclosure includes contacting tumor tissue sample with a composition including one or more therapeutic antibodies which can be, for example, abagovomab, abatacept, abciximiab, abituzumiab, abrilumab, actoxumiab, adalimumab, adecatumab, aducanumab, aflibercept, afutuzymab, alacizumab, alefacept, alemtuzumab, alirocumab, altumomab, amatixumab, anatumomab, anetumab, anifromumab, anrukinzumab, apolizumab, arcitumomab, ascrinvacumab, aselizumab, atezolizumab, atinumab, altizumab, atorolimumab, bapineuzumab, basiliximab, bavituximab, bectumomab, begelomab, belatacept, belimumab, benralizumab, bertilinmumab, besilesomab, bevacizumab, bezlotoxumab, biciromiab, bimagrumab, bimekizumab, bivatuzumab, blinatumomab, blosozumab, bococizumab, brentuximab, briakimumab, brodalumab, brolucizumab, bronticizumab, canakinumab, cantuzumab, caplacizumab, capromab, carlumab, catumaxomab, cedelizumab, certolizumab, cetixumab, citatuzumab, cixutumumab, clazakizumab, clenoliximab, clivatuzumab, codrituzumab, coltuximab, conatumumab, concizumab, crenezumab, dacetuzumab, daclizumab, dalotuzumab, dapirolizumab, daratumumab, dectrekumab, demcizumab, denintuzumab, denosumab, derlotixumab, detumomab, dinutuximab, diridavumab, dorlinomab, drozitumab, dupilumab, durvalumab, dusigitumab, ecromeximab, eculizumab, edobacomab, edrecolomab, efalizumab, efungumab, eldelumab, elgemtumab, elotuzumab, elsilimomab, emactuzumab, emibetuzumab, enavatuzumab, enfortumab, enlimomab, enoblituzumab, enokizumab, enoticumab, ensituximab, epitumomab, epratuzomab, erlizumab, ertumaxomab, etanercept, etaracizumab, etrolizumab, evinacumab, evolocumab, and exbivirumab.

Additional therapeutic antibodies suitable for the compositions, systems, and methods described herein include, but are not limited to, fanolesomab, faralimomab, farletuzomab, fasimumab, felvizumab, fezkimumab, ficlatuzumab, figitumumab, firivumab, flanvotumab, fletikumab, fontolizumab, foralumab, foravirumab, fresolimumab, fulramumab, futuximab, galiximab, ganitumab, gantenerumab, gavilimomab, gemtuzumab, gevokizumab, girentuximab, glembatumumab, golimumab, gomiliximab, guselkumab, ibalizumab, Iibritumomab, icrucumab, idarucizumab, igovomab, imalumab, imciromab, imgatuzumab, inclacumab, indatuximab, indusatumab, infliximab, intetumumab, inolimomab, inotuzumab, ipilimumab, iratumumab, isatuximab, itolizumab, ixekizumab, keliximab, labetuzumab, lambrolizumab, lampalizumab, lebrikizumab, lemalesomab, lenzilumab, lerdelimumab, lexatumumab, libivirumab, lifastuzumab, ligelizumab, lilotomab, lintuzumab, lirilumab, lodelcizumab, lokivetmab, lorvotuzumab, lucatumumab, lulizumab, lumiliximab, lumretuzumab, mapatumumab, margetuximab, maslimomab, mavrilimumab, matuzumab, mepolizumab, metelimumab, milatuzumab, minetumomab, mirvetuximab, mitumomab, mogamulizumab, morolimumab, motavizumab, moxetumomab, muromonab-CD3, nacolomab, namilumab, naptumomab, narnatumab, natalizumab, nebacumab, necitumumab, nemolizumab, nerelimomab, nesvacumab, nimotuzumab, nivolumab, nofetumomab, obiltoxaximab, obinutuzumab, ocaratuzumab, ocrelizumab, odulimomab, ofatumumab, olaratumab, olokizumab, omalizumab, onartuzumab, ontuxizumab, opicinumab, oportuzumab, oregovomab, orticumab, otelixizumab, oltertuzumab, oxelumab, ozanezumab, ozoralizumab, pagibaximab, palivizumab, panitumumab, pankomab, panobacumab, parsatuzumab, pascolizumab, pasotuxizumab, pateclizumab, patritumab, pembrolizumab, pemtumomab, perakizumab, pertuzumab, pexelizumab, pidilizumab, pinatuzumab, pintumomab, polatuzumab, ponezumab, priliximab, and pritumumab.

Further non-limiting examples of suitable therapeutic antibodies include quilizumab, racotumomab, radretumab, rafivirumab, ralpancizumab, ramucirumab, ranibizumab, raxibacumab, refanezumab, regavirumab, reslizumab, rilonacept, rilotumumab, rinucumab, rituximab, robatumumab, roledumab, romosozumab, rontalizumab, rovelizumab, ruplizumab, sacituzumab, samalizumab, sarilumab, satumomab, secukimumab, seribantumab, setoxaximab, sevirumab, sibrotuzumab, sifalimumab, siltuximab, siplizumab, sirukumab, sofituzumab, solanezumab, solitomab, sonepcizumab, sontuzumab, stamulumab, sulesomab, suvizumab, tabalumab, tacatuzumab, tadocizumab, talizumab, tanezumab, taplitumomab, tarextumab, tefibazumab, telimomab aritox, tenatumomab, teneliximab, teplizumab, tesidolumab, TGN 1412, ticlimumab, tildrakizumab, tigatuzumab, TNX-650, tocilizumab, toralizumab, tosatoxumab, tositumomab, tovetumab, tralokimumab, trastuzumab, TRBS07, tregalizumab, tremelimumab, trevogrumab, tucotuzumab, tuvirumab, ublituximab, ulocuplumab, urelumab, urtoxazumab, ustekimumab, vandortuzumab, vantictumab, vanucizumab, vapaliximab, varlimumab, vatelizumab, vedolizumab, veltuzumab, vepalimomab, vesencumab, visilizumab, volocixumab, vorsetuzumab, votumumab, zalutumimab, zanolimumab, zatuximab, ziralimumab, ziv-aflibercept, and zolimomab.

In some embodiments, the therapeutic antibodies are selected from the group consisting of abciximab, abciximab, adalimumab, aducanumab, alacizumab, alemtuzumab, alirocumab, alirocumab, ascrinvacumab, atezolizumab, atinumab, bapineuzumab, basiliximab, basiliximab, belimumab, bevacizumab, blinatumomab, blosozumab, bococizumab, brentuximab, canakinumab, caplacizumab, capromab, certolizumab, cetuximab, crenezumab, daclizumab, daratumumab, demcizumab, denosumab, denosumab, dinutuximab, ecukinumab, eculizumab, eculizumab, efalizumab, elotuzumab, enoticumab, etaracizumab, evinacumab, evolocumab, evolocumab, fasinumab, fulranumab, gantenerumab, golimumab, ibritumomab, icrucumab, idarucizumab, idarucizumab, inciacumab, infliximab, ipilimumab, mepolizumab, natalizumab, necitumumab, nesvacumab, nivolumab, obinutuzumab, ofatumumab, omalizumab, opicinumab, orticumab, ozanezumab, palivizumab, palivizumab, panitumumab, pembrolizumab, pertuzumab, ponezumab, ralpancizumab, ramucirumab, ramucirumab, ranibizumab, raxibacumab, refanezumab, rinucumab, rituximab, romosozumab, siltuximab, solanezumab, stamulumab, tadocizumab, tanezumab, tocilizumab, trastuzumab, ustekinumab, vedolizumab, and vesencumab.

### Immune-Cell Marker Antibodies

In some embodiments, the methods of the disclosure include contacting the tumor sample with a composition including one or more barcoded immune-cell marker antibodies, e.g., antibodies having specificity for one or more immune cells. For example, the specificity of the recombinant antibodies as described herein for an immune cell can be for a marker expressed on the surface of the immune cell. Examples of immune-cell marker antibodies include, but are not limited to, antibodies having specificity for one or more molecular markers of B cells, T cells, monocytes, macrophages, granulocytes (e.g., basophil, eosinophil, and neutrophil), dendritic cells, natural killer (NK) cells, and/or natural killer T (NKT) cells. For example, exemplary extracellular markers for B cells can include, but are not limited to, CD2, CD5, CD19, CD20, CD21/CD35 (CR2/CR1), CD22, CD23, CD40, CD45R/B220, CD69, CD70, CD74, CD79a (Igα), CD79b (Igo), CD80, CD86, CD93 (C1Rqp), CD137 (4-1BB), CD138 (Syndecan-1), CD252 (OX40L), CD267, CD268 (BAFF-R), CD279 (PD1), HLA-DR, IgG, IgD, and IgM. For T cells, exemplary extracellular markers suitable for the compositions and methods of the disclosure can include, but are not limited to, CD3, CD4, CD8, CD25, CD39, CD43, CD45RO, CD62L, CD73, CD103, CD134, CD152 (CTLA-4), CD194 (CCR4), and CD223. For monocytes, exemplary extracellular markers can include, but are not limited to, CD14 and CD16.

In some embodiments, one or more immune-cell marker antibodies have specificity for a molecular marker of macrophages. Exemplary extracellular markers suitable for the compositions, systems, and methods described herein can include, but are not limited to, CD11a, CD11b, CD11c, CD14, CD15 (SSEA-1), CD16/32, CD33, CD64, CD68, CD80, CD85k (ILT3), CD86, CD105 (Endoglin), CD107b, CD115, CD163, CD195 (CCR5), CD282 (TLR2), and CD284 (TLR4). For basophils, exemplary extracellular markers can include, but are not limited to, CD13, CD44, CD54, CD63, CD69, CD107a, CD123, CD193 (CCR3), CD203c, FcεRIα, IgE, and TLR4.

In some embodiments, one or more immune-cell marker antibodies have specificity for a molecular marker of granulocytes, e.g., eosinophil. Exemplary extracellular markers for eosinophil that are suitable for the compositions and methods of the disclosure can include, but are not limited to, C3AR, CD15 (SSEA-1), CD23, CD49d, CD52, CD53, CD88, CD129, CD183, CD191, CD193, CD244 (2B4), CD294, and CD305. For neutrophils, exemplary extracellular markers can include, but are not limited to, CD10, CD11b, CD11c, CD13, CD14, CD15 (SSEA-1), CD16/32, CD31, CD33, CD62L, CD64, CD66b, CD88, and CD114 (G-CSFR). For myeloid dendritic cells, exemplary extracellular markers can include, but are not limited to, CD1a, CD1b, CD1c, CD4, CD11b, CD11c, CD40, CD49d, CD80, CD83, CD86, CD197 (CCR7), CD205 (DEC-205), CD207 (Langerin), CD209 (DC-SIGN), CD273 (B7-DC, PD-L2), and CD304 (Neuropilin-1).

In some embodiments, one or more immune-cell marker antibodies have specificity for a molecular marker of NK cells. Exemplary extracellular markers for NK cells suitable for the compositions and methods described herein can include, but are not limited to, CD11b, CD11c, CD16/32, CD49b, CD56 (NCAM), CD57, CD69, CD94, CD122, CD158 (Kir), CD161 (NK-1.1), CD244 (2B4), CD314 (NKG2D), CD319 (CRACC), CD328 (Siglec-7), CD335 (NKp46), Ly49, and Ly108. For NKT cells, exemplary extracellular markers can include, but are not limited to, the same markers as for NK cells, as well as CD3 and subunits of invariant TCRα including Vα24 and Jα18 TCR (iNKT). In some embodiments, the specificity of the recombinant antibodies as described herein can be for a molecular marker expressed on a dendritic cell. Non-limiting examples of dendritic cell markers include CD1C, CD8, CD11C, CD24, CD123, CD141, Necl-2, CD11c, HLADR, and BDCA3. Additional dendritic cell markers suitable for the systems and methods disclosed herein can be found in, for example, Villani et al., Science, 21 Apr. 2017: Vol. 356, Issue 6335.

### Tumor-Cell Marker Antibodies

Tumor cell markers that can be used include any marker that is expressed on tumors. In some aspects, the tumor cell markers can be a marker that is expressed more in/on cancerous cells, such as tumors, at a higher level than in/on non-cancerous cells. Exemplary markers include, but are not limited to, ALK, alpha-fetoprotein (AFP), beta-2-microglobulin (B2M), beta-human chorionic gonadotropin (Beta-hCG), bladder tumor antigen (BTA), BRCA1, BRCA2, BCR-ABL fusion gene (Philadelphia chromosome), BRAF V600 mutations, C-kit/CD117, CA15-3/CA27.29, CA-125, CA 27.29, carcinoembryonic antigen (CEA), CD20, CD22, CD25, CD30, CD31, CD33, CD44, CD133, CD176, CD276, estrogen receptor (ER), E-cadherin, ESPR, EGFR, EPCAM, GD2, progesterone receptor (PR), fibrin/fibrinogen, HE4 gene variants, HER2 gene variants, JAK2 gene variants, KRAS gene variants, nuclear matrix protein 22, PCA3, PML/RARα fusion gene, programmed death-ligand 1 (PD-L1 or CD274), prostate-specific antigen (PSA), TEM7, TEM8, and VEGF receptor family members.

### D. Methods for Identifying Tumor-Specific Antibodies

In one aspect, provided herein are methods for identifying a tumor-specific antibody, the methods include: a) providing a tumor tissue sample comprising one or more cells expressing an antigen-binding molecule (ABM); b) attaching an analyte of an ABM-expressing cell of the tumor tissue sample to a capture domain of a first capture probe of a substrate comprising an array of capture probes attached thereto, the first capture probe comprising (i) a spatial barcode sequence and (ii) the capture domain, the capture domain comprising a first capture sequence, wherein the analyte of the ABM-expressing cell comprises a sequence or portion of a sequence encoding the ABM expressed by the ABM-expressing cell or a reverse complement thereof, c) using the analyte of the ABM-expressing cell and the first capture probe attached thereto to generate a spatially barcoded polynucleotide comprising (i) a sequence of first analyte of the ABM-expressing cell or reverse complement thereof and (ii) the spatial barcode sequence or reverse complement thereof and determining all or a part of the nucleic acid sequences of the spatially barcoded polynucleotide; d) using the determined nucleic acid sequences to produce a recombinant antibody; e) coupling the recombinant antibody to a reporter oligonucleotide comprising a reporter barcode sequence to generate a barcoded recombinant antibody; and f) contacting the barcoded recombinant antibody with a second tumor sample (e.g., tumor tissue sample), and identifying the recombinant antibody as an antibody having specificity for the second tumor tissue sample if the barcoded recombinant antibody is capable of binding to an antigen associated with the second tumor sample.

In another aspect, a method for identifying a tumor-specific antibody includes a) providing a first tumor tissue sample comprising one or more cells expressing an antigen-binding molecule (ABM), b) determining all or a part of the nucleic acid sequence encoding the ABM produced by one or more cells of the first tumor tissue sample; and c) using the determined nucleic acid sequences to produce a recombinant antibody, d) coupling the recombinant antibody to a reporter oligonucleotide comprising a reporter barcode sequence to generate a barcoded recombinant antibody; and e) contacting the barcoded recombinant antibody with a second tumor sample (e.g., tumor tissue sample), and identifying the recombinant antibody as an antibody having specificity for the second tumor tissue sample if the barcoded recombinant antibody is capable of binding to an antigen associated with the second tumor sample. In some embodiments, the step of determining all or a part of the nucleic acid sequence encoding the ABM produced by one or more cells of the first tumor tissue sample in step (b) is performed in accordance with a spatial analysis method disclosed herein, including, the exemplary spatial methodologies for immune profiling.

In some embodiments, identifying the produced antibody as an antibody having specificity for the second tumor sample further includes quantifying levels of gene expression and/or protein marker expression in the tumor tissue, which can include the functional characteristics (e.g., the transcriptomic or proteomic) of the B cells and/or tumor cells associated with the recombinant antibodies. These functional characteristics can include transcription of cytokine, chemokine, or cell-surface associated molecules, such as, costimulatory molecules, checkpoint inhibitors, cell surface maturation markers, or cell-adhesion molecules. Such analysis allows a B cell or B-cell population expressing a given antibody to be associated with certain functional characteristics.

In some embodiments, the method further includes using the quantified levels for identification of biomarkers specific for the second tumor tissue sample and/or a subject from whom the second tumor sample is obtained. In some embodiments, the method further includes quantifying binding affinity of the one or more therapeutic antibodies to the tumor tissue, for example by measuring the number of tumor cells that express at least one antigen that binds to the one or more therapeutic antibodies.

In some embodiments, the method further includes using the quantified binding affinity as an indicator of efficacy of treating a tumor with the one or more therapeutic antibodies. In some embodiments, the method further includes using the quantified binding affinity to monitor antigen escape of a tumor from the one or more therapeutic antibodies over time.

In some embodiments, identifying the produced antibody as an antibody having specificity for the second tumor tissue sample further includes comparing the determined nucleic acid sequences encoding the antibody to a genomic DNA sequence from the second tumor tissue sample to confirm antigen specificity of the antibody. In some embodiments, the genomic DNA sequence is obtained from a single cell in the second tumor sample. In some embodiments, the genomic DNA sequence is obtained from a plurality of cells in the second tumor sample. In some embodiments, the genomic DNA sequence is obtained by whole-genome sequencing.

In some embodiments, the identifying of the produced antibody as an antibody having specificity for the second tumor tissue sample further includes comparing the determined nucleic acid sequences encoding the barcoded antibody to a sequence of a ribonucleic acid (RNA) molecule from the second tumor tissue sample to confirm antigen specificity of the antibody.

In some embodiments, the RNA molecule is obtained from a single cell in the second tumor sample. In some embodiments, the RNA molecule is obtained from a plurality of cells in the second tumor sample. In some embodiments, the method further includes obtaining the sequence of the RNA molecule. In some embodiments, the method further includes determining a nucleic acid sequence of a messenger RNA (mRNA) molecule from the single B cell and/or from the single tumor cell.

In some embodiments, the one or more nucleic acid barcode molecules includes one or more barcode sequences and the cDNAs resulting from the reverse transcription step will contain one or more barcode sequences corresponding to the barcode sequences of the nucleic acid barcode molecules.

In some embodiments, the determination of the mRNA sequences and the complementary DNA (cDNA) sequences includes whole transcriptome sequencing (e.g., whole-exome sequencing). In some embodiments, the determination of the mRNA sequences and the complementary DNA (cDNA) sequences includes next-generation sequencing (NGS).

In some embodiments, the method further includes administering the recombinant antibody to a subject in need thereof. In some embodiments, the method further includes administering an immune cell expressing the recombinant antibody to a subject in needed thereof. In some embodiments, the method further includes comparing the determined nucleic acid sequence of the recombinant antibody to sequences of known antibodies in order to identify the antibody as a tumor-specific antibody.

In some embodiments, the method further includes using a filter that takes into account clonal expansions to identify the antibody as a tumor-specific antibody. For example, from a set of antibodies, downselection can be performed by combining 1 or more of the following filters: (1) Retain cells with clonally related sequences observed more than once (filter out antibody lineages only present in a single cell). (2) Retain cells with a given isotype, e.g. IGHG3, which has enhanced complement deposition activity against a target-expressing cell. (3) Retain cells with antibodies that are 5%, 10%, 15%, or 20% mutated from germline (indicates that repeated antigen stimulation/binding has occurred).

In some embodiments, the methods of the disclosure further include using a filter that takes into account gene expression profiles of the B cell within the tissue sample to identify the antibody as a tumor-specific antibody. An exemplary method may comprise classifying B cells within a sample as naïve, transitional memory, class-switched memory, plasmablast, or plasma cell, and filtering for or selecting antibodies only present in memory, class-switched memory, or plasma cells.

In some embodiments, the determined nucleic acid sequence of the recombinant antibody can be used to generate an immune receptor, such as a chimeric antigen receptor (CAR). For example, the determined nucleic acid sequence of the recombinant antibody can be used to generate an antigen-specific receptor, e.g., a receptor that can immunologically recognize and/or specifically bind to an antigen, or an epitope thereof, such that binding of the antigen-specific receptor to antigen, or the epitope thereof, elicits an immune response. In some embodiments, the antigen-specific receptor has antigenic specificity for a cancer antigen, such as a tumor-specific antigen (TSA) or a tumor-associated antigen (TAA).

In some embodiments, the antigen-specific receptor is a chimeric antigen receptor (CAR). Generally, a CAR includes an antigen binding domain, e.g., a single-chain variable fragment (scFv) of an antibody, fused to a transmembrane domain and an intracellular domain. In this case, the antigenic specificity of a CAR can be encoded by a scFv which specifically binds to the antigen, or an epitope thereof. CARs, and methods of making them, are known in the art.

### E. Methods for Characterizing Antibody Specificity or Target Specificity

A particular valuable application of the methods and compositions described herein is in the characterization of antibody specificity and/or target specificity. Accordingly, some embodiments of the disclosure relate to methods for characterizing antibody specificity and/or target specificity. As described in greater detail in Example 1, the methods can include contacting a tumor tissue sample with a substrate comprising an array of capture probes. Analytes (or proxies or intermediates thereof) from an ABM-expressing cell of the tissue sample, as well as reporter oligonucleotides, and optionally reporter oligonucleotides from a panel of additional labeling agents, are attached to capture probes disclosed herein, e.g., according to a spatial analysis method disclosed herein. This is followed by a determination of the nucleic acid sequences encoding V_{H} and V_{L} regions of one or more antibodies produced by the tumor tissue sample. Subsequently, the determined nucleic acid sequences are used to produce recombinant antibodies that are determined above as being expressed in the original tumor tissue sample. In some embodiments, for subsequent characterization and validation of the phenotypic properties of the produced recombinant antibodies, the produced recombinant antibodies are then coupled to a reporter oligonucleotide including a reporter barcode sequence to generate barcoded recombinant antibodies.

In some embodiments, the methods of the disclosure include a step of selecting B-cell derived antibodies suitable for production of recombinant antibodies and optionally for production of barcoded recombinant antibodies. A non-limiting exemplary approach suitable for antibody selection includes the comparative analysis of antibody repertoires between the tumor sample and one or both of (i) normal associated tissue, and (ii) peripheral blood. These comparator populations can be sequenced, for example, by using bulk sequencing to reduce cost and to gain a larger number of sequences. This comparative analysis can enable one to identify antibodies that are enriched within the tumor compared with other compartments. Another non-limiting approach suitable for antibody selection includes quantification of the amount of somatic hypermutation that has occurred within the potential antibody candidates by comparing against either a reference genome or the donor's own germline sequences. Antibodies with the largest amount of somatic hypermutations (SHM) can be selected based on the assumption that they will have already been selected to be high affinity. In addition, antibodies with the least affinity can be selected based on the assumption that they are novel infiltrating cells with new specificity. The above approaches of antibody selection can be employed individually or in combination.

In some embodiments, the barcoded recombinant antibodies generated as described above are then contacted with a second tumor sample, followed by identification of one or more recombinant antibodies having specificity for the second tumor sample, as indicated by the ability of the corresponding barcoded recombinant antibodies to bind to an antigen associated with the second tumor tissue sample.

In some embodiments, gene expression and protein marker expression analyses are additionally performed on (1) the tumor sample from which the B cell is derived, and (2) the tumor sample from which the V_{H} and V_{L} mRNAs are derived. In some embodiments, comparative analysis of the gene expression and protein marker expression datasets from to (1) and (2) is subsequently performed to determine the recombinant antibodies' specificity and target specificity.

### F. Methods for Enhanced Identification of Patient-Specific or Population-Specific Biomarkers

In some embodiments, the methods, compositions and systems disclosed herein are utilized to enhance the identification of patient-specific or population-specific biomarkers on circulating tumor cells. As described in greater detail in Example 2, the methods begin by spatial analysis of ABMs produced by one or more cells in tumor tissue. This is followed, for example, by a determination of the nucleic acid sequences encoding V_{H} and V_{L} regions of one or more antibodies produced by one or more cells in the tumor tissue. Subsequently, the determined nucleic acid sequences are used to produce recombinant antibodies that are determined above as being expressed in the tumor tissue. In some embodiments, for subsequent characterization and validation of the phenotypic properties of the produced recombinant antibodies, the produced recombinant antibodies are then coupled to a reporter oligonucleotide including a reporter barcode sequence to generate barcoded recombinant antibodies.

In some embodiments, the barcoded recombinant antibodies generated as described above are then contacted with a second tumor sample (e.g., tumor tissue sample), followed by identification of one or more recombinant antibodies having specificity for the second tumor sample, as indicated by the ability of the corresponding barcoded recombinant antibodies to bind to an antigen associated with the second tumor tissue sample.

In some embodiments, comparative analysis of in vitro and/or in vivo characterization the barcoded recombinant antibodies as well as gene expression and protein marker expression analysis of a population of tumor samples are subsequently performed to identify biomarkers specific for individual tumor sample or for a population of tumor samples.

G. Methods for Monitoring Antigen Escape in an Individual Who has been Treated with an Antibody-Based Therapy

In another aspect, some embodiments of the disclosure relate to methods for monitoring antigen escape in an individual who has been treated with an antibody-based therapy. As described in greater detail in Example 3, the methods begin by performing spatial analysis of tumor tissue to identify nucleic acid sequences encoding ABMs produced by one or more cells in the tumor tissue. This can involve a determination of the nucleic acid sequences encoding V_{H} and V_{L} regions of one or more antibodies produced by the one or more cells in tumor tissue. Subsequently, the determined nucleic acid sequences are used to produce recombinant antibodies that are determined above as being expressed in the original tumor tissue. In some embodiments, for subsequent characterization and validation of the phenotypic properties of the produced recombinant antibodies, the produced recombinant antibodies are then coupled to a reporter oligonucleotide including a reporter barcode sequence to generate barcoded recombinant antibodies.

In some embodiments, the barcoded recombinant antibodies generated as described above are then contacted with a second tumor tissue sample, followed by identification of one or more recombinant antibodies having specificity for the second tumor tissue sample, as indicated by the ability of the corresponding barcoded recombinant antibodies to bind to a tumor cell of the second tumor sample and/or an antigen associated with the second tumor sample. Tumor cells in the second tumor sample can be identified and/or enriched using antibodies specific for one or more tumor-cell markers, e.g., those expressed more on cancerous cells at a higher level than on non-cancerous cells. Suitable antibodies include, but are not limited to, those specific for ALK, alpha-fetoprotein (AFP), beta-2-microglobulin (B2M), beta-human chorionic gonadotropin (Beta-hCG), bladder tumor antigen (BTA), BRCA1, BRCA2, BCR-ABL fusion gene (Philadelphia chromosome), BRAF V600 mutations, C-kit/CD117, CA15-3/CA27.29, CA-125, CA 27.29, carcinoembryonic antigen (CEA), CD20, CD22, CD25, CD30, CD31, CD33, CD44, CD133, CD176, CD276, estrogen receptor (ER), E-cadherin, ESPR, EGFR, EPCAM, GD2, progesterone receptor (PR), fibrin/fibrinogen, HE4 gene variants, HER2 gene variants, JAK2 gene variants, KRAS gene variants, nuclear matrix protein 22, PCA3, PML/RARα fusion gene, programmed death-ligand 1 (PD-L1 or CD274), prostate-specific antigen (PSA), TEM7, TEM8, and VEGF receptor family members.

In some embodiments, tumor cells in the second tumor sample can be identified by using unbiased genome-wide sequence analysis or whole transcriptome gene expression profiling of the cells for cancer-related mRNAs. In some embodiments, tumor cells in the second tumor sample can be identified using targeted gene expression profiling of the cells for cancer-related mRNAs. In some embodiments, whole transcriptome libraries are selectively enriched for cancer-related transcripts and the enriched libraries subjected to sequencing. Approaches, systems, and kits suitable for use in targeted characterization and enrichment of cancer-related transcripts are known in the art and/or commercially available. For example, in some embodiments, whole transcriptome libraries can be selectively enriched for cancer-related transcripts by using 10× Genomics Human Pan-Caner Panel kit (Cat #PN-1000247 and PN-1000260) with reagents for use in targeted gene expression analysis of >1,200 cancer-related biomarkers to identify, characterize, enrich, and/or profile a pre-designed set of transcripts for a target cancer of interest.

In some embodiments, the binding affinity of the barcoded recombinant antibody to a tumor sample is subsequently evaluated by measuring the number of tumor cells expressing a target antigen of the barcoded recombinant antibody that are capable to binding to the barcoded recombinant antibody. In these experiments, the quantified binding affinity of the barcoded recombinant antibody to the tumor sample is indicative of the therapeutic antibody's efficacy in treating the tumor.

### Temporal Analysis

In some embodiments, the binding affinity of the barcoded recombinant antibody to an antigen expressed by the tumor sample is monitored over time, and is used as an indication of antigen escape from the recombinant antibody over time (e.g., before or after treatment with a therapeutic agent or different stages of differentiation). In some examples, the methods described herein can be performed on multiple similar tumor tissue samples obtained from the same subject at a different time points (e.g., before or after treatment with a therapeutic agent, different stages of differentiation, different stages of disease progression, different ages of the subject, or before or after development of resistance to a therapeutic agent).

In some embodiments, the methods described herein can be performed on multiple similar tumor tissue samples obtained from the subject at 2, 3, 4, 5, 6, 7, 8, 9, 10, or more times. For example, the multiple similar tumor tissue samples can be repetitive samples from the same subject and the same tissue, In some embodiments, the same tumor tissue sample is contacted with different barcoded recombinant antibodies at each time point. In some embodiments, samples can be obtained from the same subject as a routine monitoring on a monthly basis, or at the shortest time interval 10-14 days. This timeline can be used when monitoring for de novo immune responses against the developed antibody therapeutics. Monthly monitoring can also be used for circulating tumor cell content and circulating tumor DNA (ctDNA).

### H. Methods for Characterizing a Potential Antigen

In another aspect, some embodiments of the disclosure relate to methods for characterizing a potential antigen for an antibody or fragment thereof. As described in greater detail in Example 4, the methods can include performing spatial analysis of ABMs produced by one or more cells in tumor tissue. This is followed by a determination of the nucleic acid sequences encoding V_{H} and V_{L} regions of one or more antibodies produced by the one or more cells in the tumor tissue. Subsequently, the determined nucleic acid sequences are used to produce a recombinant antibody that is determined as being expressed in the tumor tissue. In some embodiments, for subsequent characterization and validation of the phenotypic properties of the produced recombinant antibodies, one or more produced recombinant antibodies obtained from the first tumor tissue sample are then coupled to a reporter oligonucleotide including a reporter barcode sequence to generate a set of one or more barcoded recombinant antibodies. In some embodiments, variants, e.g., mutants, of the individual antibodies can also be used as part of the set to identify paratopes/residues on the antibody required for antigen recognition. Vice versa, in some embodiments, variants of the target proteins used for epitopes/recognized portions of the antigen.

In some embodiments, the binding affinity of the barcoded recombinant antibodies to a second tumor sample is subsequently evaluated by measuring the number of tumor cells expressing a target antigen of the barcoded recombinant antibodies that are capable to binding to the barcoded recombinant antibodies, followed by using the quantified binding affinity to determine if the recombinant antibodies compete with one another for binding to the second tumor sample. In this case, the second tumor sample or cells of the second tumor are known to express a particular target antigen of interest, or else the recombinant antibody from the B cell above is thought to bind to a particular target antigen.

In some embodiments, barcoded recombinant antibodies from the set are indicated as competing for binding to an antigen if it is determined that they bind to different cells in the second tumor sample in a mutually exclusive manner. For example, competitive binding assays can be perform to identify mutually exclusive detection of antibodies that bind to different cells in a tumor population. In addition or alternatively, binding assays can be performed on cell replicates with differing concentrations of barcoded antibodies and detect tighter binding of the antibodies versus each other. In some embodiments, dose response curves, wherein cells of the second tumor sample are contacted with varying concentrations one or more barcoded recombinant antibodies of the set, are used to evaluate whether the barcoded recombinant antibodies of the set compete for binding. For example, in dose response curve varying the concentrations of both antibodies, if increasing dose of one antibody results in less binding to the other, this may indicate a level of competition between the two tested antibodies. In some embodiments, the quantified binding affinity of the recombinant antibodies are also co-associated with RNA expression analysis to identify potential antigen.

I. Methods of treatment

In some embodiments, the methods of the disclosure further include administering a therapeutic composition including a recombinant antibody as described herein and/or an immune system cell expressing the recombinant antibody as described herein to a subject in need thereof. Non-limiting examples of immune system cells include B cells, monocytes, NK cells, natural killer T (NKT) cells, basophil, eosinophil, neutrophil, dendritic cells, macrophages, regulatory T cells, helper T cells (T_{H}), cytotoxic T cells (T_{CTL}), memory T cells, gamma delta (γδ) T cells, hematopoietic stem cells, and hematopoietic stem cell progenitors. In some embodiments, the immune system cell is a T cell. In some embodiments, the methods of the disclosure further include administering a therapeutic composition including one or more CAR T cells. In some embodiments, the therapeutic composition is formulated to be compatible with its intended route of administration. For example, the recombinant antibodies of the disclosure may be given orally or by inhalation, but it is more likely that they will be administered through a parenteral route. Examples of parenteral routes of administration include, for example, intravenous, intradermal, subcutaneous, transdermal (topical), transmucosal, and rectal administration. Solutions or suspensions used for parenteral application can include the following components: a sterile diluent such as water for injection, saline solution, fixed oils, polyethylene glycols, glycerine, propylene glycol or other synthetic solvents; antibacterial agents such as benzyl alcohol or methyl parabens; antioxidants such as ascorbic acid or sodium bisulfite; chelating agents such as ethylenediaminetetraacetic acid (EDTA); buffers such as acetates, citrates or phosphates and agents for the adjustment of tonicity such as sodium chloride or dextrose. pH can be adjusted with acids or bases, such as mono- and/or di-basic sodium phosphate, hydrochloric acid or sodium hydroxide (e.g., to a pH of about 7.2-7.8, e.g., 7.5). The parenteral preparation can be enclosed in ampoules, disposable syringes or multiple dose vials made of glass or plastic.

Dosage, toxicity and therapeutic efficacy of such subject recombinant antibodies of the disclosure can be determined by standard pharmaceutical procedures in cell cultures or experimental animals, e.g., for determining the LD50 (the dose lethal to 50% of the population) and the ED₅₀ (the dose therapeutically effective in 50% of the population). The dose ratio between toxic and therapeutic effects is the therapeutic index and it can be expressed as the ratio LD₅₀/ED₅₀. Compounds that exhibit high therapeutic indices are generally suitable. While compounds that exhibit toxic side effects may be used, care should be taken to design a delivery system that targets such compounds to the site of affected tissue in order to minimize potential damage to uninfected cells and, thereby, reduce side effects.

For example, the data obtained from the cell culture assays and animal studies can be used in formulating a range of dosage for use in humans. The dosage of such compounds lies generally within a range of circulating concentrations that include the EDso with little or no toxicity. The dosage may vary within this range depending upon the dosage form employed and the route of administration utilized. For any compound used in the method of the disclosure, the therapeutically effective dose can be estimated initially from cell culture assays. A dose may be formulated in animal models to achieve a circulating plasma concentration range that includes the IC₅₀ (e.g., the concentration of the test compound which achieves a half-maximal inhibition of symptoms) as determined in cell culture. Such information can be used to more accurately determine useful doses in humans. Levels in plasma may be measured, for example, by high performance liquid chromatography.

### Systems and Methods for Spatial Analysis

Exemplary methods disclosed herein comprising providing a biological sample, e.g., a tissue sample comprising one or more cells expressing an ABM. In some embodiments, the biological sample is a tissue sample. In some embodiments, the biological sample is a healthy or diseased tissue sample. In some embodiments, the tissue sample is a tissue section. The tissue section can be a fresh frozen tissue section, a fixed tissue section, or an FFPE tissue section. In some embodiments, the tissue sample is fixed and/or stained (e.g., a fixed and/or stained tissue section). Non-limiting examples of stains include histological stains (e.g., hematoxylin and/or eosin) and immunological stains (e.g., fluorescent stains). In some embodiments, the biological sample (e.g., tissue sample) is a tissue microarray (TMA). A tissue microarray contains multiple representative tissue samples—which can be from different tissues or organisms—assembled on a single histologic slide. The TMA can therefore allow for high throughput analysis of multiple specimens at the same time. Tissue microarrays are paraffin blocks produced by extracting cylindrical tissue cores from different paraffin donor blocks and re-embedding these into a single recipient (microarray) block at defined array coordinates.

In some embodiments, a biological sample (e.g., a fixed and/or stained biological sample) can be imaged. Suitable tissue samples are also described in Section (I)(d) of WO 2020/176788 and/or U.S. Patent Application Publication No. 2020/0277663, which are hereby incorporated by reference in their entireties. In some embodiments, the tissue sample is subjected to spatial analysis. Systems and methods for spatial analysis are disclosed herein.

Non-limiting aspects of spatial analysis methodologies and compositions are described in U.S. Pat. Nos. 10,774,374, 10,724,078, 10,480,022, 10,059,990, 10,041,949, 10,002,316, 9,879,313, 9,783,841, 9,727,810, 9,593,365, 8,951,726, 8,604,182, 7,709,198, U.S. Patent Application Publication Nos. 2020/239946, 2020/080136, 2020/0277663, 2020/024641, 2019/330617, 2019/264268, 2020/256867, 2020/224244, 2019/194709, 2019/161796, 2019/085383, 2019/055594, 2018/216161, 2018/051322, 2018/0245142, 2017/241911, 2017/089811, 2017/067096, 2017/029875, 2017/0016053, 2016/108458, 2013/171621, WO 2018/091676, WO 2020/176788, Rodriques et al., Science 363(6434):1463-1467, 2019; Lee et al., Nat. Protoc. 10(3):442-458, 2015; Trejo et al., PLOS ONE 14(2):e0212031, 2019; Chen et al., Science 348(6233):aaa6090, 2015; Gao et al., BMC Biol. 15:50, 2017; and Gupta et al., Nature Biotechnol. 36:1197-1202, 2018; the Visium Spatial Gene Expression Reagent Kits User Guide (e.g., Rev D, dated October 2020), the Visium Spatial Gene Expression for FFPE User Guide (e.g., Rev A, dated June 2021), and/or the Visium Spatial Tissue Optimization Reagent Kits User Guide (e.g., Rev D, dated October 2020), both of which are available at the 10× Genomics Support Documentation website, and can be used herein in any combination. Some exemplary spatial analysis workflows are described in the Exemplary Embodiments section of WO 2020/176788 and/or U.S. Patent Application Publication No. 2020/0277663. See, for example, the Exemplary embodiment starting with “In some non-limiting examples of the workflows described herein, the sample can be immersed . . . ” of WO 2020/176788 and/or U.S. Patent Application Publication No. 2020/0277663. Further non-limiting aspects of spatial analysis methodologies and compositions are described herein.

Analytes can be broadly classified into one of two groups: nucleic acid analytes, and non-nucleic acid analytes. Examples of non-nucleic acid analytes include, but are not limited to, lipids, carbohydrates, peptides, proteins, glycoproteins (N-linked or O-linked), lipoproteins, phosphoproteins, specific phosphorylated or acetylated variants of proteins, amidation variants of proteins, hydroxylation variants of proteins, methylation variants of proteins, ubiquitylation variants of proteins, sulfation variants of proteins, viral proteins (e.g., viral capsid, viral envelope, viral coat, viral accessory, viral glycoproteins, viral spike, etc.), extracellular and intracellular proteins, antibodies, and antigen binding fragments. In some embodiments, the analyte(s) can be localized to subcellular location(s), including, for example, organelles, e.g., mitochondria, Golgi apparatus, endoplasmic reticulum, chloroplasts, endocytic vesicles, exocytic vesicles, vacuoles, lysosomes, etc. In some embodiments, analyte(s) can be peptides or proteins, including without limitation antibodies and enzymes. Examples of nucleic acid analytes include, but are not limited to, DNA (e.g., genomic DNA, cDNA) and RNA, including coding and non-coding RNA (e.g., mRNA, rRNA, tRNA, ncRNA). Additional examples of analytes can be found in Section (I)(c) of PCT Publication No. WO2020/176788 and/or U.S. Patent Application Publication No. 2020/0277663. In some embodiments, an analyte can be detected indirectly, such as through detection of an intermediate agent, for example, a ligation product or an analyte capture agent (e.g., an oligonucleotide-conjugated antibody), such as those described herein.

The biological sample as used herein can be any suitable biological sample described herein or known in the art. In some embodiments, the biological sample is a tissue sample. In some embodiments, the tissue sample is a solid tissue sample. In some embodiments, the biological sample is a tissue section (e.g., a fixed tissue section). In some embodiments, the tissue is flash-frozen and sectioned. Any suitable method described herein or known in the art can be used to flash-freeze and section the tissue sample. In some embodiments, the biological sample, e.g., the tissue, is flash-frozen using liquid nitrogen before sectioning.

In some embodiments, the biological sample, e.g., a tissue sample, is flash-frozen using nitrogen (e.g., liquid nitrogen), isopentane, or hexane. In some embodiments, the biological sample, e.g., the tissue, is embedded in a matrix e.g., optimal cutting temperature (OCT) compound to facilitate sectioning. OCT compound is a formulation of clear, water-soluble glycols and resins, providing a solid matrix to encapsulate biological (e.g., tissue) specimens. In some embodiments, the sectioning is performed using cryosectioning.

Subjects from which biological samples can be obtained can be healthy or asymptomatic individuals, individuals that have or are suspected of having a disease (e.g., cancer) or a pre-disposition to a disease, and/or individuals that are in need of therapy or suspected of needing therapy. Biological samples can include one or more diseased cells. A diseased cell can have altered metabolic properties, gene expression, protein expression, and/or morphologic features. Examples of diseases include inflammatory disorders, metabolic disorders, nervous system disorders, and cancer. Cancer cells can be derived from solid tumors, hematological malignancies, cell lines, or obtained as circulating tumor cells.

In some embodiments, the biological sample, e.g., the tissue sample, is fixed in a fixative including alcohol, for example methanol. In some embodiments, instead of methanol, acetone, or an acetone-methanol mixture can be used. In some embodiments, the biological sample, e.g., the tissue sample, is fixed e.g., immediately after being harvested from a subject. In such embodiments, the fixative is preferably an aldehyde fixative, such as paraformaldehyde (PFA) or formalin. In some embodiments, the fixative induces crosslinks within the biological sample. In some embodiments, the biological sample can be fixed using PAXgene.

In some embodiments, a biological sample is permeabilized with one or more permeabilization reagents. For example, permeabilization of a biological sample can facilitate analyte capture. Exemplary permeabilization agents and conditions are described in Section (I)(d)(ii)(13) or the Exemplary Embodiments Section of WO 2020/176788 and/or U.S. Patent Application Publication No. 2020/0277663.

Array-based spatial analysis methods can involve the transfer of one or more analytes from a biological sample to an array of features on a substrate, where each feature is associated with a unique spatial location on the array. Subsequent analysis of the transferred analytes includes determining the identity of the analytes and the spatial location of the analytes within the biological sample. The spatial location of an analyte within the biological sample is determined based on the feature to which the analyte is bound (e.g., directly or indirectly) on the array, and the feature's relative spatial location within the array.

A “capture probe” refers to any molecule capable of capturing (directly or indirectly) and/or labelling an analyte (e.g., an analyte of interest) in a biological sample. In some embodiments, the capture probe is a nucleic acid or a polypeptide. In some embodiments, the capture probe includes a barcode (e.g., a spatial barcode and/or a unique molecular identifier (UMI)) and a capture domain). In some embodiments, a capture probe can include a cleavage domain and/or a functional domain (e.g., a primer-binding site, such as for next-generation sequencing (NGS)).

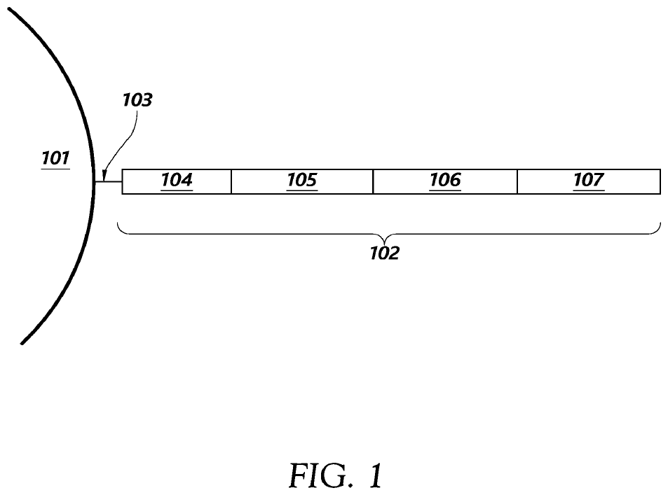

FIG. 1 is a schematic diagram showing an exemplary capture probe, as described herein. As shown, the capture probe 102 is optionally coupled to a feature 101 by a cleavage domain 103, such as a disulfide linker. The capture probe can include a functional sequence 104 (also referred to herein as “adapter” or “adaptor”) that is useful for subsequent processing. The functional sequence 104 can include all or a part of sequencer specific flow cell attachment sequence (e.g., a P5 or P7 sequence), all or a part of a sequencing primer sequence, (e.g., a R1 primer binding site, a R2 primer binding site), or combinations thereof. The capture probe can also include a spatial barcode 105. The capture probe can also include a unique molecular identifier (UMI) sequence 106. While FIG. 1 shows the spatial barcode 105 as being located upstream (5′) of UMI sequence 106, it is to be understood that capture probes wherein UMI sequence 106 is located upstream (5′) of the spatial barcode 105 is also suitable for use in any of the methods described herein. The capture probe can also include a capture domain 107 to facilitate capture of a target analyte. The capture domain can have a sequence complementary to a sequence of a nucleic acid analyte. The capture domain can have a sequence complementary to a connected probe described herein. The capture domain can have a sequence complementary to a capture handle sequence present in an analyte capture agent. The capture domain can have a sequence complementary to a splint oligonucleotide. Such splint oligonucleotide, in addition to having a sequence complementary to a capture domain of a capture probe, can have a sequence of a nucleic acid analyte, a sequence complementary to a portion of a connected probe described herein, and/or a capture handle sequence described herein.

The functional sequences can generally be selected for compatibility with any of a variety of different sequencing systems, e.g., Ion Torrent Proton or PGM, Illumina sequencing instruments, PacBio, Oxford Nanopore, etc., and the requirements thereof. In some embodiments, functional sequences can be selected for compatibility with non-commercialized sequencing systems. Examples of such sequencing systems and techniques, for which suitable functional sequences can be used, include (but are not limited to) Ion Torrent Proton or PGM sequencing, Illumina sequencing, PacBio SMRT sequencing, and Oxford Nanopore sequencing. Further, in some embodiments, functional sequences can be selected for compatibility with other sequencing systems, including non-commercialized sequencing systems.

In some embodiments, the spatial barcode 105 and functional sequences 104 are common to all of the probes attached to a given feature. In some embodiments, the UMI sequence 106 of a capture probe attached to a given feature is different from the UMI sequence of a different capture probe attached to the given feature.

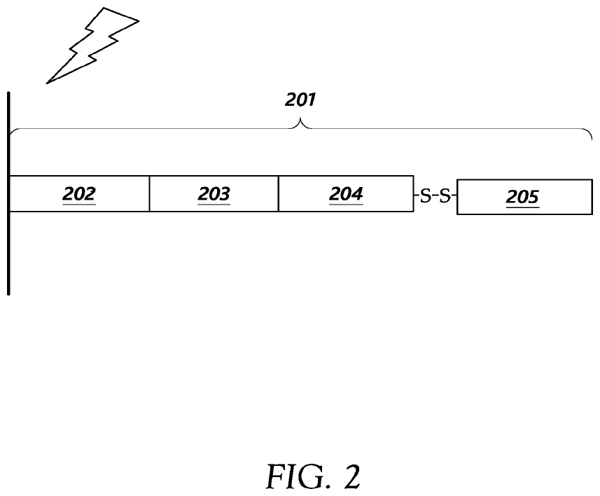

FIG. 2 is a schematic illustrating a cleavable capture probe, wherein the cleaved capture probe can enter into a non-permeabilized cell and bind to analytes within the sample. The capture probe 201 contains a cleavage domain 202, a cell penetrating peptide 203, a reporter molecule 204, and a disulfide bond (—S—S—). 205 represents all other parts of a capture probe, for example a spatial barcode and a capture domain.

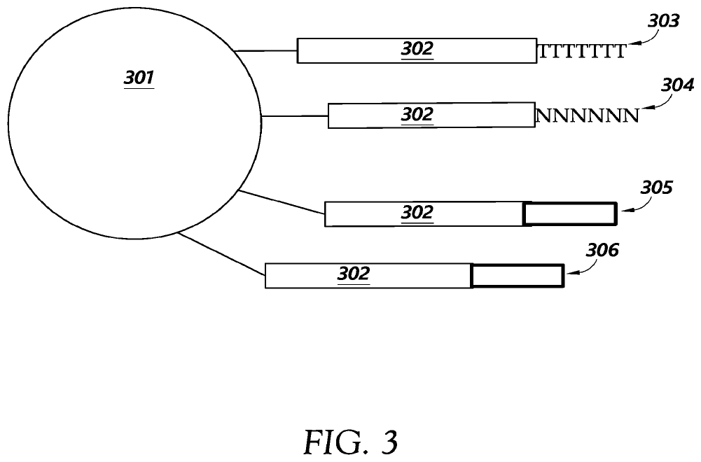

FIG. 3 is a schematic diagram of an exemplary multiplexed spatially-barcoded feature. In FIG. 3, the feature 301 can be coupled to spatially-barcoded capture probes, wherein the spatially-barcoded probes of a particular feature can possess the same spatial barcode, but have different capture domains designed to associate the spatial barcode of the feature with more than one target analyte. For example, a feature may be coupled to four different types of spatially-barcoded capture probes, each type of spatially-barcoded capture probe possessing the spatial barcode 302. One type of capture probe associated with the feature includes the spatial barcode 302 in combination with a poly(T) capture domain 303, designed to capture mRNA target analytes. A second type of capture probe associated with the feature includes the spatial barcode 302 in combination with a random N-mer capture domain 304 for gDNA analysis. A third type of capture probe associated with the feature includes the spatial barcode 302 in combination with a capture domain complementary to a capture handle sequence of an analyte capture agent of interest 305. A fourth type of capture probe associated with the feature includes the spatial barcode 302 in combination with a capture domain that can specifically bind a nucleic acid molecule 306 that can function in a CRISPR assay (e.g., CRISPR/Cas9).

In some embodiments, a feature can be coupled to (i) a first capture probe comprising spatial barcode sequence 302 and a first capture domain comprising a first capture sequence. In some embodiments, the feature can be coupled to (ii) a second capture probe comprising the same spatial barcode sequence 302 and a second capture domain comprising a second capture sequence. In some embodiments, the first capture sequence of the first capture domain and the second capture sequence of the second capture domain are identical. In some embodiments, the first capture sequence of the first capture domain and the second capture sequence of the second capture domain are different. In some embodiments, the first capture sequence of the first capture domain is a homopolymeric sequence. In some embodiments, the first capture sequence of the first capture domain is a defined non-homopolymeric sequence. In some embodiments, the defined non-homopolymeric sequence is a sequence that binds to the first analyte. In some embodiments, the defined non-homopolymeric sequence specifically binds to a nucleic acid sequence encoding a region of an ABM. Wherein the ABM is selected from: a TCR alpha chain, a TCR beta chain, a TCR gamma chain, a TCR delta chain an immunoglobulin kappa light chain, an immunoglobulin lambda light chain, an immunoglobulin heavy chain. In some embodiments, the region of the ABM is a constant region of the ABM or a variable region of the ABM. In some embodiments, the second capture sequence of the second capture domain is a homopolymeric sequence. In some embodiments, the second capture sequence of the second capture domain is a defined non-homopolymeric sequence. In some embodiments, the homopolymeric sequence is a polyT sequence.

While four different capture probe-barcoded constructs are shown in FIG. 3, capture-probe barcoded constructs can be tailored for analyses of any given analyte associated with a nucleic acid and capable of binding with such a construct. For example, the schemes shown in FIG. 3 can also be used for concurrent analysis of other analytes disclosed herein, including, but not limited to: (a) mRNA, a lineage tracing construct, cell surface or intracellular proteins and metabolites, and gDNA; (b) mRNA, accessible chromatin (e.g., ATAC-seq, DNase-seq, and/or MNase-seq) cell surface or intracellular proteins and metabolites, and a perturbation agent (e.g., a CRTSPR crRNA/sgRNA, TALEN, zinc finger nuclease, and/or antisense oligonucleotide as described herein); (c) mRNA, cell surface or intracellular proteins and/or metabolites, a barcoded labelling agent (e.g., the MHC multimers described herein), and a V(D)J sequence of an immune cell receptor (e.g., T-cell receptor). In some embodiments, a perturbation agent can be a small molecule, an antibody, a drug, an aptamer, a miRNA, a physical environmental (e.g., temperature change), or any other known perturbation agents. See, e.g., Section (II)(b) (e.g., subsections (i)-(vi)) of WO 2020/176788 and/or U.S. Patent Application Publication No. 2020/0277663. Generation of capture probes can be achieved by any appropriate method, including those described in Section (II)(dxii) of WO 2020/176788 and/or U.S. Patent Application Publication No. 2020/0277663.

In some embodiments, more than one analyte type (e.g., nucleic acids and proteins) from a biological sample can be detected (e.g., simultaneously or sequentially) using any appropriate multiplexing technique, such as those described in Section (IV) of WO 2020/176788 and/or U.S. Patent Application Publication No. 2020/0277663.

In some embodiments, detection of one or more analytes (e.g., protein analytes) can be performed using one or more analyte capture agents. As used herein, an “analyte capture agent” (also referred to herein as “labeling agent”) refers to an agent that interacts with an analyte (e.g., an analyte in a biological sample) and with a capture probe (e.g., a capture probe attached to a substrate or a feature) to identify the analyte. In some embodiments, the analyte capture agent includes: (i) an analyte binding moiety (e.g., that binds to an analyte), for example, an antibody or antigen-binding fragment thereof; (ii) analyte binding moiety barcode; and (iii) a capture handle sequence. As used herein, the term “analyte binding moiety barcode” refers to a barcode that is associated with or otherwise identifies the analyte binding moiety. As used herein, the term “analyte capture sequence” or “capture handle sequence” refers to a region or moiety configured to hybridize to, bind to, couple to, or otherwise interact with a capture domain of a capture probe. In some embodiments, a capture handle sequence is complementary to a capture domain of a capture probe. In some cases, an analyte binding moiety barcode (or portion thereof) may be able to be removed (e.g., cleaved) from the analyte capture agent.

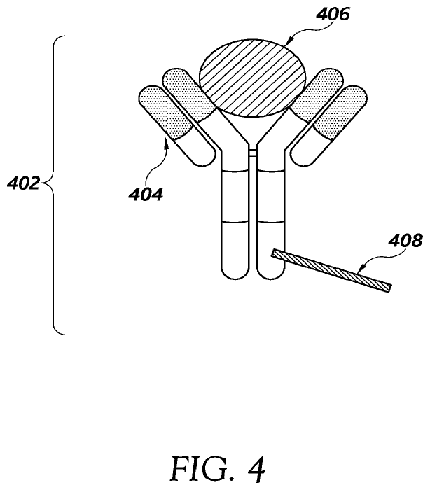

FIG. 4 is a schematic diagram of an exemplary analyte capture agent 402 comprised of an analyte-binding moiety 404 and an analyte-binding moiety barcode domain 408. The exemplary analyte-binding moiety 404 is a molecule capable of binding to an analyte 406 and the analyte capture agent is capable of interacting with a spatially-barcoded capture probe. The analyte-binding moiety can bind to the analyte 406 with high affinity and/or with high specificity. The analyte capture agent can include an analyte-binding moiety barcode domain 408, a nucleotide sequence (e.g., an oligonucleotide), which can hybridize to at least a portion or an entirety of a capture domain of a capture probe. It should be noted that a reporter oligonucleotide, can be considered an analyte-binding moiety barcode domain 408. The analyte-binding moiety barcode domain 408 (also referred to herein as a reporter oligonucleotide) can comprise an analyte binding moiety barcode (also referred to herein as a “reporter barcode sequence”) and a capture handle sequence described herein. The analyte-binding moiety 404 can include a polypeptide and/or an aptamer. The analyte-binding moiety 404 can include an antibody or antibody fragment (e.g., an antigen-binding fragment).

In some embodiments, the analyte capture agent includes a capture agent barcode domain that is conjugated or otherwise attached to the analyte binding moiety. In some embodiments, the capture agent barcode domain is covalently-linked to the analyte binding moiety. In some embodiments, a capture agent barcode domain is a nucleic acid sequence. In some embodiments, a capture agent barcode domain includes an analyte binding moiety barcode and an analyte capture sequence.

As used herein, the term “analyte binding moiety barcode” refers to a barcode that is associated with or otherwise identifies the analyte binding moiety. In some embodiments, by identifying an analyte binding moiety and its associated analyte binding moiety barcode, the analyte to which the analyte binding moiety binds can also be identified. An analyte binding moiety barcode can be a nucleic acid sequence of a given length and/or sequence that is associated with the analyte binding moiety. An analyte binding moiety barcode can generally include any of the variety of aspects of barcodes described herein. For example, an analyte capture agent that is specific to one type of analyte can have coupled thereto a first capture agent barcode domain (e.g., that includes a first analyte binding moiety barcode), while an analyte capture agent that is specific to a different analyte can have a different capture agent barcode domain (e.g., that includes a second barcode analyte binding moiety barcode) coupled thereto. In some aspects, such a capture agent barcode domain can include an analyte binding moiety barcode that permits identification of the analyte binding moiety to which the capture agent barcode domain is coupled. The selection of the capture agent barcode domain can allow significant diversity in terms of sequence, while also being readily attachable to most analyte binding moieties (e.g., antibodies or aptamers) as well as being readily detected, (e.g., using sequencing or array technologies).

In some embodiments, the capture agent barcode domain of an analyte capture agent includes an analyte capture sequence. As used herein, the term “analyte capture sequence” and “capture handle sequence” may be used interchangeably herein to refer to a region or moiety configured to hybridize to, bind to, couple to, or otherwise interact with a capture domain of a capture probe. In some embodiments, a capture handle sequence includes a nucleic acid sequence that is complementary to or substantially complementary to a capture sequence of the capture domain of a capture probe such that the capture handle sequence hybridizes to the capture domain of the capture probe. In some embodiments, a capture handle sequence comprises a poly(A) nucleic acid sequence that hybridizes to a capture domain that comprises a poly(T) nucleic acid sequence. In some embodiments, a capture handle sequence comprises a poly(T) nucleic acid sequence that hybridizes to a capture domain that comprises a poly(A) nucleic acid sequence. In some embodiments, a capture handle sequence comprises a non-homopolymeric nucleic acid sequence that hybridizes to a capture domain that comprises a non-homopolymeric nucleic acid sequence that is complementary (or substantially complementary) to the non-homopolymeric nucleic acid sequence of the analyte capture region.

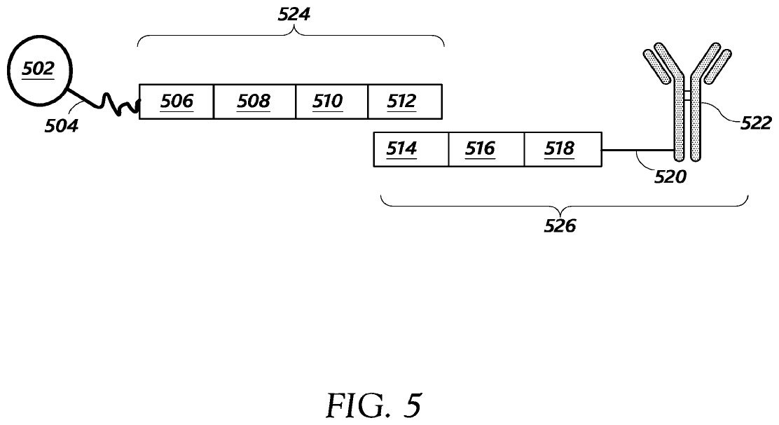

FIG. 5 is a schematic diagram depicting an exemplary interaction between a feature-immobilized capture probe 524 and an analyte capture agent 526 (also referred to herein as a labeling agent). The feature-immobilized capture probe 524 can include a spatial barcode 508 as well as functional sequences 506 and UMI 510, as described elsewhere herein. The capture probe can also include a capture domain 512 that is capable of binding to an analyte capture agent 526. The analyte capture agent 526 can include a functional sequence 518, analyte binding moiety barcode 516 (also referred to herein as a reporter barcode sequence), and a capture handle sequence 514 that is capable of binding to the capture domain 512 of the capture probe 524. For example, the capture handle sequence 514 may be complementary to a capture sequence of the capture domain 512. The analyte capture agent can also include a linker 520 that allows the capture agent barcode domain 516 to couple to the analyte binding moiety 522.

Additional description of analyte capture agents can be found in Section (II)(b)(ix) of WO 2020/176788 and/or Section (II)(b)(viii) U.S. Patent Application Publication No. 2020/0277663.

There are at least two methods to associate a spatial barcode with one or more neighboring cells, such that the spatial barcode identifies the one or more cells, and/or contents of the one or more cells, as associated with a particular spatial location. One method is to promote analytes or analyte proxies (e.g., intermediate agents) from a cell towards a spatially-barcoded array (e.g., including spatially-barcoded capture probes). Another method is to cleave spatially-barcoded capture probes from an array and promote the spatially-barcoded capture probes towards and/or into or onto the biological sample.

In some cases, capture probes may be configured to prime, replicate, and consequently yield optionally barcoded extension products from a template (e.g., a DNA or RNA template, such as an analyte or an intermediate agent (e.g., a connected probe (e.g., a ligation product) or an analyte capture agent), or a portion thereof), or derivatives thereof (see, e.g., Section (II)(b)(vii) of WO 2020/176788 and/or U.S. Patent Application Publication No. 2020/0277663 regarding extended capture probes). In some cases, capture probes may be configured to form a connected probe (e.g., a ligation product) with a template (e.g., a DNA or RNA template, such as an analyte or an intermediate agent, or portion thereof), thereby creating ligations products that serve as proxies for a template. As used herein, an “extended capture probe” refers to a capture probe having additional nucleotides added to the terminus (e.g., 3′ or 5′ end) of the capture probe thereby extending the overall length of the capture probe. For example, an “extended 3′ end” indicates additional nucleotides were added to the most 3′ nucleotide of the capture probe to extend the length of the capture probe, for example, by polymerization reactions used to extend nucleic acid molecules including templated polymerization catalyzed by a polymerase (e.g., a DNA polymerase or a reverse transcriptase). In some embodiments, extending the capture probe includes adding to a 3′ end of a capture probe a nucleic acid sequence that is complementary to a nucleic acid sequence of an analyte or intermediate agent specifically bound to the capture domain of the capture probe. In some embodiments, the capture probe is extended using reverse transcription. In some embodiments, the capture probe is extended using one or more DNA polymerases. The extended capture probes include the sequence of the capture probe and the sequence of the spatial barcode of the capture probe. In some embodiments, extended capture probes are amplified (e.g., in bulk solution or on the array) to yield quantities that are sufficient for downstream analysis, e.g., for determining sequences of one or more analytes, e.g., via sequencing. In some embodiments, extended capture probes (e.g., DNA molecules) act as templates for an amplification reaction (e.g., a polymerase chain reaction). Additional variants of spatial analysis methods, including in some embodiments, an imaging step, are described in Section (II)(a) of WO 2020/176788 and/or U.S. Patent Application Publication No. 2020/0277663. Analysis of captured analytes (and/or intermediate agents or portions thereof), for example, including sample removal, extension of capture probes, sequencing (e.g., of a cleaved extended capture probe and/or a cDNA molecule complementary to an extended capture probe), sequencing on the array (e.g., using, for example, in situ hybridization or in situ ligation approaches), temporal analysis, and/or proximity capture, is described in Section (II)(g) of WO 2020/176788 and/or U.S. Patent Application Publication No. 2020/0277663. Some quality control measures are described in Section (II)(h) of WO 2020/176788 and/or U.S. Patent Application Publication No. 2020/0277663.

For spatial array-based methods, a substrate may function as a support for direct or indirect attachment of capture probes to features of the array. A “feature” is an entity that acts as a support or repository for various molecular entities used in spatial analysis. In some embodiments, some or all of the features in an array are functionalized for analyte capture. Exemplary substrates are described in Section (II)(c) of WO 2020/176788 and/or U.S. Patent Application Publication No. 2020/0277663. Exemplary features and geometric attributes of an array can be found in Sections (II)(d)(i), (II)(d)(iii), and (II)(d)(iv) of WO 2020/176788 and/or U.S. Patent Application Publication No. 2020/0277663.

Generally, analytes and/or intermediate agents (or portions thereof) can be captured when contacting a biological sample (e.g., a tissue sample) with a substrate including capture probes (e.g., a substrate with capture probes embedded, spotted, printed, fabricated on the substrate, or a substrate with features (e.g., beads, wells) comprising capture probes). As used herein, “contact,” “contacted,” and/or “contacting,” a biological sample with a substrate refers to any contact (e.g., direct or indirect) such that capture probes can interact (e.g., bind covalently or non-covalently (e.g., hybridize)) with analytes from the biological sample. Capture can be achieved actively (e.g., using electrophoresis) or passively (e.g., using diffusion). Analyte capture is further described in Section (II)(e) of WO 2020/176788 and/or U.S. Patent Application Publication No. 2020/0277663.

In some cases, spatial analysis can be performed by attaching and/or introducing a molecule (e.g., a peptide, a lipid, or a nucleic acid molecule) having a barcode (e.g., a spatial barcode) to a biological sample (e.g., a tissue sample). In some embodiments, a plurality of molecules (e.g., a plurality of nucleic acid molecules) having a plurality of barcodes (e.g., a plurality of spatial barcodes) are introduced to a biological sample (e.g., to a plurality of cells in a biological sample) for use in spatial analysis. In some embodiments, after attaching and/or introducing a molecule having a barcode to a biological sample, the biological sample can be physically separated (e.g., dissociated) into single cells or cell groups for analysis. Some such methods of spatial analysis are described in Section (III) of WO 2020/176788 and/or U.S. Patent Application Publication No. 2020/0277663.

In some cases, spatial analysis can be performed by detecting multiple oligonucleotides that hybridize to an analyte. In some instances, for example, spatial analysis can be performed using RNA-templated ligation (RTL). Methods of RTL have been described previously. See, e.g., Credle et al., Nucleic Acids Res. 2017 Aug. 21; 45(14):e128. Typically, RTL includes hybridization of two oligonucleotides to adjacent sequences on an analyte (e.g., an RNA molecule, such as an mRNA molecule). In some instances, the oligonucleotides are DNA molecules. In some instances, one of the oligonucleotides includes at least two ribonucleic acid bases at the 3′ end and/or the other oligonucleotide includes a phosphorylated nucleotide at the 5′ end. In some instances, one of the two oligonucleotides includes a capture domain (e.g., a poly(A) sequence, a non-homopolymeric sequence). After hybridization to the analyte, a ligase (e.g., SplintR ligase) ligates the two oligonucleotides together, creating a connected probe (e.g., a ligation product). In some instances, the two oligonucleotides hybridize to sequences that are not adjacent to one another. For example, hybridization of the two oligonucleotides creates a gap between the hybridized oligonucleotides. In some instances, a polymerase (e.g., a DNA polymerase) can extend one of the oligonucleotides prior to ligation. After ligation, the connected probe (e.g., a ligation product) is released from the analyte. In some instances, the connected probe (e.g., a ligation product) is released using an endonuclease. In some embodiments, the endonuclease is an RNAse. In some embodiments, the endonuclease is one of RNase A, RNase C, RNase H, and RNase I. In some embodiments, the endonuclease is RNAse H. In some embodiments, the RNase H is RNase H1 or RNase H2. The released connected probe (e.g., a ligation product) can then be captured by capture probes (e.g., instead of direct capture of an analyte) on an array, optionally amplified, and sequenced, thus determining the location and optionally the abundance of the analyte in the biological sample. Accordingly, the released connected probe can be considered an “analyte”.

During analysis of spatial information, sequence information for a spatial barcode associated with an analyte is obtained, and the sequence information can be used to provide information about the spatial distribution of the analyte in the biological sample. Various methods can be used to obtain the spatial information. In some embodiments, specific capture probes and the analytes they capture are associated with specific locations in an array of features on a substrate. For example, specific spatial barcodes can be associated with specific array locations prior to array fabrication, and the sequences of the spatial barcodes can be stored (e.g., in a database) along with specific array location information, so that each spatial barcode uniquely maps to a particular array location.

Alternatively, specific spatial barcodes can be deposited at predetermined locations in an array of features during fabrication such that at each location, only one type of spatial barcode is present so that spatial barcodes are uniquely associated with a single feature of the array. Where necessary, the arrays can be decoded using any of the methods described herein so that spatial barcodes are uniquely associated with array feature locations, and this mapping can be stored as described above.

When sequence information is obtained for capture probes and/or analytes during analysis of spatial information, the locations of the capture probes and/or analytes can be determined by referring to the stored information that uniquely associates each spatial barcode with an array feature location. In this manner, specific capture probes and captured analytes are associated with specific locations in the array of features. Each array feature location represents a position relative to a coordinate reference point (e.g., an array location, a fiducial marker) for the array. Accordingly, each feature location has an “address” or location in the coordinate space of the array.

Suitable systems for performing spatial analysis can include components such as a chamber (e.g., a flow cell or sealable, fluid-tight chamber) for containing a biological sample. The biological sample can be mounted for example, in a biological sample holder. One or more fluid chambers can be connected to the chamber and/or the sample holder via fluid conduits, and fluids can be delivered into the chamber and/or sample holder via fluidic pumps, vacuum sources, or other devices coupled to the fluid conduits that create a pressure gradient to drive fluid flow. One or more valves can also be connected to fluid conduits to regulate the flow of reagents from reservoirs to the chamber and/or sample holder.

The systems can optionally include a control unit that includes one or more electronic processors, an input interface, an output interface (such as a display), and a storage unit (e.g., a solid state storage medium such as, but not limited to, a magnetic, optical, or other solid state, persistent, writeable and/or re-writeable storage medium). The control unit can optionally be connected to one or more remote devices via a network. The control unit (and components thereof) can generally perform any of the steps and functions described herein. Where the system is connected to a remote device, the remote device (or devices) can perform any of the steps or features described herein. The systems can optionally include one or more detectors (e.g., CCD, CMOS) used to capture images. The systems can also optionally include one or more light sources (e.g., LED-based, diode-based, lasers) for illuminating a sample, a substrate with features, analytes from a biological sample captured on a substrate, and various control and calibration media.

The systems can optionally include software instructions encoded and/or implemented in one or more of tangible storage media and hardware components such as application specific integrated circuits. The software instructions, when executed by a control unit (and in particular, an electronic processor) or an integrated circuit, can cause the control unit, integrated circuit, or other component executing the software instructions to perform any of the method steps or functions described herein.

In some cases, the systems described herein can detect (e.g., register an image) the biological sample on the array. Exemplary methods to detect the biological sample on an array are described in PCT Application No. 2020/061064 and/or U.S. patent application Ser. No. 16/951,854.

Prior to transferring analytes from the biological sample to the array of features on the substrate, the biological sample can be aligned with the array. Alignment of a biological sample and an array of features including capture probes can facilitate spatial analysis, which can be used to detect differences in analyte presence and/or level within different positions in the biological sample, for example, to generate a three-dimensional map of the analyte presence and/or level. Exemplary methods to generate a two- and/or three-dimensional map of the analyte presence and/or level are described in PCT Application No. 2020/053655 and spatial analysis methods are generally described in WO 2020/061108 and/or U.S. patent application Ser. No. 16/951,864. In some cases, a map of analyte presence and/or level can be aligned to an image of a biological sample using one or more fiducial markers, e.g., objects placed in the field of view of an imaging system which appear in the image produced, as described in the Substrate Attributes Section, Control Slide for Imaging Section of WO 2020/123320, PCT Application No. 2020/061066, and/or U.S. patent application Ser. No. 16/951,843. Fiducial markers can be used as a point of reference or measurement scale for alignment (e.g., to align a sample and an array, to align two substrates, to determine a location of a sample or array on a substrate relative to a fiducial marker) and/or for quantitative measurements of sizes and/or distances.

### Sandwiching Processes

In some embodiments of a method disclosed herein, following the providing of a tumor tissue sample comprising one or more cells expressing an antigen-binding molecule (ABM), one or more analytes from the tissue sample are released from the tissue sample and migrate to a substrate comprising an array of capture probes for attachment to the capture probes of the array.

In some embodiments, the tissue sample is mounted on a first substrate during the providing step, and the substrate comprising the array of capture probes is a second substrate. In some embodiments, the method can include a “sandwiching process” using a device, sample holder, sample handling apparatus, or system described in, e.g., PCT/US2019/065100, PCT/US2021/036788, or PCT/US2021/050931 for the release and migration of the analytes to the array of capture probes in a manner that preserves their spatial context.

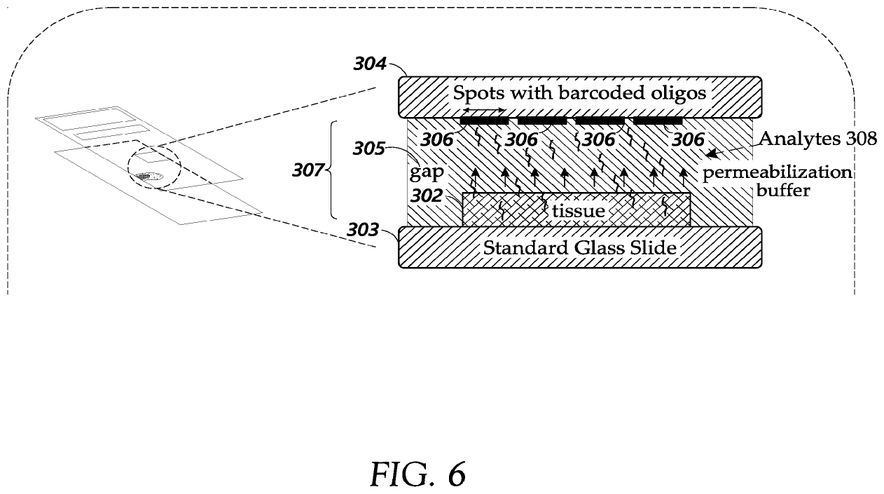

FIG. 6 is a schematic diagram depicting an exemplary sandwiching process 104 between a first substrate comprising a biological sample (e.g., a tissue section 302 on a slide 303) and a second substrate comprising a spatially barcoded array, e.g., a slide 304 that is populated with spatially-barcoded capture probes 306. During the exemplary sandwiching process, the first substrate is aligned with the second substrate, such that at least a portion of the biological sample is aligned with at least a portion of the array (e.g., aligned in a sandwich configuration). As shown, the slide 304 is in a superior position to the pathology slide 303. In some embodiments, the pathology slide 303 may be positioned superior to the slide 304. A permeabilization solution 305 within a gap 307 between the pathology slide 303 and the slide 304 creates a permeabilization buffer which permeabilizes or digests the sample 302 and the analytes (e.g., mRNA transcripts) 308 of the tissue sample 302 may release, actively or passively migrate (e.g., diffuse) across the gap 307 toward the capture probes 306, and bind on the capture probes 306.

After the analytes (e.g., transcripts) 308 bind on the capture probes 306, an extension reaction may occur, thereby generating a spatially barcoded library. For example, in the case of mRNA transcripts, reverse transcription may be used to generate a cDNA library associated with a particular spatial barcode. Barcoded cDNA libraries may be mapped back to a specific spot on a capture area of the capture probes 306. This gene expression data may be subsequently layered over a high-resolution microscope image of the tissue section, making it possible to visualize the expression of any mRNA, or combination of mRNAs, within the morphology of the tissue in a spatially-resolved manner. In some embodiments, the extension reaction can be performed separately from the sample handling apparatus described herein that is configured to perform the exemplary sandwiching process 104. The sandwich configuration of the sample 302, the pathology slide 303 and the slide 304 may provide advantages over other methods of spatial analysis and/or analyte capture. For example, the sandwich configuration may reduce a burden of users to develop in house tissue sectioning and/or tissue mounting expertise. Further, the sandwich configuration may decouple sample preparation/tissue imaging from the barcoded array (e.g., spatially-barcoded capture probes 306) and enable selection of a particular region of interest of analysis (e.g., for a tissue section larger than the barcoded array). The sandwich configuration also beneficially enables spatial analysis without having to place a tissue section 302 directly on the array slide (e.g., slide 304).

In some embodiments, the sandwiching process comprises: mounting the first substrate on a first member of a support device, the first member configured to retain the first substrate; mounting the second substrate on a second member of the support device, the second member configured to retain the second substrate, applying a reagent medium to the first substrate and/or the second substrate, the reagent medium comprising a permeabilization agent, operating an alignment mechanism of the support device to move the first member and/or the second member such that a portion of the tissue sample comprising the ABM-expressing cell is aligned (e.g., vertically aligned) with a portion of the array of capture probes and within a threshold distance of the array of capture probes, and such that the portion of the tissue sample and the capture probe contact the reagent medium, wherein the permeabilization agent releases the analyte (e.g., a nucleic acid encoding an ABM) from the ABM-expressing cell.

The sandwiching process methods described above can be implemented using a variety of hardware components.

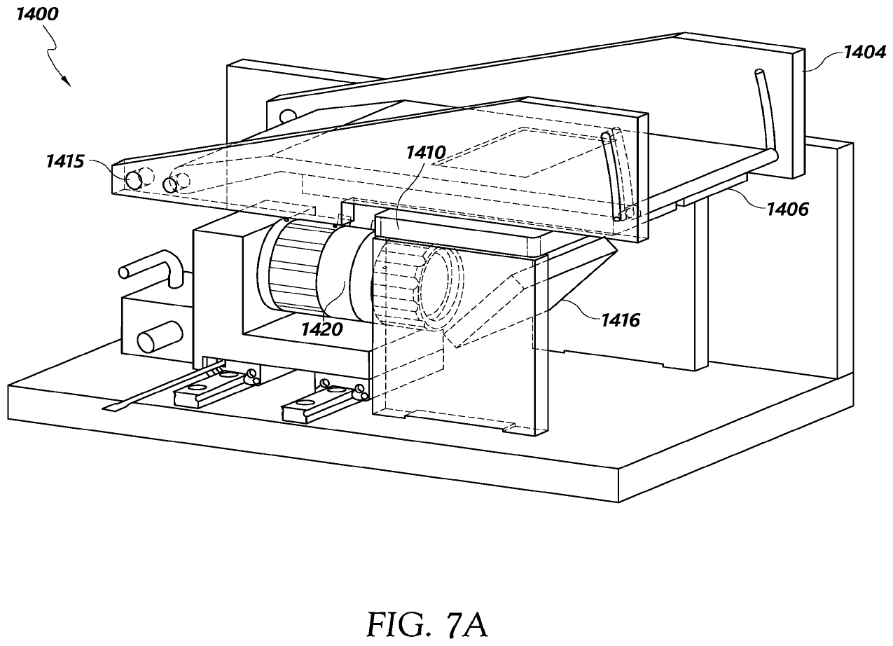

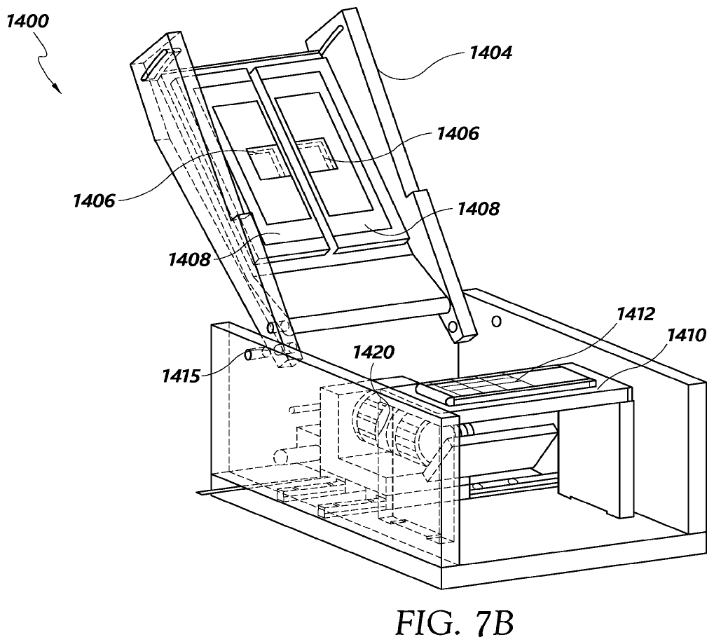

FIG. 7A is a perspective view of an example sample handling apparatus 1400 in a closed position in accordance with some example implementations. As shown, the sample handling apparatus 1400 includes a first member 1404, a second member 1410, an image capture device 1420, a first substrate 1406, a hinge 1415, and a mirror 1416. The hinge 1415 may be configured to allow the first member 1404 to be positioned in an open or closed configuration by opening and/or closing the first member 1404 in a clamshell manner along the hinge 1415.

FIG. 7B is a perspective view of the example sample handling apparatus 1400 in an open position in accordance with some example implementations. As shown, the sample handling apparatus 1400 includes one or more first retaining mechanisms 1408 configured to retain one or more first substrates 1406. In the example of FIG. 7B, the first member 1404 is configured to retain two first substrates 1406, however the first member 1404 may be configured to retain more or fewer first substrates 1406.

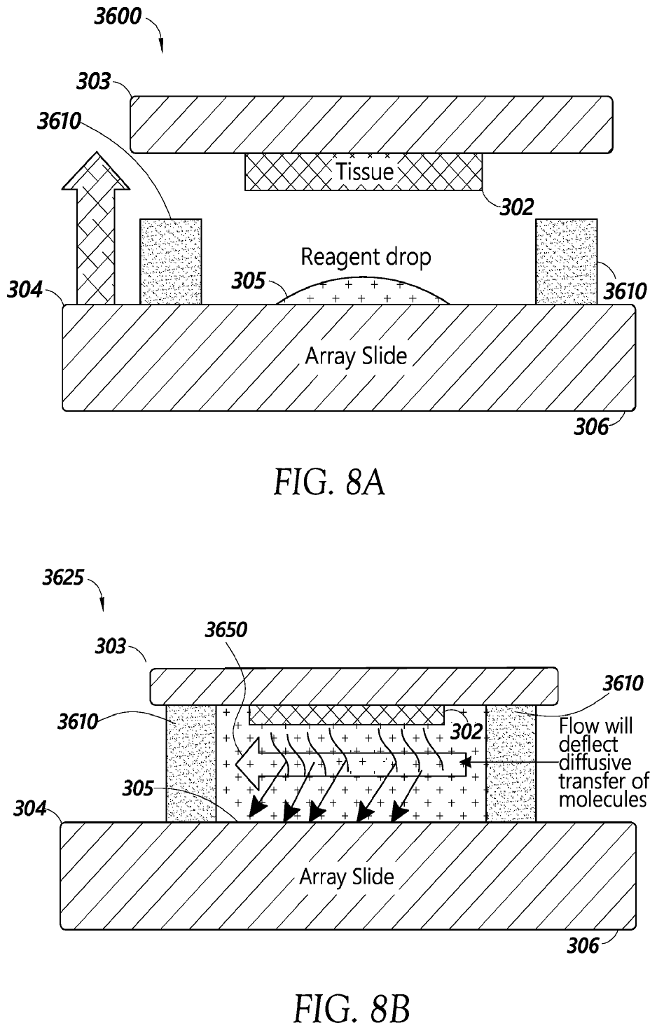

In some aspects, when the sample handling apparatus 1400 is in an open position (as in FIG. 8B), the first substrate 1406 and/or the second substrate 1412 may be loaded and positioned within the sample handling apparatus 1400 such as within the first member 1404 and the second member 1410, respectively. As noted, the hinge 1415 may allow the first member 1404 to close over the second member 1410 and form a sandwich configuration (e.g., the sandwich configuration shown in FIG. 6).

In some aspects, after the first member 1404 closes over the second member 1410, an adjustment mechanism (not shown) of the sample handling apparatus 1400 may actuate the first member 1404 and/or the second member 1410 to form the sandwich configuration for the permeabilization step (e.g., bringing the first substrate 1406 and the second substrate 1412 closer to each other and within a threshold distance for the sandwich configuration). The adjustment mechanism may be configured to control a speed, an angle, or the like of the sandwich configuration.

In some embodiments, the tissue sample (e.g., sample 302) may be aligned within the first member 1404 (e.g., via the first retaining mechanism 1408) prior to closing the first member 1404 such that a desired region of interest of the sample 302 is aligned with the barcoded array of the spatially barcoded array slide (e.g., the slide 304), e.g., when the first and second substrates are aligned in the sandwich configuration. Such alignment may be accomplished manually (e.g., by a user) or automatically (e.g., via an automated alignment mechanism). After or before alignment, spacers may be applied to the first substrate 1406 and/or the second substrate 1412 to maintain a minimum spacing between the first substrate 1406 and the second substrate 1412 during sandwiching. In some aspects, the permeabilization solution (e.g., permeabilization solution 305) may be applied to the first substrate 1406 and/or the second substrate 1412. The first member 1404 may then close over the second member 1410 and form the sandwich configuration. Analytes (e.g., mRNA transcripts) 308 may be captured by the capture probes 306 and may be processed for spatial analysis.

In some embodiments, during the permeabilization step, the image capture device 1420 may capture images of the overlap area (e.g., overlap area 710) between the tissue 302 and the capture probes 306. If more than one first substrates 1406 and/or second substrates 1412 are present within the sample handling apparatus 1400, the image capture device 1420 may be configured to capture one or more images of one or more overlap areas 710. Further details on support devices, sample holders, sample handling apparatuses, or systems for implementing a sandwiching process are described in, e.g., PCT/US2019/065100 and PCT/US2021/050931, each of which are incorporated by reference in their entirety.

Analytes within a biological sample are generally released through disruption (e.g., permeabilization, digestion, etc.) of the biological sample or may be released without disruption. Various methods of permeabilizing (e.g., any of the permeabilization reagents and/or conditions described herein) a biological sample are described herein, including for example including the use of various detergents, buffers, proteases, and/or nucleases for different periods of time and at various temperatures. Additionally, various methods of delivering fluids (e.g., a buffer, a permeabilization solution) to a biological sample are described herein including the use of a substrate holder (e.g., sandwich assembly, sandwich configuration, as described herein)

Provided herein are methods for delivering a fluid to a biological sample disposed on an area of a first substrate and an array disposed on a second substrate.

In some embodiments and with reference to FIG. 6, the sandwich configuration described herein between a tissue sample slide (e.g., pathology slide 303) and a spatially barcoded array slide (e.g., slide 304 with barcoded capture probes 306) may require the addition of a liquid reagent (e.g., permeabilization solution 305 or other target molecule release and capture solution) to fill a gap (e.g., gap 307). It may be desirable that the liquid reagent be free from air bubbles between the slides to facilitate transfer of target molecules with spatial information. Additionally, air bubbles present between the slides may obscure at least a portion of an image capture of a desired region of interest. Accordingly, it may be desirable to ensure or encourage suppression and/or elimination of air bubbles between the two slides during a permeabilization step (e.g., step 104).

In some aspects, it may be possible to reduce or eliminate bubble formation between the slides using a variety of filling methods and/or closing methods.

Workflows described herein may include contacting a drop of the liquid reagent disposed on a first substrate or a second substrate with at least a portion of a first substrate or second substrate, respectively. In some embodiments, the contacting comprises bringing the two substrates into proximity such that the sample on the first substrate is aligned with the barcode array of capture probes on the second substrate.

In some embodiments, the drop includes permeabilization reagents (e.g., any of the permeabilization reagents described herein). In some embodiments, the rate of permeabilization of the biological sample is modulated by delivering the permeabilization reagents (e.g., a fluid containing permeabilization reagents) at various temperatures.

In the example sandwich maker workflows described herein, a liquid reagent (e.g., the permeabilization solution 305) may fill a gap (e.g., the gap 307) between a tissue slide (e.g., slide 303) and a capture slide (e.g., slide 304 with barcoded capture probes 306) to warrant or enable transfer of target molecules with spatial information. Described herein are examples of filling methods that may suppress bubble formation and suppress undesirable flow of transcripts and/or target molecules or analytes. Robust fluidics in the sandwich making described herein may preserve spatial information by reducing or preventing deflection of molecules as they move from the tissue slide to the capture slide.

FIG. 8A shows an exemplary sandwiching process 3600 where a first substrate (e.g., pathology slide 303), including a biological sample 302 (e.g., a tissue section), and a second substrate (e.g., slide 304 including spatially barcoded capture probes 306) are brought into proximity with one another. As shown in FIG. 8A a liquid reagent drop (e.g., permeabilization solution 305) is introduced on the second substrate in proximity to the capture probes 306 and in between the biological sample 302 and the second substrate (e.g., slide 304). The permeabilization solution 305 may release analytes that can be captured by the capture probes 306 of the array. As further shown, one or more spacers 3610 may be positioned between the first substrate (e.g., pathology slide 303) and the second substrate (e.g., slide 304). The one or more spacers 3610 may be configured to maintain a separation distance between the first substrate and the second substrate. While the one or more spacers 3610 is shown as disposed on the second substrate, the spacer may additionally or alternatively be disposed on the first substrate.

In some embodiments, the one or more spacers 3610 is configured to maintain a separation distance between first and second substrates that is between about 2 microns and 1 mm (e.g., between about 2 microns and 800 microns, between about 2 microns and 700 microns, between about 2 microns and 600 microns, between about 2 microns and 500 microns, between about 2 microns and 400 microns, between about 2 microns and 300 microns, between about 2 microns and 200 microns, between about 2 microns and 100 microns, between about 2 microns and 25 microns, or between about 2 microns and 10 microns), measured in a direction orthogonal to the surface of first substrate that supports the sample. In some instances, the separation distance is about 2, 3, 4, 5, 6, 7, 8, 9, 10, 11, 12, 13, 14, 15, 16, 17, 18, 19, 20, 21, 22, 23, 24, or 25 microns. In some embodiments, the separation distance is less than 50 microns. In some embodiments, the separation distance is less than 25 microns. In some embodiments, the separation distance is less than 20 microns. The separation distance may include a distance of at least 2 μm.

FIG. 8B shows a fully formed sandwich configuration creating a chamber 3650 formed from the one or more spacers 3610, the first substrate (e.g., the pathology slide 303), and the second substrate (e.g., the slide 304) in accordance with some example implementations. In the example of FIG. 8B, the liquid reagent (e.g., the permeabilization solution 305) fills the volume of the chamber 3650 and may create a permeabilization buffer that allows analytes (e.g., mRNA transcripts and/or other molecules) to diffuse from the biological sample 302 toward the capture probes 306 of the slide 304. In some aspects, any flow of the permeabilization buffer may deflect transcripts and/or molecules from the biological sample 302 and may affect diffusive transfer of analytes for spatial analysis. A partially or fully sealed chamber 3650 resulting from the one or more spacers 3610, the first substrate, and the second substrate may reduce or prevent flow from undesirable convective movement of transcripts and/or molecules over the diffusive transfer from the biological sample 302 to the capture probes 306.

In some instances, the first substrate and the second substrate are arranged in an angled sandwich assembly as described herein. For example, during the sandwiching of the two slides (e.g., the pathology slide 303 and the slide 304) it may be possible to provide an angled closure of the slides to suppress or eliminate bubble formation.

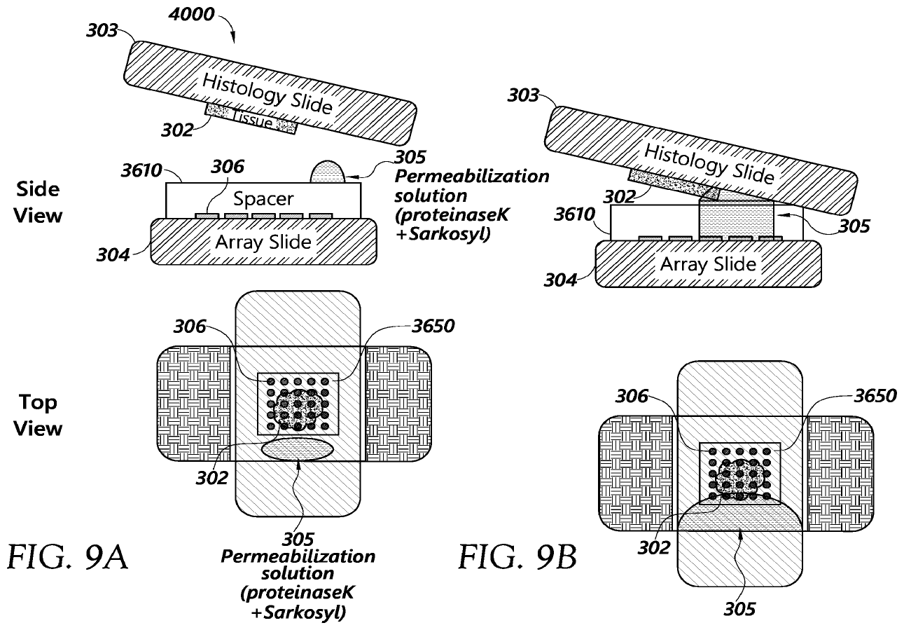

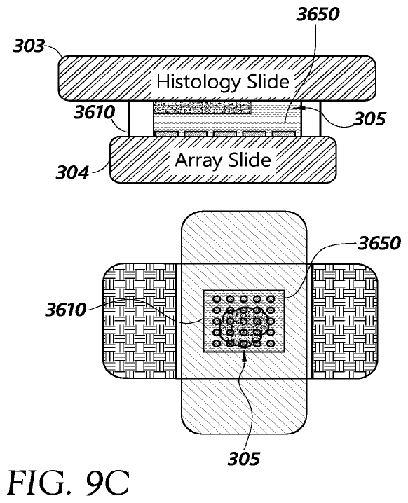

FIGS. 9A-9C depict a side view and a top view of an angled closure workflow 4000 for sandwiching a first substrate (e.g., pathology slide 303) having a tissue sample 302 and a second substrate (e.g., slide 304 having capture probes 306) in accordance with some example implementations.

FIG. 9A depicts the first substrate (e.g., the pathology slide 303 including sample 302) angled over (superior to) the second substrate (e.g., slide 304). As shown, a drop of the permeabilization solution 305 is located on top of the spacer 3610 toward the right-hand side of the side view in FIG. 9A.

FIG. 9B shows that as the first substrate lowers, or as the second substrate rises, the dropped side of the first substrate (e.g., a side of the slide 303 angled inferior to the opposite side) may contact the drop of the permeabilization solution 305. The dropped side of the first substrate may urge the permeabilization solution 305 toward the opposite direction. For example, in the side view of FIG. 9B the permeabilization solution 305 may be urged from right to left as the sandwich is formed.

FIG. 9C depicts a full closure of the sandwich between the first substrate and the second substrate with the spacer 3610 contacting both the first substrate and the second substrate and maintaining a separation distance between the two. As shown in the top view of FIG. 9C, the spacer 3610 fully encloses and surrounds the tissue sample 302 and the capture probes 306, and the spacer 3610 forms the sides of chamber 2650 which holds a volume of the permeabilization solution 305.

In some aspects, the alignment of the tissue sample 302 with the capture probes 306 shown in FIGS. 9A-9C may be performed by an alignment mechanism of a sample handling apparatus (e.g., as described in PCT/US2021/050931, which is hereby incorporated by reference in its entirety.

Further details on angled closure workflows, and devices and systems for implementing an angled closure workflow, are described in PCT/US2021/036788 and PCT/US2021/050931, which are hereby incorporated by reference in their entirety.

Additional configurations for reducing or eliminating bubble formation, and/or for reducing unwanted fluid flow, are described in PCT/US2021/036788, which is hereby incorporated by reference in its entirety.

Exemplar Spatial Methodologies for immune profiling

In some embodiments of any of the spatial profiling methods described herein, the methods are used to identify immune cell profiles. Immune cells express various adaptive immunological receptors relating to immune function, such as T cell receptors (TCRs) and B cell receptors (BCRs). T cell receptors and B cell receptors play a part in the immune response by specifically recognizing and binding to antigens and aiding in their destruction.

The T cell receptor, or TCR, is a molecule found on the surface of T cells that is generally responsible for recognizing fragments of antigen as peptides bound to major histocompatibility complex (MHC) molecules. The TCR is generally a heterodimer of two chains, each of which is a member of the immunoglobulin superfamily, possessing an N-terminal variable (V) domain, and a C terminal constant domain. In humans, in 95% of T cells, the TCR consists of an alpha (α) and beta (β) chain, whereas in 5% of T cells, the TCR consists of gamma and delta (γ/δ) chains. This ratio can change during ontogeny and in diseased states as well as in different species. When the TCR engages with antigenic peptide and MHC (peptide/MHC or pMHC), the T lymphocyte is activated through signal transduction.

Each of the two chains of a TCR contains multiple copies of gene segments—a variable ‘V’ gene segment, a diversity ‘D’ gene segment, and a joining ‘J’ gene segment. The TCR alpha chain (TCRa) is generated by recombination of V and J segments, while the beta chain (TCRb) is generated by recombination of V, D, and J segments. Similarly, generation of the TCR gamma chain involves recombination of V and J gene segments, while generation of the TCR delta chain occurs by recombination of V, D, and J gene segments. The intersection of these specific regions (V and J for the alpha or gamma chain, or V, D and J for the beta or delta chain) corresponds to the CDR3 region that is important for antigen-MHC recognition. Complementarity determining regions (e.g., CDR1, CDR2, and CDR3), or hypervariable regions, are sequences in the variable domains of antigen receptors (e.g., T cell receptor and immunoglobulin) that can complement an antigen. Most of the diversity of CDRs is found in CDR3, with the diversity being generated by somatic recombination events during the development of T lymphocytes. A unique nucleotide sequence comprising a specific combination of CDR sequences that arises during the gene arrangement process can be referred to as a clonotype.

The B cell receptor, or BCR, is a molecule found on the surface of B cells. The antigen binding portion of a BCR is composed of a membrane-bound antibody that, like most antibodies (e.g., immunoglobulins), has a unique and randomly determined antigen-binding site. The antigen binding portion of a BCR includes membrane-bound immunoglobulin molecule of one isotype (e.g., IgD, IgM, IgA, IgG, or IgE). When a B cell is activated by its first encounter with a cognate antigen, the cell proliferates and differentiates to generate a population of antibody-secreting plasma B cells and memory B cells. The various immunoglobulin isotypes differ in their biological features, structure, target specificity, and distribution.

Where immune cells are to be analyzed, primer sequences useful in any of the various operations for attaching barcode sequences and/or amplification reactions can include gene specific sequences which target genes or regions of genes of immune cell proteins, for example antigen binding molecules, such as immune receptors. Such gene sequences include, but are not limited to, sequences of various T cell receptor alpha variable genes (TRAV genes), T cell receptor alpha joining genes (TRAJ genes), T cell receptor alpha constant genes (TRAC genes), T cell receptor beta variable genes (TRBV genes), T cell receptor beta diversity genes (TRBD genes), T cell receptor beta joining genes (TRBJ genes), T cell receptor beta constant genes (TRBC genes), T cell receptor gamma variable genes (TRGV genes), T cell receptor gamma joining genes (TRGJ genes), T cell receptor gamma constant genes (TRGC genes), T cell receptor delta variable genes (TRDV genes), T cell receptor delta diversity genes (TRDD genes), T cell receptor delta joining genes (TRDJ genes), and T cell receptor delta constant genes (TRDC genes).

A fundamental understanding of spatial heterogeneity with respect to T-cell receptor (TCR) and B-cell receptor (BCR) clonotypes within a biological sample is needed to understand multiple facets of their functionality, including, for example, which cells a particular TCR or BCR may be interacting with within the biological sample, the identity of TCR and/or BCR clonotypes in a given biological sample, and/or the identity of TCR and/or BCR clonotypes that are autoreactive in different autoimmune disorders. Numerous single-cell sequencing approaches can identify TCR and BCR clonotypes from a biological sample, however, at present methods are needed to link TCR and BCR sequences to spatial locations within a biological sample. Additionally, identifying the clonal regions, that is, regions defined by the places where variable (V), diverse (D), and joining (J) segments join to form the complementarity determining regions, including CDR1, CDR2, and CDR3, which provide specificity to the TCRs and/or BCRs, would greatly benefit the scientific arts. By coupling clonal information to spatial information, it is possible to understand which T-cell and B-cell clonotypes may be specifically interacting with given cell types within a biological sample.

However, capturing analytes encoding immune cell receptors can provide unique challenges. For example, spatially capturing the TCR and BCR gene components with sufficient efficiency to profile the majority of clonotypes in a given tissue is difficult. Capturing analytes encoding immune cell receptors with conventional short-read sequencing methods can result in a loss of sequenced regions that are more than about 1 kb away from the point where sequencing starts (e.g., 5′ end proximal regions comprising CDR sequences, such as CDR3). Linking separate TCR or BCR gene components that together form a complete receptor using sequencing data from spots containing multiple different cells are challenges addressed by the methods described herein.

Exemplary methods for the spatial analysis of ABMs (e.g., TCRs and/or BCRs) which can be used in accordance with the methods disclosed herein are described in, for example, PCT Publication No. WO 2021/247568A1, PCT Publication No. WO 2021/247543A2, and PCT Application No. PCT/US2022/079628, each of which are entirely incorporated herein by reference for all purposes.

In some embodiments of a spatial immune profiling method, the method may comprise: attaching an analyte of an ABM-expressing cell of a tissue sample to a capture domain of a first capture probe of a substrate comprising an array of capture probes attached thereto, the first capture probe comprising (i) a spatial barcode sequence and (ii) the capture domain, the capture domain comprising a capture sequence, wherein the analyte of the ABM-expressing cell comprises a sequence or portion of a sequence encoding the ABM expressed by the ABM-expressing cell or a reverse complement thereof, using the analyte of the ABM-expressing cell and the first capture probe attached thereto to generate a spatially barcoded polynucleotide comprising (i) all or a portion of a sequence of the analyte of the ABM-expressing cell or reverse complement thereof and (ii) the spatial barcode sequence or reverse complement thereof, and determining all or a part of the nucleic acid sequences of the spatially barcoded polynucleotide; and using the determined nucleic acid sequences to identify the ABM and/or its location in the tissue sample, and optionally, to produce a recombinant ABM encoded by the determined sequences.

In some embodiments of any of the methods disclosed herein, analytes or reporter oligonucleotides can be attached to a capture domain of a capture probe via hybridization, or by ligation, e.g., splint-mediated ligation. Preferably, the analyte is a nucleic acid analyte, e.g., DNA (such as genomic DNA, cDNA) or RNA (e.g., mRNA).

### ABM Analysis Using a Spatial Transcriptomics, for Antigen Receptors Approach

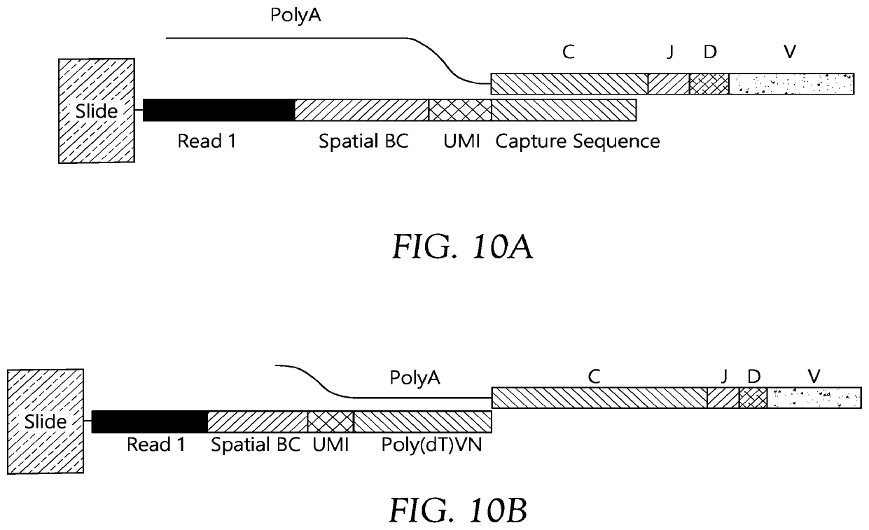

In particular embodiments, the capture sequence binds specifically to a nucleic acid sequence in the analyte encoding a region of an ABM. An exemplary capture probe with a capture sequence that specifically binds to a nucleic acid sequence encoding a constant region of an ABM is depicted in FIG. 10A. In some embodiments, the ABM is selected from: a TCR alpha chain, a TCR beta chain, a TCR gamma chain, a TCR delta chain an immunoglobulin kappa light chain, an immunoglobulin lambda light chain, an immunoglobulin heavy chain. In some embodiments, the capture sequence binds specifically to a sequence in a nucleic acid analyte encoding a constant region of the T cell receptor alpha chain. In some embodiments, the capture sequence binds specifically to a sequence in a nucleic acid analyte encoding a constant region of the T cell receptor beta chain. In some embodiments, the capture sequence binds specifically to a sequence in a nucleic acid analyte encoding a constant region of the T cell receptor delta chain. In some embodiments, the capture sequence binds specifically to a sequence in a nucleic acid analyte encoding a constant region of the T cell receptor gamma chain. In some embodiments, the capture sequence binds specifically to a sequence in a nucleic acid analyte encoding a constant region of the immunoglobulin kappa light chain. In some embodiments, the capture sequence binds specifically to a sequence in a nucleic acid analyte encoding a constant region of the immunoglobulin lambda light chain. In some embodiments, the capture sequence binds specifically to a sequence in a nucleic acid analyte encoding a constant region of the immunoglobulin heavy chain.

In other embodiments, the capture sequence is a homopolymeric sequence, e.g., a polyT sequence. FIG. 10B shows an exemplary poly(A) capture with a poly(T) capture domain. A poly(T) capture domain can capture other analytes, such as during global mRNA capture, including analytes encoding ABMs within the tissue sample.

In some embodiments, following capture of analytes by capture probes, capture probes can be extended, e.g., via reverse transcription. Second strand synthesis can generate double stranded cDNA products that are spatially barcoded. The double stranded cDNA products, which may comprise ABM encoding sequences and non-ABM related analytes, can be enriched for ABM encoding sequences.

An exemplary enrichment workflow may comprise amplifying the cDNA products (or amplicons thereof) with a first primer that specifically binds to a functional sequence of the first capture probe or reverse complement thereof and a second primer that binds to a nucleic acid sequence encoding a variable region of the ABM expressed by the ABM-expressing cell or reverse complement thereof. In some embodiments, the first primer and the second primer flank the spatial barcode of the first spatially barcoded polynucleotide or amplicon thereof. In some embodiments, the first primer and the second primer flank a J junction, a D junction, and/or a V junction.

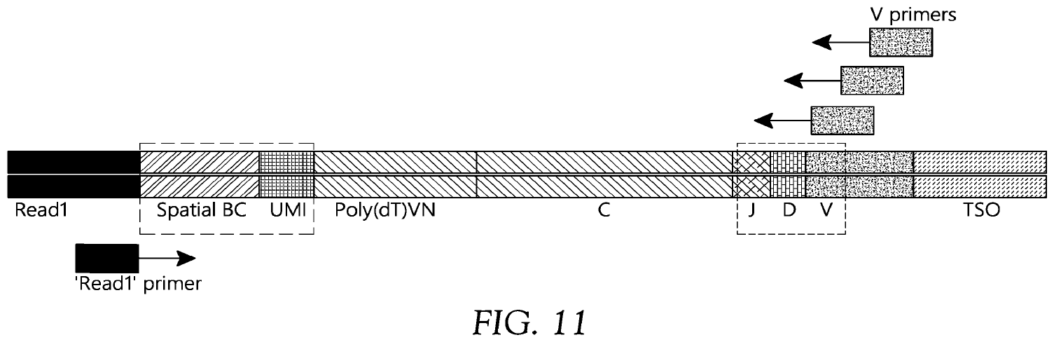

FIG. 11 shows an exemplary analyte enrichment strategy following analyte capture on the array. The portion of the immune cell analyte of interest includes the sequence of the V(D)J region, including CDR sequences. As described herein, a poly(T) capture probe captures an analyte encoding an ABM, an extended capture probe is generated by a reverse transcription reaction, and a second strand is generated. The resulting nucleic acid library can be enriched by the exemplary scheme shown in FIG. 11, where an amplification reaction including a Read 1 primer complementary to the Read 1 sequence of the capture probe and a primer complementary to a portion of the variable region of the immune cell analyte, can enrich the library via PCR. While FIG. 11 depicts a Read 1 primer, it is understood that a primer complementary to other functional sequences, such as other sequencing primer sequences, or sequencer specific flow cell attachment sequences, or portions of such functional sequences, may also be used. While FIG. 11 depicts a polyT capture sequence, it is understood that other capture sequences disclosed herein may be present in library members. The enriched library can be further enriched by nested primers complementary to a portion of the variable region internal (e.g., 5′) to the initial variable region primer for practicing nested PCR.

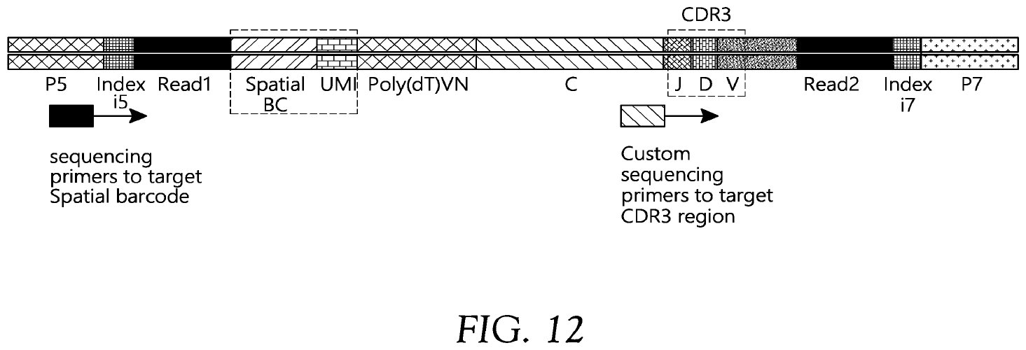

FIG. 12 shows a sequencing strategy with a primer specific complementary to the sequencing flow cell attachment sequence (e.g., P5) and a custom sequencing primer complementary to a portion of the constant region of the analyte. This sequencing strategy targets the constant region to obtain the sequence of the CDR regions, including CDR3, while concurrently or sequentially sequencing the spatial barcode (BC) and/or unique molecular identifier (UMI) of the capture probe. By capturing the sequence of a spatial barcode, UMI and a V(D)J region the receptor is not only determined, but its spatial location and abundance within a cell or tissue is also identified.

In some embodiments, the method includes (a) contacting a biological sample with an array comprising a plurality of capture probes, wherein a capture probe of the plurality of capture probes comprises (i) a spatial barcode and (ii) a capture domain that hybridizes to a poly(A) sequence of a nucleic acid encoding an immune cell receptor expressed by an immune cell in the biological sample; (b) hybridizing the capture domain to the nucleic acid encoding the immune cell receptor; (c) extending the capture probe using the nucleic acid encoding the immune cell receptor as a template to generate an extended capture probe comprising a sequence encoding a CDR3, or a complement thereof, of the immune cell receptor of the immune cell clonotype; (d) hybridizing one or more probes to the extended capture probe, or a complement thereof, in a portion encoding a constant region of the immune cell receptor of the immune cell clonotype, wherein the one or more probes comprises a binding moiety capable of binding a capture moiety; (e) enriching the extended capture probe or the complement thereof via an interaction between the binding moiety in the one or more probes and the capture moiety; and (f) determining (i) the sequence of the spatial barcode or a complement thereof, and (ii) all or a portion of the sequence of the nucleic acid encoding the immune cell receptor or a complement thereof, and using the determined sequences of (i) and (ii) to determine the presence of the immune cell clonotype at a location in the biological sample.

In some embodiments, and the one or more probes hybridizes to a nucleic acid sequence encoding a constant region of the T cell receptor alpha chain, or a complement thereof. In some embodiments, step (f) comprises determining a sequence encoding one or more of CDR1, CDR2, and CDR3 of the immune cell receptor, and optionally, determining a sequence encoding a full-length variable domain of the immune cell receptor. In some embodiments, the method further includes generating the complement of the extended capture probe using the extended capture probe as a template, wherein the complement of the extended capture probe comprises (i) a sequence that is complementary to the spatial barcode, and (ii) a sequence that corresponds to all or a portion of the sequence of the nucleic acid encoding the immune cell receptor. In some embodiments, the binding moiety comprises biotin and the capture moiety comprises streptavidin.

In some embodiments, the determining in step (f) comprises sequencing the extended capture probe or the complement thereof to determine (i) the sequence of the spatial barcode, or the complement thereof, and (ii) all or a portion of the sequence of the nucleic acid encoding the immune cell receptor of the immune cell clonotype or the complement thereof. In some embodiments, the sequencing comprises long read sequencing.

In some embodiments, the capture probe further comprises an adaptor domain and the method further comprises after step (e), performing a polymerase chain reaction using i) a first primer complementary to the adaptor domain of the capture probe, and ii) a second primer complementary to a portion of a nucleic acid sequence encoding a variable region of the immune cell receptor. In some embodiments, the second primer is complementary to a nucleic acid sequence 5′ to the sequence encoding CDR3 of the immune cell receptor. In some embodiments, generating the complement of the extended capture probe comprises use of a template switch oligonucleotide. By using capture domains that hybridize to a poly(A) sequences, the method is advantageous in that it allows for spatial analysis of global mRNA expression as well as a targeted spatial analysis of ABMs at once.

### ABM Analysis Using Nucleic Acid Library Methods to Remove Portion of Analyte Sequences

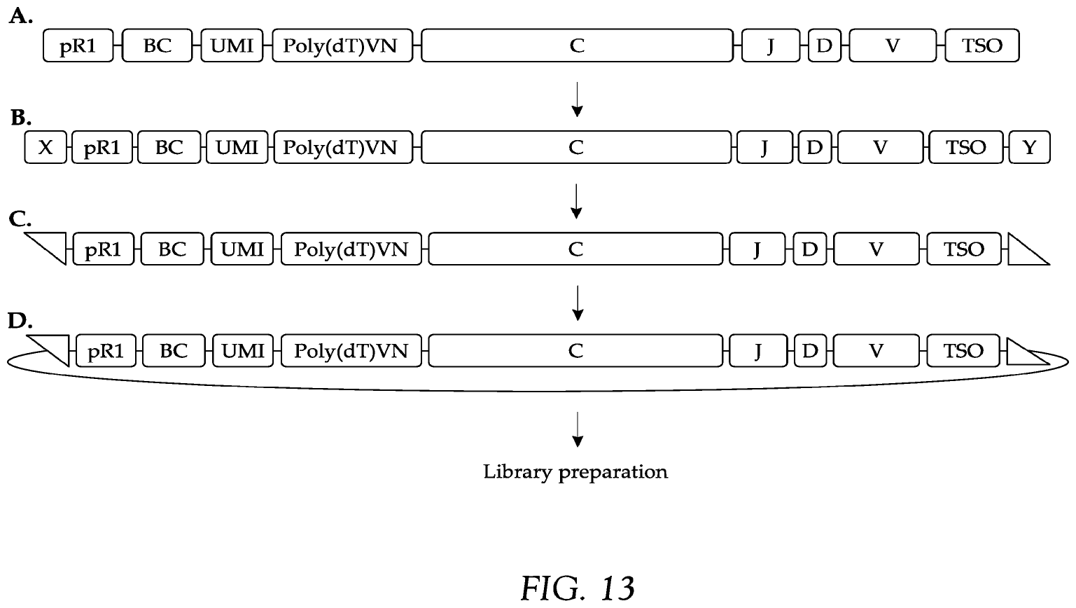

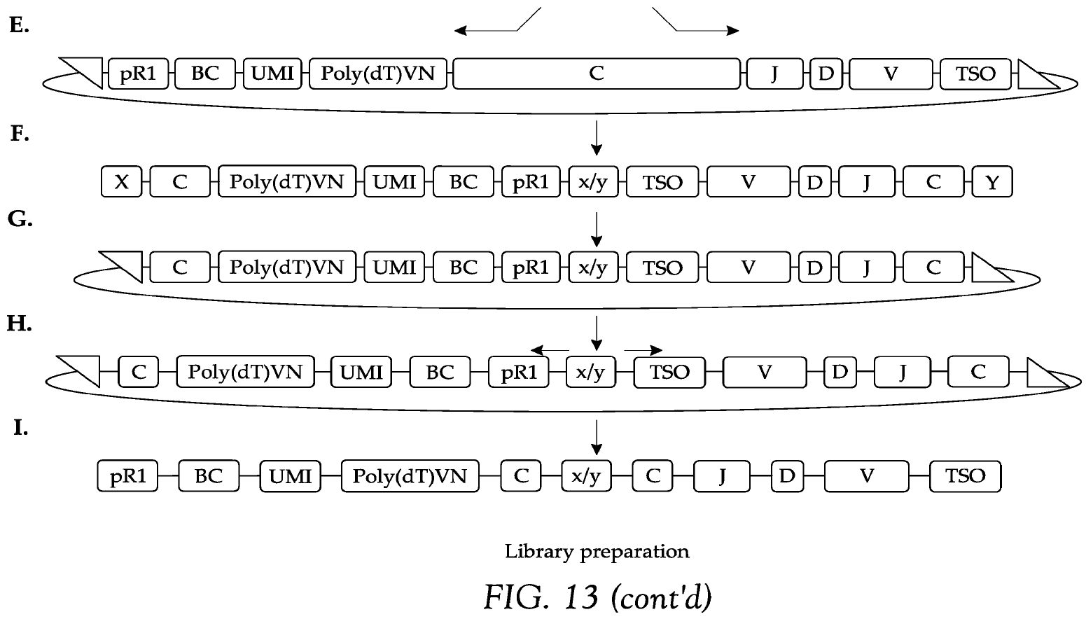

FIG. 13 shows an exemplary nucleic acid library preparation method to remove a portion of an analyte sequence via double circularization of a member of a nucleic acid library. Panel A shows an exemplary member of a nucleic acid library including, in a 5′ to 3′ direction, a first adaptor (e.g., primer sequence R1, pR1 (e.g., Read 1)), a barcode (e.g., a spatial barcode or a cell barcode), a unique molecular identifier (UMI), a capture domain (e.g., poly(T) VN sequence), a sequence complementary to a nucleic analyte encoding an ABM (e.g., encoding C, J, D and V regions of a BCR or TCR), and a second adaptor (e.g., template switching oligonucleotide sequence (TSO)). For purposes of this example an analyte including a constant region (C) and V(D)J sequence are shown, however, the methods described herein can be equally applied to other analyte sequences in a nucleic acid library. Panel B shows the exemplary member of a nucleic acid library where additional sequences can be added to both the 3′ and 5′ ends of the nucleic acid member (shown as a X and Y) via a PCR reaction. The additional sequences added can include a recognition sequence for a restriction enzyme (e.g., restriction endonuclease). The restriction recognition sequence can be for a rare restriction enzyme. The exemplary member of the nucleic acid library shown in Panel B can be digested with a restriction enzyme to generate sticky ends shown in Panel C (shown as triangles) and can be intramolecularly circularized by ligation to generate the circularized member of the nucleic acid library shown in Panel D. The ligation can be performed with a DNA ligase. The ligase can be T4 ligase. A primer pair can be hybridized to a circularized nucleic acid member, where a first primer hybridizes to a 3′ portion of a sequence encoding the constant region (C) and includes a second restriction enzyme (e.g., restriction endonuclease) sequence that is non-complementary to the analyte sequence, and where a second primer hybridized to a 5′ portion of a sequence encoding the constant region (C), and where the second primer includes a second restriction enzyme sequence (Panel E). The first primer and the second primer can generate a linear amplification product (e.g., a first double-stranded nucleic acid product) as shown in Panel F, which includes the second restriction enzyme recognition sequences (shown as X and Y end sequences). The linear amplification product (Panel F) can be digested with a second restriction enzyme to generate sticky ends and can be intramolecularly ligated with a ligase (e.g., T4 DNA ligase) to generate a second double-stranded circularized nucleic acid product as shown in Panel G. The second double-stranded circularized nucleic product (Panel G) can be amplified with a third primer, pR1, substantially complementary to the first adaptor (e.g., Read 1) sequence and a fourth primer substantially complementary to the second adapter (e.g., TSO) as shown in Panel H to generate a version of the double-stranded member of the nucleic acid library lacking all, or a portion of, the sequence encoding the constant region (C) of the ABM encoded by the nucleic acid analyte (Panel I). The resulting double-stranded member of the nucleic acid library lacking all or a portion of the analyte sequence encoding the constant region can undergo library preparation methods, such as library preparation methods used in single-cell or spatial analyses. For example, the double-stranded member of the nucleic acid library lacking all, or a portion of, the analyte sequence encoding the constant region of the ABM can be fragmented, followed by end repair, a-tailing, adaptor ligation, and/or additional amplification (e.g., PCR). The fragments can then be sequenced using, for example, paired-end sequencing using TruSeq Read 1 and TruSeq Read 2 as sequencing primer sites or any other sequencing method described herein. As such, sequences can be determined from regions more than about 1 kb away from the end of a nucleic acid analyte encoding an ABM (e.g., 3′ end) and can link such a sequence to a barcode sequence (e.g., a spatial barcode, a cell barcode) in library preparation methods (e.g., sequencing preparation). For purposes of this example a nucleic acid analyte encoding a constant region (C) and V(D)J region of an ABM (e.g., BCR or TCR) are shown, however, the methods described herein can be equally applied to other analyte sequences in a nucleic acid library.

An exemplary member of a nucleic acid library can be prepared as shown in FIG. 13 to generate a first double-stranded circularized nucleic acid product shown in Panel D of FIG. 13 as previously described.

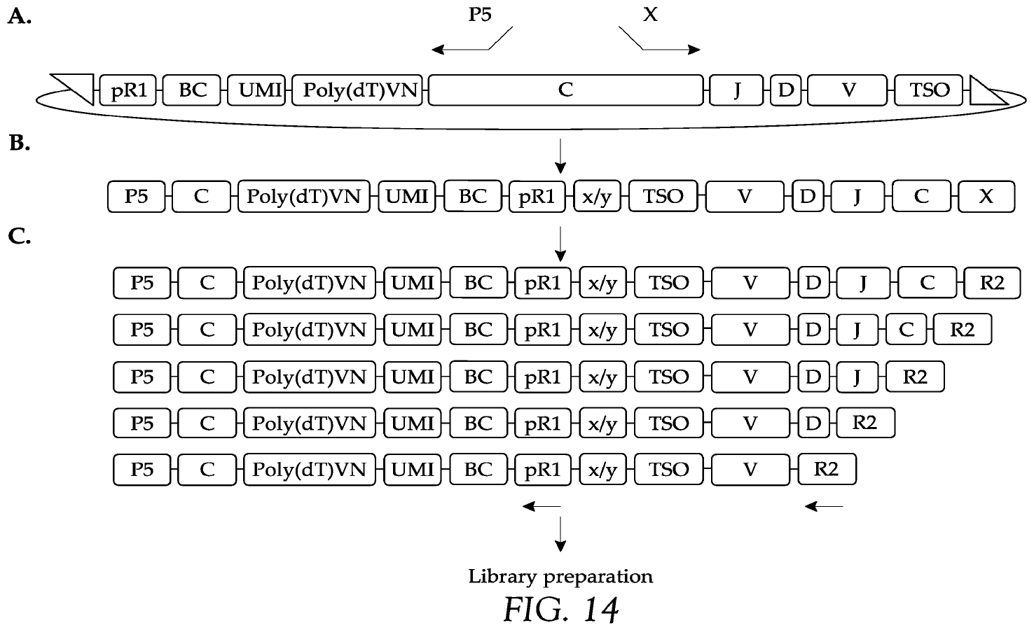

FIG. 14 depicts another exemplary workflow for processing such double-stranded circularized nucleic acid product. A primer pair can be contacted with the double-stranded circularized nucleic acid produce with a first primer that can hybridize to a sequence from a 3′ region of the analyte sequence encoding the constant region of the ABM and a sequence including a first functional domain (e.g., P5). The second primer can hybridize to a sequence from a 5′ region of the analyte sequence encoding the constant region of the ABM, and includes a sequence including a second functional domain (shown as “X”) as shown in Panel A. Amplification of the double-stranded circularized nucleic acid product results in a linear product as shown in Panel B, where all, or a portion of, the sequence encoding a constant region (C) of the ABM is removed. The first functional domain can include a sequencer specific flow cell attachment sequence (e.g., P5). The second functional domain can include an amplification domain such as a primer sequence to amplify the nucleic acid library prior to further sequencing preparation. The resulting double-stranded member of the nucleic acid library lacking all or a portion of the constant region can undergo library preparation methods, such as library preparation methods used in single-cell or spatial analyses. For example, the double-stranded member of the nucleic acid library lacking all, or a portion of, the analyte sequence encoding the constant region of the ABM can be fragmented, followed by end repair, A-tailing, adaptor ligation, and/or amplification (e.g., PCR) (Panel C). The fragments can then be sequenced using, for example, paired-end sequencing using TruSeq Read 1 and TruSeq Read 2 as sequencing primer sites (Panel C, arrows), or any other sequencing method described herein. After library preparation methods described herein, a different sequencing primer for the first adaptor (e.g., Read 1) is used since the orientation of the first adaptor (e.g., Read 1) sequence will be reversed. Accordingly, sequences can be determined from regions more than about 1 kb away from the end of a nucleic acid analyte encoding an ABM (e.g., 3′ end) and can link such a sequence to a barcode sequence (e.g., a spatial barcode, a cell barcode) in further library preparation methods (e.g., sequencing preparation). For purposes of this example a nucleic acid analyte encoding a constant region (C) and V(D)J region of an ABM (e.g., BCR or TCR) is shown, however, the methods described herein can be applied to other analyte sequences in a nucleic acid library as well.

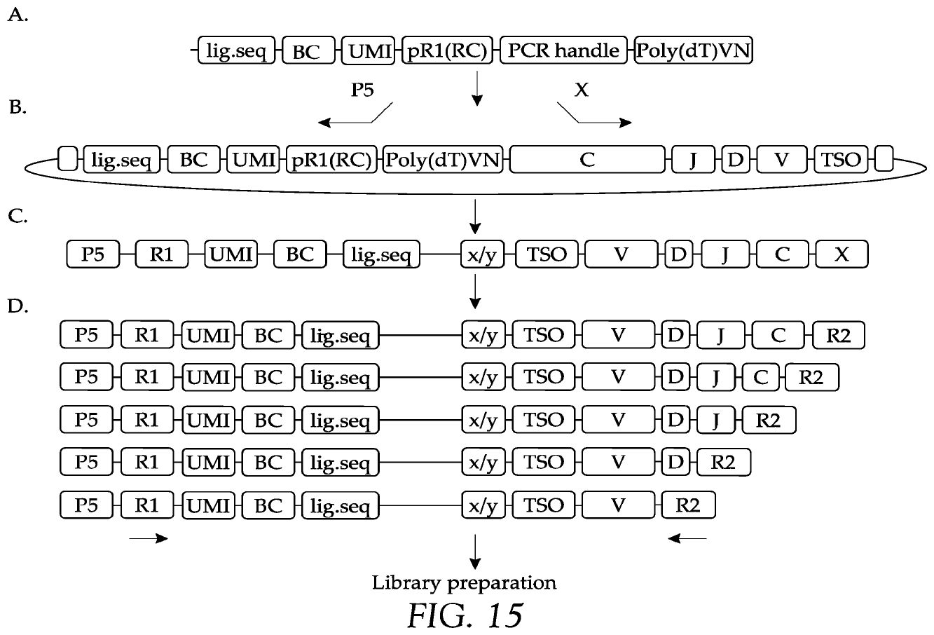

FIG. 15 shows an exemplary nucleic acid library preparation method to remove all or a portion of a constant sequence of a nucleic analyte from a member of a nucleic acid library via circularization. Panels A and B shows an exemplary member of a nucleic acid library including, in a 5′ to 3′ direction, a ligation sequence, a barcode sequence, a unique molecular identifier, a reverse complement of a first adaptor (e.g., primer sequence pR1 (e.g., Read 1)), a capture domain, a sequence complementary to the nucleic acid analyte encoding an ABM, and a second adapter (e.g., TSO sequence). The ends of the double-stranded nucleic acid can be ligated together via a ligation reaction where the ligation sequence splints the ligation to generate a circularized double-stranded nucleic acid as shown in Panel B. The circularized double-stranded nucleic acid can be amplified with a pair of primers to generate a linear nucleic acid product lacking all or a portion of the analyte sequence encoding the constant region of the ABM (Panels B and C). The first primer can include a sequence substantially complementary to the reverse complement of the first adaptor and a first functional domain. The first functional domain can be a sequencer specific flow cell attachment sequence (e.g., P5). The second primer can include a sequence substantially complementary to a sequence from a 5′ region of the analyte sequence encoding the constant region of the ABM, and a second functional domain. The second functional domain can include an amplification domain such as a primer sequence to amplify the nucleic acid library prior to further sequencing preparation. The resulting double-stranded member of the nucleic acid library lacking all or a portion of the constant region can undergo library preparation methods, such as library preparation methods used in single-cell or spatial analyses. For example, the double-stranded member of the nucleic acid library lacking all, or a portion of, the analyte sequence encoding the constant region of the ABM can be fragmented, followed by end repair, A-tailing, adaptor ligation, and/or amplification (e.g., PCR) (Panel C). The fragments can then be sequenced using, for example, paired-end sequencing using TruSeq Read 1 and TruSeq Read 2 as sequencing primer sites, or any other sequencing method described herein (Panel D). After library preparation methods (e.g., described herein), sequencing primers can be used since the orientation of Read 1 will be in the proper orientation for sequencing primer pR1. Accordingly, sequences can be determined from regions more than about 1 kb away from the end of a nucleic acid analyte encoding an ABM (e.g., 3′ end) and can link such a sequence to a barcode sequence (e.g., a spatial barcode, a cell barcode) in further library preparation methods (e.g., sequencing preparation). For purposes of this example a nucleic acid analyte encoding a constant region (C) and V(D)J region is shown, however, the methods described herein can be applied to other analyte sequences in a nucleic acid library as well.

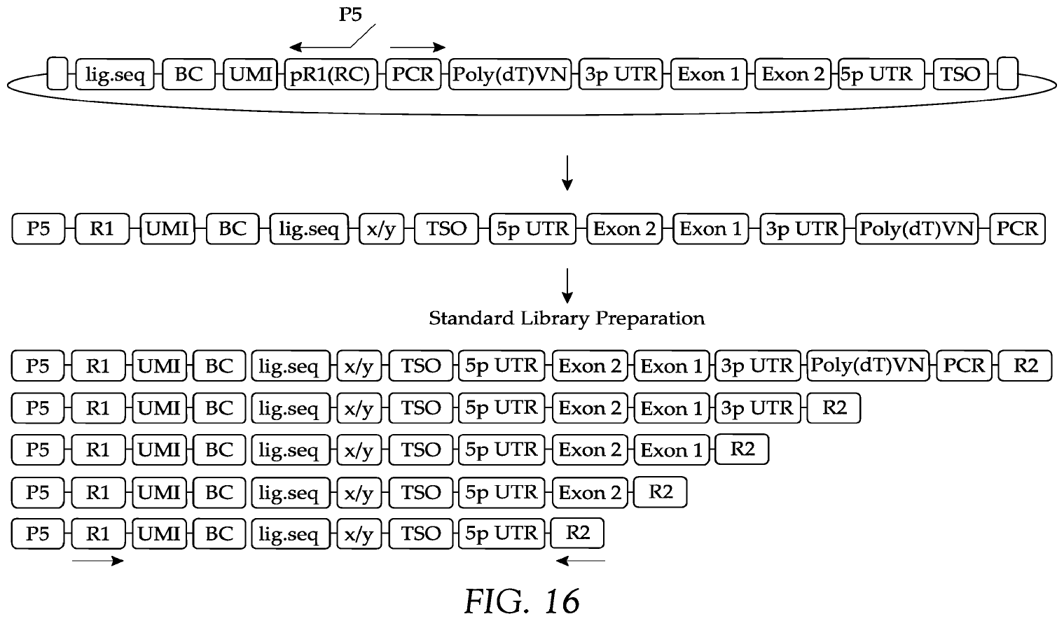

FIG. 16 shows an exemplary nucleic acid library method to reverse the orientation of an analyte sequence in a member of a nucleic acid library. Panel A shows an exemplary member of a nucleic acid library including, in a 5′ to 3′ direction, a ligation sequence, a barcode (e.g., a spatial barcode or a cell barcode), unique molecular identifier, a reverse complement of a first adaptor, an amplification domain, a capture domain, a sequence of a nucleic acid analyte encoding an ABM, and a second adapter. The ends of the double-stranded nucleic acid can be ligated together via a ligation reaction where the ligation sequence splints the ligation to generate a circularized double-stranded nucleic acid also shown in Panel A. The circularized double-stranded nucleic acid can be amplified to generate a linearized double-stranded nucleic acid product, where the orientation of the nucleic acid analyte is reversed such that the 5′ sequence (e.g., 5′ UTR) is brought in closer proximity to the barcode (e.g., a spatial barcode or a cell barcode) (Panel B). The first primer includes a sequence substantially complementary to the reverse complement of the first adaptor and a functional domain. The functional domain can be a sequencer specific flow cell attachment sequence (e.g., P5). The second primer includes a sequence substantially complementary to the amplification domain. The resulting double-stranded member of the nucleic acid library including a reversed analyte sequence (e.g., the 5′ end of the analyte sequence is brought in closer proximity to the barcode) can undergo library preparation methods, such as library preparation methods used in single-cell or spatial analyses. For example, the double-stranded member of the nucleic acid library lacking all, or a portion of, the nucleic acid analyte sequence encoding the constant region of the ABM can be fragmented, followed by end repair, A-tailing, adaptor ligation, and/or amplification (e.g., PCR) (Panel C). The fragments can then be sequenced using, for example, paired-end sequencing using TruSeq Read 1 and TruSeq Read 2 as sequencing primer sites, or any other sequencing method described herein. Accordingly, sequences from the 5′ end of a nucleic analyte encoding an ABM will be included in sequencing libraries (e.g., paired end sequencing libraries). Any type of analyte sequence in a nucleic acid library can be prepared by the methods described in this Example (e.g., reversed).

### Spatial Analysis Using Array Features Comprising Poly(T) and Poly(GI) Capture Domains

Provided herein are methods of determining a location of a target nucleic acid encoding an ABM in a biological sample that include: (a) contacting the biological sample with an array comprising a feature, where the feature comprises an attached first and second probe, wherein: a 5′ end of the first probe is attached to the feature; the first probe comprises in a 5′ to a 3′ direction: a spatial barcode and a poly(T) capture domain, where the poly(T) capture domain binds specifically to the target nucleic acid; a 5′ end of the second probe is attached to the feature; a 3′ end of the second probe is reversibly blocked; and the second probe comprises a poly(GI) capture domain; (b) extending a 3′ end of the first probe to add a sequence that is complementary to a portion of the target nucleic acid; (c) ligating an adapter to the 5′ end of the target nucleic acid specifically bound to the first probe; (d) adding a sequence complementary to the adapter to the 3′ end of the first probe; (e) adding non-templated cytosines to the 3′ end of the first probe to generate a poly(C) sequence, where the poly(C) sequence specifically binds to the poly(GI) capture domain of the second probe; (f) unblocking the 3′ end of the second probe and extending the 3′ end of the second probe to add a sequence comprising a sequence in the target nucleic acid and a sequence that is complementary to the spatial barcode; (g) cleaving a region of the second probe at a cleavage site that is 5′ to the poly(GI) capture domain, thereby releasing the second probe from the feature; and (h) determining (i) all or a part of the sequence of the spatial barcode, or a complement thereof, and (ii) all or a part of the sequence of the target nucleic acid, or a complement thereof, and using the sequences of (i) and (ii) to determine the location of the target nucleic acid in the biological sample.

In some embodiments, a feature can include two or more pairs of a first and a second probe (e.g., any of the first and second probes described in this section). A first pair of a first and a second probe at a feature, as compared to a second pair of a first and a second probe at the feature, can have a different first and/or second probe as compared to first and/or second probe of the second pair (e.g., a different capture domain in the first probe and/or a different barcode in the first and/or second probes). In some embodiments, the spatial barcode in the first probe of the first pair and the spatial barcode in the first probe of the second pair are the same. In some embodiments, the spatial barcode in the first probe of the first pair and the spatial barcode in the first probe of the second pair are different. In some embodiments, the capture domain of the first probe of the first pair is the same as the capture domain of the first probe of the second pair. In some embodiments, the capture domain of the first probe of the first pair is different from the capture domain of the first probe of the second pair.

In some embodiments, the capture domain on the first probe has a poly(T) capture domain, where the poly(T) capture domain is configured to interact with the target nucleic acid (e.g., positioned at the 3′ end of the first probe). For example, the poly(T) capture domain specifically binds to a messenger RNA (mRNA), via the poly(A) tail of the mRNA. For example, a poly(T) capture domain can include at least 3, at least 4, at least 5, at least 6, at least 7, at least 8, at least 9, at least 10, at least 11, at least 12, at least 13, at least 14, at least 15, at least 16, at least 17, at least 18, at least 19, at least 20, at least 25, or at least 30 contiguous thymidines.

In some embodiments, the poly(GI) capture domain of the second probe is configured to interact with a poly(C) tail of an oligonucleotide, e.g., a poly(C) tail added to the 3′ end of the extended first probe. In some embodiments, the poly(C) tail is added to the 3′ end of the first probe after the extension of the first probe to add a sequence that is complementary to a portion of the target nucleic acid. In some embodiments, the poly(GI) capture domain comprises a sequence of at least 5 contiguous guanosine(s) and/or inosine(s). For example, a poly(GI) capture domain comprises a sequence of (GGI)n, wherein n is about 3 to about 20. In some embodiments, the poly(GI) capture domain comprises a sequence of (GGI)n, wherein n is 1, 2, 3, 4, 5, 6, 7, 8, 9, 10, 11, 12, 13, 14, 15, 16, 17, 18, 19, or 20. For example, a poly(GI) capture domain comprises a sequence of (GI)n, wherein n is about 4 to about 30. For example, a poly(GI) capture domain comprises a sequence of (GI)n, wherein n is 4, 5, 6, 7, 8, 9, 10, 11, 12, 13, 14, 15, 16, 17, 18, 19, 20, 21, 22, 23, 24, 25, 26, 27, 28, 29, or 30. For example, a poly(GI) capture domain comprises a sequence of (IG)n, wherein n is about 4 to about 30. For example, a poly(GI) capture domain comprises a sequence of (IG)n, wherein n is 4, 5, 6, 7, 8, 9, 10, 11, 12, 13, 14, 15, 16, 17, 18, 19, 20, 21, 22, 23, 24, 25, 26, 27, 28, 29, or 30.

In some embodiments, the second probe can comprise a spatial barcode, which is positioned 5′ to the poly(GI) capture domain. In some embodiments, the spatial barcode in the first probe is different from the spatial barcode sequence in the second probe. In some embodiments, the spatial barcode in the first probe is the same as the spatial barcode sequence in the second probe.

In some embodiments, both the first and the second probes are cleavable. In some embodiments, the first probe and the second probe have different cleavage sites and are cleavable using different methods. In some embodiments, the first probe and the second probe have the same cleavable site and are cleavable using the same method. In some embodiments, the cleavage domain of the first probe is 5′ to the poly(T) capture domain and/or the cleavage domain of the second probe is 5′ to the poly(GI) capture domain.

In some embodiments, the first probe is not cleavable and the second probe is cleavable. In some embodiments, the cleavage site of the second probe is 5′ to the poly(GI) capture domain of the second probe. In some embodiments, the cleavage site on the second probe is a uracil. In some embodiments, the uracil is cleaved by USER (Uracil-Specific Excision Reagent).

In some embodiments, the first probe further comprises a unique molecular identifier (UMI). In some embodiments, the second probe further comprises a unique molecular identifier (UMI). In some embodiments, the UMI in the first probe and the UMI in the second probe comprise different sequences. In some embodiments, the UMI in the first probe and the UMI in the second probe comprise the same sequence.

In some embodiments, step (h) includes sequencing all or a part of the sequence of the spatial barcode, or a complement thereof, and sequencing all of a part of the sequence of the target nucleic acid, or a complement thereof. The sequencing can be performed using any of the aforementioned methods. In some embodiments, step (h) includes sequencing the full-length sequence of the spatial barcode, or a complement thereof. In some embodiments, step (h) includes sequencing a part of the sequence of the spatial barcode, or a complement thereof. In some embodiments, step (h) includes sequencing the full-length sequence of the target nucleic acid, or a complements thereof. In some embodiments, step (h) includes sequencing a part of the target nucleic acid, or a complement thereof. In some embodiments, the sequencing is performed using high throughput sequencing. In some embodiments, the target nucleic acid is sequenced from the 5′ end of the target nucleic acid. In some embodiments, the target nucleic acid is sequenced from the 3′ end of the target nucleic acid. In some embodiments, the target nucleic acid is sequenced from both the 3′ end and the 5′ end of the target nucleic acid.

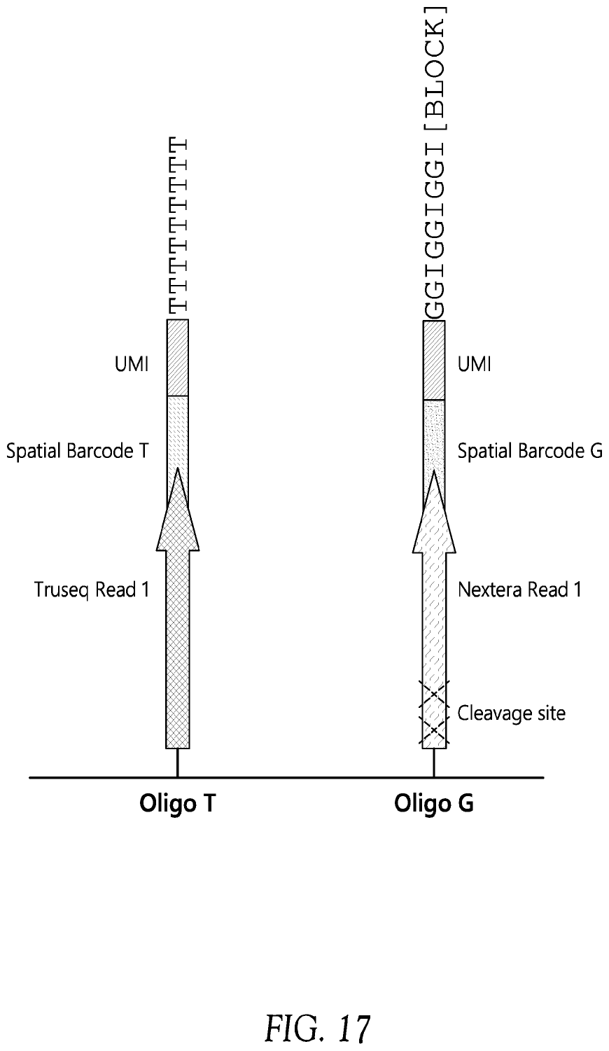

FIG. 17 is a schematic diagram showing an exemplary feature comprising an attached first and second probe. The first probe comprises in a 5′ to 3′ direction: a functional domain comprising a Truseq Read 1 primer, a spatial barcode, a UMI, and a poly(T) capture domain, where the poly(T) capture domain binds specifically to the target nucleic acid. The 5′ end of the first probe is attached to the feature.

The second probe comprises in a 5′ to 3′ direction: a cleavage domain, a functional domain comprising a Nextera Read 1 primer, a spatial barcode, a UMI, and a poly(GI) capture domain. The 5′ end of the second probe is attached to the feature. In some embodiments, the poly(GI) capture domain comprises a sequence of (GGI)n, wherein n is about 3 to about 20. In some embodiments, the poly(GI) capture domain comprises a sequence of (GGI)n, wherein n is 1, 2, 3, 4, 5, 6, 7, 8, 9, 10, 11, 12, 13, 14, 15, 16, 17, 18, 19, or 20. In some embodiments, the 3′ end of the second probe is reversibly blocked.

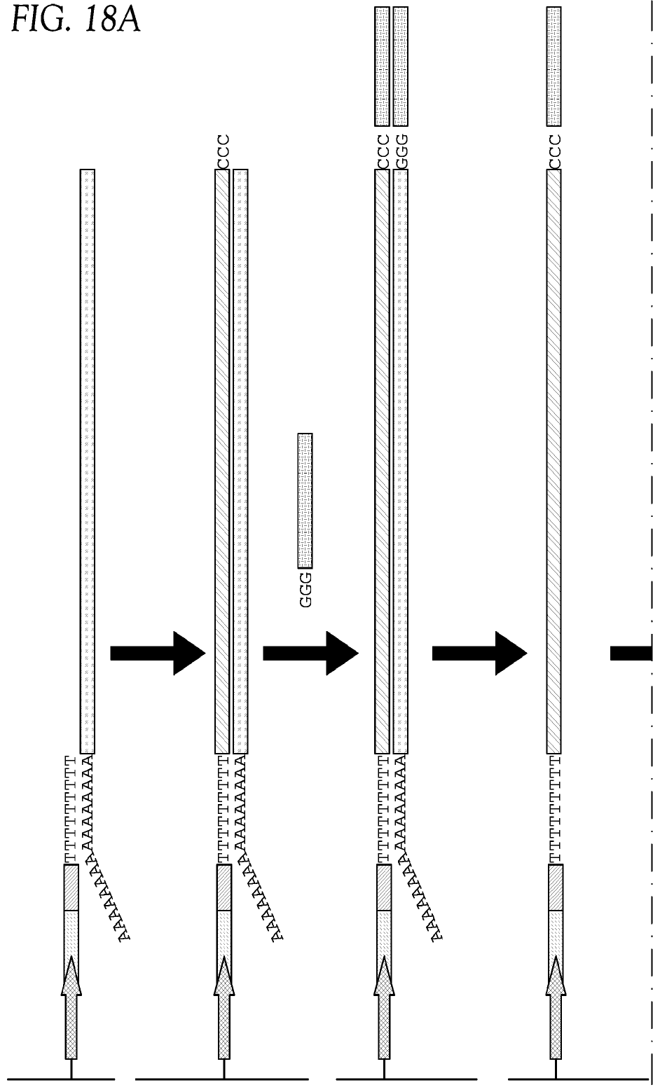

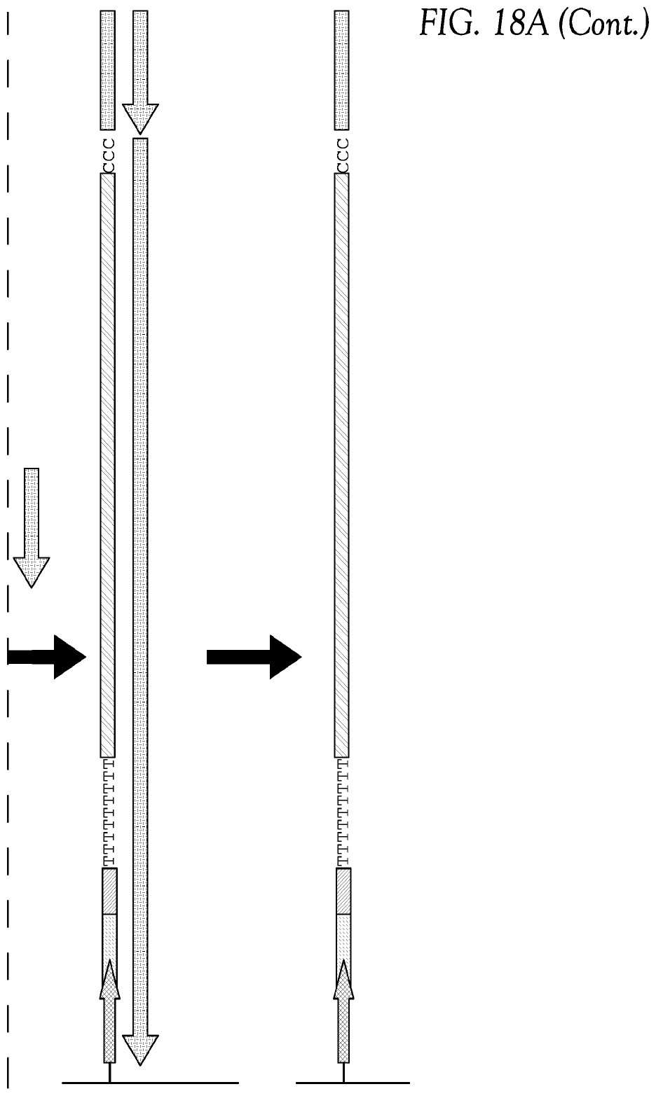

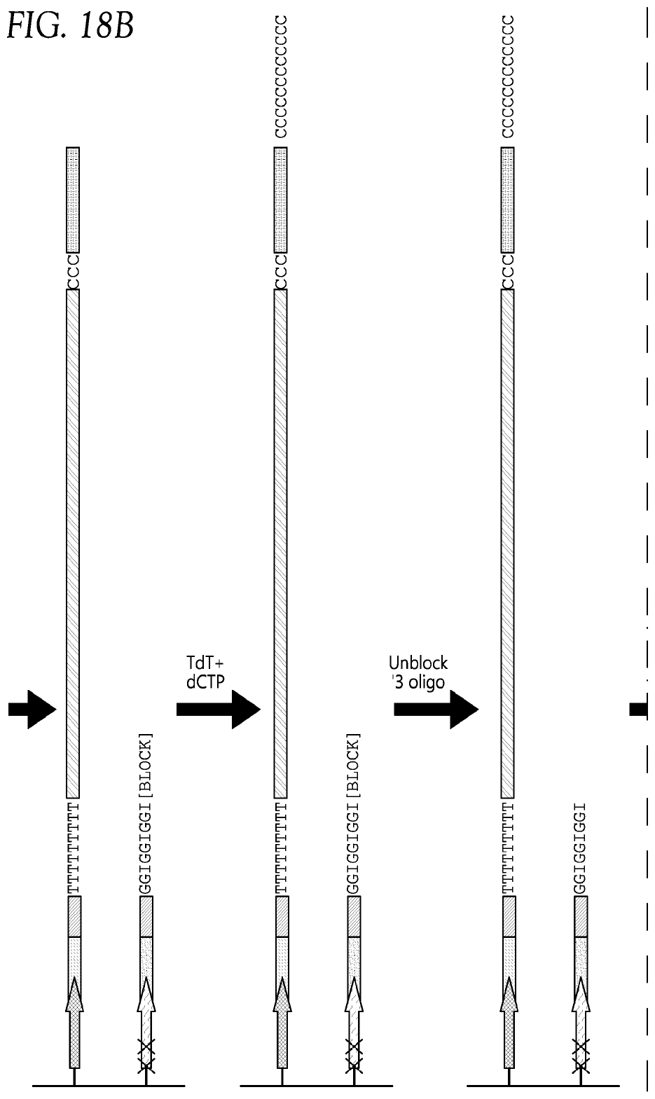

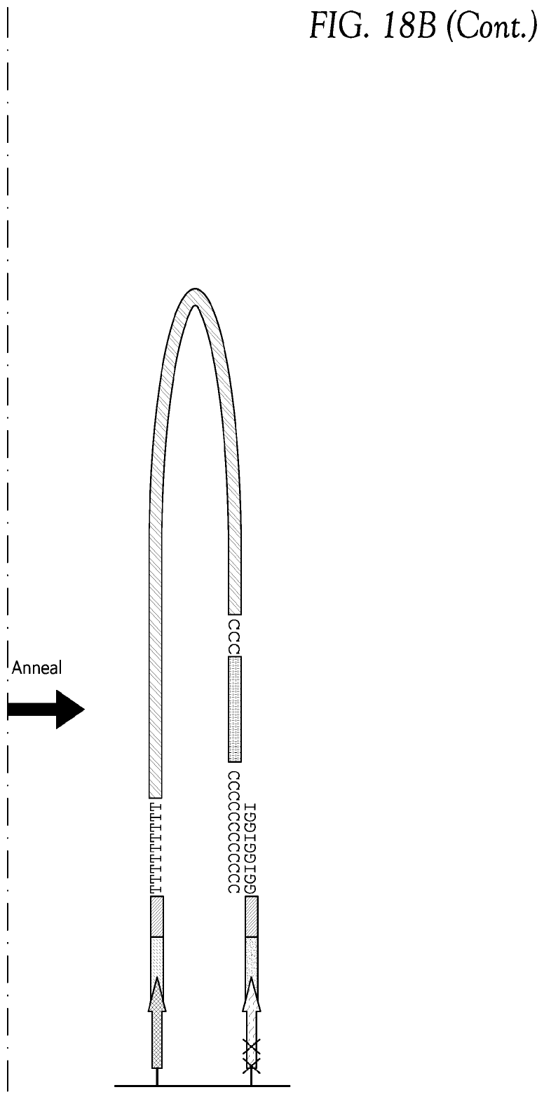

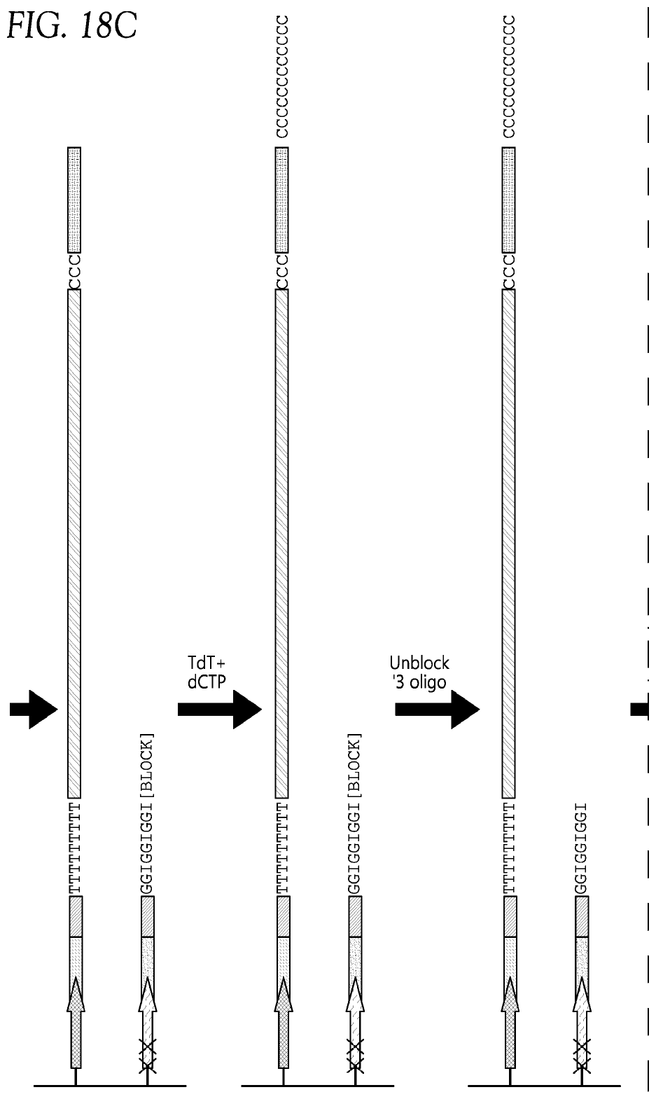

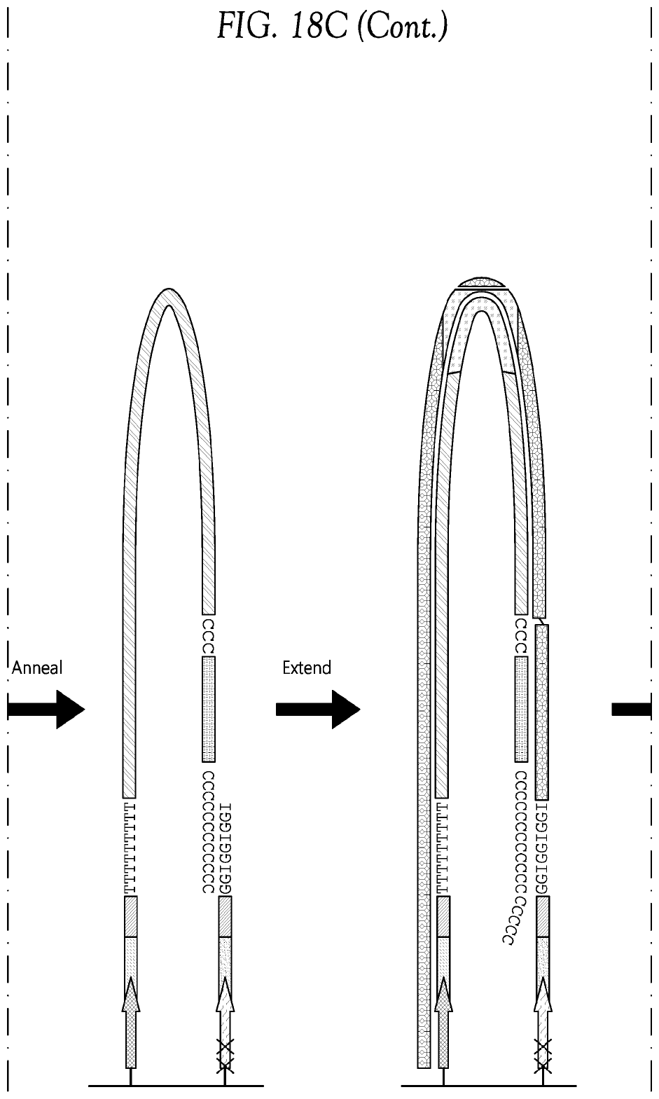

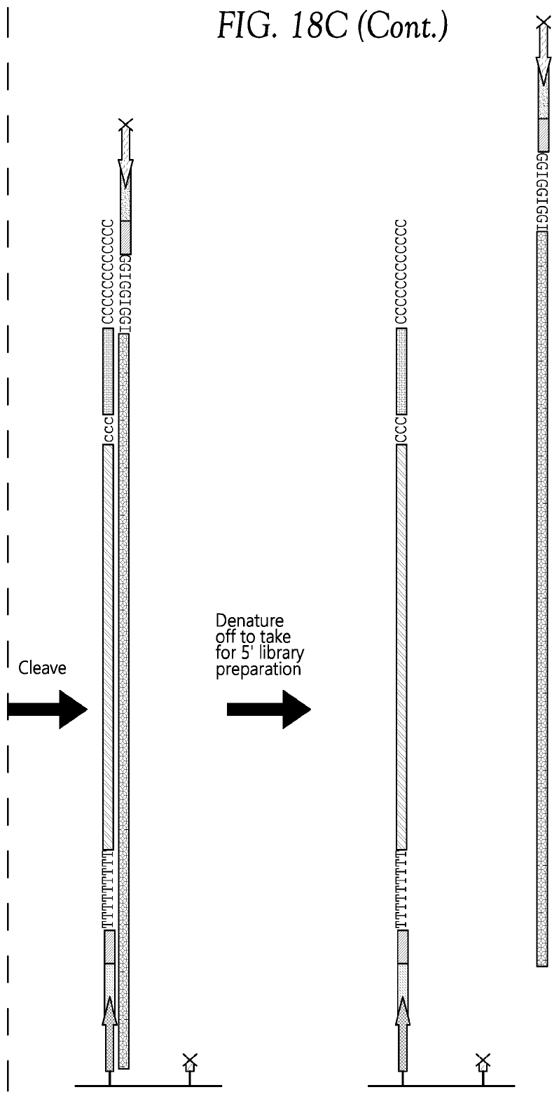

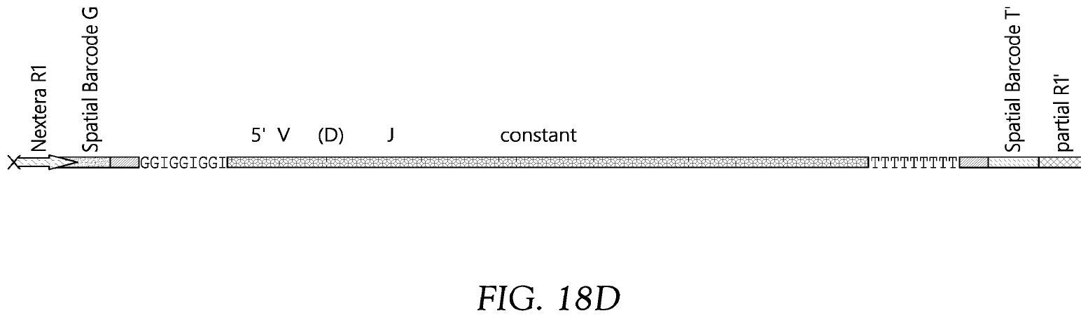

FIG. 18A is an exemplary diagram showing, from left to right, the annealing of the target analyte (e.g., target nucleic acid) to the poly(T) capture domain of the first probe; the extension of the first probe to add a sequence that is complementary to a portion of the target nucleic acid; the ligation of an adaptor to the 5′ end of the target nucleic acid specifically bound to the first probe; the addition of a sequence complementary to the adaptor to the 3′ end of the first probe; the releasing of the target nucleic acid from the first probe; the generation of a complement of the extended first probe; and the releasing of the complement of the extended first probe. In some embodiments, the released target nucleic acid is sequenced. In some embodiments, the released complement of the extended first probe is sequenced.

FIG. 18B is an exemplary diagram showing, from left to right, the addition of non-templated cytosines to the 3′ end of the extended first probe (e.g., extended to include a sequence that is complementary to part of a sequence of a target nucleic acid) to generate a poly(C) sequence, where the poly(C) sequence specifically binds to the poly(GI) capture domain of the second probe; the unblocking of the 3′ end of the second capture probe; and the hybridization of the poly(C) sequence on the 3′ end of the first probe to the poly(GI) capture domain at the 3′ end of the second probe.

In alternative embodiments, the second probe comprises a poly(T) capture domain and a poly(A) sequence is added to the 3′ end of the extended first probe (e.g., extended to add a sequence that is complementary to a portion of the sequence of a target nucleic acid), and the poly(A) sequence hybridizes to the poly(T) capture domain of the second probe.

FIG. 18C is an exemplary diagram showing from left to right, the addition of non-templated cytosines to the 3′ end of the extended first probe (e.g., extended to include a sequence that is complementary to a portion of the sequence of a target nucleic acid, and optionally, further comprising an adaptor sequence or a functional domain) to generate a poly(C) sequence, where the poly(C) sequence specifically binds to the poly(GI) capture domain of the second probe; the unblocking of the 3′ end of the second probe; the hybridizing the poly(C) sequence on the first probe to the poly(GI) capture domain on the second capture probe; the extension of the 3′ end of the second probe to add a sequence complementary to the extended first capture probe. The final step depicted in FIG. 18C is the releasing of the extended second probe sequence from the feature.

FIG. 18D is a schematic diagram showing an example of a sequence generated by the methods described in this section. The exemplary sequence shown comprises, from 5′ end to 3′ end, the functional domain of the second probe, which comprises a sequencing primer; the spatial barcode of the second probe; the UMI sequence of the second probe; the poly(GI) sequence of the second probe; the target nucleic acid sequence (from 5′ end to 3′ end); a sequence complementary to the UMI sequence of the first probe; a sequence complementary to the spatial barcode of the first probe; and a sequence complementary to part or the full sequence of the functional domain of the first probe, which comprises a sequencing primer. In some embodiments, the two sequencing primers have the same sequence. In some embodiments, the two sequencing primers have different sequences.

Further steps of the methods described in this section include, for example, determining (i) all or a part of the sequence of the spatial barcode on either end of the sequence depicted in FIG. 18D, or a complement thereof, and (ii) all or a part of the sequence of the target nucleic acid, or a complement thereof, and using the sequences of (i) and (ii) to determine the location of the target nucleic acid in the biological samples.

The methods described in this section allows for the sequencing of the target nucleic acid from either the 3′ end or the 5′ end, or both the 3′ and the 5′ ends of the target nucleic acid. For target nucleic acids that have large sizes (e.g., larger than 1 kb), the methods allow more accurate spatial sequence information to be obtained.

Also described in this section is an array comprising a feature, where the feature comprises an attached first and second probe, wherein: a 5′ end of the first probe is attached to the feature; the first probe comprises in a 5′ to a 3′ direction: a spatial barcode and a poly(T) capture domain, wherein the poly(T) capture domain binds specifically to the target nucleic acid; a 5′ end of the second probe is attached to the feature; a 3′ end of the second probe is reversibly blocked; and the second probe comprises a poly(GI) capture domain.

In some embodiments of any of the arrays described in this section, a feature can include two or more pairs of a first and a second probe (e.g., any of the first and second probes described in this section). A first pair of a first and a second probe at a feature, as compared to a second pair of a first and a second probe at the feature, can have a different first and/or second probe as compared to first and/or second probe of the second pair (e.g., a different capture domain in the first probe and/or a different barcode in the first and/or second probes). In some embodiments of any of the arrays described in this section, the spatial barcode in the first probe of the first pair and the spatial barcode in the first probe of the second pair are the same. In some embodiments of any of the arrays described in this section, the spatial barcode in the first probe of the first pair and the spatial barcode in the first probe of the second pair are different. In some embodiments of any of the arrays described in this section, the capture domain of the first probe of the first pair is the same as the capture domain of the first probe of the second pair. In some embodiments of any of the arrays described in this section, the capture domain of the first probe of the first pair is different from the capture domain of the first probe of the second pair.

In some embodiments of any of the arrays described in this section, the capture domain on the first probe has a poly(T) capture domain, where the poly(T) capture domain is configured to interact with a target nucleic acid (e.g., positioned at the 3′ end of the first probe). For example, the poly(T) capture domain specifically binds to a messenger RNA (mRNA), via the poly(A) tail of the mRNA. For example, a poly(T) capture domain can include at least 3, at least 4, at least 5, at least 6, at least 7, at least 8, at least 9, at least 10, at least 11, at least 12, at least 13, at least 14, at least 15, at least 16, at least 17, at least 18, at least 19, at least 20, at least 25, or at least 30 contiguous thymidines.

In some embodiments of any of the arrays described in this section, the poly(GI) capture domain comprises a sequence of at least 5 contiguous guanosine(s) and/or inosine(s). For example, a poly(GI) capture domain comprises a sequence of (GGI)n, wherein n is about 3 to about 20. In some embodiments, the poly(GI) capture domain comprises a sequence of (GGI)n, wherein n is 1, 2, 3, 4, 5, 6, 7, 8, 9, 10, 11, 12, 13, 14, 15, 16, 17, 18, 19, or 20. For example, a poly(GI) capture domain comprises a sequence of (GI)n, wherein n is about 4 to about 30. For example, a poly(GI) capture domain comprises a sequence of (GI)n, wherein n is 4, 5, 6, 7, 8, 9, 10, 11, 12, 13, 14, 15, 16, 17, 18, 19, 20, 21, 22, 23, 24, 25, 26, 27, 28, 29, or 30. For example, a poly(GI) capture domain comprises a sequence of (IG)n, wherein n is about 4 to about 30. For example, a poly(GI) capture domain comprises a sequence of (IG)n, wherein n is 4, 5, 6, 7, 8, 9, 10, 11, 12, 13, 14, 15, 16, 17, 18, 19, 20, 21, 22, 23, 24, 25, 26, 27, 28, 29, or 30.

In some embodiments of any of the arrays described in this section, the second probe can comprise a spatial barcode, which is positioned 5′ to the poly(GI) capture domain. In some embodiments, the spatial barcode in the first probe is different from the spatial barcode sequence in the second probe. In some embodiments, the spatial barcode in the first probe is the same as the spatial barcode sequence in the second probe.

In some embodiments of any of the arrays described in this section, both the first and the second probes are cleavable. In some embodiments, the first probe and the second probe have different cleavage site and are cleavable using different methods. In some embodiments, the first probe and the second probe have the same cleavable site and are cleavable using the same method. In some embodiments, the cleavage domain of the first probe is 5′ to the poly(T) capture domain and/or the cleavage domain of the second probe is 5′ to the poly(GI) capture domain.

In some embodiments of any of the arrays described in this section, the first probe is not cleavable and the second probe is cleavable. In some embodiments, the cleavage site of the second probe is 5′ to the poly(GI) capture domain of the second probe. In some embodiments, the cleavage site on the second probe is a uracil. In some embodiments, the uracil is cleaved by USER (Uracil-Specific Excision Reagent).

In some embodiments of any of the arrays described in this section, the first probe further comprises a unique molecular identifier (UMI). In some embodiments, the second probe further comprises a unique molecular identifier (UMI). In some embodiments, the UMI in the first probe and the UMI in the second probe comprise different sequences. In some embodiments, the UMI in the first probe and the UMI in the second probe comprise the same sequence.

In some embodiments of any of the arrays or methods described in this section, the first and/or second probe can further include a functional domain (e.g., a sequencing handle). In some embodiments, the first and second probe comprise a functional domain. In some embodiments, the functional domain the first and second probes is the same. In some embodiments, the functional domain in the first probe and the functional domain in the second probe are different.

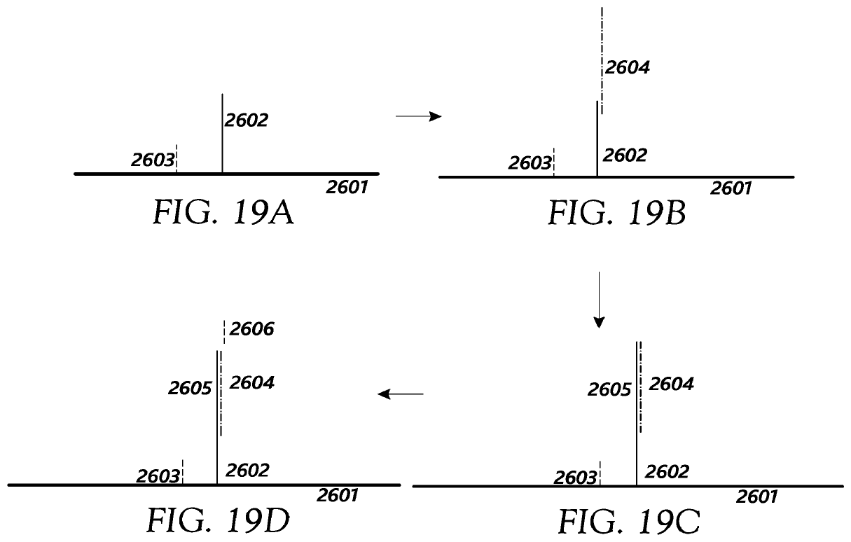

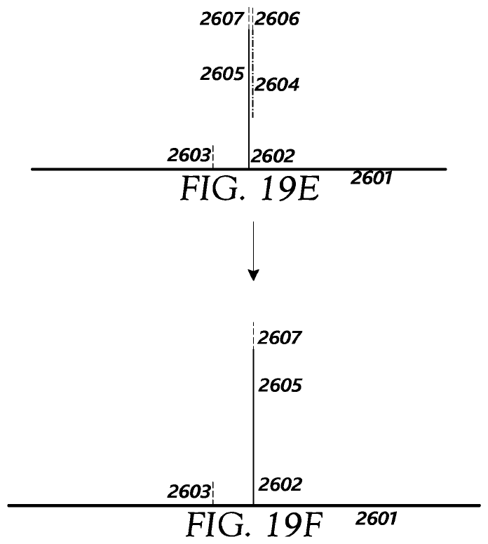

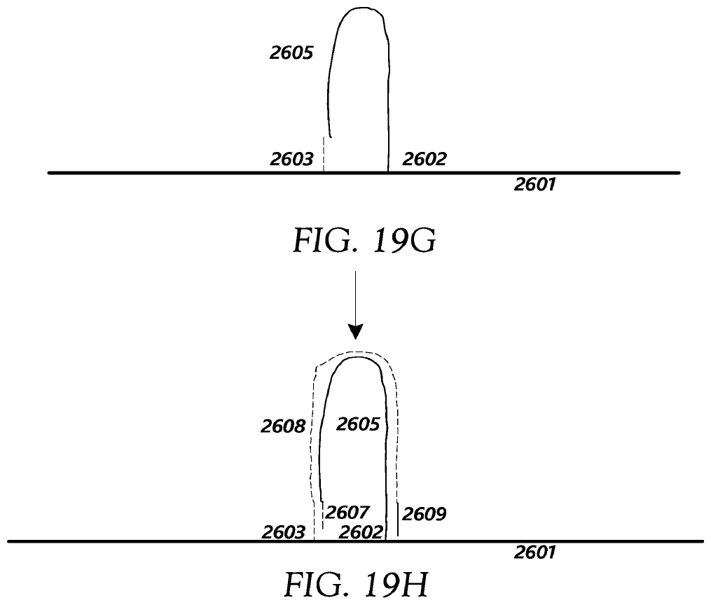

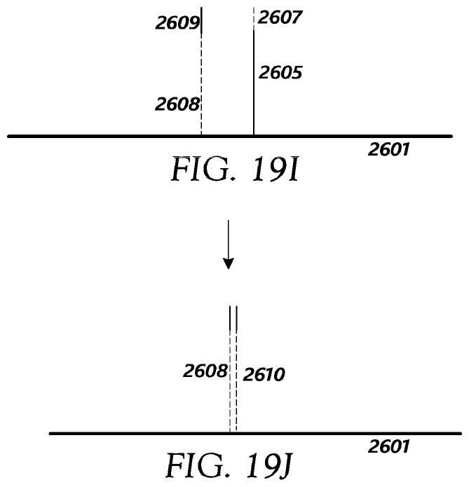

An exemplary, non-limiting workflow is depicted in FIGS. 19A-19J. In the workflow depicted in FIGS. 19A-19J, presence and/or location (e.g., spatial location) of a biological analyte may be determined and/or a 5′ sequence (e.g., sequence of a 5′ region) of a polynucleotide sequence of a biological analyte may be detected and/or determined. A “5′ region” refers to a sequence that is at or near the 5′ end of a polynucleotide sequence, or a sequence that is closer in proximity to the 5′ end than the 3′ end of a polynucleotide sequence. A biological sample is contacted with a substrate 2601. The substrate includes an attached first polynucleotide probe 2602 and an attached second polynucleotide probe 2603. (FIG. 19A) In one embodiment, the first polynucleotide probe 2602 includes, in a 5′-3′ direction: a barcode (e.g., spatial barcode); a first capture domain; and an extendible 3′ end. In the embodiment depicted in FIGS. 19A-19J, the second polynucleotide probe 2603 includes a second capture domain and an extendible 3′ end, and both the first polynucleotide probe 2602 and the second polynucleotide 2603 are attached at their 5′ ends to the substrate 2601.

A biological sample is contacted with the substrate 2601 under conditions in which a target polynucleotide sequence 2604 of a biological analyte binds (e.g., hybridizes) to the first capture domain of the first polynucleotide probe 2602. (FIG. 19B) In some instances, the first capture domain includes a sequence specific for an RNA molecule. In some instances, the first capture domain includes a poly-T sequence. In some instances, the first capture domain includes a sequence complementary to a region of an immune molecule, such as the constant region of a TCR or BCR sequence (as described herein). In some instances, the first capture domain includes a sequence complementary to a region of an immunoglobulin molecule, such as one or more CDRs of an immunoglobulin heavy or light chain.

The extendible 3′ end of the first polynucleotide probe 2602 is extended to produce a first extension product 2605. (FIG. 19C) An adapter is attached to the 3′ end of the first extension product 2605. In one embodiment, depicted in FIG. 19D, untemplated nucleotides are added to the 3′ end of the first extension product 2605. A template switch oligonucleotide (TSO) 2606 binds (e.g., hybridizes) to the untemplated nucleotides, and then the 3′ end of the first extension product is extended, producing a polynucleotide sequence 2607 that is complementary to the TSO sequence. (FIG. 19E). The target polynucleotide sequence 2604 and TSO 2606 are stripped away (e.g., denatured). (FIG. 19F)

In the embodiment depicted in FIG. 19G, the second capture domain of the second capture probe 2603 includes a sequence that is complementary to the adapter sequence 2607, i.e., the second capture domain contains the TSO sequence or a partial sequence thereof. The adapter 2607 at the 3′ end of the first extension product 2605 binds to the second capture domain sequence at the 3′ end of the second capture probe 2603. The 3′ end of the second capture probe 2603 is extended, producing a second extension product 2608, which includes a 3′ sequence that is complementary to the sequence of the first capture probe or a portion thereof 2609. (FIG. 19H) The first extension product 2605 with 3′ adapter sequence 2607 includes a 3′ sequence complementary to the target polynucleotide proximal to the first capture domain sequence, and may be used for preparation of a 3′ sequence library; and/or the second extension product 2608 with 3′ sequence complementary to the first polynucleotide probe 2607 includes a 5′ sequence of the target polynucleotide proximal to the second capture domain sequence, and may be used for preparation of a 5′ sequence library. (FIG. 19I) In one embodiment, depicted in FIG. 19J, a copy 2610 of the second extension product (e.g., amplification product) 2608 is produced. The first and/or second extension product, and/or copy (e.g., amplification product thereof) may be detected and/or sequenced, and the resulting information obtained may be used to determine presence and/or location (e.g., spatial location) of the biological analyte in the biological sample.

In some embodiments of a spatial immune profiling method, the analyte of the ABM-expressing cell is a cDNA of an mRNA transcript encoding the ABM. In some embodiments, the cDNA is generated by in situ reverse transcription of the mRNA encoding the ABM.

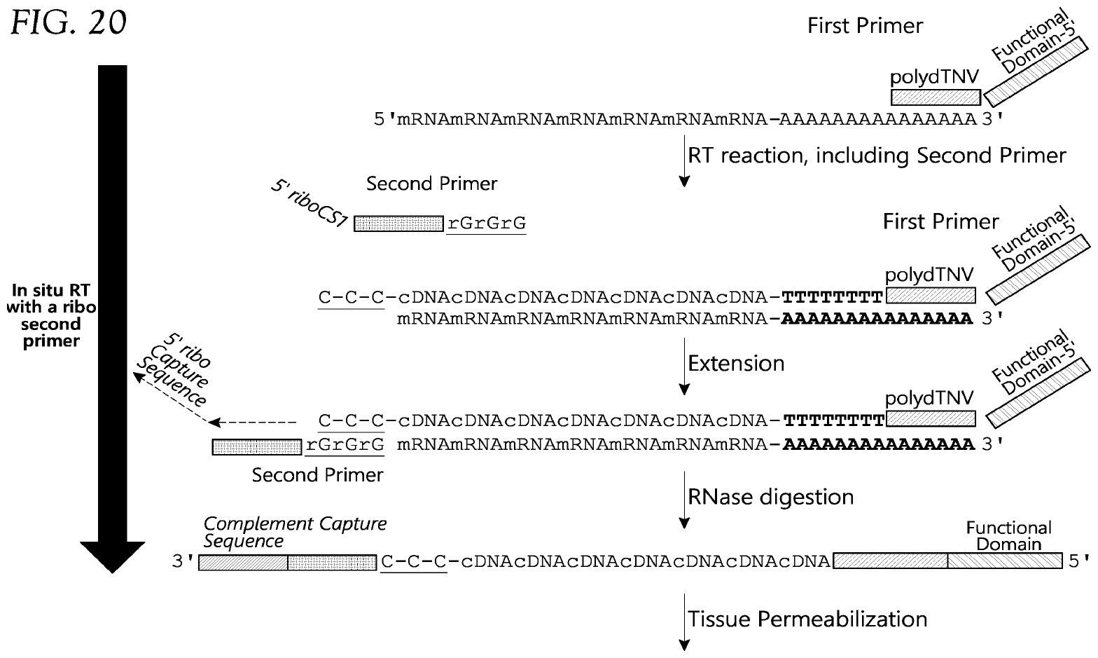

FIG. 20 is a schematic showing generation of a cDNA by in situ reverse transcription of a target nucleic acid (e.g., mRNA) from a first primer including a sequence complementary to the target nucleic acid and a functional domain and a second primer that includes a capture sequence and a sequence complementary a homopolynucleotide sequence.

More specifically, target nucleic acids are contacted with a first primer that includes a sequence complementary to the target nucleic acid (e.g., poly(dT) sequence, a poly(dTNV) sequence) and a functional domain. In some examples, the functional domain is a primer binding site. In some examples, the functional domain is a sequencing specific site (e.g., Read2 site). The target nucleic acid is reverse transcribed and a homopolynucleotide sequence is added to the 3′ end of the cDNA.

A second primer is added where the second primer includes a sequence complementary to the homopolynucleotide sequence added to the 3′ end of the cDNA and a capture sequence. In some examples, the second primer includes an RNA sequence (e.g., a ribo-functional sequence such as a linker sequence, a primer binding sequence, a sequence for use in next generation sequencing, etc.). After reverse transcription and extension of the 3′ end of the cDNA using the second primer as an extension template, an RNase (e.g., RNase H) is contacted with the biological sample (e.g., a tissue section). The RNase degrades the RNA strand of the RNA/cDNA duplex, leaving a single-stranded cDNA product (e.g., an extension product) that includes the first primer at its 5′ end and a capture sequence capable of hybridizing a capture domain of a capture probe.

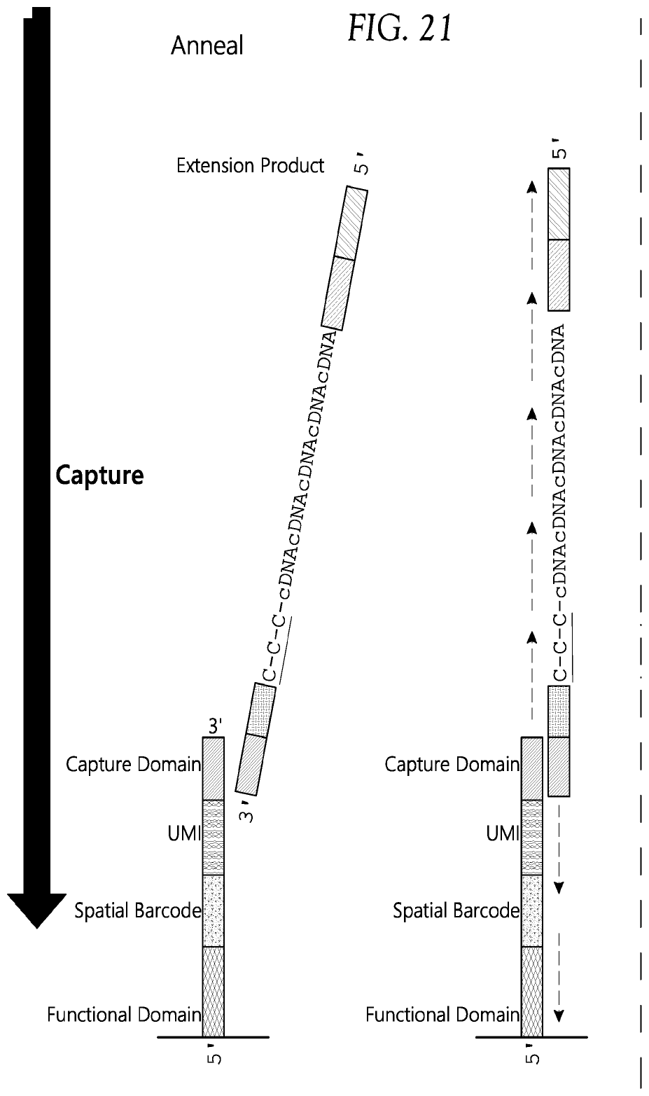

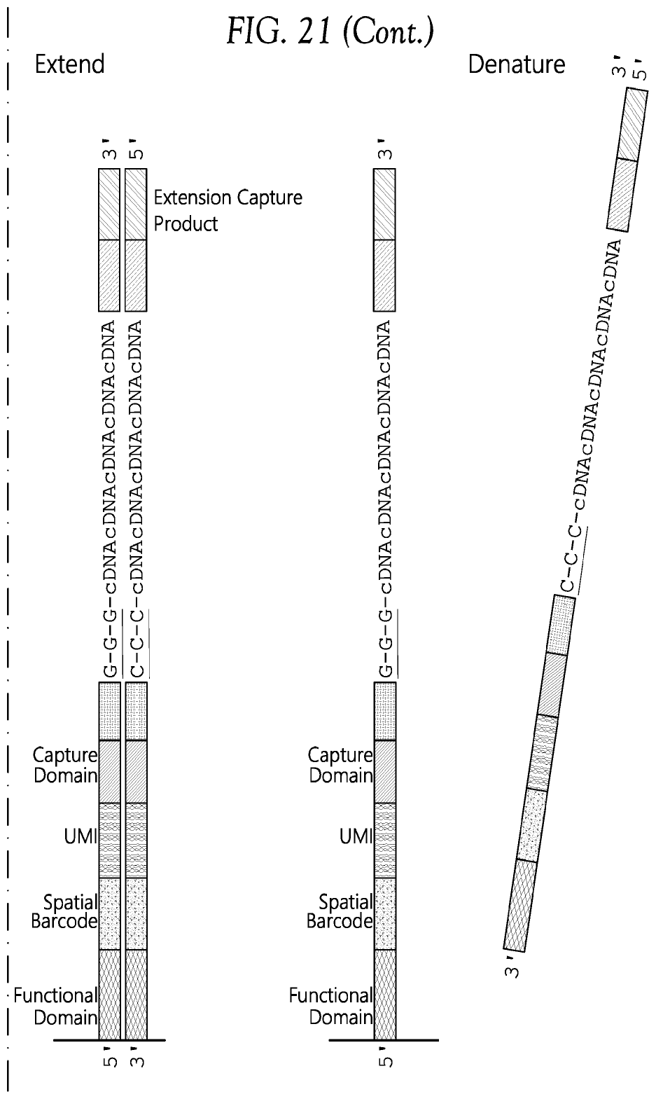

FIG. 21 is a schematic showing capture of the extension product (e.g., the single-stranded cDNA product shown in FIG. 20) by a capture probe on the substrate. The capture probe is attached to the substrate via its 5′ end and can include one or more functional domains, a spatial barcode, a unique molecular identifier, and a capture domain. In some examples, the capture probe also includes a cleavage domain. The capture domain hybridizes to the capture sequence on the extension product (e.g., single-stranded cDNA product) from FIG. 20. In some examples, the 3′ end of the capture probe is extended using the extension product as a template. In some examples, the 3′ end of the extension product (e.g., single-stranded cDNA product) is extended using the capture probe as a template thereby generating an extended capture product. In some examples, the 3′ end of the capture probe is extended using the extension product as a template and the 3′ end of the extension product is simultaneously extended using the capture probe as a template (e.g., generating an extended capture product). In some examples, the extended capture product is released from the capture probe. In some examples, the extended capture product is released via heat. In some examples, the extended capture product is denatured from the capture probe. In some examples, the extended capture product is denatured from the capture probe with KOH.

The released, extended captured products can be prepared for downstream applications, such as generation of a sequencing library and next-generation sequencing.

### ABM Analysis Using 5′ Capture of Target Nucleic Acids

Since capture on a spatial array generally biases the 3′ end of nucleic acid analytes, methods are needed to determine the sequence of the 5′ end of nucleic acid analytes (e.g., by capturing the 5′ end of the nucleic acid analyte), or a complement thereof (e.g., a proxy of the analyte). For example, target nucleic acid analytes (e.g., RNA) can be reverse transcribed with a first primer including a sequence complementary to the target nucleic acid and a functional domain, such as a primer binding site or a sequencing specific site to generate an RNA/DNA (e.g., cDNA) duplex. An enzyme such as a reverse transcriptase or terminal transferase can add non-templated nucleotides to the 3′ end of the cDNA. For example, a reverse transcriptase or terminal transferase enzyme can add at least 3 nucleotides (e.g., a polynucleotide sequence (e.g., a heteropolynucleotide sequence (e.g., CGC), a homopolynucleotide sequence (e.g., CCC))) to the 3′ end of the cDNA. A second primer that includes a sequence complementary to the non-templated nucleotides (e.g., the polynucleotide sequence) and a capture sequence can hybridize to the non-templated nucleotides (e.g., the polynucleotide sequence) added to the end of the cDNA. In some embodiments, the second primer includes an RNA sequence (e.g., one or more ribonucleotides). The cDNA is extended using the second primer as a template thereby incorporating the complement of the capture sequence into the cDNA. The complement of the capture sequence can hybridize to the capture domain of the capture probe on the substrate. The target nucleic acid with the ribo-second primer can be removed (e.g., digested, denatured, etc.) resulting in a single-stranded DNA product. The single-stranded DNA product can include the functional domain at its 5′ end, a copy of the target analyte (e.g., cDNA), and a complement of the capture sequence that is capable of binding (e.g., hybridizing) to a capture domain of a capture probe on the array at its 3′ end.

Target nucleic acid analytes (also referred to herein as analytes or target nucleic acids) can include a nucleic acid molecule with a nucleic acid sequence encoding at least a portion of a V-J sequence or a V(D)J sequence of an immune cell receptor (e.g., a T cell receptor or a B cell receptor). Target nucleic acids can include a nucleic acid molecule with a nucleic acid sequence encoding an antibody. In some embodiments, the target nucleic acid is RNA. In some embodiments, the RNA is mRNA. In some embodiments, the target nucleic acids are nucleic acids encoding immune cell receptors. In some embodiments, target nucleic acids encoding immune cell receptors identify clonotype populations from a biological sample. In some embodiments, target nucleic acids include a constant region, such as a sequence encoding a constant region of an immune cell receptor (e.g., antibody). In some embodiments, target nucleic acids include a variable region, such as a sequence encoding a variable region of an immune cell receptor (e.g., antibody).

In some embodiments, the target nucleic acid encodes an immune cell receptor. In some embodiments, the immune cell receptor is a B cell receptor. In some embodiments, the B cell receptor includes an immunoglobulin kappa light chain. In some embodiments, the target nucleic acid includes a sequence encoding a CDR3 region of the immunoglobulin kappa light chain. In some embodiments, the target nucleic acid includes a sequence encoding one or both of CDR1 and CDR2 of the immunoglobulin kappa light chain. In some embodiments, the target nucleic acid includes a sequence encoding a full-length variable domain of the immunoglobulin kappa light chain.

In some embodiments, the B cell receptor includes an immunoglobulin lambda light chain. In some embodiments, the target nucleic acid includes a sequence encoding a CDR3 of the immunoglobulin lambda light chain. In some embodiments, the target nucleic acid includes a sequence encoding one or both of CDR1 and CDR2 of the immunoglobulin lambda light chain. In some embodiments, the target nucleic acid includes a sequence encoding a full-length variable domain of the immunoglobulin lambda light chain.

In some embodiments, the B cell receptor includes an immunoglobulin heavy chain. In some embodiments, the target nucleic acid includes a sequence encoding a CDR3 of the immunoglobulin heavy chain. In some embodiments, the target nucleic acid includes a sequence encoding one or both of CDR1 and CDR2 of the immunoglobulin heavy chain. In some embodiments, the target nucleic acid includes a sequence encoding a full-length variable domain of the immunoglobulin heavy chain.

In some embodiments, the immune cell receptor is a T cell receptor. In some embodiments, the T cell receptor includes a T cell receptor alpha chain. In some embodiments, the target nucleic acid includes a sequence encoding a CDR3 of the T cell receptor alpha chain. In some embodiments, the target nucleic acid includes a sequence encoding one or both of CDR1 and CDR2 of the T cell receptor alpha chain. In some embodiments, the target nucleic acid includes a sequence encoding a full-length variable domain of the T cell receptor alpha chain.

In some embodiments, the T cell receptor includes a T cell receptor beta chain. In some embodiments, the target nucleic acid includes a sequence encoding a CDR3 of the T cell receptor beta chain. In some embodiments, the target nucleic acid includes one or both of CDR1 and CDR2 of the T cell receptor beta chain. In some embodiments, the target nucleic acid further includes a full-length variable domain of the T cell receptor beta chain.

Provided herein are methods for determining a location of a target nucleic acid in a biological sample, the method including: (a) contacting the biological sample with a first primer including a nucleic acid sequence that hybridizes to a complementary sequence in the target nucleic acid and a functional domain; (b) hybridizing the first primer to the target nucleic acid and extending the first primer using the target nucleic acid as a template to generate an extension product; (c) adding a non-templated sequence (e.g., a polynucleotide sequence including at least three nucleotides) to the 3′ end of the extension product; (d) hybridizing a second primer to the non-templated sequence (e.g., polynucleotide sequence comprising at least three nucleotides of the extension product of (c)), where the second primer comprises a capture sequence; (e) extending the extension product using the second primer as a template, thereby incorporating a complement of the capture sequence into the extension product; (f) hybridizing the complement of the capture sequence of the extension product to a capture domain on an array, wherein the array includes a plurality of capture probes, and wherein a capture probe of the plurality of capture probes comprises a spatial barcode and the capture domain; and (g) determining (i) the sequence of the spatial barcode, or a complement thereof, and (ii) all or a portion of the sequence of the target nucleic acid, or a complement thereof, and using the determined sequences of (i) and (ii) to determine the location of the target nucleic acid in the biological sample.

The methods described herein can also use a plurality of primers, wherein the first primer is comprised in the plurality of primers. For example, the plurality of primers can hybridize to a target nucleic acid at different locations in the target nucleic acid and subsequently be extended. Extending the plurality of primers generates one or more extension products that include a complement of the capture sequence as described herein. For example, the methods provided herein can include providing a plurality of primers wherein each primer includes a sequence that hybridizes to a complementary sequence in the target nucleic acid and a functional domain, wherein the first primer is comprised in the plurality of primers and (a) hybridizing the plurality of primers to the target nucleic acid and extending one or more primers from the plurality of primers using the target nucleic acid as a template to generate one or more extension products; (b) attaching a non-templated sequence (e.g., a polynucleotide sequence including at least three nucleotides) to the 3′ end of the one or more extension products; (c) hybridizing the second primer to the non-templated sequence (e.g., polynucleotide sequence) of the one or more extension products of (b), where the second primer includes a capture sequence; (d) extending the one or more extension products using the second primer as a template, thereby incorporating a complement of the capture sequence into the one or more extension products; (e) hybridizing the complement of the capture sequence of the one or more extension products to a capture domain on an array, where the array includes a plurality of capture probes, and where a capture probe of the plurality of capture probes comprises a spatial barcode and the capture domain; and (f) determining (i) the sequence of the spatial barcode, or a complement thereof, and (ii) all or a portion of the sequence of the target nucleic acid, or a complement thereof, and using the determined sequences of (i) and (ii) to determine the location of the target nucleic acid in the biological sample.

Also provided herein are methods for determining locations of target nucleic acids in a biological sample, the method including: (a) contacting the biological sample with a plurality of primers, where the plurality of primers comprise nucleic acid sequences that hybridize to complementary sequences in the target nucleic acids and a functional domain; (b) hybridizing the plurality of primers to the target nucleic acids and extending one or more of the plurality of primers using the target nucleic acids as a template to generate one or more extension products; (c) adding a non-templated sequence (e.g., a polynucleotide sequence comprising at least three nucleotides) to the 3′ end of the one or more extension products; (d) hybridizing a second primer to the non-templated sequence (e.g., a polynucleotide sequence including the at least three nucleotides) of the one or more extension products of (c), wherein the second primer comprises a capture sequence; (e) extending the one or more extension products using the second primer as a template, thereby incorporating a complement of the capture sequence into the one or more extension products; (f) hybridizing the complement of the capture sequence of the one or more extension products to a plurality of capture domains on an array, wherein the array comprises a plurality of capture probes, and wherein a capture probe in the plurality of capture probes comprise a spatial barcode and a capture domain; and (g) determining (i) the sequences of the spatial barcodes, or a complement thereof, and (ii) all or a portion of the sequence of the target nucleic acids, or a complement thereof, and using the determined sequences of (i) and (ii) to determine the locations of the target nucleic acids in the biological sample.

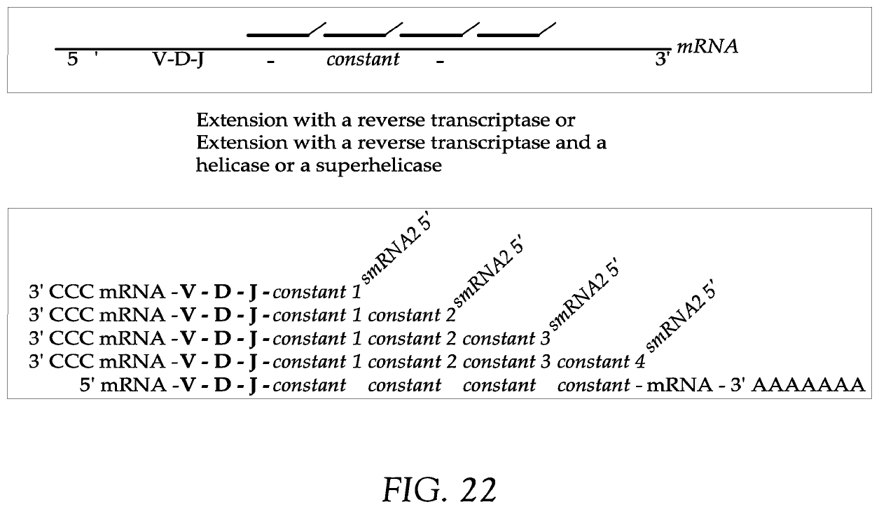

In embodiments where a plurality of primers are used, the plurality of primers can hybridize to different sequences in the target nucleic acid. For example, the plurality of primers can hybridize to adjacent sequences (e.g., “tiling”) on the target nucleic acid. Adjacent sequences can be, but are not necessarily, contiguous. In some embodiments, the plurality of primers hybridize to non-adjacent sequences on the target nucleic acid. In some embodiments, two or more primers of the plurality of primers hybridize to different sequences in the target nucleic acid. In some embodiments, ten or more primers of the plurality of primers hybridize to different sequences in the target nucleic acid. In some embodiments, fifty or more primers of the plurality of primers hybridize to different sequences in the target nucleic acid. In some embodiments, one hundred or more primers of the plurality of primers hybridize to different sequences in the target nucleic acid. In some embodiments, a plurality of extension products may be generated by extension of the plurality of primers. The resulting extension products can have differing lengths depending on the exact location in the target nucleic acid from which they were primed. Thus, in some embodiments, extension products (e.g., cDNA) of different lengths and/or sequences can be generated from the same target nucleic acid (e.g., mRNA). See, e.g., FIG. 22.

In embodiments where the target nucleic acid includes a sequence that encodes an immune cell receptor, the plurality of primers preferably hybridize to a region of the target nucleic acid that encodes a constant region of the immune cell receptor.

Some steps of the methods described herein can be performed in a biological sample (e.g., in situ) prior to contacting the biological sample with the array including a plurality of capture probes. In some embodiments, the biological sample is disposed or placed on the array including the plurality of capture probes prior to step (a). In some embodiments, the biological sample is disposed or placed on the array including the plurality of capture probes prior to step (f). In some embodiments, the biological sample is not disposed or placed on the array. For example, the biological sample can be placed on a substrate (e.g., a slide) that does not include a spatial array. In some embodiments, the substrate including the biological sample can be aligned with the array (e.g., “sandwiched”) such that at least a portion of the biological sample is aligned with at least a portion of the array. In embodiments where the biological sample is disposed or placed on a substrate, steps (a)-(e) can be performed prior to aligning the substrate with the array as described herein. In embodiments where the biological sample is disposed or placed on a substrate, steps (a)-(e) can be performed after aligning the substrate with the array as described herein.

In some embodiments, the extension product(s) that hybridize to the capture domains of the plurality of capture probes can migrate (e.g., diffuse) towards the capture probes through passive migration such as gravity. In some embodiments, the extension product(s) that hybridize to the capture domains of the plurality of capture probes can migrate toward the capture probes through active migration. In some embodiments, the active migration includes electrophoresis.

In some embodiments, the array includes one or more features (e.g., any of the features described herein). In some embodiments, the one or more features includes a bead.

As described herein, a capture probe refers to any molecule capable of capturing (directly or indirectly) and/or labelling an analyte (e.g., an analyte of interest, such as a nucleic acid) in a biological sample. In some embodiments, the capture probe is a nucleic acid. In some embodiments, the capture probe is DNA. In some embodiments, the capture probe includes a barcode (e.g., a spatial barcode) and/or a unique molecular identifier (UMI)) and a capture domain. In some embodiments, a capture probe can include a cleavage domain and/or one or more functional domains (e.g., a primer-binding site or a sequencing specific site, such as for next-generation sequencing (NGS)).

In some embodiments, the extending in step (a) includes the use of a reverse transcriptase. In some embodiments, the reverse transcriptase has strand displacement activity. In some embodiments, the strand displacement activity of the reverse transcriptase displaces one or more primers of the plurality of primers from the target nucleic acid. In some embodiments, the strand displacement activity of the reverse transcriptase displaces the one or more extension products from the target nucleic acid.

In some embodiments, the extending in step (a) includes the use of a reverse transcriptase and a helicase. As defined herein, “helicases” are enzymes that catalyze the reaction of separating or unwinding the helical structure of nucleic acid complexes, e.g. double-stranded DNA, double-stranded RNA, or DNA:RNA complexes, into single stranded components. Helicases generally are known to use a nucleoside triphosphate (NTP) (e.g., ATP) hydrolysis as a source of energy. In some embodiments, the method includes one or more single-stranded DNA binding proteins. In some embodiments, the one or more single-stranded DNA binding proteins comprises one or more of: Tth RecA, E. coli RecA, T4 gp32 and ET-SSB. As defined herein, “single-stranded DNA binding proteins” or “SSBs” are proteins that bind to single-stranded DNA. SSBs, or functional equivalents, are found in a variety of organisms, including eukaryotes and bacteria. Single-stranded DNA is produced, for example, during aspects of DNA metabolism, DNA replication, DNA recombination, and DNA repair. As well as stabilizing single-stranded DNA, SSB proteins bind to and modulate the function of numerous proteins involved in the aforementioned processes. In addition, SSB proteins can destabilize ends of double-stranded nucleic acid (e.g., dsDNA). In some embodiments, the helicase has strand displacement activity. For example, the helicases and single-stranded DNA binding proteins described herein can unwind DNA:RNA complexes that allow a reverse transcriptase to reverse transcribe the target nucleic acid. In some embodiments, a helicase can unwind a first target nucleic acid: extension product complex (e.g., mRNA:cDNA) that is downstream of a second target nucleic acid: extension product complex, such that the target nucleic acid from the first complex (with or without the help of the SSBs) becomes available as template for further extension of the extension product in the first complex.

In some embodiments, helicases, including superhelicases, are also used in conjunction with SSB proteins during nucleic acid amplification. In some embodiments, the extending in step (a) comprises the use of a superhelicase and a reverse transcriptase. In some embodiments, the superhelicase is selected from the group consisting of: Rep, PrcA, UvrB, RecBCD, and Tte-Uvrd. As defined herein a “superhelicase” is a mutant and/or a derivative of a helicase. Superhelicases can have increased processivity compared to helicases due to one or more derivations that can include mutated gene or substituted polypeptide sequences and/or cross-linked protein domains. Additionally, superhelicases can unwind double-stranded nucleic acid complexes without single-stranded DNA binding protein(s). In some embodiments, amplification of nucleic acid includes a DNA polymerase and a helicase. In some embodiments, the helicase is a superhelicase. In some embodiments, superhelicases have increased processivity relative to helicases. In some embodiments, the superhelicase has strand displacement activity. For example, superhelicases can unwind double-stranded nucleic acid complexes longer than 150 base pairs.

In some embodiments, the method includes generating two or more extension products from the primer of the plurality of primers. For example, a single primer can template reverse transcription for more than a single extension reaction resulting in 2, 3, 4, 5, 6, 7, 8, 9, 10, 20, 30, 40, 50, 100, 200, 300, 400, 500, 600, 700, 800, 900, 1000, or more extension products produced from a single primer. In some embodiments, the one or more extension products comprise different sequence lengths. For example, when a plurality of primers are hybridized to a target nucleic acid and extended, the one or more extension products can have varying lengths.

In some embodiments, the extending in step (b) includes the use of a reverse transcriptase (e.g., any suitable reverse transcriptase known in the art). In some embodiments, adding the polynucleotide sequence to the 3′ end of the extension product in step (c) includes the use of the reverse transcriptase. In some embodiments, adding the polynucleotide sequence to the 3′ end of the extension product in step (c) includes the use of a terminal transferase. In some embodiments, the terminal transferase is terminal deoxynucleotidyl transferase. In some embodiments, the reverse transcriptase or the terminal transferase adds at least three nucleotides to the extension product. In some embodiments, the reverse transcriptase or the terminal transferase adds 4, 5, 6, 7, 8, 9, 10, or more nucleotides (e.g., a polynucleotide sequence) to the 3′ end of the extension product(s) in step (c). In some embodiments, the polynucleotide sequence added to the 3′ end of the extension product(s) is 5′-CCC-3′. In some embodiments, the polynucleotide sequence added to the 3′ end of the extension product(s) is 5′CGC-3′.

In some embodiments, the extending in step (d) includes using the second primer as a template. In some embodiments, the second primer comprises RNA.

In some embodiments, a second primer is added before, contemporaneously with, or after reverse transcription or other terminal transferase-based reaction. In certain embodiments, methods of sample analysis using a second primer can involve the generation of nucleic acid products from target nucleic acids of the biological sample, followed by further processing of the nucleic acid products with the second primer.

In some embodiments, the method includes removing the target nucleic acid, or any other nucleic acid hybridized to the extension product(s) (e.g., extended cDNA product) before the complement of the capture sequence of the extension product(s) hybridizes to the capture domain of the capture probe. In some embodiments, the method includes removing the target nucleic acid and the ribo-second primer hybridized to the extension product (e.g., extended cDNA product) before the capture sequence of the second primer hybridizes to the capture domain of the capture probe. In some embodiments, the removing includes the use of an RNase. In some embodiments, the RNase is RNase A. In some embodiments, the RNase is RNase P. In some embodiments, the RNase is RNase T1. In some embodiments, the RNase is RNase H. In some embodiments, the removing includes heat.

In some embodiments, the method includes removing the target nucleic acid and/or any other nucleic acid hybridized to the extension product (e.g., extended DNA product) before the complement of the capture sequence of the second primer (incorporated into the extension product) hybridizes to the capture domain of the capture probe.

In some embodiments, the extension product(s) (e.g., the single-stranded extension product(s)) hybridizes to the capture domain of the capture probe on the substrate. In some embodiments, the 3′ end of the capture probe is extended using the extension product as a template. In some examples, the 3′ end of the extension product (e.g., single-stranded cDNA product) is extended using the capture probe as a template, thereby generating an extended capture product. In some embodiments, both the capture probe, and the extension products hybridized thereto, are extended from the 3′ ends. In some embodiments, the extending includes the use of a polymerase. Any suitable polymerase can be used (e.g., Kapa Hifi). In some examples, the 3′ end of the capture probe is extended using the extension product as a template and the 3′ end of the extension product is simultaneously extended using the capture probe as a template (e.g., generating an extended capture product). In some examples, the extended capture product is released from the capture probe. In some examples, the extended capture product is released via heat. In some examples, the extended capture product is denatured from the capture probe. In some examples, the extended capture product is denatured from the capture probe with KOH.

In some embodiments, the released, extended captured products can be prepared for downstream applications, such as generation of a sequencing library and next-generation sequencing. Generating sequencing libraries are known in the art. For example, the extended captured products can be purified and collected for downstream amplification steps. The extended amplification products can be amplified using PCR, where primer binding sites flank the spatial barcode and target nucleic acid, or a complement thereof, generating a library associated with a particular spatial barcode. In some embodiments, the library preparation can be quantitated and/or quality controlled to verify the success of the library preparation steps. The library amplicons are sequenced and analyzed to decode spatial information and the target nucleic acid sequence.

Alternatively or additionally, the amplicons can then be enzymatically fragmented and/or size-selected in order to provide for desired amplicon size. In some embodiments, when utilizing an Illumina® library preparation methodology, for example, P5 and P7, sequences can be added to the amplicons thereby allowing for capture of the library preparation on a sequencing flowcell (e.g., on Illumina sequencing instruments). Additionally, i7 and i5 can index sequences be added as sample indexes if multiple libraries are to be pooled and sequenced together. Further, Read 1 and Read 2 sequences can be added to the library preparation for sequencing purposes. The aforementioned sequences can be added to a library preparation sample, for example, via End Repair, A-tailing, Adaptor Ligation, and/or PCR. The cDNA fragments can then be sequenced using, for example, paired-end sequencing using TruSeq Read 1 and TruSeq Read 2 as sequencing primer sites, although other methods are known in the art.

In some embodiments, the biological sample is a tissue sample. In some embodiments, the tissue sample is a fixed tissue sample. For example, fixing the biological sample can include the use of a fixative including: ethanol, methanol, acetone, formaldehyde, paraformaldehyde-Triton, glutaraldehyde, and combinations thereof. In some embodiments, the fixed tissue sample is a formalin-fixed paraffin embedded tissue sample, paraformaldehyde fixed tissue sample, a methanol fixed tissue sample, or an acetone fixed tissue sample. In some embodiments, the tissue sample is a fresh frozen tissue sample. In some embodiments, the biological sample is a tissue section. In some embodiments, the biological sample is a fixed tissue section (e.g., a fixed tissue section prepared by any of the methods described herein).

In some embodiments, the method includes generating a sequencing library. In some embodiments, the determining in step (f) includes sequencing. Methods and systems for sequencing are known in the art and are described herein. In some embodiments, the sequencing is high-throughput sequencing.

### Methods and Systems for Multiplexing

In some embodiments, the present disclosure provides methods and systems for multiplexing, and otherwise increasing throughput of samples for analysis. For example, a single or integrated process workflow may permit the processing, identification, and/or analysis of more or multiple analytes, more or multiple types of analytes, and/or more or multiple types of analyte characterizations. For example, in the methods and systems described herein, one or more labeling agents capable of binding to or otherwise coupling to one or more cells or cell features can be used to characterize cells and/or cell features. In some instances, cell features include cell surface features. Cell features can include, but are not limited to, a receptor, an antigen, a surface protein, a transmembrane protein, a cluster of differentiation protein, a protein channel, a protein pump, a carrier protein, a phospholipid, a glycoprotein, a glycolipid, a cell-cell interaction protein complex, an antigen-presenting complex, a major histocompatibility complex, an engineered T-cell receptor, an ABM, an antibody, a T-cell receptor, a B-cell receptor, a chimeric antigen receptor, a gap junction, an adherens junction, or any combination thereof. In some instances, cell features can include intracellular analytes, such as proteins, protein modifications (e.g., phosphorylation status or other post-translational modifications), nuclear proteins, nuclear membrane proteins, or any combination thereof. In some instances, a cell feature can comprise an ABM, e.g., an antibody such as a secreted antibody, an immune receptor (e.g., a TCR, a BCR, or an FcR). A labeling agent can include, but is not limited to, a protein, a peptide, an antibody (or an epitope binding fragment thereof), a lipophilic moiety (such as cholesterol), a cell surface receptor binding molecule, a receptor ligand, a small molecule, a bi-specific antibody, a bi-specific T-cell engager, a T-cell receptor engager, a B-cell receptor engager, a pro-body, an aptamer, an antigen, a monobody, an affimer, a Darpin, and a protein scaffold, or any combination thereof. In particular embodiments, the labeling agent includes an antigen such as an AbTx or ADC. In some embodiments, the AbTx or ADC is bound to its antigenic target (AgTx).

The labeling agents can include (e.g., be attached to or conjugated with) a reporter oligonucleotide that is indicative of the cell feature to which the labeling agent binds. For example, the reporter oligonucleotide can include a barcode sequence that permits identification of the labeling agent. For example, a labeling agent that is specific to one type of cell feature (e.g., a first cell feature) can have a first reporter oligonucleotide comprising a first reporter barcode sequence coupled thereto, while a labeling agent that is specific to a different cell feature (e.g., a second cell feature) can have a different reporter oligonucleotide comprising a different reporter barcode sequence coupled thereto. For a description of exemplary labeling agents, reporter oligonucleotides, and methods of use, see, e.g., U.S. Pat. No. 10,550,429; U.S. Pat. Pub. 20190177800; and U.S. Pat. Pub. 20190367969, each of which is herein entirely incorporated by reference for all purposes.

In a particular example, a library of potential cell feature labeling agents can be provided, where the respective cell feature labeling agents are associated with reporter oligonucleotides, such that a different reporter oligonucleotide sequence is associated with each labeling agent capable of binding to a specific cell feature. In other aspects, different members of the library can be characterized by the presence of a different oligonucleotide sequence label. For example, an antibody capable of binding to a first protein can have associated with it a first reporter oligonucleotide sequence, while an antibody capable of binding to a second protein can have a different reporter oligonucleotide sequence associated with it. For other example, a first antigen that is an AbTx or ADC can have associated with it a first reporter barcode sequence, while a second antigen (that is a different AbTx or ADC) can have associated with it a second reporter barcode sequence. The presence of the particular barcode sequence can be indicative of the presence of a particular cell feature which can be recognized or bound by the particular labeling agent. Labeling agents capable of binding to or otherwise coupling to one or more cells can be used to characterize a cell as belonging to a particular set of cells. For example, labeling agents can be used to label a sample of cells or a group of cells. In this way, a group of cells can be labeled as different from another group of cells. In an example, a first group of cells can originate from a first sample and a second group of cells can originate from a second sample. Labeling agents can allow the first group and second group to have a different labeling agent (or reporter oligonucleotide associated with the labeling agent). This can, for example, facilitate multiplexing, where cells of the first group and cells of the second group can be labeled separately and then pooled together for downstream analysis. The downstream detection of a label can indicate analytes as belonging to a particular group.

For example, a reporter oligonucleotide can be linked to an antibody or an epitope binding fragment thereof, and labeling a cell can include subjecting the antibody-linked barcode molecule or the epitope binding fragment-linked barcode molecule to conditions suitable for binding the antibody to a molecule present on a surface of the cell. The binding affinity between the antibody or the epitope binding fragment thereof and the molecule present on the surface can be within a desired range to ensure that the antibody or the epitope binding fragment thereof remains bound to the molecule. For example, the binding affinity can be within a desired range to ensure that the antibody or the epitope binding fragment thereof remains bound to the molecule during various sample processing steps, such as partitioning and/or nucleic acid amplification or extension. A dissociation constant (Kd) between the antibody or an epitope binding fragment thereof and the molecule to which it binds can be less than about 100 μM, 90 μM, 80 μM, 70 μM, 60 μM, 50 μM, 40 μM, 30 μM, 20 μM, 10 μM, 9 μM, 8 μM, 7 μM, 6 μM, 5 μM, 4 μM, 3 μM, 2 μM, 1 μM, 900 nM, 800 nM, 700 nM, 600 nM, 500 nM, 400 nM, 300 nM, 200 nM, 100 nM, 90 nM, 80 nM, 70 nM, 60 nM, 50 nM, 40 nM, 30 nM, 20 nM, 10 nM, 9 nM, 8 nM, 7 nM, 6 nM, 5 nM, 4 nM, 3 nM, 2 nM, 1 nM, 900 μM, 800 μM, 700 μM, 600 μM, 500 μM, 400 μM, 300 μM, 200 μM, 100 μM, 90 μM, 80 μM, 70 μM, 60 μM, 50 μM, 40 μM, 30 μM, 20 μM, 10 μM, 9 μM, 8 μM, 7 μM, 6 μM, 5 μM, 4 μM, 3 μM, 2 μM, or 1 μM. For example, the dissociation constant can be less than about 10 μM.

In another example, a reporter oligonucleotide can be coupled to a cell-penetrating peptide (CPP), and labeling cells can include delivering the CPP coupled reporter oligonucleotide into an biological particle. Labeling biological particles can include delivering the CPP conjugated oligonucleotide into a cell and/or cell bead by the cell-penetrating peptide. A CPP that can be used in the methods provided herein can include at least one non-functional cysteine residue, which can be either free or derivatized to form a disulfide link with an oligonucleotide that has been modified for such linkage. Non-limiting examples of CPPs that can be used in embodiments herein include penetratin, transportan, plsl, TAT(48-60), pVEC, MTS, and MAP. Cell-penetrating peptides useful in the methods provided herein can have the capability of inducing cell penetration for at least about 30%, 40%, 50%, 60%, 70%, 80%, 90%, 95%, 96%, 97%, 98%, 99%, or 100% of cells of a cell population. The CPP can be an arginine-rich peptide transporter. The CPP can be Penetratin or the Tat peptide.

In another example, a reporter oligonucleotide can be coupled to a fluorophore or dye, and labeling cells can include subjecting the fluorophore-linked barcode molecule to conditions suitable for binding the fluorophore to the surface of the cell. In some instances, fluorophores can interact strongly with lipid bilayers and labeling cells can include subjecting the fluorophore-linked barcode molecule to conditions such that the fluorophore binds to or is inserted into a membrane of the cell. In some cases, the fluorophore is a water-soluble, organic fluorophore. In some instances, the fluorophore is Alexa 532 maleimide, tetramethylrhodamine-5-maleimide (TMR maleimide), BODIPY-TMR maleimide, Sulfo-Cy3 maleimide, Alexa 546 carboxylic acid/succinimidyl ester, Atto 550 maleimide, Cy3 carboxylic acid/succinimidyl ester, Cy3B carboxylic acid/succinimidyl ester, Atto 565 biotin, Sulforhodamine B, Alexa 594 maleimide, Texas Red maleimide, Alexa 633 maleimide, Abberior STAR 635P azide, Atto 647N maleimide, Atto 647 SE, or Sulfo-Cy5 maleimide. See, e.g., Hughes L D, et al. PLoS One. 2014 Feb. 4; 9(2):e87649, which is hereby incorporated by reference in its entirety for all purposes, for a description of organic fluorophores.

In some embodiments, a labeling agent, e.g., a reporter oligonucleotide conjugated antigen, can comprise a detectable label, e.g., a fluorophore or dye. In some embodiments, different labeling agents, e.g., different reporter oligonucleotide conjugated antigens, can comprise different detectable labels, e.g., different fluorophores or dyes.

A reporter oligonucleotide can be part of a nucleic acid molecule including any number of functional sequences, as described elsewhere herein, such as a target capture sequence, a random primer sequence, and the like, and coupled to another nucleic acid molecule that is, or is derived from, the analyte.

Prior to attachment of analytes from a tissue sample to capture probes, as disclosed herein, the tissue sample can be contacted with (e.g., incubated with) a library of labeling agents, that can be labeling agents to a broad panel of different cell features and which include their associated reporter oligonucleotides. Unbound labeling agents can be washed from the tissue sample, and the tissue sample can then be subjected to spatial analysis, as described in any one of the spatial analysis methods disclosed herein. As a result, analytes and reporter oligonucleotides from the labeling agents can be associated with spatial barcodes which can provide information on the spatial location of the analytes and labeling agents in the tissue sample.

In other instances, e.g., to facilitate sample multiplexing, an antigen that is specific to a particular cell feature can have a first plurality of the antigen (e.g., an antibody or lipophilic moiety) coupled to a first reporter oligonucleotide with a first reporter barcode and a second plurality of the antigen coupled to a second reporter oligonucleotide with a second reporter barcode. In this way, different samples or groups can be independently processed and subsequently combined together for pooled analysis (e.g., spatial analysis as described elsewhere herein). See, e.g., U.S. Pat. Pub. 20190323088, which is hereby incorporated by reference its entirety.

In some instances, these reporter oligonucleotides can include nucleic acid barcode sequences that permit identification of the labeling agent which the reporter oligonucleotide is coupled to. For example, reporter oligonucleotides include nucleic acid barcode sequences that permit identification of the antigen (e.g., AbTx or ADC) which the reporter oligonucleotide is coupled to. The use of oligonucleotides as the reporter can provide advantages of being able to generate significant diversity in terms of sequence, while also being readily attachable to most biomolecules, e.g., antibodies, etc., as well as being readily detected, e.g., using sequencing or array technologies.

Attachment (e.g., coupling, conjugation) of the reporter oligonucleotides to the labeling agents can be achieved through any of a variety of direct or indirect, covalent or non-covalent associations or attachments. For example, oligonucleotides can be covalently attached to a portion of a labeling agent (such a protein, e.g., an antigen, an antibody or antibody fragment) using chemical conjugation techniques (e.g., Lightning-Link® antibody labeling kits available from Innova Biosciences), as well as other non-covalent attachment mechanisms, e.g., using biotinylated antibodies and oligonucleotides (or beads that include one or more biotinylated linker, coupled to oligonucleotides) with an avidin or a streptavidin linker in monomeric or multimeric form (e.g., tetramic form of streptavidin). Those of skill in the art will recognize that a streptavidin monomer encompasses streptavidin molecules with 1 biotin binding site, while a streptavidin multimer encompasses strepatavidin molecules with more than 1 biotin binding site. For example, a streptavidin tetramer has 4 biotin binding sites. However, a skilled artisan will also recognize that in a streptavidin tetramer does not necessarily comprise 4 streptavidins complexed together. Two molecules may be “covalently linked” or “covalently attached” to one another when at least one atom in the first molecule shares at least one electron pair with at least one atom in the second molecule. In some embodiments, a covalent linkage between two molecules can involve one or more intermediary molecules. For example, a first molecule and a second molecule may be considered covalently linked, if they are each covalently linked to a linker molecule. In such a circumstance, all three molecules (the first molecule, the second molecule, and the linker molecule) are covalently linked to one another.

Antibody and oligonucleotide biotinylation techniques are available. See, e.g., Fang, et al., “Fluoride-Cleavable Biotinylation Phosphoramidite for 5′-end-Labeling and Affinity Purification of Synthetic Oligonucleotides,” Nucleic Acids Res. Jan. 15, 2003; 31(2):708-715, which is entirely incorporated herein by reference for all purposes. Likewise, protein and peptide biotinylation techniques have been developed and are readily available. See, e.g., U.S. Pat. No. 6,265,552, which is entirely incorporated herein by reference for all purposes. Furthermore, click reaction chemistry such as a Methyltetrazine-PEG5-NHS Ester reaction, a TCO-PEG4-NHS Ester reaction, or the like, can be used to couple reporter oligonucleotides to labeling agents. Commercially available kits, such as those from Thunderlink and Abcam, and techniques common in the art can be used to couple reporter oligonucleotides to labeling agents as appropriate. In another example, a labeling agent is indirectly (e.g., via hybridization) coupled to a reporter oligonucleotide including a barcode sequence that identifies the labeling agent. For instance, the labeling agent can be directly coupled (e.g., covalently bound) to a hybridization oligonucleotide that includes a sequence that hybridizes with a sequence of the reporter oligonucleotide. Hybridization of the hybridization oligonucleotide to the reporter oligonucleotide couples the labeling agent to the reporter oligonucleotide. In some embodiments, the reporter oligonucleotides are releasable from the labeling agent, such as upon application of a stimulus. For example, the reporter oligonucleotide can be attached to the labeling agent through a labile bond (e.g., chemically labile, photolabile, thermally labile, etc.) as generally described for releasing molecules from supports elsewhere herein. In some instances, the reporter oligonucleotides described herein can include one or more functional sequences that can be used in subsequent processing, such as an adapter sequence, a unique molecular identifier (UMI) sequence, a sequencer specific flow cell attachment sequence (such as an P5, P7, or partial P5 or P7 sequence), a primer or primer binding sequence, a sequencing primer or primer biding sequence (such as an R1, R2, or partial R1 or R2 sequence).

In some cases, the labelling agent can include a reporter oligonucleotide and a label. A label An antigen (e.g., a therapeutic antibody or antibody drug complex) can be conjugated to a reporter oligonucleotide. The reporter oligonucleotide can be conjugated anywhere along the amino acid chain of the antigen. In some embodiments, the reporter oligonucleotide can be conjugated to the N terminus, the C terminus, or between the N terminus and the C terminus of the antigen.

In some embodiments, the reporter oligonucleotide conjugated to the antigen does not interfere with binding of the antigen to an antigen binding molecule. A reporter oligonucleotide can be conjugated away from a site on the antigen that binds to the antigen binding molecule (e.g., an epitope). In some embodiments, such as when the binding site is unknown, a reporter oligonucleotide can be conjugated to different parts of the antigen on different copies of the antigen.

Either end (e.g., the 3′ end or the 5′ end) of the reporter oligonucleotide can be conjugated to the antigen. In some embodiments, more than one reporter oligonucleotide can be conjugated to the antigen.

Conjugation of a reporter oligonucleotide to an antigen can preserve the tertiary and/or quaternary structure of the antigen. In some embodiments, the structure of the antigen can be completely preserved. In some embodiments, the structure of a binding site (e.g., a site where the antigen can bind to an antigen binding molecule such as an antibody) can be preserved. In some embodiments, the location and/or orientation of surface residues of the antigen can be preserved.

In some embodiments, the link between an antigen and a reporter oligonucleotide can be stable. Stability can be, for example, under physiological conditions (e.g., physiological pH, temperature, etc.), or under conditions of an assay. In some embodiments, such a link can remain stable for at least 1 hour, at least 6 hours, at least 12 hours, at least 1 day, at least 1 week, at least 1 month, at least 1 year, or a range between any two foregoing values. In some embodiments, the affinity between an antigen and antigen binding molecule is not compromised by the conjugation of a reporter oligonucleotide to the antigen. In some such embodiments, the presence of the oligonucleotide or the process of conjugating the oligonucleotide to the antigen may not increase or decrease the affinity of the antigen to the antigen binding molecule.

In some embodiments, a reporter oligonucleotide can be conjugated to an antigen directly using any suitable chemical moiety on the antigen. In some embodiments, a reporter oligonucleotide can be conjugated to an antigen enzymatically, e.g., by ligation. In some cases, a reporter oligonucleotide can be linked indirectly to an antigen, for example via a non-covalent interaction such as a biotin/streptavidin interaction or an equivalent thereof, via an aptamer or secondary antibody, or via a protein-protein interaction such as a leucine-zipper tag interaction or the like.

In some embodiments, a reporter oligonucleotide can be conjugated to an antigen using click chemistry, or a similar method. Click chemistry can refer to a class of biocompatible small molecule reactions that can allow the joining of molecules, such as a reporter oligonucleotide and an antigen. A click reaction can be a one pot reaction, and in some cases is not disturbed by water. A click reaction can generate minimal byproducts, non-harmful byproducts, or no byproducts. A click reaction can be driven by a large thermodynamic force. In some cases, a click reaction can be driven quickly and/or irreversibly to a high yield of a single reaction product (e.g., a reporter oligonucleotide conjugated to an antigen), and can have high reaction specificity. Click reactions can include but are not limited to [3+2] cycloadditions, thiol-ene reactions, Diels-Alder reactions, inverse electron demand Diels-Alder reactions, [4+1] cycloadditions, nucleophilic substitutions, carbonyl-chemistry-like formation of ureas, or addition reactions to carbon-carbon double bonds (e.g., dihydroxylation).

In some embodiments, an antigen can be conjugated to a reporter oligonucleotide by a redox activated chemical tagging (ReACT) reaction. A react reaction can be a chemoselective methionine-bioconjugation that can employ redox reactivity. In some embodiments, for example, oxaziridine-based reagents can enable highly selective, rapid, and robust conjugation. Further description of ReACT chemistry can be found, for example, in (Makishma, Akio. Biochemistry for Materials Science. Elsevier, 2019).

In some embodiments, an antigen can be conjugated to a reporter oligonucleotide by a site-specific sortase motif-dependent conjugation. Site-specific sortase motif-dependent conjugation can be a highly specific platform for conjugation that can rely on the specificity of Sortase A for short peptide sequences (e.g., LPXTG AND GGG).

Sortase A can be a transpeptidase that can be adopted for site-specific protein modification. A reaction catalyzed by Sortase A can result in the formation of an amide bond between a C terminal sorting motif (e.g., LPXTG, where X can be any amino acid) and an N terminal oligoglycine. Such a conjugation reaction can proceed by first cleaving the peptide bond between the threonine and glycine residues with the sorting motif of Sortase A. Sortase A can be used to conjugate an oligonucleotide to either an N terminus or a C terminus of an antigen. Sortase A can retain its specificity while accepting a wide range of potential substrates. In some embodiments, an antigen can be conjugated to a reporter oligonucleotide by a site-specific photo-crosslinking-dependent conjugation. For example, such photo-crosslinking dependent conjugation can utilize unnatural amino acids or chemical crosslinking. Such photo-crosslinking can be mediated or directed by a peptide in some cases. For example, a peptide or other photosensitive molecule on the antigen can form a covalent bond with a molecule on the oligonucleotide upon activation by a specified wavelength of light. In some embodiments, a peptide or other photosensitive molecule on the reporter oligonucleotide can form a covalent bond with a residue on the antigen upon activation by a specified wavelength of light. In some embodiments, an antigen can be conjugated to a reporter oligonucleotide by site-specific conformation-dependent conjugation (e.g., glycan-dependent Fc conjugation or GlyCLICK). Such conjugation can generate a reporter oligonucleotide conjugated antigen. For example, deglycosylation of the antigen can allow for site specific conjugation using click chemistry techniques. In some embodiment, an antigen can be conjugated to a reporter oligonucleotide by nitrilotriacetate conjugation

An oligonucleotide can be conjugated to a constant region of an antigen. For example, an oligonucleotide can be conjugated to a constant region of a heavy chain or a constant region of a light chain of an antigen that is an antibody or antigen binding fragment thereof.

An oligonucleotide can be conjugated to a variable region of an antigen. For example, an oligonucleotide can be conjugated to a variable region of a heavy chain or a variable region of a light chain of an antigen that is an antibody or antigen binding fragment thereof. The reporter oligonucleotide conjugated antigen can include one or more detectable tags. For example, in some instances, the reporter oligonucleotide conjugated antigen can include a fluorophore, metal ion, or other detectable tag. The detectable tag can be conjugated to the reporter oligonucleotide, the antigen, or both.

In some cases, the labeling agent can include a reporter oligonucleotide and a detectable tag. A detectable tag can be fluorophore, a radioisotope, a molecule capable of a colorimetric reaction, a magnetic particle, or any other suitable molecule or compound capable of detection. The detectable tag can be conjugated to a labeling agent (or reporter oligonucleotide) either directly or indirectly (e.g., the label can be conjugated to a molecule that can bind to the labeling agent or reporter oligonucleotide). In some cases, a detectable tag is conjugated to an oligonucleotide that is complementary to a sequence of the reporter oligonucleotide, and the oligonucleotide can be allowed to hybridize to the reporter oligonucleotide.

Additional methods and compositions suitable for barcoding cDNA generated from mRNA transcripts including those encoding V(D)J regions of an immune cell receptor and/or barcoding methods and composition including a template switch oligonucleotide are described in International Patent Application WO2018/075693, U.S. Patent Publication No. 2018/0105808, U.S. Patent Publication No. 2015/0376609, filed Jun. 26, 2015, and U.S. Patent Publication No. 2019/0367969, each of which applications is herein entirely incorporated by reference for all purposes.

### M. Exemplary Methods of the Disclosure

A particular valuable application of the methods and compositions described herein is the generation of sequences encoding BCR and TCR from a tumor sample of interest. Accordingly, some embodiments of the disclosure relate to methods for generating nucleic acid sequences, e.g., paired, full-length T cell receptor sequences and/or B cell receptor sequences identified from tumor samples. In some embodiments, such methods comprise identifying paired, full-length T cell receptor sequences and/or B cell receptor sequences from a tumor sample. As described in Example 6, the methods can begin by preparing a tumor tissue sample (e.g., a fresh frozen tissue section comprising tumor tissue) as described in the Visium Spatial Gene Expression Reagent Kits User Guide (e.g., Rev D, dated October 2020. The prepared samples are run according to spatial analysis methods described herein to generate Gene Expression, TCR Amplified, and/or BCR Amplified sequencing libraries. Data can be analyzed, e.g., using Cell Ranger 6.0, Loupe 5.0, and Enclone. The analysis can identify paired full-length TCR and/or BCR sequences from the tumor samples.

In some embodiments, the methods further comprise production of barcoded recombinant antibodies or TCRs. As described in greater detail in Example 7, by using the BCR sequences identified according to the methods described herein (e.g., as described in Example 6), nucleotide sequences encoding variable heavy chain and light chain domains of antibodies may be reformatted (for example, to IgG1) and synthesized and cloned into a mammalian expression vector. Exemplary mammalian expression vectors are commercially available, e.g., pTwist CMV BG WPRE Neo (Twist Bioscience eCommerce portal), AddGene, InvivoGen, and Human IgG Vector Set from SigmaAldrich. Light chain variable domains may be reformatted into kappa and lambda frameworks accordingly. Clonal genes may be delivered as purified plasmid DNA ready for transfection in human embryonic kidney (HEK) Expi293 cells (Thermo Scientific). Alternatively, ExpiCHO cells may be used for transfection. Cultures may be grown, harvested, and purified using a suitable purification technique such as, Protein A resin (PhyNexus) on the Hamilton Microlab STAR platform to produce a recombinant antibody.

Additionally or alternatively, using the TCR sequences identified according to the methods described herein (e.g., as described in Example 6), nucleotide sequences encoding TCR alpha and TCR beta chains may be synthesized and cloned into a mammalian expression vector. Clonal genes can then be delivered as purified plasmid DNA ready for introduction in cultured cells, e.g., Jurkat cells. Such constructs may be introduced via using classical transformation techniques, e.g., transfection, transduction, or using more precise techniques such as guide RNA (gRNA)-directed CRISPR/Cas genome editing, DNA-guided endonuclease genome editing with NgAgo (Natronobacterium gregoryi Argonaute), or TALENs genome editing (transcription activator-like effector nucleases). Cultures can be grown, harvested, and purified to produce a recombinant TCR. A TCR generally includes two polypeptides (e.g., polypeptide chains), such as a α-chain of a TCR, a β-chain of a TCR, a γ-chain of a TCR, a δ-chain of a TCR, or a combination thereof. Several approaches, techniques, and associated reagents for construction of recombinant TCR are known in the art. In some cases, the TCR constant region may be further altered to remove one or more domains thereof, which can be achieved by a known genome editing technique (e.g., CRISPR/Cas or TALENs discussed herein), via either homology directed repair, non-homologous end joining (NHEJ), and/or or microhomology-mediated end joining.

In some embodiments, the methods further comprise coupling a reporter oligonucleotide comprising a reporter barcode sequence to the recombinant antibody or TCR. A reporter oligonucleotide comprising a reporter barcode sequence can be coupled to the recombinant antibody or TCR according to available methods. The reporter barcode sequence can be used as an identifier sequence for the antibody or TCR coupled thereto. In some instances, the reporter oligonucleotide is coupled to the recombinant antibody or TCR using chemical conjugation techniques (e.g., Lightning-Link® antibody labelling kits available from Innova Biosciences). In some instances, the reporter oligonucleotide is coupled to the recombinant antibody or TCR using non-covalent attachment mechanisms, e.g., using biotinylated antibodies and oligonucleotides (or beads that include one or more biotinylated linker, coupled to oligonucleotides) with an avidin or a streptavidin linker in monomeric or multimeric form (e.g., tetramic form of streptavidin). Antibody and oligonucleotide biotinylation techniques are available. See, e.g., Fang, et al., “Fluoride-Cleavable Biotinylation Phosphoramidite for 5′-end-Labelling and Affinity Purification of Synthetic Oligonucleotides,” Nucleic Acids Res. Jan. 15, 2003; 31(2):708-715. Likewise, protein and peptide biotinylation techniques have been developed and are readily available. See, e.g., U.S. Pat. No. 6,265,552. In some instances, the reporter oligonucleotide may be coupled to the recombinant antibody or TCR using click reaction chemistry such as a Methyltetrazine-PEG5-NHS Ester reaction, a TCO-PEG4-NHS Ester reaction. In some instances, the reporter oligonucleotide may be coupled to the recombinant antibody or TCR using a commercially available kit, such as from Thunderlink or Abcam. In some instances, the reporter oligonucleotide is coupled to the recombinant antibody or TCR indirectly (e.g., via hybridization). In some instances, the recombinant antibody or TCR may be directly coupled (e.g., covalently bound) to a hybridization oligonucleotide that includes a sequence that hybridizes with a sequence of the reporter oligonucleotide. Hybridization of the hybridization oligonucleotide to the reporter oligonucleotide couples the labelling agent to the reporter oligonucleotide. In some embodiments, the reporter oligonucleotides may be releasable from the labelling agent, such as upon application of a stimulus. For example, the reporter oligonucleotide can be attached to the labeling agent through a labile bond (e.g., chemically labile, photolabile, thermally labile, etc.) as generally described for releasing molecules from supports elsewhere herein. In some instances, the reporter oligonucleotides described herein can include one or more functional sequences that can be used in subsequent processing, such as an adapter sequence, a unique molecular identifier (UMI) sequence, a sequencer specific flow cell attachment sequence (such as an P5, P7, or partial P5 or P7 sequence), a primer or primer binding sequence, a sequencing primer or primer biding sequence (such as an R1, R2, or partial R1 or R2 sequence).

In some embodiments, the methods further comprise analysis of the barcoded recombinant antibodies or TCRs. In particular embodiments, the analysis comprises contacting one of more of the barcoded recombinant antibodies or TCRs with a second tumor sample that is a tumor tissue sample, e.g., a fresh frozen tissue section comprising tumor tissue. In some embodiments, the sample is incubated with a cocktail of recombinant barcoded antibodies and/or TCRs. Optionally, the cocktail includes barcoded antibodies for known immune cell markers. Optionally, the cocktail includes barcoded antibodies for known tumor cell markers. Optionally, the cocktail includes one or more barcoded therapeutic antibodies. In some embodiments, the barcoded antibodies (e.g., barcoded antibodies for known immune cell markers, barcoded antibodies for known tumor cell markers, barcoded therapeutic antibodies) are coupled to reporter oligonucleotides comprising reporter barcode sequences that identify the antibody coupled thereto. Methods for contacting one or more of the barcoded recombinant antibodies or TCRs with a second tumor tissue sample (e.g., comprising tumor cells) are described in further detail in Examples. In particular embodiments, the analysis comprises contacting one of more of the barcoded recombinant antibodies or TCRs with a second tumor sample that is an intact tumor sample, e.g., a fresh frozen tissue section comprising tumor tissue. The sample can be mounted on a slide including an array of spatially barcoded capture probes (e.g., a Visium Spatial Gene Expression slide as described in the Visium Spatial Gene Expression Reagent Kits User Guide (e.g., Rev D, dated October 2020). The sample can be subjected to fixation. The sample can be subjected to a blocking step. The sample can be incubated with a cocktail of recombinant barcoded antibodies and/or TCRs. Optionally, the cocktail can include barcoded antibodies for known immune cell markers. Optionally, the cocktail includes barcoded antibodies for known tumor cell markers. Optionally, the cocktail can include one or more barcoded therapeutic antibodies. The barcoded antibodies (e.g., barcoded antibodies for known immune cell markers, barcoded antibodies for known tumor cell markers, barcoded therapeutic antibodies) can be coupled to reporter oligonucleotides comprising reporter barcode sequences that identify the antibody coupled thereto. The sample can be stained (e.g., with H&E) and imaged according to any of the methods described herein. Optionally, if any of the barcoded antibodies include a fluorescence detection agent, the sample may be imaged via immunofluorescence. The sample can be permeabilized, e.g., according to methods described in the Visium Spatial Gene Expression Reagent Kits User Guide (e.g., Rev D, dated October 2020). Transcripts and reporter oligonucleotides can be released during permeabilization for capture onto the spatially barcoded array. The captured transcripts and reporter oligonucleotides can be used in an extension reaction to produce spatially barcoded extension products comprising sequences corresponding to the captured transcripts and/or reporter oligonucleotides, respectively. The spatially barcoded extension products can be used to produce gene expression and reporter oligonucleotide libraries. Sequence analysis can be used to identify one or more recombinant barcoded antibodies and/or barcoded recombinant TCRs (e.g., as produced according to the methods of Example 7) as having specificity for the tumor. Comparative analysis of gene expression, the reporter oligonucleotide, and the image datasets, can be performed to determine the recombinant antibodies' specificity and target specificity.

In particular embodiments, the analysis comprises contacting one of more of the barcoded recombinant antibodies or TCRs with a second tumor sample that is an intact tumor sample, e.g., a formalin-fixed paraffin embedded (FFPE) sample comprising tumor tissue. The sample can be mounted on a slide including an array of spatially barcoded capture probes (e.g., a spatially barcoded array slide as described in the Visium Spatial Gene Expression for FFPE User Guide (e.g., Rev A, dated June 2021)). The slide-mounted sample can be dried overnight in a desiccator. The sample can be heated to 60° C., followed by deparaffinization and rehydration. H&E staining can be performed and the sample can be imaged. The sample can be destained using a suitable buffer (e.g., HCl and decrosslinked for 1 hour in citrate buffer (pH 6.0) at 95° C.). After decrosslinking, the sample can be incubated overnight with RTL (templated ligation) probe sets at 50° C., e.g., according to methods described in the the Visium Spatial Gene Expression for FFPE User Guide (e.g., Rev A, dated June 2021). The sample can be washed to remove un-hybridized probes, then treated with ligase to ligate the RTL probes. The sample can be washed, then blocked with antibody blocking buffer. The sample can be incubated overnight with a cocktail of recombinant barcoded antibodies and/or TCRs. Optionally, the cocktail can include barcoded antibodies for known immune cell markers. Optionally, the cocktail can include barcoded antibodies for known tumor cell markers. Optionally, the cocktail includes one or more barcoded therapeutic antibodies. The barcoded antibodies (e.g., barcoded antibodies for known immune cell markers, barcoded antibodies for known tumor cell markers, barcoded therapeutic antibodies) can be coupled to reporter oligonucleotides comprising reporter barcode sequences that identify the antibody coupled thereto. The sample can be washed with PBST, and washed with SSC. The sample can be subjected to a 30 minute probe release step with RNase, followed by a 1 hour permeabilization step with a permeabilization buffer including Proteinase K and detergent. Accordingly, the ligation products and reporter oligonucleotides of the barcoded antibodies can be captured by the capture probes of the spatially barcoded array slide. The slide can be washed twice (e.g., with 2×SSC) and subjected to probe extension, denaturation, and pre-amplification followed by amplification and sequencing of the templated ligation and reporter oligonucleotide libraries. Sequence analysis can be used to identify one or more recombinant barcoded antibodies and/or barcoded recombinant TCRs (e.g., as produced according to the methods of Example 7) as having specificity for the tumor. Comparative analysis of the templated ligation, the reporter oligonucleotide, and the image datasets can performed to determine the recombinant antibodies' specificity and target specificity.

### Compositions of the Disclosure

As described in greater detail below, one aspect of the present disclosure relates to recombinant antibodies or functional fragments thereof generated or identified by a method disclosed herein. Also provided, in other related aspects of the disclosure, are nucleic acids encoding the recombinant antibodies as disclosed herein or functional fragments thereof, recombinant cells expressing the recombinant antibodies as disclosed herein or functional fragments thereof, pharmaceutical compositions containing the nucleic acids and/or recombinant cells as disclosed herein.

### A. Recombinant Nucleic Acids

In discussed above, one aspect of the disclosure relates to recombinant nucleic acids including a nucleic acid sequence that encode the recombinant antibody of the disclosure or a functional fragment thereof. In some embodiments, the recombinant nucleic acids of the disclosure can be configured as expression cassettes or vectors containing these nucleic acid molecules operably linked to heterologous nucleic acid sequences such as, for example, regulatory sequences which allow in vivo expression of the receptor in a host cell.

Nucleic acid molecules of the present disclosure can be of any length, including for example, between about 1.5 Kb and about 50 Kb, between about 5 Kb and about 40 Kb, between about 5 Kb and about 30 Kb, between about 5 Kb and about 20 Kb, or between about 10 Kb and about 50 Kb, for example between about 15 Kb to 30 Kb, between about 20 Kb and about 50 Kb, between about 20 Kb and about 40 Kb, about 5 Kb and about 25 Kb, or about 30 Kb and about 50 Kb.

Accordingly, in some embodiments, provided herein is a nucleic acid molecule including a nucleotide sequence encoding a recombinant antibody of the disclosure or a functional fragment thereof. In some embodiments, the nucleotide sequence is incorporated into an expression cassette or an expression vector. It will be understood that an expression cassette generally includes a construct of genetic material that contains coding sequences and enough regulatory information to direct proper transcription and/or translation of the coding sequences in a recipient cell, in vivo and/or ex vivo. Generally, the expression cassette can be inserted into a vector for targeting to a desired host cell and/or into an individual. As such, in some embodiments, an expression cassette of the disclosure include a coding sequence for a recombinant antibody of the disclosure or a functional fragment thereof, which is operably linked to expression control elements, such as a promoter, and optionally, any or a combination of other nucleic acid sequences that affect the transcription or translation of the coding sequence.

In some embodiments, the nucleotide sequence is incorporated into an expression vector. It will be understood by one skilled in the art that the term “vector” generally refers to a recombinant polynucleotide construct designed for transfer between host cells, and that can be used for the purpose of transformation, e.g., the introduction of heterologous DNA into a host cell. As such, in some embodiments, the vector can be a replicon, such as a plasmid, phage, or cosmid, into which another DNA segment can be inserted so as to bring about the replication of the inserted segment. In some embodiments, the expression vector can be an integrating vector.

In some embodiments, the expression vector can be a viral vector. As will be appreciated by one of skill in the art, the term “viral vector” is widely used to refer either to a nucleic acid molecule (e.g., a transfer plasmid) that includes virus-derived nucleic acid elements that typically facilitate transfer of the nucleic acid molecule or integration into the genome of a cell or to a viral particle that mediates nucleic acid transfer. Viral particles will typically include various viral components and sometimes also host cell components in addition to nucleic acid(s). The term viral vector can refer either to a virus or viral particle capable of transferring a nucleic acid into a cell or to the transferred nucleic acid itself. Viral vectors and transfer plasmids contain structural and/or functional genetic elements that are primarily derived from a virus. The term “retroviral vector” refers to a viral vector or plasmid containing structural and functional genetic elements, or portions thereof, that are primarily derived from a retrovirus. The term “lentiviral vector” refers to a viral vector or plasmid containing structural and functional genetic elements, or portions thereof, including LTRs that are primarily derived from a lentivirus, which is a genus of retrovirus.

The nucleic acid sequences encoding the recombinant antibodies as disclosed herein can be optimized for expression in the host cell of interest. For example, the G-C content of the sequence can be adjusted to average levels for a given cellular host, as calculated by reference to known genes expressed in the host cell. Methods for codon usage optimization are known in the art. Codon usages within the coding sequence of the recombinant antibodies disclosed herein can be optimized to enhance expression in the host cell, such that about 1%, about 5%, about 10%, about 25%, about 50%, about 75%, or up to 100% of the codons within the coding sequence have been optimized for expression in a particular host cell.

Some embodiments disclosed herein relate to vectors or expression cassettes including a recombinant nucleic acid molecule encoding the recombinant antibodies disclosed herein. The expression cassette generally contains coding sequences and sufficient regulatory information to direct proper transcription and/or translation of the coding sequences in a recipient cell, in vivo and/or ex vivo. The expression cassette can be inserted into a vector for targeting to a desired host cell and/or into an individual. An expression cassette can be inserted into a plasmid, cosmid, virus, autonomously replicating polynucleotide molecule, phage, as a linear or circular, single-stranded or double-stranded, DNA or RNA polynucleotide molecule, derived from any source, capable of genomic integration or autonomous replication, including a nucleic acid molecule where one or more nucleic acid sequences has been linked in a functionally operative manner, i.e., operably linked.

Also provided herein are vectors, plasmids, or viruses containing one or more of the nucleic acid molecules encoding any recombinant antibody or a functional fragment thereof as disclosed herein. The nucleic acid molecules can be contained within a vector that is capable of directing their expression in, for example, a cell that has been transformed/transduced with the vector. Suitable vectors for use in eukaryotic and prokaryotic cells are known in the art and are commercially available, or readily prepared by a skilled artisan. See for example, Sambrook, J., & Russell, D. W. (2012). Molecular Cloning. A Laboratory Manual(4th ed.). Cold Spring Harbor, NY: Cold Spring Harbor Laboratory and Sambrook, J., & Russel, D. W. (2001). Molecular Cloning. A Laboratory Manual (3rd ed.). Cold Spring Harbor, NY: Cold Spring Harbor Laboratory (jointly referred to herein as “Sambrook”); Ausubel, F. M. (1987). Current Protocols in Molecular Biology. New York, NY: Wiley (including supplements through 2014); Bollag, D. M. et al. (1996). Protein Methods. New York, NY: Wiley-Liss; Huang, L. et al. (2005). Nonviral Vectors for Gene Therapy. San Diego: Academic Press; Kaplitt, M. G. et al. (1995). Viral Vectors: Gene Therapy and Neuroscience Applications. San Diego, CA: Academic Press; Lefkovits, I. (1997). The Immunology Methods Manual: The Comprehensive Sourcebook of Techniques. San Diego, CA: Academic Press; Doyle, A. et al. (1998). Cell and Tissue Culture: Laboratory Procedures in Biotechnology. New York, NY: Wiley; Mullis, K. B., Ferré, F. & Gibbs, R. (1994). PCR: The Polymerase Chain Reaction. Boston: Birkhauser Publisher; Greenfield, E. A. (2014). Antibodies: A Laboratory Manual (2nd ed.). New York, NY: Cold Spring Harbor Laboratory Press; Beaucage, S. L. et al. (2000). Current Protocols in Nucleic Acid Chemistry. New York, NY: Wiley, (including supplements through 2014); and Makrides, S. C. (2003). Gene Transfer and Expression in Mammalian Cells. Amsterdam, NL: Elsevier Sciences B.V., the disclosures of which are incorporated herein by reference).

DNA vectors can be introduced into eukaryotic cells via conventional transformation or transfection techniques. Suitable methods for transforming or transfecting host cells can be found in Sambrook et al. (2012, supra) and other standard molecular biology laboratory manuals, such as, calcium phosphate transfection, DEAE-dextran mediated transfection, transfection, microinjection, cationic lipid-mediated transfection, electroporation, transduction, scrape loading, ballistic introduction, nucleoporation, hydrodynamic shock, and infection.

Viral vectors that can be used in the disclosure include, for example, retrovirus vectors, adenovirus vectors, and adeno-associated virus vectors, lentivirus vectors, herpes virus, simian virus 40 (SV40), and bovine papilloma virus vectors (see, for example, Gluzman (Ed.), Eukaryotic Viral Vectors, CSH Laboratory Press, Cold Spring Harbor, N.Y.).

For example, a recombinant antibody or a functional fragment thereof as disclosed herein can be produced in a eukaryotic host, such as a mammalian cells (e.g., COS cells, NIH 3T3 cells, or HeLa cells). These cells are available from many sources, including the American Type Culture Collection (Manassas, VA). In selecting an expression system, it matters only that the components are compatible with one another. Artisans or ordinary skill are able to make such a determination. Furthermore, if guidance is required in selecting an expression system, skilled artisans can consult P. Jones, “Vectors: Cloning Applications”, John Wiley and Sons, New York, N.Y., 2009).

The nucleic acid molecules provided can contain naturally occurring sequences, or sequences that differ from those that occur naturally, but, due to the degeneracy of the genetic code, encode the same polypeptide, e.g., antibody. These nucleic acid molecules can consist of RNA or DNA (for example, genomic DNA, cDNA, or synthetic DNA, such as that produced by phosphoramidite-based synthesis), or combinations or modifications of the nucleotides within these types of nucleic acids. In addition, the nucleic acid molecules can be double-stranded or single-stranded (e.g., either a sense or an antisense strand).

The nucleic acid molecules are not limited to sequences that encode polypeptides (e.g., antibodies); some or all of the non-coding sequences that lie upstream or downstream from a coding sequence (e.g., the coding sequence of an antibody) can also be included. Those of ordinary skill in the art of molecular biology are familiar with routine procedures for isolating nucleic acid molecules. In the event the nucleic acid molecule is a ribonucleic acid (RNA), molecules can be produced, for example, by in vitro transcription.

### B. Recombinant Cells and Cell Culture

The nucleic acid of the present disclosure can be introduced into a host cell, such as, for example, a human T lymphocyte, to produce a recombinant cell containing the nucleic acid molecule. Introduction of the nucleic acid molecules of the disclosure into cells can be achieved by methods known to those skilled in the art such as, for example, viral infection, transfection, conjugation, protoplast fusion, lipofection, electroporation, nucleofection, calcium phosphate precipitation, polyethyleneimine (PEI)-mediated transfection, DEAE-dextran mediated transfection, liposome-mediated transfection, particle gun technology, calcium phosphate precipitation, direct micro-injection, nanoparticle-mediated nucleic acid delivery, and the like.

Accordingly, in some embodiments, the nucleic acid molecules can be delivered by viral or non-viral delivery vehicles known in the art. For example, the nucleic acid molecule can be stably integrated in the host genome, or can be episomally replicating, or present in the recombinant host cell as a mini-circle expression vector for transient expression. Accordingly, in some embodiments, the nucleic acid molecule is maintained and replicated in the recombinant host cell as an episomal unit. In some embodiments, the nucleic acid molecule is stably integrated into the genome of the recombinant cell. Stable integration can be achieved using classical random genomic recombination techniques or with more precise techniques such as guide RNA-directed CRISPR/Cas genome editing, or DNA-guided endonuclease genome editing with NgAgo (Natronobacterium gregoryi Argonaute), or TALENs genome editing (transcription activator-like effector nucleases). In some embodiments, the nucleic acid molecule is present in the recombinant host cell as a mini-circle expression vector for transient expression.

The nucleic acid molecules can be encapsulated in a viral capsid or a lipid nanoparticle, or can be delivered by viral or non-viral delivery means and methods known in the art, such as electroporation. For example, introduction of nucleic acids into cells can be achieved by viral transduction. In a non-limiting example, adeno-associated virus (AAV) is engineered to deliver nucleic acids to target cells via viral transduction. Several AAV serotypes have been described, and all of the known serotypes can infect cells from multiple diverse tissue types. AAV is capable of transducing a wide range of species and tissues in vivo with no evidence of toxicity, and it generates relatively mild innate and adaptive immune responses.

Lentiviral-derived vector systems are also useful for nucleic acid delivery and gene therapy via viral transduction. Lentiviral vectors offer several attractive properties as gene-delivery vehicles, including: (i) sustained gene delivery through stable vector integration into host genome; (ii) the capability of infecting both dividing and non-dividing cells; (iii) broad tissue tropisms, including important gene- and cell-therapy-target cell types; (iv) no expression of viral proteins after vector transduction; (v) the ability to deliver complex genetic elements, such as polycistronic or intron-containing sequences; (vi) a potentially safer integration site profile; and (vii) a relatively easy system for vector manipulation and production.

In some embodiments, host cells can be genetically engineered (e.g., transduced or transformed or transfected) with, for example, a vector construct of the present application that can be, for example, a viral vector or a vector for homologous recombination that includes nucleic acid sequences homologous to a portion of the genome of the host cell, or can be an expression vector for the expression of the polypeptides of interest. Host cells can be either untransformed cells or cells that have already been transfected with at least one nucleic acid molecule.

In some embodiments, the recombinant cell is a prokaryotic cell or a eukaryotic cell. In some embodiments, the cell is in vivo. In some embodiments, the cell is ex vivo. In some embodiments, the cell is in vitro. In some embodiments, the recombinant cell is a eukaryotic cell. In some embodiments, the recombinant cell is an animal cell. In some embodiments, the animal cell is a mammalian cell. In some embodiments, the animal cell is a human cell. In some embodiments, the cell is a non-human primate cell. In some embodiments, the mammalian cell is an immune cell, a neuron, an epithelial cell, and endothelial cell, or a stem cell. In some embodiments, the cell is a stem cell. In some embodiments, the cell is a hematopoietic stem cell.

In some embodiments, the recombinant cell is an immune system cell, e.g., a lymphocyte (e.g., a T cell or NK cell), or a dendritic cell. In some embodiments, the immune cell is a B cell, a monocyte, a natural killer (NK) cell, a natural killer T (NKT) cell, a basophil, an eosinophil, a neutrophil, a dendritic cell, a macrophage, a regulatory T cell, a helper T cell (T_{H}), a cytotoxic T cell (T_{CTL}), or other T cell. In some embodiments, the immune system cell is a T lymphocyte. In some embodiments, the cell is a precursor T cell or a T regulatory (Treg) cell. In some embodiments, the cell is a CD34+, CD8+, or a CD4+ cell. In some embodiments, the cell is a CD8+ T cytotoxic lymphocyte cell selected from the group consisting of naïve CD8+ T cells, central memory CD8+ T cells, effector memory CD8+ T cells, and bulk CD8+ T cells. In some embodiments of the cell, the cell is a CD4+ T helper lymphocyte cell selected from the group consisting of naïve CD4+ T cells, central memory CD4+ T cells, effector memory CD4+ T cells, and bulk CD4+ T cells. In some embodiments, the cell can be obtained by leukapheresis performed on a sample obtained from an individual. In some embodiments, the subject is a human patient.

In another aspect, some embodiments of the disclosure relate to methods for making a recombinant cell, including (a) providing a cell capable of protein expression and (b) contacting the provided cell with a recombinant nucleic acid of the disclosure.

In another aspect, provided herein are cell cultures including at least one recombinant cell as disclosed herein, and a culture medium. Generally, the culture medium can be any suitable culture medium for culturing the cells described herein. Techniques for transforming a wide variety of the above-mentioned host cells and species are known in the art and described in the technical and scientific literature. Accordingly, cell cultures including at least one recombinant cell as disclosed herein are also within the scope of this application. Methods and systems suitable for generating and maintaining cell cultures are known in the art.

### C. Compositions and Pharmaceutical Compositions

The recombinant antibodies, nucleic acids, recombinant cells, and/or cell cultures of the disclosure can be incorporated into compositions, including pharmaceutical compositions.

In one aspect, as described in greater detail below, the barcoded recombinant antibodies as described herein can be included in compositions suitable for various downstream applications. In general, the compositions of the disclosure can include at least one barcoded recombinant antibody of the disclosure and one or more of the following: (i) one or more barcoded immune-cell marker antibodies and/or functional fragments thereof, (ii) one or more barcoded tumor-cell marker antibodies and functional fragments thereof, (iii) one or more barcoded therapeutic antibodies and functional fragments thereof, and (iv) one or more barcoded recombinant antibodies identified in the present disclosure as having specificity for a tumor sample. In some embodiments, the barcoded antibodies are each coupled to a reporter oligonucleotide including a reporter barcode sequence. In some embodiments, to facilitate downstream analyses, the reporter barcode sequence coupled to a barcoded antibody is distinguishable from coupled to the other barcoded antibodies.

In some embodiments, one or more of the antibodies are monoclonal antibodies. In some embodiments, one or more of the antibodies are polyclonal antibodies. In some embodiments, one or more of the antibodies are multi-specific antibodies (e.g., bispecific antibodies). Functional fragments of the antibodies suitable for the methods described herein can include F(ab) fragments, Fab′ fragments, F(ab′)2 fragments, Fv domains, and Fc domains.

In another aspect, the recombinant antibodies, nucleic acids, recombinant cells, and/or cell cultures of the disclosure can be incorporated into pharmaceutical compositions. Exemplary compositions of the disclosure include pharmaceutical compositions which generally include one or more of the recombinant antibodies, nucleic acids, recombinant cells, and/or cell cultures as described herein and a pharmaceutically acceptable excipient, e.g., carrier.

In one aspect, provided herein are compositions including a pharmaceutically acceptable excipient and one or more of the following: (a) a recombinant antibody of the disclosure; (b) a recombinant nucleic acid of the disclosure; and (c) a recombinant cell of the disclosure.

The pharmaceutical compositions provided herein can be in any form that allows for the composition to be administered to an individual. In some specific embodiments, the pharmaceutical compositions are suitable for human administration. As used herein, the term “pharmaceutically acceptable” means approved by a regulatory agency of the Federal or a state government or listed in the U.S. Pharmacopeia or other generally recognized pharmacopeiae for use in animals, and more particularly in humans. The carrier can be a diluent, adjuvant, excipient, or vehicle with which the pharmaceutical composition is administered. Saline solutions and aqueous dextrose and glycerol solutions can also be employed as liquid carriers, including injectable solutions. Suitable excipients include starch, glucose, lactose, sucrose, gelatin, malt, rice, flour, chalk, silica gel, sodium stearate, glycerol monostearate, talc, sodium chloride, dried skim milk, glycerol, propylene, glycol, water, ethanol and the like. Examples of suitable pharmaceutical carriers are described in “Remington's Pharmaceutical Sciences” by E. W. Martin. In some embodiments, the pharmaceutical composition is sterilely formulated for administration into an individual or an animal (some non-limiting examples include a human, or a mammal). In some embodiments, the individual is a human.

The terms “administration” and “administering”, as used herein, refer to the delivery of a bioactive composition or formulation by an administration route comprising, but not limited to, intranasal, transdermal, intravenous, intra-arterial, intramuscular, intraperitoneal, subcutaneous, intramuscular, oral, and topical administration, or combinations thereof. The term includes, but is not limited to, administering by a medical professional and self-administering.

In some embodiments, the pharmaceutical compositions of the present disclosure are formulated to be suitable for the intended route of administration to an individual. For example, the pharmaceutical composition can be formulated to be suitable for parenteral, intraperitoneal, colorectal, intraperitoneal, and intratumoral administration. In some embodiments, the pharmaceutical composition can be formulated for intravenous, oral, intraperitoneal, intratracheal, subcutaneous, intramuscular, topical, or intratumoral administration. One of ordinary skilled in the art will appreciate that the formulation should suit the mode of administration.

For example, pharmaceutical compositions suitable for injectable use include sterile aqueous solutions or dispersions and sterile powders for the extemporaneous preparation of sterile injectable solutions or dispersion. For intravenous administration, suitable carriers include physiological saline, bacteriostatic water, Cremophor EL™ (BASF, Parsippany, N.J.), or phosphate buffered saline (PBS). In some embodiments, the composition should be sterile and should be fluid to the extent that easy syringability exists. It can be stabilized under the conditions of manufacture and storage, and can be preserved against the contaminating action of microorganisms such as bacteria and fungi. The carrier can be a solvent or dispersion medium containing, for example, water, ethanol, polyol (for example, glycerol, propylene glycol, and liquid polyethylene glycol, and the like), and suitable mixtures thereof. The proper fluidity can be maintained, for example, by the use of a coating such as lecithin, by the maintenance of the required particle size in the case of dispersion and by the use of surfactants, e.g., sodium dodecyl sulfate. Prevention of the action of microorganisms can be achieved by various antibacterial and antifungal agents, for example, parabens, chlorobutanol, phenol, ascorbic acid, thimerosal, and the like. In many cases, it will be generally to include isotonic agents, for example, sugars, polyalcohols such as mannitol, sorbitol, and/or sodium chloride in the composition. Prolonged absorption of the injectable compositions can be brought about by including in the composition an agent which delays absorption, for example, aluminum monostearate and gelatin.

Sterile injectable solutions can be prepared by incorporating the active compound in the required amount in an appropriate solvent with one or a combination of ingredients enumerated above, as required, followed by filtered sterilization. Generally, dispersions are prepared by incorporating the active compound into a sterile vehicle, which contains a basic dispersion medium and the required other ingredients from those enumerated above.

### D. Kits

Also provided herein are kits for the practice of a method described herein. A kit can include instructions for use thereof and one or more of the recombinant antibodies or functional fragments thereof, recombinant nucleic acids, recombinant cells, and compositions as described and provided herein. For examples, some embodiments of the disclosure provide kits that include one or more of the recombinant antibodies described herein and/or functional fragments thereof, and instructions for use. In some embodiments, provided herein are kits that include one or more recombinant nucleic acids, recombinant cells, and compositions as described herein and instructions for use thereof.

In some embodiments, the components of a kit can be in separate containers. In some other embodiments, the components of a kit can be combined in a single container.

In some embodiments, a kit can further include instructions for using the components of the kit to practice a method described herein. The instructions for practicing the method are generally recorded on a suitable recording medium. For example, the instructions can be printed on a substrate, such as paper or plastic, etc. The instructions can be present in the kit as a package insert, in the labeling of the container of the kit or components thereof (e.g., associated with the packaging or sub-packaging), etc. The instructions can be present as an electronic storage data file present on a suitable computer readable storage medium, e.g. CD-ROM, diskette, flash drive, etc. In some instances, the actual instructions are not present in the kit, but means for obtaining the instructions from a remote source (e.g., via the internet), can be provided. An example of this embodiment is a kit that includes a web address where the instructions can be viewed and/or from which the instructions can be downloaded. As with the instructions, this means for obtaining the instructions can be recorded on a suitable substrate.

D. Computer systems

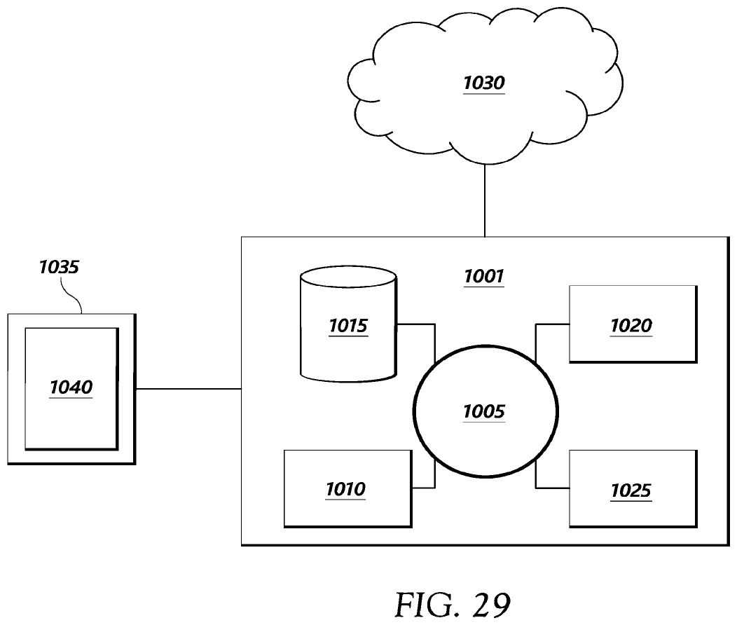

The present disclosure provides computer systems that are programmed to implement methods of the disclosure. FIG. 29 shows a computer system 1001 that is programmed or otherwise configured to perform sequencing applications, generate and maintain libraries of cytokine or other analyte specific antibody barcode sequences, MHC multimer barcode sequences, cell surface protein barcode sequences, and cDNAs generated from mRNAs respectively, and/or analyze such libraries. The computer system 1001 can regulate various aspects of the present disclosure, such as, for example, regulating fluid flow rate in one or more channels in a microfluidic structure, regulating polymerization application units, regulating sequence application unit, etc. The computer system 1001 can be an electronic device of a user or a computer system that is remotely located with respect to the electronic device. The electronic device can be a mobile electronic device.

The computer system 1001 includes a central processing unit (CPU, also “processor” and “computer processor” herein) 1005, which can be a single core or multi core processor, or a plurality of processors for parallel processing. The computer system 1001 also includes memory or memory location 1010 (e.g., random-access memory, read-only memory, flash memory), electronic storage unit 1015 (e.g., hard disk), communication interface 1020 (e.g., network adapter) for communicating with one or more other systems, and peripheral devices 1025, such as cache, other memory, data storage and/or electronic display adapters. The memory 1010, storage unit 1015, interface 1020 and peripheral devices 1025 are in communication with the CPU 1005 through a communication bus (solid lines), such as a motherboard. The storage unit 1015 can be a data storage unit (or data repository) for storing data. The computer system 1001 can be operatively coupled to a computer network (“network”) 1030 with the aid of the communication interface 1020. The network 1030 can be the Internet, an internet and/or extranet, or an intranet and/or extranet that is in communication with the Internet. The network 1030 in some cases is a telecommunication and/or data network. The network 1030 can include one or more computer servers, which can enable distributed computing, such as cloud computing. The network 1030, in some cases with the aid of the computer system 1001, can implement a peer-to-peer network, which can enable devices coupled to the computer system 1001 to behave as a client or a server.

The CPU 1005 can execute a sequence of machine-readable instructions, which can be embodied in a program or software. The instructions can be stored in a memory location, such as the memory 1010. The instructions can be directed to the CPU 1005, which can subsequently program or otherwise configure the CPU 1005 to implement methods of the present disclosure. Examples of operations performed by the CPU 1005 can include fetch, decode, execute, and writeback.

The CPU 1005 can be part of a circuit, such as an integrated circuit. One or more other components of the system 1001 can be included in the circuit. In some cases, the circuit is an application specific integrated circuit (ASIC).

The storage unit 1015 can store files, such as drivers, libraries and saved programs. The storage unit 1015 can store user data, e.g., user preferences and user programs. The computer system 1001 in some cases can include one or more additional data storage units that are external to the computer system 1001, such as located on a remote server that is in communication with the computer system 1001 through an intranet or the Internet.

The computer system 1001 can communicate with one or more remote computer systems through the network 1030. For instance, the computer system 1001 can communicate with a remote computer system of a user (e.g., operator). Examples of remote computer systems include personal computers (e.g., portable PC), slate or tablet PC's (e.g., Apple® iPad, Samsung® Galaxy Tab), telephones, Smart phones (e.g., Apple® iPhone, Android-enabled device, Blackberry®), or personal digital assistants. The user can access the computer system 1001 via the network 1030.

Methods as described herein can be implemented by way of machine (e.g., computer processor) executable code stored on an electronic storage location of the computer system 1001, such as, for example, on the memory 1010 or electronic storage unit 1015. The machine executable or machine readable code can be provided in the form of software. During use, the code can be executed by the processor 1005. In some cases, the code can be retrieved from the storage unit 1015 and stored on the memory 1010 for ready access by the processor 1005. In some situations, the electronic storage unit 1015 can be precluded, and machine-executable instructions are stored on memory 1010.

The code can be pre-compiled and configured for use with a machine having a processor adapted to execute the code, or can be compiled during runtime. The code can be supplied in a programming language that can be selected to enable the code to execute in a pre-compiled or as-compiled fashion.

Aspects of the systems and methods provided herein, such as the computer system 1001, can be embodied in programming. Various aspects of the technology can be thought of as “products” or “articles of manufacture” typically in the form of machine (or processor) executable code and/or associated data that is carried on or embodied in a type of machine readable medium. Machine-executable code can be stored on an electronic storage unit, such as memory (e.g., read-only memory, random-access memory, flash memory) or a hard disk. “Storage” type media can include any or all of the tangible memory of the computers, processors or the like, or associated modules thereof, such as various semiconductor memories, tape drives, disk drives and the like, which can provide non-transitory storage at any time for the software programming. All or portions of the software can at times be communicated through the Internet or various other telecommunication networks. Such communications, for example, can enable loading of the software from one computer or processor into another, for example, from a management server or host computer into the computer platform of an application server. Thus, another type of media that can bear the software elements includes optical, electrical and electromagnetic waves, such as used across physical interfaces between local devices, through wired and optical landline networks and over various air-links. The physical elements that carry such waves, such as wired or wireless links, optical links or the like, also can be considered as media bearing the software. As used herein, unless restricted to non-transitory, tangible “storage” media, terms such as computer or machine “readable medium” refer to any medium that participates in providing instructions to a processor for execution.

Hence, a machine readable medium, such as computer-executable code, can take many forms, including but not limited to, a tangible storage medium, a carrier wave medium or physical transmission medium. Non-volatile storage media include, for example, optical or magnetic disks, such as any of the storage devices in any computer(s) or the like, such as can be used to implement the databases, etc. shown in the drawings. Volatile storage media include dynamic memory, such as main memory of such a computer platform. Tangible transmission media include coaxial cables; copper wire and fiber optics, including the wires that comprise a bus within a computer system. Carrier-wave transmission media can take the form of electric or electromagnetic signals, or acoustic or light waves such as those generated during radio frequency (RF) and infrared (IR) data communications. Common forms of computer-readable media therefore include for example: a floppy disk, a flexible disk, hard disk, magnetic tape, any other magnetic medium, a CD-ROM, DVD or DVD-ROM, any other optical medium, punch cards paper tape, any other physical storage medium with patterns of holes, a RAM, a ROM, a PROM and EPROM, a FLASH-EPROM, any other memory chip or cartridge, a carrier wave transporting data or instructions, cables or links transporting such a carrier wave, or any other medium from which a computer can read programming code and/or data. Many of these forms of computer readable media can be involved in carrying one or more sequences of one or more instructions to a processor for execution.

The computer system 1001 can include or be in communication with an electronic display 1035 that includes a user interface (UI) 1040 for providing, for example, results of sequencing analysis, etc. Examples of UIs include, without limitation, a graphical user interface (GUI) and web-based user interface.

Methods and systems of the present disclosure can be implemented by way of one or more algorithms. An algorithm can be implemented by way of software upon execution by the central processing unit 1005. The algorithm can, for example, perform nucleotide sequence amplification, sequencing sorting based on barcode sizes, sequencing amplified barcode sequences, analyzing sequencing data, etc.

Devices, systems, compositions and methods of the present disclosure can be used for various applications, such as, for example, processing a single analyte (e.g., RNA, DNA, or protein) or multiple analytes (e.g., DNA and RNA, DNA and protein, RNA and protein, or RNA, DNA and protein) from a single cell within a tissue sample. This can enable, for example, simultaneous proteomic, transcriptomic and genomic analysis of the cell.

### E. Systems

The methods described above for analyzing biological samples can be implemented using a variety of hardware components. In this section, examples of such components are described. However, it should be understood that in general, the various steps and techniques discussed herein can be performed using a variety of different devices and system components, not all of which are expressly set forth.

In another aspect, some embodiments of the disclosure relate to systems for antibody discovery/management, the systems including: (a) a processor, e.g., a CPU, computer processor, or logic processor; (b) a data compiler communicatively coupled to the processor; (c) a stored program code that is executable by the processor; and (d) a report engine communicatively coupled to the processor, wherein reports produced by the report engine depend upon results from execution of the program code, wherein the program code configures the processor to receive from the data compiler information input pertaining to an antibody profile including a preselected set of data input in order to assign a relative performance score to the antibody's tumor specificity based at least in part on the antibody profile, whereby determining the likelihood of the antibody to exhibit one or more tumor specificity attributes as indicated by the assigned relative performance score.

Non-limiting exemplary embodiments of the systems of the disclosure can include one or more of the following features. In some embodiments, the data input includes one or more of the following: (a) antibody sequence data; (b) expression data of biomarkers in the B cell from which the antibody is derived; (c) transcriptomic data for the B cell from which the antibody is derived; and (d) genomic DNA sequence data from whole-exome sequencing. In some embodiments, the systems of the disclosure further include generating an antibody profile report that contains information relevant to the antibody identified as a tumor-specific antibody. In some embodiments, the antibody profile report is characterized as having an encoding selected from the group consisting of “.doc”; “.pdf”; “.xml”; “.html”; “.jpg”; “.aspx”; “.php”, and a combination of any thereof.

In yet another aspect, provided herein is a non-transitory computer readable medium containing machine executable instructions that when executed cause a processor to perform operations including: receiving an antibody profile including a preselected set of data input; assigning, based at least in part on the antibody profile, a relative performance score to the antibody's tumor specificity; and outputting an antibody profile report for the antibody based upon the assigned performance score. Accordingly, antibody profile reports generated by the systems of the disclosure are also with the scope of this disclosure.

All publications and patent applications mentioned in this disclosure are herein incorporated by reference to the same extent as if each individual publication or patent application was specifically and individually indicated to be incorporated by reference.

No admission is made that any reference cited herein constitutes prior art. The discussion of the references states what their authors assert, and the Applicant reserves the right to challenge the accuracy and pertinence of the cited documents. It will be clearly understood that, although a number of information sources, including scientific journal articles, patent documents, and textbooks, are referred to herein; this reference does not constitute an admission that any of these documents forms part of the common general knowledge in the art.

The discussion of the general methods given herein is intended for illustrative purposes only. Other alternative methods and alternatives will be apparent to those of skill in the art upon review of this disclosure, and are to be included within the spirit and purview of this application.

Additional embodiments are disclosed in further detail in the following examples, which are provided by way of illustration and are not in any way intended to limit the scope of this disclosure or the claims.

### EXAMPLES

### Example 1

Characterization of Antibody Specificity and/or Target Specificity

The methods provided herein can be implemented to identify antibodies that recognize tumor tissue antigens via barcoding.

A tumor tissue sample is obtained from a patient having a tumor.

The tissue sample is processed for spatial analysis according to a method disclosed herein. For example, the tissue sample can be contacted with a substrate comprising an array of capture probes. Analytes from an ABM-expressing cell of the tissue sample, as well as reporter oligonucleotides, and optionally reporter oligonucleotides from a panel of additional labeling agents, are attached (e.g., hybridized) to capture probes disclosed herein, e.g., according to a spatial analysis method disclosed herein. This can generate a reporter barcode library, a V(D)J library, and optionally a gene expression library (e.g., for global mRNA expression). The reporter barcode library can include spatially barcoded polynucleotides or amplicons or library members thereof comprising (i) a reporter barcode sequence or a reverse complement thereof and (ii) a spatial barcode sequence or a reverse complement thereof. The V(D)J library can include native sequences of ABMs.

The generated libraries are sequenced. Sequencing can be performed by various systems currently available, such as, without limitation, a sequencing system by Illumina®, Pacific Biosciences (PacBio®), Oxford Nanopore®, or Life Technologies (Ion Torrent®).

This is followed by gene synthesis, cloning, and production of selected recombinant antibodies. For subsequent characterization and validation of the phenotypic properties of the selected recombinant antibodies, the recombinant antibodies produced as described above are coupled to a reporter oligonucleotide including a reporter barcode sequence to generate barcoded recombinant antibodies.

The barcoded recombinant antibodies produced as described above are subsequently contacted with a tumor sample to identify those having specificity for the tumor sample as determined by their capability of binding to a tumor cell of the tumor sample and/or an antigen associated with the tumor sample.

### Example 2

### Identification of Patient-Specific or Population-Specific Biomarkers of Cancer

This Example describes experiments performed to identify patient-specific or population-specific biomarkers of cancer in accordance with some embodiments of the disclosure.

In these experiments, a collection of barcoded recombinant antibodies is generated as described in Example 1 above. A tumor sample (e.g., second tumor sample as described herein) taken from a cancer patient (e.g., the same patient that provided the tumor sample in Example 1) is subsequently contacted with a mixture of barcoded antibodies. In some embodiments, a plurality of tumor samples taken from multiple cancer patients suffering from the same cancer type is contacted with the mixture of barcoded antibodies. The mixture of barcoded antibodies can comprise any one of or more of (i) one or more barcoded immune-cell marker antibodies and/or functional fragments thereof, (ii) one or more barcoded tumor-cell marker antibodies and functional fragments thereof, (iii) one or more barcoded therapeutic antibodies and functional fragments thereof; and (iv) one or more barcoded recombinant antibodies identified and produced according to Example 1. Comparative analysis of in vitro and/or in vivo characterization the barcoded recombinant antibodies as well as gene expression and protein marker expression analysis of the tumor samples are performed to identify antibodies specific for a patient or a population of patients.

Additionally, comparative analysis of in vitro and/or in vivo characterization the barcoded recombinant antibodies as well as gene expression and protein marker expression analysis of a population of tumor samples are performed to identify biomarkers specific for individual tumor sample or for a population of tumor samples. In some embodiments, sequencing analysis of barcode sequences corresponding to (i) the one or more barcoded immune-cell marker antibodies and/or functional fragments thereof, and/or (ii) the one or more barcoded tumor-cell marker antibodies and functional fragments thereof, is used to identify patient-specific or population-specific biomarkers for the tumor or cancer.

### Example 3

### Monitoring Antigen Escape

This Example describes experiments performed to monitor antigen escape in an individual who has been treated with an antibody-based therapy in accordance with some embodiments of the disclosure.

In these experiments, a barcoded recombinant antibody having specificity for a tumor sample is generated as described in Example 1 above. The binding affinity of the barcoded recombinant antibody to a second tumor sample is subsequently evaluated by measuring the number of tumor cells expressing a target antigen of the barcoded recombinant antibody that are capable to binding to the barcoded recombinant antibody. In these experiments, the quantified binding affinity of the barcoded recombinant antibody to the second tumor sample is indicative of the recombinant antibody's efficacy in treating the tumor.

Additionally, the binding affinity of the barcoded recombinant antibody to an antigen expressed by the tumor sample is monitored over time, and is used as an indication of antigen escape from the recombinant antibody over time.

### Example 4

### Characterization of Potential Antigens

This Example describes experiments performed to identify and characterize potential antigens in accordance with some embodiments of the disclosure.

In these experiments, a collection of barcoded recombinant antibodies is generated as described in Example 1 above. The binding affinity of the barcoded recombinant antibodies to a second tumor sample is subsequently evaluated by quantifying binding affinity of the barcoded therapeutic antibodies to the second tumor sample. This is accomplished by measuring the number of tumor cells that express at least one antigen that binds to the one or more barcoded therapeutic antibodies. The quantified binding affinity is then used to determine if the recombinant antibodies compete with one another for binding to the second tumor sample. In addition, the quantified binding affinity of the recombinant antibodies is also used to co-associate with RNA expression analysis in identifying potential antigens capable of binding to the tested recombinant antibodies.

### Example 5

Identification of BCR and/or TCR Sequences from First Tumor Samples

Experiments are performed to generate paired, full-length T cell receptor sequences and/or B cell receptor sequences from tumor samples. A first tumor sample (e.g., a fresh frozen tissue section comprising tumor tissue) are prepared as described in the Visium Spatial Gene Expression Reagent Kits User Guide (e.g., Rev D, dated October 2020. The prepared samples are run according to spatial analysis methods described herein. Data is analyzed using Cell Ranger 6.0, Loupe 5.0, and Enclone. The analysis identifies paired full-length TCR and/or BCR sequences from the tumor samples.

### Example 6

### Production of Barcoded Recombinant Antibodies or TCRs

Using the BCR sequences identified from Example 5, nucleotide sequences encoding variable heavy chain and light chain domains of antibodies are reformatted to IgG1 and synthesized and cloned into a mammalian expression vector. Exemplary mammalian expression vectors are commercially available, e.g., pTwist CMV BG WPRE Neo (Twist Bioscience eCommerce portal), AddGene, InvivoGen, and Human IgG Vector Set from SigmaAldrich. Light chain variable domains are reformatted into kappa and lambda frameworks accordingly. Clonal genes are delivered as purified plasmid DNA ready for transfection in human embryonic kidney (HEK) Expi293 cells (Thermo Scientific). Alternatively, ExpiCHO cells may be used for transfection. Cultures in a volume of 1.2 ml are grown to four days, harvested, and purified using Protein A resin (PhyNexus) on the Hamilton Microlab STAR platform into 43 mM Citrate 148 mM HEPES, pH 6 to produce a recombinant antibody.

Alternatively, using the TCR sequences identified from Example 5, nucleotide sequences encoding TCR alpha and TCR beta chains are synthesized and cloned into a mammalian expression vector. Clonal genes are delivered as purified plasmid DNA ready for introduction in cultured cells, e.g., Jurkat cells. Such constructs may be introduced via using classical transformation techniques, e.g., transfection, transduction, or using more precise techniques such as guide RNA (gRNA)-directed CRISPR/Cas genome editing, DNA-guided endonuclease genome editing with NgAgo (Natronobacterium gregoryi Argonaute), or TALENs genome editing (transcription activator-like effector nucleases). Cultures are grown, harvested, and purified to produce a recombinant TCR. A TCR generally includes two polypeptides (e.g., polypeptide chains), such as a α-chain of a TCR, a β-chain of a TCR, a γ-chain of a TCR, a δ-chain of a TCR, or a combination thereof. Several approaches, techniques, and associated reagents for construction of recombinant TCR are known in the art. In some embodiments, the TCR constant region may be further 151iscuss to remove one or more domains thereof, which can be achieved by a known genome editing technique (e.g., CRISPR/Cas or TALENs discussed herein), via either homology directed repair, non-homologous end joining (NHEJ), and/or or microhomology-mediated end joining.

A reporter oligonucleotide comprising a reporter barcode sequence is coupled to the recombinant antibody or TCR according to available methods. The reporter barcode sequence is used as an identifier sequence for the antibody or TCR coupled thereto. In some instances, the reporter oligonucleotide is coupled to the recombinant antibody or TCR using chemical conjugation techniques (e.g., Lightning-Link® antibody labelling kits available from Innova Biosciences). In some instances, the reporter oligonucleotide is coupled to the recombinant antibody or TCR using non-covalent attachment mechanisms, e.g., using biotinylated antibodies and oligonucleotides (or beads that include one or more biotinylated linker, coupled to oligonucleotides) with an avidin or an streptavidin linker in monomeric or multimeric form (e.g., tetramic form of streptavidin). Antibody and oligonucleotide biotinylation techniques are available. See, e.g., Fang, et al., “Fluoride-Cleavable Biotinylation Phosphoramidite for 5′-end-Labelling and Affinity Purification of Synthetic Oligonucleotides,” Nucleic Acids Res. Jan. 15, 2003; 31(2):708-715, which is entirely incorporated herein by reference for all purposes. Likewise, protein and peptide biotinylation techniques have been developed and are readily available. See, e.g., U.S. Pat. No. 6,265,552, which is entirely incorporated herein by reference for all purposes. In some instances, the reporter oligonucleotide is coupled to the recombinant antibody or TCR using click reaction chemistry such as a Methyltetrazine-PEG5-NHS Ester reaction, a TCO-PEG4-NHS Ester reaction. In some instances, the reporter oligonucleotide is coupled to the recombinant antibody or TCR using a commercially available kit, such as from Thunderlink or Abcam. In some instances, the reporter oligonucleotide is coupled to the recombinant antibody or TCR indirectly (e.g., via hybridization). In some instances, the recombinant antibody or TCR is directly coupled (e.g., covalently bound) to a hybridization oligonucleotide that includes a sequence that hybridizes with a sequence of the reporter oligonucleotide. Hybridization of the hybridization oligonucleotide to the reporter oligonucleotide couples the labelling agent to the reporter oligonucleotide. In some embodiments, the reporter oligonucleotides are releasable from the labelling agent, such as upon application of a stimulus. For example, the reporter oligonucleotide can be attached to the labeling agent through a labile bond (e.g., chemically labile, photolabile, thermally labile, etc.) as generally described for releasing molecules from supports elsewhere herein. In some instances, the reporter oligonucleotides described herein can include one or more functional sequences that can be used in subsequent processing, such as an adapter sequence, a unique molecular identifier (UMI) sequence, a sequencer specific flow cell attachment sequence (such as an P5, P7, or partial P5 or P7 sequence), a primer or primer binding sequence, a sequencing primer or primer biding sequence (such as an R1, R2, or partial R1 or R2 sequence).

### Example 7

Analysis of Barcoded Recombinant Antibodies and/or TCRs Using Spatial Analysis Methodologies for Fresh Frozen Tumor Tissue Samples.

Recombinant barcoded antibodies and/or TCRs (e.g., produced according to methods described in Example 5), are further analyzed as follows.

A second tumor sample (e.g., a fresh frozen tissue section comprising tumor tissue) is mounted on a slide including an array of spatially barcoded capture probes (e.g., a Visium Spatial Gene Expression slide as described in the Visium Spatial Gene Expression Reagent Kits User Guide (e.g., Rev D, dated October 2020). After fixation (e.g., with 2% formalin, or with methanol) and blocking (e.g., with Triton-X), the second tumor sample is incubated with a cocktail of recombinant barcoded antibodies and/or TCRs. Optionally, the cocktail includes barcoded antibodies for known immune cell markers. Optionally, the cocktail includes barcoded antibodies for known tumor cell markers. Optionally, the cocktail includes one or more barcoded therapeutic antibodies. It is to be understood that the barcoded antibodies (e.g., barcoded antibodies for known immune cell markers, barcoded antibodies for known tumor cell markers, barcoded therapeutic antibodies) are coupled to reporter oligonucleotides comprising reporter barcode sequences that identify the antibody coupled thereto.

The sample is then stained (e.g., with H&E) and imaged according to any of the methods described herein. Optionally, if any of the barcoded antibodies include a fluorescence detection agent, the sample may be imaged via immunofluorescence.

The sample is permeabilized, e.g., according to methods described in the Visium Spatial Gene Expression Reagent Kits User Guide (e.g., Rev D, dated October 2020). Transcripts and reporter oligonucleotides are released during permeabilization for capture onto the spatially barcoded array. The captured transcripts and reporter oligonucleotides are used in an extension reaction to produce spatially barcoded extension products comprising sequences corresponding to the captured transcripts and/or reporter oligonucleotides, respectively. The spatially barcoded extension products are used to produce gene expression and reporter oligonucleotide libraries.

Sequence analysis is used to identify one or more recombinant barcoded antibodies and/or barcoded recombinant TCRs (e.g., as produced according to the methods of Example 7) as having specificity for the tumor. In some instances, comparative analysis of gene expression, the reporter oligonucleotide, and the image datasets, is performed to determine the recombinant antibodies' specificity and target specificity.

### Example 8

Analysis of Barcoded Recombinant Antibodies and/or TCRs Using Spatial Analysis Methodologies for FFPE Tumor Tissue Samples.

Recombinant barcoded antibodies and/or TCRs (e.g., produced according to methods described in Example 5), are further analyzed as follows.

A second tumor sample (e.g., a formalin-fixed paraffin embedded (FFPE) sample comprising tumor tissue) is mounted on a slide including an array of spatially barcoded capture probes (e.g., a spatially barcoded array slide as described in the Visium Spatial Gene Expression for FFPE User Guide (e.g., Rev A, dated June 2021)). The slide-mounted sample is dried overnight in a desiccator. The following day, the sample is heated to 60° C., followed by deparaffinization and rehydration. H&E staining is performed and the sample is imaged. The sample is destained using HCl and decrosslinked for 1 hour in citrate buffer (pH 6.0) at 95° C. After decrosslinking, the sample is incubated overnight with RTL (templated ligation) probe sets at 50° C., e.g., according to methods described in the Visium Spatial Gene Expression for FFPE User Guide (e.g., Rev A, dated June 2021). The following day, the sample is washed to remove un-hybridized probes, then treated with ligase to ligate the RTL probes. After another wash step, the sample is blocked with antibody blocking buffer. The sample is incubated overnight with a cocktail of recombinant barcoded antibodies and/or TCRs. Optionally, the cocktail includes barcoded antibodies for known immune cell markers. Optionally, the cocktail includes barcoded antibodies for known tumor cell markers. Optionally, the cocktail includes one or more barcoded therapeutic antibodies. It is to be understood that the barcoded antibodies (e.g., barcoded antibodies for known immune cell markers, barcoded antibodies for known tumor cell markers, barcoded therapeutic antibodies) are coupled to reporter oligonucleotides comprising reporter barcode sequences that identify the antibody coupled thereto.

The sample is washed with PBST, and washed with SSC. The sample is subjected to a 30 minute probe release step with Rnase, followed by a 1 hour permeabilization step with a permeabilization buffer including Proteinase K and detergent. Accordingly, the ligation products and reporter oligonucleotides of the barcoded antibodies are captured by the capture probes of the spatially barcoded array slide. The slide is washed twice with 2×SSC and subjected to probe extension, denaturation, and pre-amplification followed by amplification and sequencing of the templated ligation and reporter oligonucleotide libraries.

Sequence analysis is used to identify one or more recombinant barcoded antibodies and/or barcoded recombinant TCRs (e.g., as produced according to the methods of Example 7) as having specificity for the tumor. In some instances, comparative analysis of the templated ligation, the reporter oligonucleotide, and the image datasets, is performed to determine the recombinant antibodies' specificity and target specificity.

### Example 9. 5′ Capture of Target Nucleic Acids

FIG. 20 is a schematic showing generation of a cDNA by in situ reverse transcription of a target nucleic acid (e.g., mRNA) from a first primer including a sequence complementary to the target nucleic acid and a functional domain and a second primer that includes a capture sequence and a sequence complementary to a homopolynucleotide sequence.

More specifically, target nucleic acids are contacted with a first primer that includes a sequence complementary to the target nucleic acid (e.g., poly(dT) sequence, a poly(dTNV) sequence, a random sequence, a sequence encoding a constant region of an antibody, a B cell receptor, or a T cell receptor) and a functional domain. In some examples, the functional domain is a primer binding site. In some examples, the functional domain is a sequencing specific site (e.g., Read2 site). The target nucleic acid is reverse transcribed into cDNA and a polynucleotide sequence is added to the 3′ end of the cDNA. FIG. 20 shows a homopolynucleotide sequence comprising cytosines, however, it is appreciated that other polynucleotide sequences can be added to the 3′ end of the cDNA, including a heteropolynucleotide sequence. In some examples, the polynucleotide sequence is added by the reverse transcriptase. In some examples, the polynucleotide sequence is added by a terminal transferase (e.g., terminal deoxynucleotidyl transferase).

A second primer is added where the second primer includes a sequence complementary to the polynucleotide (e.g., a homopolynucleotide sequence, a heteropolynucleotide sequence) sequence added to the 3′ end of the cDNA and a capture sequence. In some examples, the second primer is RNA. After reverse transcription and extension of the 3′ end of the cDNA using the second primer as an extension template, an Rnase (e.g., Rnase H) is contacted with the biological sample (e.g., a tissue section). The Rnase degrades the RNA strand of the RNA/cDNA duplex, leaving a single-stranded cDNA product (e.g., an extension product) that includes the first primer at its 5′ end and the complement of the capture sequence capable of hybridizing a capture domain of a capture probe.

FIG. 21 is a schematic showing capture of the extension product (e.g., the single-stranded cDNA product shown in FIG. 20) by a capture probe on the substrate. The capture probe is attached to the substrate via its 5′ end and can include one or more functional domains, a spatial barcode, a unique molecular identifier, a capture domain, or a combination thereof. In some examples, the capture probe also includes a cleavage domain. The capture domain hybridizes to the complement of the capture sequence within the extension product (e.g., single-stranded cDNA product) from FIG. 20. In some examples, the 3′ end of the capture probe is extended using the extension product as a template. In some examples, the 3′ end of the extension product (e.g., single-stranded cDNA product) is extended using the capture probe as a template thereby generating an extended capture product. In some examples, the 3′ end of the capture probe is extended using the extension product as a template and the 3′ end of the extension product is extended using the capture probe as a template (e.g., generating an extended capture product). In some examples, the extended capture product is released from the capture probe. In some examples, the extended capture product is released via heat. In some examples, the extended capture product is denatured from the capture probe. In some examples, the extended capture product is denatured from the capture probe with KOH.

FIG. 22 is a schematic diagram showing an embodiment of FIG. 20 where reverse transcription of target nucleic acids is performed with a plurality of primers. In some examples, reverse transcription is performed using a reverse transcriptase with strand displacement activity. In some examples, reverse transcription is performed with a reverse transcriptase and a helicase. In some examples, reverse transcription is performed with a reverse transcriptase and a superhelicase. In some examples, reverse transcription is performed with one or more single-stranded DNA binding proteins. When a plurality of primers are used to template reverse transcription as shown in FIG. 22, the resulting extension products can be of different lengths depending on where the primer hybridized to the target nucleic acid. In some examples, a primer of the plurality of primers can template more than one reverse transcription reaction, thus resulting in two or more extension products generated from the same primer.

The released, extended captured products can be prepared for downstream applications, such as generation of a sequencing library and next-generation sequencing.

### Example 10. 5′ Capture of Target Nucleic Acids

A fresh frozen mouse brain sample was sectioned and placed on an array slide containing capture probes having a blocked capture domain. The tissue sections were fixed 5 min in 4% formaldehyde, followed by 5 min decrosslinking in 0.1N HCl.

The sections were washed in 1×PBS and reverse transcription (RT) was performed using a polydT30NV primer and an inhouse RT enzyme at 42 C overnight. Fluorescently labeled Cy3-dCTPs were spiked into the RT buffer to permit visualization of the synthesized cDNA. Additionally, a template switching ribonucleotide (rTSO) having a capture sequence as a handle was spiked in to allow the incorporation of the handle into the cDNA.

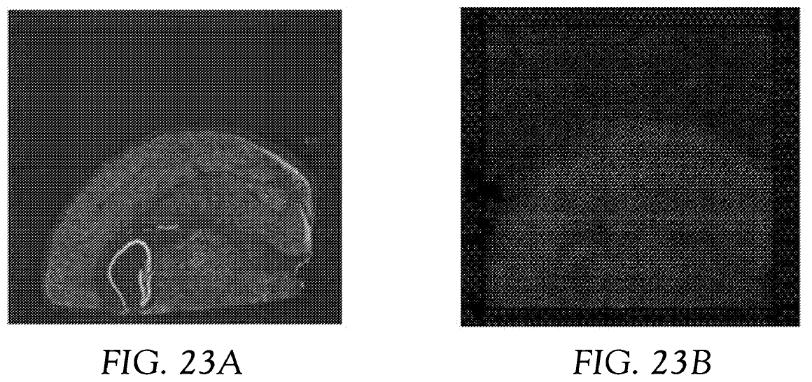

The next day, the sections were washed 0.2×SSC/20% Ethylene Carbonate at 50 C to remove any unspecific signal for the array. Afterwards the sections were imaged under the microscope (Cy3 channel, FIG. 23A). Post imaging, the RNA was digested using RnaseH, followed by tissue permeabilization.

Post permeabilization, the bound cDNA was extended using a polymerase and Cy3 was spiked into the mixture. Post extension, the slides were washed in 2×SSC followed by imaging (FIG. 23B).

FIGS. 23A-B are mouse brain images showing fluorescently labeled cDNA post reverse transcription (FIG. 23A) in situ where the reverse transcription reaction was performed overnight at 42° C. with Cy3 labeled dCTP and results in a tissue footprint. FIG. 23B shows fluorescently labeled extended cDNA post permeabilization and cDNA extension which also results in a tissue footprint.

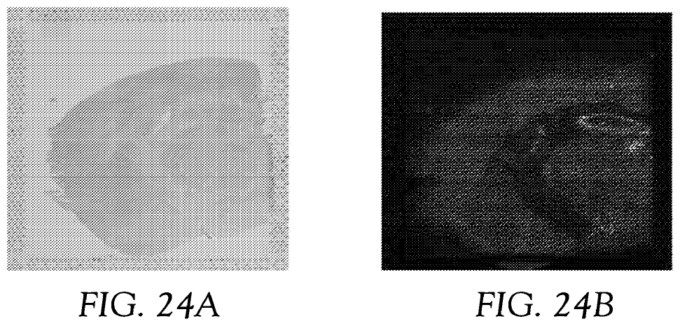

FIGS. 24A-B show mouse brain images from experiments similar to that described in FIGS. 23A-23B, but the RT reaction was performed without Cy3-dCTP spike in. FIG. 24A shows a brightfield image of a mouse brain tissue section and FIG. 24B shows fluorescently labeled extended cDNA where the capture domain of the capture probe is blocked. During extension, Cy3-dCTPs were spiked in to permit visualization of the captured cDNA.

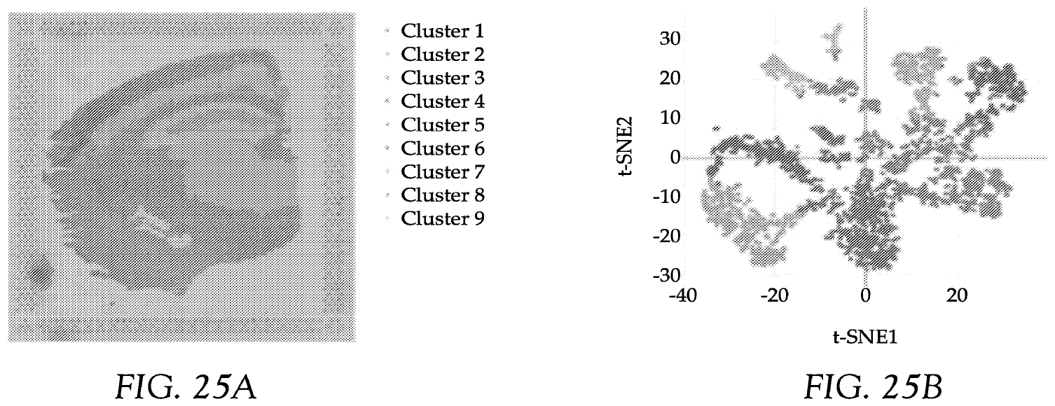

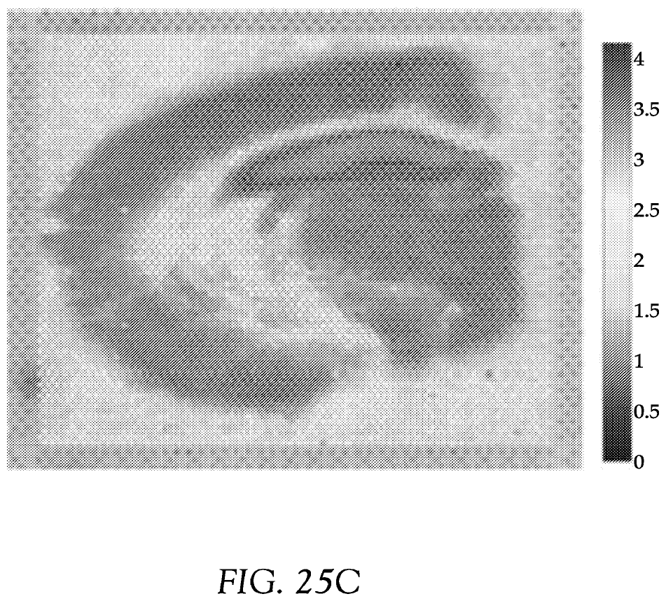

FIG. 25A shows spatial gene expression clusters, the corresponding t-SNE plot (FIG. 25B), and spatial gene expression heat map (FIG. 25C) from capture of extension products generated from experiments as described for FIGS. 23A-23B, except that RT and extension were performed without any Cy3-dCTP spike-in. The captured and extended cDNA was released using 0.08N KOH, followed by standard library preparation for next generation sequencing.

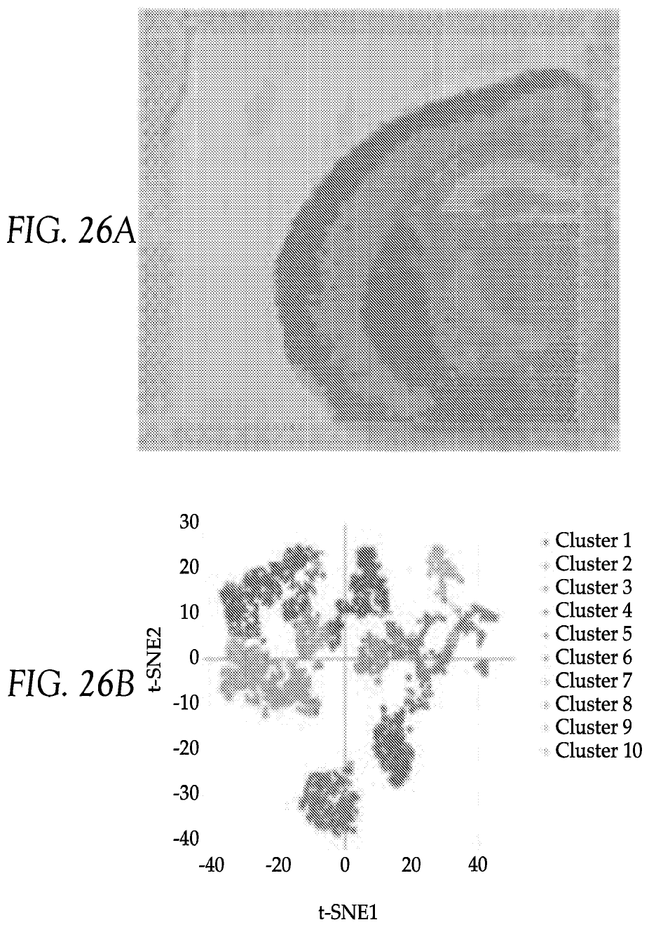

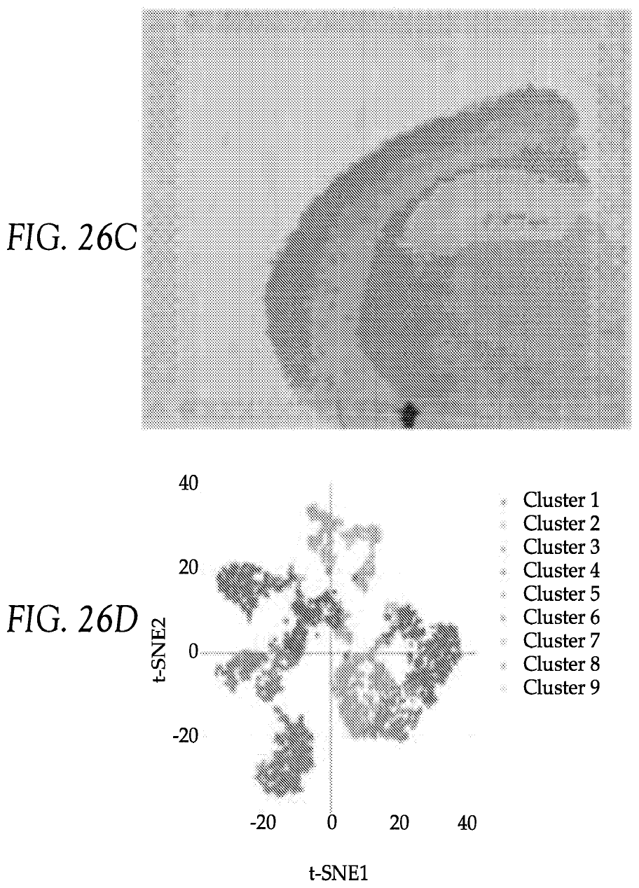

FIGS. 26A-D show spatial gene expression clustering with a first primer including a poly(T) sequence (poly(T)30NV) (FIG. 26A) and the corresponding t-SNE plot (FIG. 26B) and spatial gene expression clustering with a first primer including a random decamer (FIG. 26C) and the corresponding t-SNE plot (FIG. 26D) demonstrating that spatial gene expression information can be captured with in situ amplification with a first primer comprising either a poly(T) sequence or a random decamer sequence and where a complement of a capture sequence is incorporated into extension product(s) described herein.

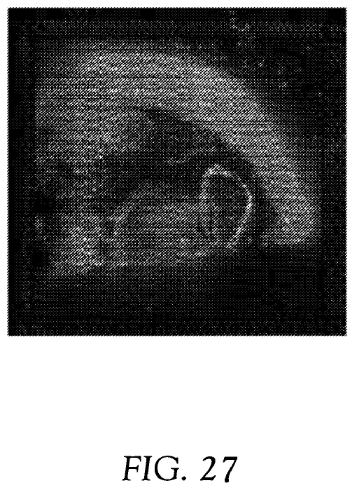

FIG. 27 shows fluorescently labeled extended probes captured in mouse brain tissue using an alternative capture sequence as the handle of the TSO, thereby demonstrating that in situ template switching in capture can work with various handles.

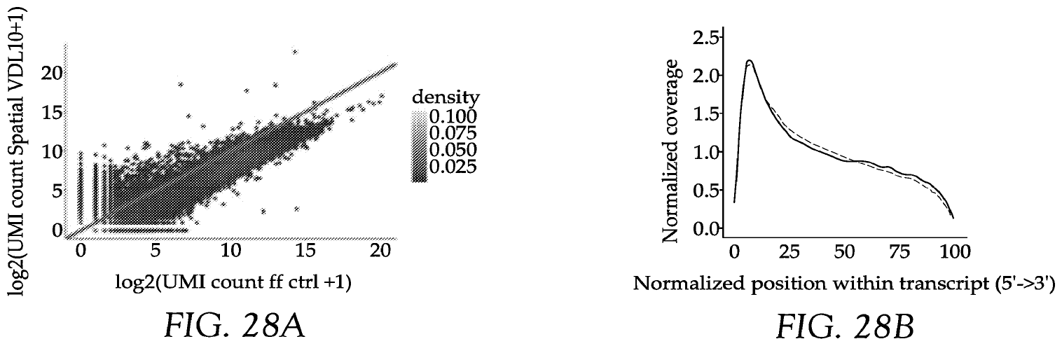

FIGS. 28A-B are graphs showing correlation between fresh frozen capture using standard Visium spatial gene expression (10× Genomics) and spatial 5′ end capture using the methods disclosed herein (FIG. 28A). Each dot represents the UMI counts for a single gene. FIG. 28B is a graph showing the normalized position of each mapped read within the full-length transcript. The data shown in the graph confirms successful 5′ capture of transcripts (e.g., regions containing CDR sequences).

The methods described herein are also able to identify sequences encoding for a complementarity determining region (“CDR”) e.g., CDR1, CDR2, and or CDR3 sequences, since CDR sequences are located at the 5′ ends of ABM encoding transcripts. Determining the sequence of CDRs can also identify clonotypes (e.g., a particular CDR combination) within a biological sample, such as a tissue sample (e.g., a tumor tissue sample).

While particular alternatives of the present disclosure have been disclosed, it is to be understood that various modifications and combinations are possible and are contemplated within the true spirit and scope of the appended claims. There is no intention, therefore, of limitations to the exact abstract and disclosure herein presented.

What is claimed is:

1. A method for identifying a tumor-specific antigen-binding molecule (ABM), the method comprising:
a) providing a tissue sample comprising one or more cells expressing an ABM;
b) attaching an analyte of an ABM-expressing cell of the tissue sample to a capture domain of a first capture probe of a substrate comprising an array of capture probes attached thereto, the first capture probe comprising (i) a spatial barcode sequence and (ii) the capture domain, the capture domain comprising a capture sequence, wherein the analyte of the ABM-expressing cell comprises a sequence or portion of a sequence encoding the ABM expressed by the ABM-expressing cell or a reverse complement thereof,
c) using the analyte of the ABM-expressing cell and the first capture probe attached thereto to generate a spatially barcoded polynucleotide comprising (i) all or part of a sequence of the analyte of the ABM-expressing cell or a reverse complement thereof and (ii) the spatial barcode sequence or a reverse complement thereof, and
d) determining all or a part of the nucleic acid sequences of (i) and (ii);
e) using the determined nucleic acid sequence of the analyte from (c) to produce a recombinant ABM;
f) coupling the recombinant ABM to a reporter oligonucleotide comprising a reporter barcode sequence, thereby generating a barcoded recombinant ABM; and
g) contacting the barcoded recombinant ABM with a tumor sample, and identifying the recombinant ABM as an ABM having specificity for the tumor sample if the barcoded recombinant antibody is capable of binding to an antigen associated with the tumor sample, optionally wherein the tumor sample is a tissue sample.

2. The method of claim 1, wherein the ABM expressed by the ABM-expressing cell is an immune cell receptor.

3. The method of claim 2, wherein the immune cell receptor is a BCR or a TCR.

4. The method of any one of claims 1-3, wherein the ABM expressed by the ABM-expressing cell is a secreted antibody.

5. The method of any one of claims 1-4, wherein the capture sequence of the capture domain is a homopolymeric sequence.

6. The method of any one of claims 1-5, wherein the capture sequence of the capture domain is a defined non-homopolymeric sequence.

7. The method of claim 6, wherein the defined non-homopolymeric sequence is a sequence that binds to the analyte.

8. The method of claim 6, wherein the defined non-homopolymeric sequence specifically binds to a nucleic acid sequence encoding a region of the ABM.

9. The method of any one of claims 1-8, wherein the ABM is selected from: a TCR alpha chain, a TCR beta chain, a TCR gamma chain, a TCR delta chain, an immunoglobulin kappa light chain, an immunoglobulin lambda light chain, an immunoglobulin heavy chain, or a combination thereof.

10. The method of claim 8 or 9, wherein the region of the ABM is a constant region of the ABM or a variable region of the ABM.

11. The method of claim 5, wherein the homopolymeric sequence is a poly(T) sequence.

12. The method of any one of claims 1-11, further comprising:
sequencing the spatially barcoded polynucleotide.

13. The method of any one of claims 1-12, wherein the tissue sample is mounted on the substrate comprising the array of capture probes during (a) or (b).

14. The method of claim 13, wherein a portion of the tissue sample comprising the ABM-expressing cell is in contact with the first capture probe of the array of capture probes.

15. The method of any one of claims 1-14, wherein the method comprises, following (a):
releasing the analyte from the ABM-expressing cell of the tissue sample; and
optionally migrating the analyte to the substrate comprising the array of capture probes attached thereto.

16. The method of any one of claims 1-12 or 15, wherein the substrate comprising the array of capture probes attached thereto is a second substrate, wherein the tissue sample is mounted on a first substrate during (a) or (b), and optionally, wherein the method comprises releasing the analyte from the ABM-expressing cell of the tissue sample; and optionally migrating the analyte to the second substrate comprising the array of capture probes attached thereto.

17. The method of any one of claims 1-16, wherein the analyte is a nucleic acid analyte.

18. The method of any one of claims 1-17, wherein the analyte comprises a sequence encoding a variable region and/or a constant region of the ABM.

19. The method of claim 18, wherein the variable region comprises a VJ or a VDJ sequence.

20. The method of claim 18 or 19, further comprising amplifying the spatially barcoded polynucleotide to generate a spatially barcoded double-stranded nucleic acid library member comprising the sequence encoding the variable region and the constant region of the ABM.

21. The method of claim 20, further comprising removing all or a portion of the sequence encoding the constant region of the ABM from the spatially barcoded double-stranded nucleic acid library member or amplicon thereof.

22. A method for identifying a tumor-specific antibody, the method comprising:
a) providing a first tumor tissue sample comprising one or more cells expressing one or more antibodies;
b) determining all or a part of the nucleic acid sequences encoding the one or more antibodies;
c) using the determined nucleic acid sequences to produce a recombinant antibody;
d) coupling the recombinant antibody to a reporter oligonucleotide comprising a reporter barcode sequence, thereby generating a barcoded recombinant antibody; and
e) contacting the barcoded recombinant antibody with a second tumor sample, and identifying the recombinant antibody as an antibody having specificity for the second tumor sample if the barcoded recombinant antibody is capable of binding to an antigen associated with the second tumor sample.

23. The method of claim 22, wherein the first tumor tissue sample and/or the second tumor sample is derived from a solid tumor, a soft tissue tumor, a metastatic lesion, a non-solid tumor, a circulating tumor cell (CTC) population, a tumor cell line, or a patient derived xenograft (PDX).

24. The method of claim 22 or 23, wherein the first tumor tissue sample and the second tumor sample are derived from the same subject or a different subject.

25. The method of any one of claims 22-24, wherein the first tumor tissue sample and the second tumor sample are derived from the same tumor.

26. The method of any one of claims 22-25, wherein step (e) further comprises contacting the barcoded recombinant antibody with a control tissue sample.

27. The method of claim 26, wherein the control sample is (i) a non-tumor tissue sample or (ii) a tissue sample that the barcoded recombinant antibody is not expected to bind.

28. The method of any one of claims 22-27, wherein the method further comprises contacting the second tumor sample with a composition comprising one or more of the following:
i) one or more barcoded immune-cell marker antibodies and/or barcoded tumor-cell marker antibodies;
ii) one or more barcoded therapeutic antibodies; and
iii) the barcoded recombinant antibody identified as having specificity for the second tumor sample.

29. The method of claim 28, wherein the one or more therapeutic antibodies is selected from the group consisting of abciximab, abciximab, adalimumab, aducanumab, alacizumab, alemtuzumab, alirocumab, alirocumab, ascrinvacumab, atezolizumab, atinumab, bapineuzumab, basiliximab, basiliximab, belimumab, bevacizumab, blinatumomab, blosozumab, bococizumab, brentuximab, canakinumab, caplacizumab, capromab, certolizumab, cetuximab, crenezumab, daclizumab, daratumumab, demcizumab, denosumab, denosumab, dinutuximab, ecukinumab, eculizumab, eculizumab, efalizumab, elotuzumab, enoticumab, etaracizumab, evinacumab, evolocumab, evolocumab, fasinumab, fulranumab, gantenerumab, golimumab, ibritumomab, icrucumab, idarucizumab, idarucizumab, inciacumab, infliximab, ipilimumab, mepolizumab, natalizumab, necitumumab, nesvacumab, nivolumab, obinutuzumab, ofatumumab, omalizumab, opicinumab, orticumab, ozanezumab, palivizumab, palivizumab, panitumumab, pembrolizumab, pertuzumab, ponezumab, ralpancizumab, ramucirumab, ramucirumab, ranibizumab, raxibacumab, refanezumab, rinucumab, rituximab, romosozumab, siltuximab, solanezumab, stamulumab, tadocizumab, tanezumab, tocilizumab, trastuzumab, ustekinumab, vedolizumab, and vesencumab.

30. The method of any one of claims 28-29, wherein the one or more immune-cell marker antibodies is selected from the group consisting of antibodies having specificity for one or more of B cells, T cells, monocytes, macrophages, granulocytes (basophil, eosinophil, neutrophil), dendritic cells, NK cells, and NKT cells.

31. The method of any one of claims 28-30, wherein the one or more tumor-cell marker antibodies is selected from the group consisting of antibodies having specificity for ALK, alpha-fetoprotein (AFP), beta-2-microglobulin (B2M), beta-human chorionic gonadotropin (Beta-hCG), bladder tumor antigen (BTA), BRCA1, BRCA2, BCR-ABL fusion gene (Philadelphia chromosome), BRAF V600 mutations, C-kit/CD117, CA15-3/CA27.29, CA-125, CA 27.29, carcinoembryonic antigen (CEA), CD20, CD22, CD25, CD30, CD31, CD33, CD44, CD133, CD176, CD276, estrogen receptor (ER), E-cadherin, ESPR, EGFR, EPCAM, GD2, progesterone receptor (PR), fibrin/fibrinogen, HE4 gene variants, HER2 gene variants, JAK2 gene variants, KRAS gene variants, nuclear matrix protein 22, PCA3, PML/RARα fusion gene, programmed death-ligand 1 (PD-L1 or CD274), prostate-specific antigen (PSA), TEM7, TEM8, and VEGF receptor family members.

32. The method of any one of claims 22-31, wherein identifying the recombinant antibody as an antibody having specificity for the second tumor sample further comprises quantifying levels of gene expression and protein marker expression in the second tumor sample.

33. The method of claim 32, further comprising using the quantified levels for identification of biomarkers specific for the second tumor sample and/or a subject from whom the second tumor sample is obtained.

34. The method of any one of claims 22-33, further comprising quantifying binding affinity of one or more therapeutic antibodies to the second tumor sample.

35. The method of claim 34, further comprising using the quantified binding affinity as an indicator of efficacy of treating a tumor with the one or more therapeutic antibodies.

36. The method of any one of claims 34-35, further comprising using the quantified binding affinity to monitor antigen escape of a tumor from the one or more therapeutic antibodies over time.

37. The method of any one of claims 23-36, wherein identifying the recombinant antibody as an antibody having specificity for the second tumor sample further comprises comparing the determined nucleic acid sequences encoding the antibody to a genomic DNA sequence from the second tumor sample to confirm antigen specificity of the antibody.

38. The method of claim 37, wherein the genomic DNA sequence is obtained from a single cell in the second tumor sample.

39. The method of claim 37, wherein the genomic DNA sequence is obtained from a plurality of cells in the second tumor sample.

40. The method of any one of claims 37-39, wherein the genomic DNA sequence is obtained by whole-genome sequencing.

41. The method of any one of claims 22-40, wherein identifying the recombinant antibody as an antibody having specificity for the second tumor sample further comprises comparing the determined nucleic acid sequences encoding the antibody to a sequence of a ribonucleic acid (RNA) molecule from the second tumor sample to confirm antigen specificity of the antibody.

42. The method of claim 41, wherein the RNA molecule is obtained from a single cell in the second tumor sample.

43. The method of claim 41, wherein the RNA molecule is obtained from a plurality of cells in the second tumor sample.

44. The method of any one of claims 41-43, further comprising obtaining the sequence of the RNA molecule.

45. The method of any one of claims 22-44, further comprising determining a nucleic acid sequence of a messenger RNA (mRNA) from a single B cell and/or from a single tumor cell within the first tumor tissue sample or the second tumor sample.

46. The method of claim 45, wherein the determining comprises binding one or more nucleic acid barcode molecules to the mRNA and optionally generating a complementary DNA (cDNA) via reverse transcription.

47. The method of claim 46, wherein the one or more nucleic acid barcode molecules independently comprise one or more barcode sequences.

48. The method of claim 47, wherein the one or more barcode sequences is selected from the group consisting of a sample barcode, a tissue barcode, a cell barcode, a spatial barcode, and a unique molecular identifier (UMI).

49. The method of any one of claims 46-48, wherein the one or more nucleic acid barcode molecules are coupled to a microcapsule.

50. The method of claim 49, wherein the microcapsule comprises a bead.

51. The method of any one of claims 45-50, wherein the determining includes whole transcriptome sequencing.

52. The method of any one of claims 45-51, wherein the determining comprises next-generation sequencing (NGS).

53. The method of any one of claims 22-52, further comprising generating a chimeric antigen receptor (CAR) using the nucleic acid sequence of the recombinant antibody.

54. The method of any one of claims 22-53, further comprising administering a composition comprising the recombinant antibody or a fragment thereof to a subject in need thereof.

55. The method of any one of claims 23-54, further comprising administering a composition comprising an immune cell expressing the recombinant antibody or a fragment thereof to a subject in need thereof.

56. The method of any one of claims 22-55, further comprising comparing the determined nucleic acid sequence of the recombinant antibody to sequences of known antibodies in order to identify the antibody as a tumor-specific antibody.

57. The method of any one of claims 22-56, further comprising using a filter that takes into account clonal expansions to identify the recombinant antibody as a tumor-specific antibody.

58. The method of any one of claims 22-57, further comprising using a filter that takes into account gene expression profiles of B cells to identify the recombinant antibody as a tumor-specific antibody.

59. The method of any one of claims 22-58, further comprising using a filter that takes into account somatic hypermutation and isotype usage to identify the recombinant antibody as a tumor-specific antibody.

60. A method for generating a recombinant antibody, the method comprising:
a) providing a first tumor tissue sample;
b) determining all or a part of the nucleic acid sequences encoding one or more antibodies produced by one or more cells of the first tumor tissue sample; and
c) using the determined nucleic acid sequences to produce a recombinant antibody.

61. The method of claim 60, further comprising coupling a reporter oligonucleotide comprising a reporter barcode sequence to the recombinant antibody to generate a barcoded recombinant antibody.

62. The method of claim 60 or 61, wherein the reporter barcode sequence of the reporter oligonucleotide comprises one or more unique identifiers for the recombinant antibody.

63. The method claim 62, further comprising determining all or a part of the nucleic acid sequence of the one or more unique identifiers to identify the barcoded recombinant antibody.

64. The method of any one of claims 60-63, wherein the reporter oligonucleotide comprises an adapter region that allows for downstream analysis of the recombinant antibody.

65. The method of claim 64, wherein the adapter region comprises a primer binding site and/or a cleavage site.

66. The method of any one of claims 60-65, further comprising contacting the barcoded recombinant antibody to a tumor cell obtained from a second tumor tissue sample.

67. The method of claim 66, further comprising identifying the recombinant antibody as an antibody having specificity for the second tumor tissue sample if the barcoded recombinant antibody is capable of binding to an antigen associated with the second tumor tissue sample.

68. The method of claim 66 or 67, wherein the first and/or the second tumor tissue sample is derived from a solid tumor, a soft tissue tumor, a metastatic lesion, a non-solid tumor, a circulating tumor cell (CTC) population, a tumor cell line, or a patient derived xenograft (PDX).

69. The method of any one of claims 66-68, wherein the first and the second tumor tissue samples are derived from the same subject or a different subject.

70. The method of any one of claims 66-68, wherein the first and the second tumor tissue samples are derived from the same tumor.

71. The method of any one of claims 66-70, wherein the method comprises contacting the second tumor tissue sample with a composition comprising one or more of the following:
i) one or more barcoded immune-cell marker antibodies and/or barcoded tumor-cell marker antibodies;
ii) one or more barcoded therapeutic antibodies; and
iii) the barcoded recombinant antibody identified as having specificity for the second tumor tissue sample.

72. The method of claim 71, wherein the one or more therapeutic antibodies is selected from the group consisting of abciximab, abciximab, adalimumab, aducanumab, alacizumab, alemtuzumab, alirocumab, alirocumab, ascrinvacumab, atezolizumab, atinumab, bapineuzumab, basiliximab, basiliximab, belimumab, bevacizumab, blinatumomab, blosozumab, bococizumab, brentuximab, canakinumab, caplacizumab, capromab, certolizumab, cetuximab, crenezumab, daclizumab, daratumumab, demcizumab, denosumab, denosumab, dinutuximab, ecukinumab, eculizumab, eculizumab, efalizumab, elotuzumab, enoticumab, etaracizumab, evinacumab, evolocumab, evolocumab, fasinumab, fulranumab, gantenerumab, golimumab, ibritumomab, icrucumab, idarucizumab, idarucizumab, inciacumab, infliximab, ipilimumab, mepolizumab, natalizumab, necitumumab, nesvacumab, nivolumab, obinutuzumab, ofatumumab, omalizumab, opicinumab, orticumab, ozanezumab, palivizumab, palivizumab, panitumumab, pembrolizumab, pertuzumab, ponezumab, ralpancizumab, ramucirumab, ramucirumab, ranibizumab, raxibacumab, refanezumab, rinucumab, rituximab, romosozumab, siltuximab, solanezumab, stamulumab, tadocizumab, tanezumab, tocilizumab, trastuzumab, ustekinumab, vedolizumab, and vesencumab.

73. The method of claim 71 or 72, wherein the one or more immune-cell marker antibodies is selected from the group consisting of antibodies having specificity for one or more of B cells, T cells, monocytes, macrophages, granulocytes (basophil, eosinophil, neutrophil), dendritic cells, NK cells, and NKT cells.

74. The method of any one of claims 71-73, wherein the one or more tumor-cell marker antibodies is selected from the group consisting of antibodies having specificity for ALK, alpha-fetoprotein (AFP), beta-2-microglobulin (B2M), beta-human chorionic gonadotropin (Beta-hCG), bladder tumor antigen (BTA), BRCA1, BRCA2, BCR-ABL fusion gene (Philadelphia chromosome), BRAF V600 mutations, C-kit/CD117, CA15-3/CA27.29, CA-125, CA 27.29, carcinoembryonic antigen (CEA), CD20, CD22, CD25, CD30, CD31, CD33, CD44, CD133, CD176, CD276, estrogen receptor (ER), E-cadherin, ESPR, EGFR, EPCAM, GD2, progesterone receptor (PR), fibrin/fibrinogen, HE4 gene variants, HER2 gene variants, JAK2 gene variants, KRAS gene variants, nuclear matrix protein 22, PCA3, PML/RARα fusion gene, programmed death-ligand 1 (PD-L1 or CD274), prostate-specific antigen (PSA), TEM7, TEM8, and VEGF receptor family members.

75. A recombinant antibody or a functional fragment thereof generated or identified by a method according to any one of claims 22 to 74.

76. A recombinant nucleic acid comprising a nucleic acid sequence that encodes the recombinant antibody or functional fragment thereof of claim 75.

77. The recombinant nucleic acid of claim 76, wherein the recombinant nucleic acid is further configured as an expression cassette in a vector.

78. The recombinant nucleic acid of claim 77, wherein the vector is a plasmid vector or a viral vector.

79. A recombinant cell comprising a recombinant nucleic acid according to any one of claims 75-78.

80. The recombinant cell of claim 79, wherein the recombinant cell is a prokaryotic cell or a eukaryotic cell.

81. A composition comprising a pharmaceutically acceptable excipient and one or more of the following:
a) a recombinant antibody or the functional fragment thereof of claim 75;
b) a recombinant nucleic acid according to any one of claims 76 to 78; or
c) a recombinant cell according to claim 79 or 80.

82. A composition comprising one or more of the following:
a) one or more barcoded immune-cell marker antibodies and/or barcoded tumor-cell marker antibodies;
b) one or more barcoded therapeutic antibodies; or
c) the recombinant antibody identified in claim 75 as having specificity for the second tumor sample, wherein the recombinant antibody is barcoded.

83. A kit comprising one or more of the following:
a) the recombinant antibody or the functional fragment thereof of claim 75;
b) a recombinant nucleic acid according to any one of claims 76 to 78; or
c) a recombinant cell according to claim 79 or 80; or
instructions for use thereof.

84. A method for characterizing antibody specificity or target specificity, the method comprising:
a) providing a first tumor tissue sample;
b) determining all or a part of one or more nucleic acid sequences encoding one or more antibodies produced by B cells in the tumor tissue sample;
c) using the determined nucleic acid sequences to produce one or more recombinant antibodies;
d) coupling the one or more recombinant antibodies to a reporter oligonucleotide comprising a reporter barcode sequence to generate one or more barcoded recombinant antibodies;
e) contacting the one or more barcoded recombinant antibodies with a second tumor sample, and identifying the one or more recombinant antibodies as having specificity for the tumor sample if the one or more barcoded recombinant antibodies is capable of binding to an antigen associated with the second tumor sample, optionally wherein the second tumor sample is a tissue sample; and
f) analyzing RNA expression and protein marker expression for the first tumor tissue samples and/or second tumor sample to determine the one or more recombinant antibody's specificity and target specificity.

85. A method for enhanced identification of patient-specific or population-specific biomarkers, the method comprising:
a) providing a tumor tissue sample comprising a plurality of B cells;
b) determining all or a part of the nucleic acid sequences encoding one or more antibodies produced by B cells in the tumor tissue sample;
c) using the determined nucleic acid sequences to produce a recombinant antibody;
d) coupling the recombinant antibody to a reporter oligonucleotide comprising a reporter barcode sequence to generate a barcoded recombinant antibody;
e) contacting the barcoded recombinant antibody with a second tumor sample, and identifying the recombinant antibody as an antibody having specificity for the second tumor sample if the barcoded recombinant antibody is capable of binding to an antigen associated with the second tumor sample, optionally wherein the second tumor sample is a tissue sample; and
f) analyzing RNA expression and protein marker expression for the second tumor sample to identify one or more biomarkers specific for the second tumor sample or for a population of tumor tissue samples.

86. A method for monitoring antigen escape in an individual who has been treated with an antibody-based therapy, the method comprising:
a) providing a tumor tissue sample comprising a plurality of B cells;
b) determining all or a part of the nucleic acid sequences encoding one or more antibodies produced by the B cells in the tumor tissue sample;
c) using the determined nucleic acid sequences to produce a recombinant antibody;
d) coupling the recombinant antibody to a reporter oligonucleotide comprising a reporter barcode sequence to generate a barcoded recombinant antibody;
e) contacting the barcoded recombinant antibody with a second tumor tissue sample, and identifying the recombinant antibody as an antibody having specificity for the tumor tissue sample if the barcoded recombinant antibody is capable of binding to an antigen associated with the second tumor tissue sample;
f) quantifying binding affinity of a barcoded therapeutic antibody to the second tumor tissue sample, wherein the quantified binding affinity is indicative of the therapeutic antibody's efficacy in treating the tumor; and
g) optionally using the quantified binding affinity to monitor antigen escape from the therapeutic antibody over time.

87. A method for characterizing a potential antigen for an antibody or fragment thereof, the method comprising:
a) providing a tumor tissue sample comprising a plurality of B cells;
b) determining all or a part of the nucleic acid sequences encoding one or more antibodies produced by the B cells in the tumor tissue sample;
c) using the determined nucleic acid sequences to produce a recombinant antibody;
d) coupling the recombinant antibody to a reporter oligonucleotide comprising a reporter barcode sequence to generate a barcoded recombinant antibody;
e) contacting the barcoded recombinant antibody with a second tumor tissue sample, and identifying the recombinant antibody as an antibody having specificity for the tumor tissue sample if the barcoded recombinant antibody is capable of binding to an antigen associated with the second tumor tissue sample; and
f) quantifying binding affinity of the one or more antibodies to the second tumor tissue sample, and using the quantified binding affinity to determine if the one or more antibodies compete with one another for binding to the second tumor tissue sample; and
g) optionally co-associating the quantified binding affinity with RNA expression analysis to identify potential antigen.

88. A method for identifying a tumor-specific antibody, comprising:
a) contacting a barcoded recombinant antibody with a tumor tissue sample; and
b) identifying the barcoded recombinant antibody as a tumor-specific antibody if the barcoded recombinant antibody is capable of binding to an antigen associated with the tumor tissue sample,
wherein the barcoded recombinant antibody comprises a recombinant antibody coupled to a reporter oligonucleotide comprising a reporter barcode sequence, wherein the recombinant antibody is identified and/or produced by (i) providing a tumor tissue sample comprising a plurality of B cells (ii) determining all or a part of the nucleic acid sequences encoding one or more antibodies produced by the B cell cells in the tumor tissue sample, and optionally (iii) using the determined nucleic acid sequences to recombinantly produce the recombinant antibody.

89. The method of claim 88, wherein the barcoded recombinant antibody is produced by coupling the recombinant antibody to a reporter oligonucleotide comprising a reporter barcode sequence.

90. The method of any one of claims 1-74 or 85-89, wherein determining all or a part of the nucleic acid sequences of the analyte or all or a part of the nucleic acid sequence encoding the one or more antibodies comprises:
a) contacting the tissue sample or tumor tissue sample with a first primer comprising a nucleic acid sequence that hybridizes to a complementary sequence in the analyte or nucleic acid sequence encoding the one or more antibodies and a functional domain;
(b) hybridizing the first primer to the analyte or the nucleic acid sequence encoding the one or more antibodies and extending the first primer using the analyte or the nucleic acid sequence encoding the one or more antibodies as a template to generate an extension product;
(c) incorporating a polynucleotide sequence comprising at least three non-templated nucleotides to the 3′ end of the extension product;
(d) hybridizing a second primer to the polynucleotide sequence comprising at least three non-templated nucleotides of the extension product of (c), wherein the second primer comprises a capture sequence;
(e) extending the extension product using the second primer as a template, thereby incorporating a complement of the capture sequence into the extension product;
(f) hybridizing the complement of the capture sequence of the extension product to a capture domain on an array, wherein the array comprises a plurality of capture probes, and wherein a capture probe of the plurality of capture probes comprises a spatial barcode and the capture domain; and
(g) determining (i) the sequence of the spatial barcode, or a complement thereof, and (ii) all or a portion of the sequence of the nucleic acid sequence, or a complement thereof, and using the determined sequences of (i) and (ii) to determine all or a part of the nucleic acid sequence of the analyte or the nucleic acid sequence encoding the one or more antibodies.

91. The method of claim 90, wherein the first primer comprises a random sequence, optionally wherein the random sequence comprises a random hexamer or random decamer.

92. The method of claim 90, wherein the first primer comprises a homopolymer sequence, optionally wherein the homopolymer sequence comprises a poly(T) sequence.

93. The method of any one of claims 90-92, wherein the first primer comprises a sequence substantially complementary to a sequence in the analyte or all or a part of the nucleic acid sequence encoding the one or more antibodies encoding a constant region of an immune cell receptor, optionally wherein the immune cell receptor comprises a B cell receptor or a T cell receptor.

94. The method of any one of claims 90-93, wherein incorporating the polynucleotide sequence to the 3′ end of the extension product in step (c) comprises the use of a terminal deoxynucleotidyl transferase or of a reverse transcriptase.

95. The method of any one of claims 90-94, wherein the second primer comprises RNA.

96. The method of any one of claims 90-95, wherein the method further comprises removing the analyte or the nucleic acid sequence encoding the one or more antibodies, or any other nucleic acid hybridized to the extension product, before the complement of the capture sequence of the extension product hybridizes to the capture domain of the capture probe on the array, optionally wherein the removing comprises the use of an RNase, optionally wherein the RNase is RNaseH.

97. The method of any one of claims 90-96, wherein the method further comprises a step of extending the 3′ end of the extension product of step (e) using the capture probe as a template, thereby generating an extended capture product, and/or extending the capture probe using the extension product of step (e) as a template.

98. The method of claim 97, wherein step (b) comprises generating one or more extension products using a plurality of primers, wherein a primer of the plurality of primers comprises a nucleic acid sequence that is substantially complementary to a sequence in the target nucleic acid and a functional domain, wherein the first primer is comprised in the plurality of primers;
(a) hybridizing the plurality of primers to the analyte or the nucleic acid sequence encoding the one or more antibodies and extending one or more primers from the plurality of primers using the target nucleic acid as a template to generate the one or more extension products;
(b) attaching a polynucleotide sequence to the 3′ end of the one or more extension products;
(c) hybridizing the second primer to the polynucleotide sequence of the one or more extension products of (b), wherein the second primer comprises a capture sequence;
(d) extending the one or more extension products using the second primer as a template, thereby incorporating a complement of the capture sequence into the one or more extension products;
(e) hybridizing the complement of the capture sequence of the one or more extension products to a capture domain on an array, wherein the array comprises a plurality of capture probes, and wherein the capture probe of the plurality of capture probes comprises a spatial barcode and the capture domain; and
(f) determining (i) the sequence of the spatial barcode, or a complement thereof, and (ii) all or a portion of the sequence of the target nucleic acid, or a complement thereof, and using the determined sequences of (i) and (ii) to determine all or a part of the nucleic acid sequence of the analyte or the nucleic acid sequence encoding the one or more antibodies.

99. The method of any one of claims 1-74 or 85-89, wherein determining all or a part of the nucleic acid sequence of the analyte or all or a part of the nucleic acid sequence encoding the one or more antibodies comprises:
(a) contacting the tissue sample or tumor tissue sample with an array comprising a plurality of capture probes, wherein a capture probe of the plurality of capture probes comprises (i) a spatial barcode and (ii) a capture domain that hybridizes to a poly(A) sequence of the analyte or the nucleic acid sequence encoding the one or more antibodies;
(b) hybridizing the capture domain to the analyte or nucleic acid sequence encoding the one or more antibodies;
(c) extending the capture probe using the analyte or nucleic acid sequence encoding the one or more antibodies as a template to generate an extended capture probe comprising a sequence encoding a CDR3, or a complement thereof, of the ABM or one or more antibodies;
(d)hybridizing one or more probes to the extended capture probe, or a complement thereof, in a portion encoding a constant region of the ABM or one or more antibodies, wherein the one or more probes comprises a binding moiety capable of binding a capture moiety;
(e) enriching the extended capture probe or the complement thereof via an interaction between the binding moiety in the one or more probes and the capture moiety; and
(f) determining (i) the sequence of the spatial barcode or a complement thereof, and (ii) all or a portion of the sequence of the analyte or nucleic acid sequence encoding the one or more antibodies, or a complement thereof, and using the determined sequences of (i) and (ii) to determine all or part of the nucleic acid sequence of the analyte or the nucleic acid sequence encoding the one or more antibodies.

100. The method of claim 99, wherein the one or more probes hybridizes to a nucleic acid sequence encoding a constant region of the ABM or one or more antibodies, or a complement thereof.

101. The method of claim 99 or 100, wherein the capture domain comprises a poly(T) sequence.

102. The method of any one of claims 99-101 further comprising generating the complement of the extended capture using the extended capture probe as a template, wherein the complement of the extended capture probe comprises (i) a sequence that is complementary to the spatial barcode, and (ii) a sequence that corresponds to all or a portion of the analyte or nucleic acid sequence encoding the one or more antibodies.

103. The method of any one of claims 99-102, wherein the binding moiety comprises biotin and the capture moiety comprises streptavidin.

104. The method of any one of claims 99-103, wherein the determining in step (f) comprises sequencing the extended capture probe or the complement thereof to determine (i) the sequence of the spatial barcode, or the complement thereof, and (ii) all or a portion of the sequence of the the analyte or nucleic acid sequence encoding the one or more antibodies, optionally wherein the sequencing comprises long read sequencing.

105. The method of any one of claims 99-104, wherein the capture probe further comprises an adaptor domain and the method further comprises after step (e), performing a polymerase chain reaction using i) a first primer complementary to the adaptor domain of the capture probe, and ii) a second primer complementary to a portion of the analyte or nucleic acid sequence encoding the one or more antibodies in a portion encoding a variable region of the immune cell receptor of the immune cell clonotype.

106. The method of claim 105, wherein the second primer is complementary to a nucleic acid sequence 5′ to the sequence encoding CDR3 of the ABM or the one or more antibodies.

107. The method of any one of claims 1-74 or 85-89, wherein determining all or a part of the nucleic acid sequences of the analyte or all or a part of the nucleic acid sequence encoding the one or more antibodies comprises:
(a) contacting the tissue sample or tumor tissue sample with an array comprising a feature, wherein the feature comprises an attached first and second probe, wherein:
a 5′ end of the first probe is attached to the feature;
the first probe comprises in a 5′ to a 3′ direction: a spatial barcode and a poly(T) capture domain, wherein the poly(T) capture domain binds specifically to the analyte or the nucleic acid sequence encoding the one or more antibodies;
a 5′ end of the second probe is attached to the feature;
a 3′ end of the second probe is reversibly blocked; and
the second probe comprises a poly(GI) capture domain;
(b) extending a 3′ end of the first probe to add a sequence that is complementary to a portion of the analyte or the nucleic acid sequence encoding the one or more antibodies;
(c) ligating an adapter to the 5′ end of analyte or the nucleic acid sequence encoding the one or more antibodies specifically bound to the first probe;
(d) adding a sequence complementary to the adapter to the 3′ end of the first probe;
(e) adding non-templated cytosines to the 3′ end of the first probe to generate a poly(C) sequence, wherein the poly(C) sequence specifically binds to the poly(GI) capture domain of the second probe;
(f) unblocking the 3′ end of the second probe and extending the 3′ end of the second probe to add a sequence comprising a sequence in the analyte or the nucleic acid sequence encoding the one or more antibodies and a sequence that is complementary to the spatial barcode;
(g) cleaving a region of the second probe at a cleavage site that is 5′ to the poly(GI) capture domain, thereby releasing the second probe from the feature; and
(h) determining (i) all or a part of the sequence of the spatial barcode, or a complement thereof, and (ii) all or a part of the sequence of the analyte or the nucleic acid sequence encoding the one or more antibodies, or a complement thereof, and using the sequences of (i) and (ii) to determine all or a part of the nucleic acid sequence of the analyte or the nucleic acid sequence encoding the one or more antibodies.

108. The method of claim 21, wherein a single strand of the double-stranded nucleic acid library member comprises: a first adaptor, a barcode, a capture domain, a sequence of the analyte or a complement thereof, and a second adaptor.

109. The method of claim 21, wherein removing all or a portion of the sequence encoding the constant region of the ABM from the spatially barcoded double-stranded nucleic acid library member comprises:
(a) ligating to each end of the double-stranded member of the nucleic acid library a first restriction endonuclease recognition sequence;
(b) contacting the double-stranded member of the nucleic acid library of step (a) with a first restriction endonuclease that cleaves the first restriction endonuclease recognition sequence at each end;
(c) ligating the ends of the double-stranded member of the nucleic acid library of step (b) to generate a first double-stranded circularized nucleic acid; and
(d) amplifying the double-stranded circularized nucleic acid using a first primer and a second primer to generate a double-stranded member of the nucleic acid library lacking all, or a portion of, the analyte sequence, wherein:
the first primer comprises: (i) a sequence substantially complementary to a 3′ region of the analyte sequence, and (ii) a first functional domain comprising a sequence for attachment to a flow cell; and
the second primer comprises: (i) a sequence substantially complementary to a 5′ region of the analyte sequence, and (ii) a second functional domain comprising a primer sequence to amplify the double-stranded member of the nucleic acid library lacking all, or a portion of, encoding the constant region of the ABM.

110. The method of claim 109, wherein the first primer comprises (i) the sequence substantially complementary to the 3′ region of the analyte sequence, and (ii) the sequence comprising the first functional domain, in 3′ to 5′ direction; and wherein the second primer comprises (i) the sequence substantially complementary to the 5′ region of the analyte sequence, and (ii) the sequence comprising the second functional domain, in a 3′ to 5′ direction.

111. The method of claim 109 or 110, wherein ligating in step (c) is performed using a DNA ligase or using template mediated ligation.

112. The method of any one of claims 109-111, wherein the nucleic acid library is a DNA library or a cDNA library.

113. The method of any one of claims 109-112, wherein the method further comprises amplifying the double-stranded member of the nucleic acid library lacking all, or a portion of, the sequence encoding the constant region of the ABM using a third primer and a fourth primer, wherein:
the third primer is substantially complementary to the first functional domain, and
the fourth primer is substantially complementary to the second functional domain.

114. A method for generating a recombinant antigen-binding molecule, the method comprising:
a) providing a tumor tissue sample comprising a plurality of immune cells;
b) determining all or a part of the nucleic acid sequences encoding one or more antigen-binding molecules expressed by the immune cells in the tumor tissue sample; and
c) using the determined nucleic acid sequences to produce a recombinant antigen-binding molecule.

115. The method of claim 92, wherein the plurality of immune cells comprises a T cell and wherein the one or more antigen-binding molecules produced by the immune cells comprises a TCR.

116. The method of claim 16, wherein the method comprises, following (a):
mounting the first substrate on a first member of a support device, the first member configured to retain the first substrate;
mounting the second substrate on a second member of the support device, the second member configured to retain the second substrate;
applying a reagent medium to the first substrate and/or the second substrate, the reagent medium comprising a permeabilization agent;
operating an alignment mechanism of the support device to move the first member and/or the second member such that a portion of the tissue sample comprising the ABM-expressing cell is aligned with a portion of the array of capture probes and within a threshold distance of the array of capture probes, and such that the portion of the tissue sample and the capture probe contact the reagent medium,
wherein the permeabilization agent releases the analyte from the ABM-expressing cell.

117. The method of claim 16, wherein the method further comprises aligning the first substrate with the second substrate, such that at least a portion of the tissue sample is aligned with at least a portion of the second substrate.

118. The method of claim 15 or 16, wherein the migrating comprises passive migration.

119. The method of claim 15 or 16, wherein the migrating comprises active migration, and optionally, wherein the active migration comprises electrophoresis.

120. The method of claim 17, wherein the analyte is RNA or DNA, optionally wherein the RNA is mRNA, or the DNA is cDNA or genomic DNA.

121. The method of anyone of claims 1-21 or 116-120, where the attaching in step (b) comprises hybridization.

122. The method of any one of claims 1-21 or 116-121, wherein the analyte encodes V and J sequences of an immune cell receptor, preferably a BCR or TCR.

123. The method of any one of claims 1-21 or 116-121, wherein the analyte encodes V, D, and J sequences of an immune cell receptor, preferably a BCR or TCR.

124. The method of any one of claims 1-21 or 116-121, wherein the tissue sample is a tissue section, and optionally, wherein the tissue section is a fixed tissue section or a fresh, frozen tissue section.

125. The method of claim 124, wherein the fixed tissue section is a formalin-fixed paraffin-embedded tissue section, a paraformaldehyde-fixed tissue section, a methanol-fixed tissue section, or an acetone-fixed tissue section.

126. The method of any one of claims 1-21 or 116-125, wherein the tissue sample is a human sample.
